(19) United States
(12) Patent Application Publication
(10) Pub. No.: US 2021/0379071 A1
(43) Pub. Date: Dec. 9, 2021

(54) INHIBITORS OF HUMAN IMMUNODEFICIENCY VIRUS REPLICATION

(71) Applicant: ViiV HEALTHCARE UK (NO.5) LIMITED, Brentford, Middlesex (GB)

(72) Inventors: Michael S. BOWSHER, Branford, CT (US); Eric P. GILLIS, Branford, CT (US); Christiana IWUAGWU, Branford, CT (US); B. Narasimhulu NAIDU, Branford, CT (US); Kyle E. PARCELLA, Branford, CT (US); Manoj PATEL, Branford, CT (US); Kevin M. PEESE, Branford, CT (US)

(21) Appl. No.: 17/288,085

(22) PCT Filed: Nov. 4, 2019
(86) PCT No.: PCT/IB2019/059450

§ 371 Date: Apr. 23, 2021

Related U.S. Application Data
(60) Provisional application No. 62/755,627, filed on Nov. 5, 2018.

(51) Int. Cl. A61K 31/517; A61K 45/06; C07D 403/14; C07D 471/04; A61P 31/18; A61K 31/519

(52) U.S. Cl. CPC A61K 31/517; A61K 45/06; A61K 31/519; C07D 471/04; A61P 31/18; C07D 403/14

(57) ABSTRACT

Compounds and pharmaceutically acceptable salts thereof, and compositions and methods for treating human immunodeficiency virus (HIV) infection are set forth.

### FIELD OF THE INVENTION

The invention relates to compounds, compositions, and methods for the treatment of human immunodeficiency virus (HIV) infection. More particularly, the invention provides novel inhibitors of HIV, pharmaceutical compositions containing such compounds, and methods for using these compounds in the treatment of HIV infection. The invention also relates to methods for making the compounds hereinafter described.

### BACKGROUND OF THE INVENTION

Acquired immunodeficiency syndrome (AIDS) is the result of infection by HIV. HIV continues to be a major global public health issue. In 2015, an estimated 36.7 million people were living with HIV (including 1.8 million children)—a global HIV prevalence of 0.8%. The vast majority of this number live in low- and middle-income countries. In the same year, 1.1 million people died of AIDS-related illnesses.

Current therapy for HIV-infected individuals consists of a combination of approved anti-retroviral agents. Close to four dozen drugs are currently approved for HIV infection, either as single agents, fixed dose combinations or single tablet regimens; the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIs), integrase strand transfer inhibitors (INSTIs), or entry inhibitors (one, maraviroc, targets the host CCR5 protein, while the other, enfuvirtide, is a peptide that targets the gp41 region of the viral gp160 protein). In addition, a pharmacokinetic enhancer (cobicistat or ritonavir) can be used in combinations with antiretroviral agents (ARVs) that require boosting.

Despite the armamentarium of agents and drug combinations, there remains a medical need for new anti-retroviral agents. High viral heterogeneity, drug-associated toxicity, tolerability problems, and poor adherence can all lead to treatment failure and may result in the selection of viruses with mutations that confer resistance to one or more antiretroviral agents or even multiple drugs from an entire class (Beyrer, C., Pozniak A. HIV drug resistance—an emerging threat to epidemic control. N. Engl. J. Med. 2017, 377, 1605-1607; Gupta, R. K., Gregson J., et al. HIV-1 drug resistance before initiation or re-initiation of first-line antiretroviral therapy in low-income and middle-income countries: a systematic review and meta-regression analysis. Lancet Infect. Dis. 2017, 18, 346-355; Zazzi, M., Hu, H., Prosperi, M. The global burden of HIV-1 drug resistance in the past 20 years. PeerJ. 2018, DOI 10.7717/peerj.4848). As a result, new drugs are needed that are easier to take, have high genetic barriers to the development of resistance and have improved safety over current agents. In this panoply of choices, novel mechanisms of action (MOAs) that can be used as part of the preferred antiretroviral therapy (ART) can still have a major role to play since they should be effective against viruses resistant to current agents.

Certain potentially therapeutic compounds have now been described in the art and set forth in Blair, Wade S. et. al. Antimicrobial Agents and Chemotherapy (2009), 53(12), 5080-5087, Blair, Wade S. et al. PLoS Pathogens (2010), 6(12), e1001220, Thenin-Houssier, Suzie; Valente, Susana T. Current HIV Research, 2016, 14, 270-282, and PCT Patent applications with the following numbers: WO 2012065062, WO 2013006738, WO 2013006792, WO 2014110296, WO 2014110297, WO 2014110298, WO 2014134566, WO 2015130964, WO2015130966, WO 2016033243, WO2018035359, WO2018203235, WO 2019161017, and WO 2019161280.

What is now needed in the art are additional compounds which are novel and useful in the treatment of HIV. Additionally, these compounds should provide advantages for pharmaceutical uses, for example, with regard to one or more of their mechanisms of action, binding, inhibition efficacy, target selectivity, solubility, safety profiles, bioavailability or reduced frequency of dosing. Also needed are new formulations and methods of treatment which utilize these compounds.

### SUMMARY OF THE INVENTION

Briefly, in one aspect, the present invention discloses a compound or salt selected from the group consisting of

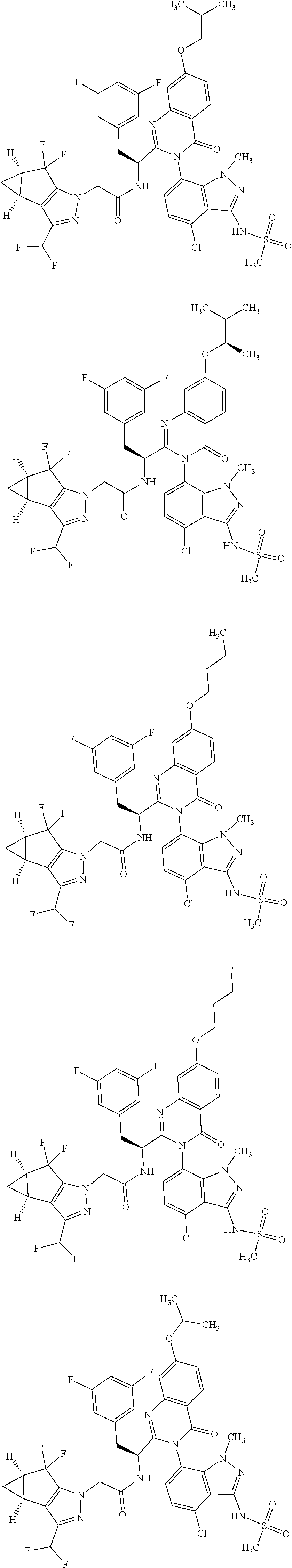

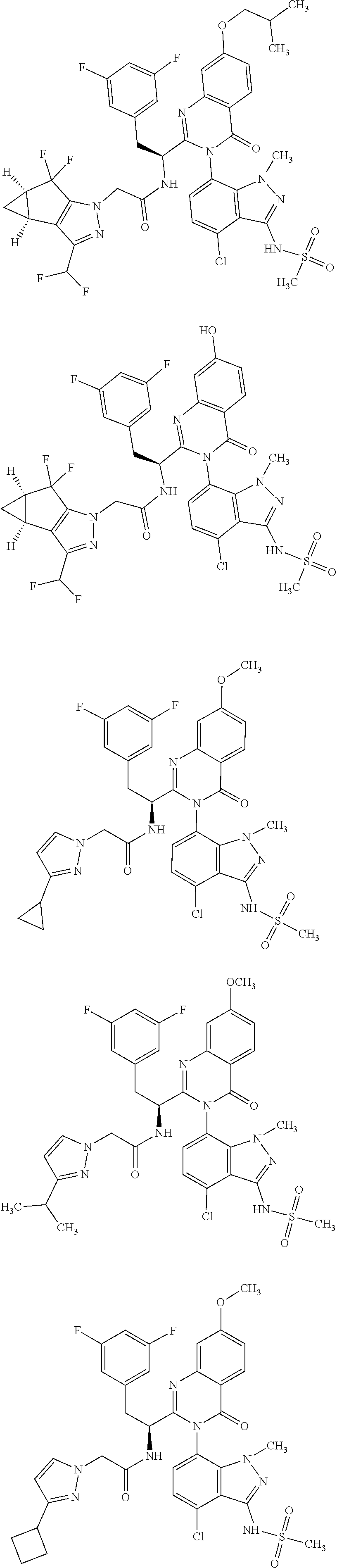

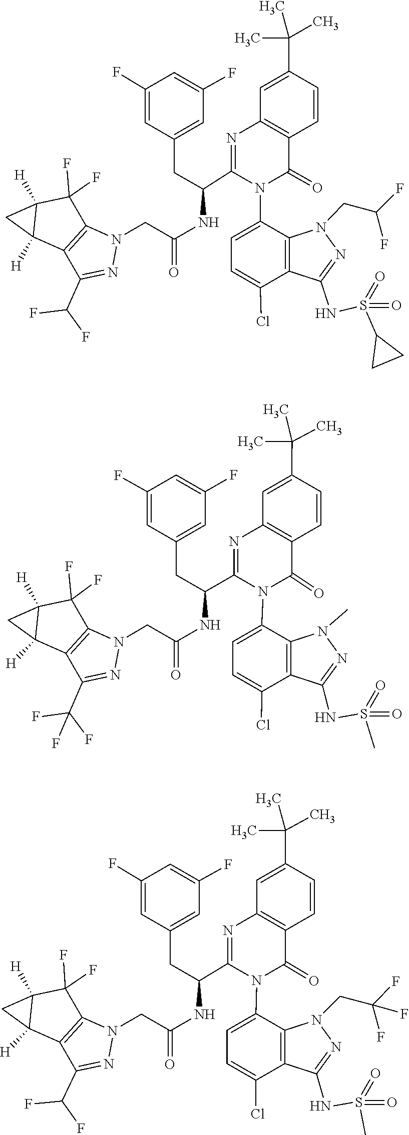 [SMILES: CC(C)(C)c1ccc2c(=O)n(-c3ccc(Cl)c4c(NS(=O)(=O)C5CC5)nn(CC(F)F)c34)c([C@H](Cc3cc(F)cc(F)c3)NC(=O)Cn3nc(C(F)F)c4c3C(F)(F)[C@@H]3C[C@H]43)nc2c1 ; Cn1nc(NS(C)(=O)=O)c2c(Cl)ccc(-n3c([C@H](Cc4cc(F)cc(F)c4)NC(=O)Cn4nc(C(F)(F)F)c5c4C(F)(F)[C@@H]4C[C@H]54)nc4cc(C(C)(C)C)ccc4c3=O)c21 ; CC(C)(C)c1ccc2c(=O)n(-c3ccc(Cl)c4c(NS(C)(=O)=O)nn(CC(F)(F)F)c34)c([C@H](Cc3cc(F)cc(F)c3)NC(=O)Cn3nc(C(F)F)c4c3C(F)(F)[C@@H]3C[C@H]43)nc2c1]

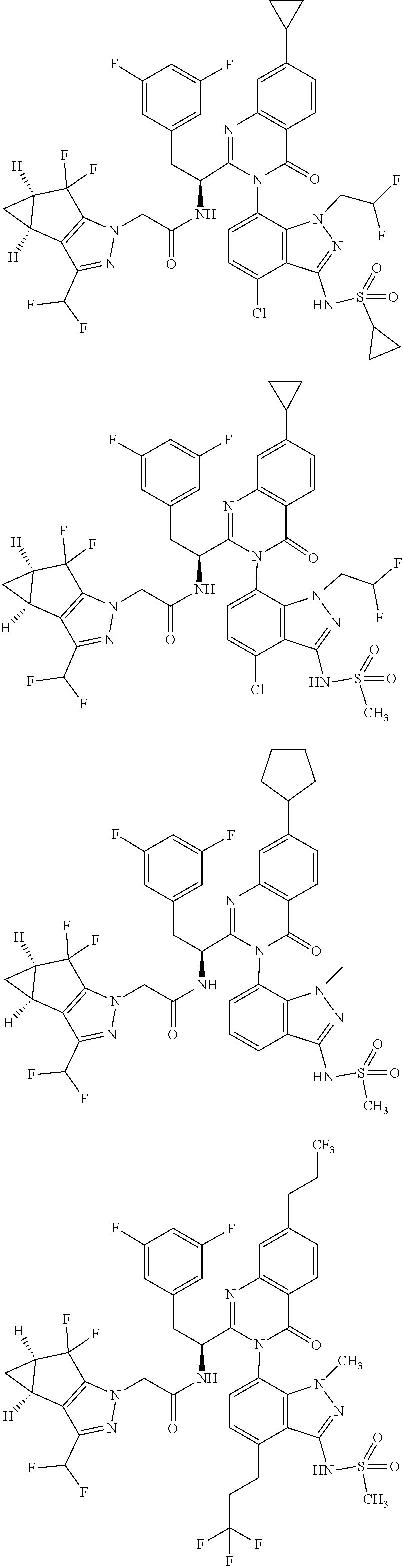

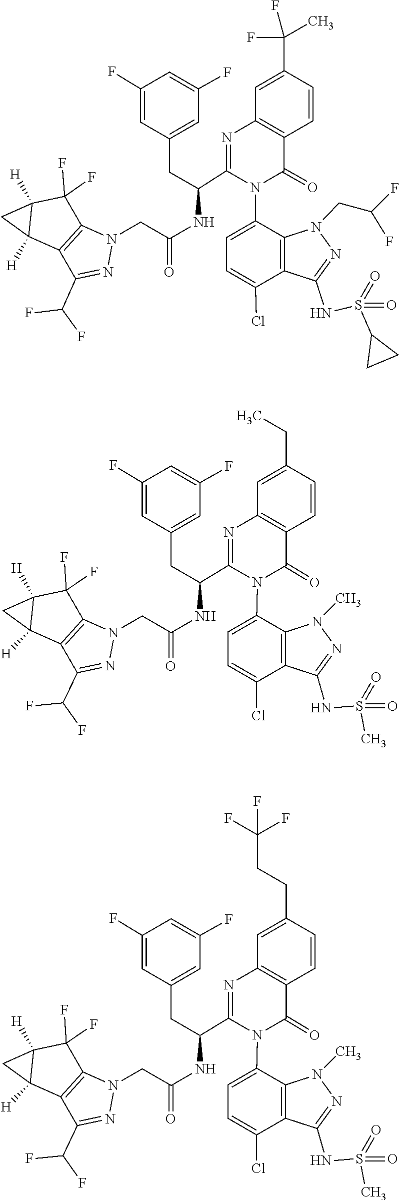 [SMILES: CC(F)(F)c1ccc2c(=O)n(-c3ccc(Cl)c4c(NS(=O)(=O)C5CC5)nn(CC(F)F)c34)c([C@H](Cc3cc(F)cc(F)c3)NC(=O)Cn3nc(C(F)F)c4c3C(F)(F)[C@@H]3C[C@H]43)nc2c1 ; CCc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1 ; Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(CCC(F)(F)F)ccc4c3=O)c21]

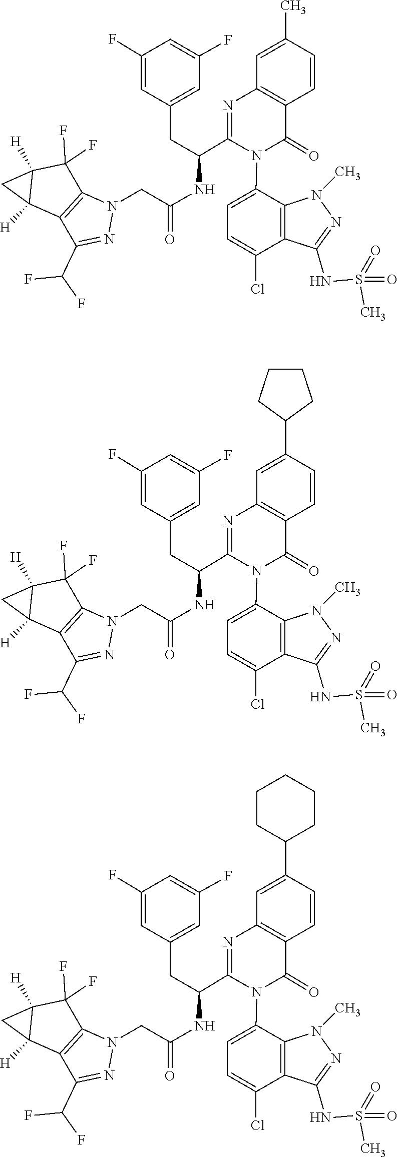 [SMILES: Cc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1 ; Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(C5CCCC5)ccc4c3=O)c21 ; Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(C5CCCCC5)ccc4c3=O)c21]

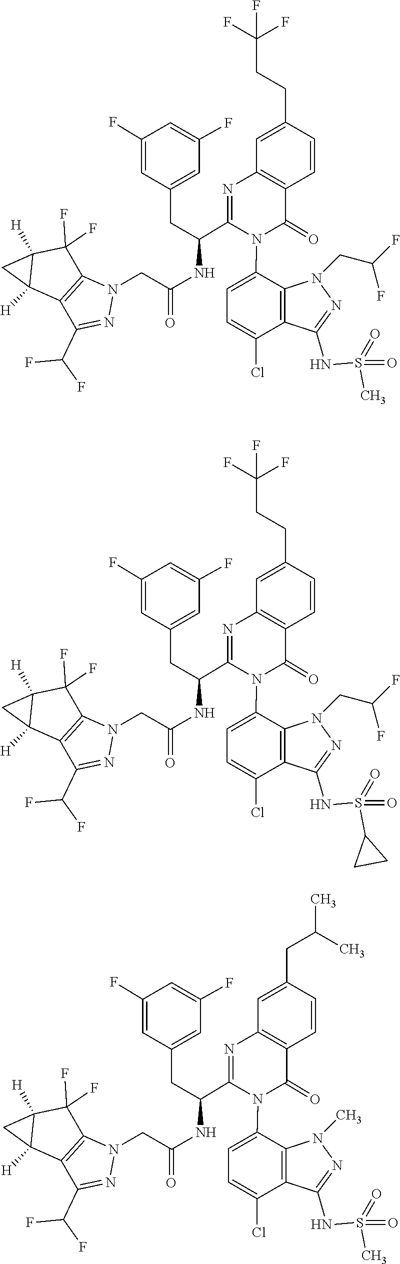 [SMILES: CS(=O)(=O)Nc1nn(CC(F)F)c2c(-n3c([C@H](Cc4cc(F)cc(F)c4)NC(=O)Cn4nc(C(F)F)c5c4C(F)(F)[C@@H]4C[C@H]54)nc4cc(CCC(F)(F)F)ccc4c3=O)ccc(Cl)c12 ; O=C(Cn1nc(C(F)F)c2c1C(F)(F)[C@@H]1C[C@H]21)N[C@@H](Cc1cc(F)cc(F)c1)c1nc2cc(CCC(F)(F)F)ccc2c(=O)n1-c1ccc(Cl)c2c(NS(=O)(=O)C3CC3)nn(CC(F)F)c12 ; CC(C)Cc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

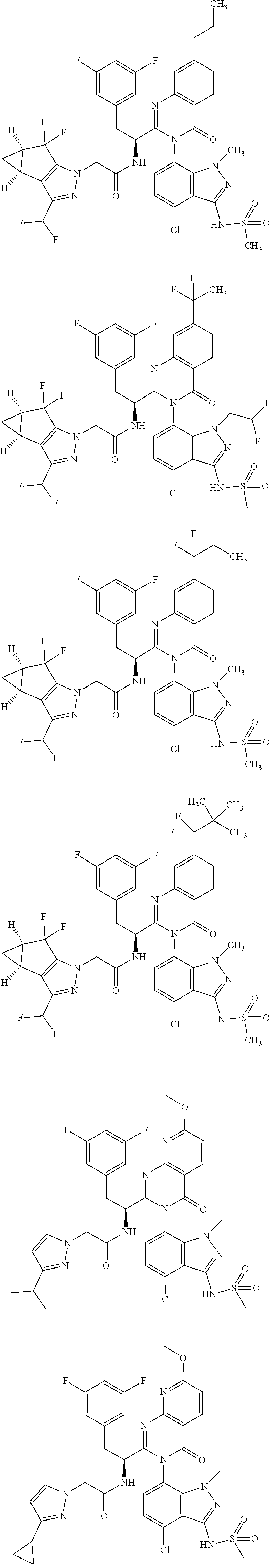

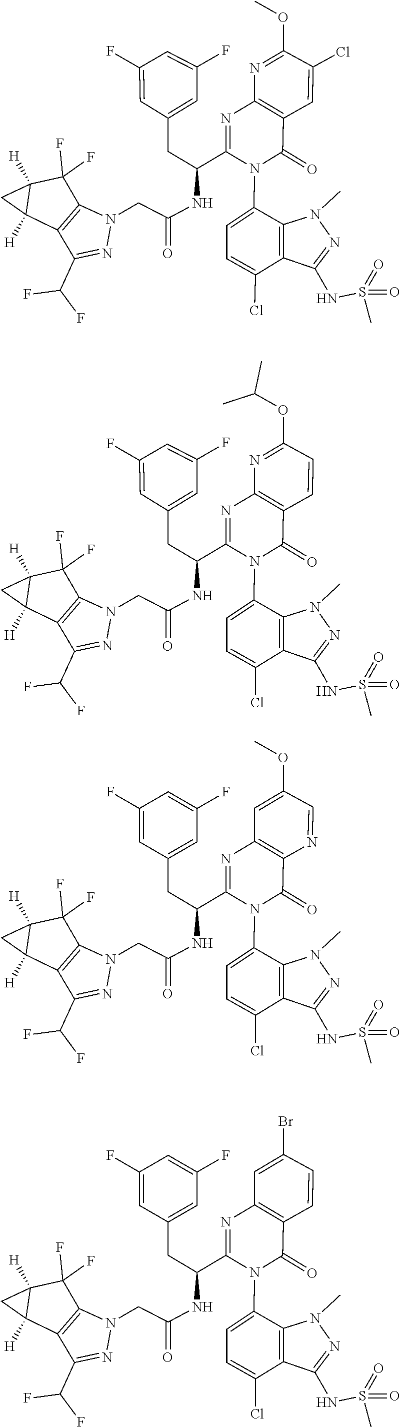

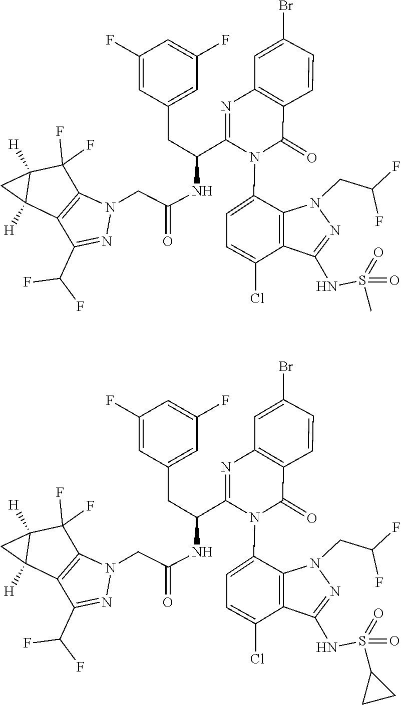 [SMILES: CS(=O)(=O)Nc1nn(CC(F)F)c2c(-n3c([C@H](Cc4cc(F)cc(F)c4)NC(=O)Cn4nc(C(F)F)c5c4C(F)(F)[C@@H]4C[C@H]54)nc4cc(Br)ccc4c3=O)ccc(Cl)c12 ; O=C(Cn1nc(C(F)F)c2c1C(F)(F)[C@@H]1C[C@H]21)N[C@@H](Cc1cc(F)cc(F)c1)c1nc2cc(Br)ccc2c(=O)n1-c1ccc(Cl)c2c(NS(=O)(=O)C3CC3)nn(CC(F)F)c12]

and pharmaceutically acceptable salts thereof.

In another aspect, the present invention discloses a composition comprising a compound or salt of this invention.

In another aspect, the present invention discloses a method of treating HIV infection comprising administering a composition comprising a compound or salt of this invention to a patient.

In another aspect, the present invention discloses a compound or salt of this invention for use in therapy.

In another aspect, the present invention discloses a compound or salt of this invention for use in treating HIV infection.

In another aspect, the present invention discloses the use of a compound or salt of this invention in the manufacture of a medicament for the treatment of HIV infection.

### DETAILED DESCRIPTION OF THE INVENTION

In one embodiment, the present invention discloses compounds and salts selected from the group consisting of

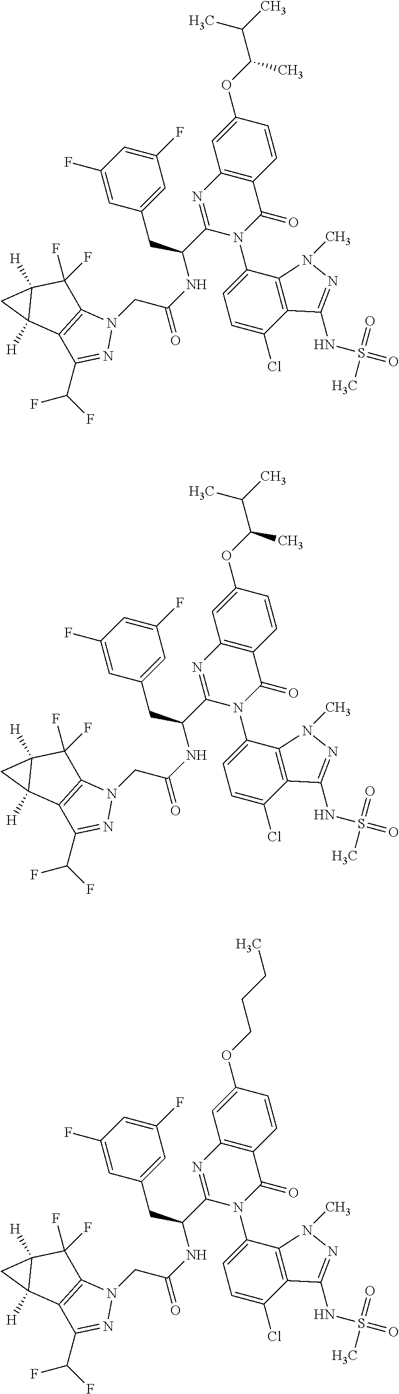 [SMILES: CC(C)[C@H](C)Oc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1 ; CC(C)[C@@H](C)Oc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1 ; CCCCOc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

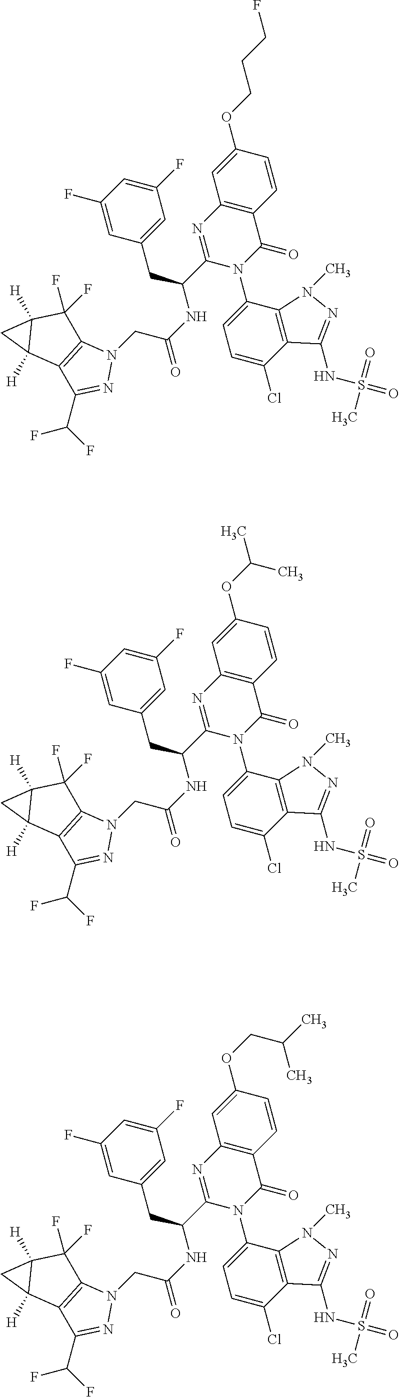 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(OCCCF)ccc4c3=O)c21 ; CC(C)Oc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1 ; CC(C)COc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

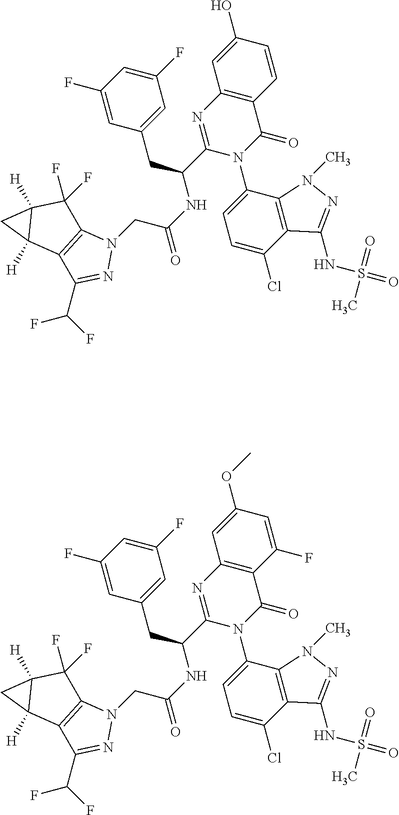 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(O)ccc4c3=O)c21 ; COc1cc(F)c2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

and pharmaceutically acceptable salts thereof.

In one embodiment, the present invention discloses compounds and salts selected from the group consisting of

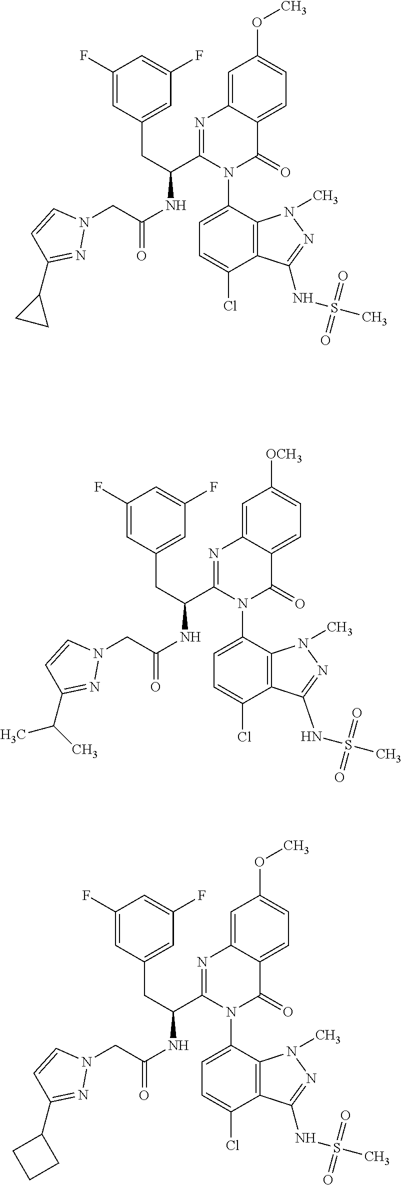

and pharmaceutically acceptable salts thereof.

In one embodiment, the present invention discloses compounds and salts selected from the group consisting of

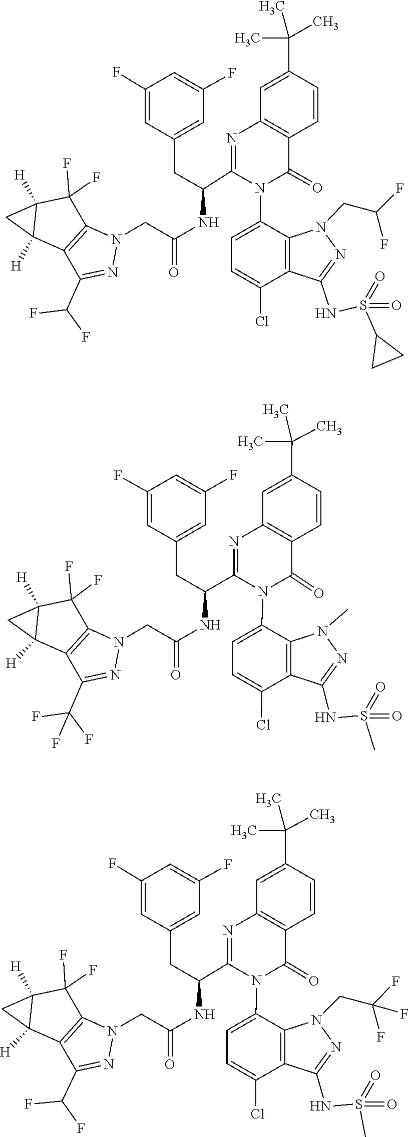 [SMILES: CC(C)(C)c1ccc2c(=O)n(-c3ccc(Cl)c4c(NS(=O)(=O)C5CC5)nn(CC(F)F)c34)c([C@H](Cc3cc(F)cc(F)c3)NC(=O)Cn3nc(C(F)F)c4c3C(F)(F)[C@@H]3C[C@H]43)nc2c1 ; Cn1nc(NS(C)(=O)=O)c2c(Cl)ccc(-n3c([C@H](Cc4cc(F)cc(F)c4)NC(=O)Cn4nc(C(F)(F)F)c5c4C(F)(F)[C@@H]4C[C@H]54)nc4cc(C(C)(C)C)ccc4c3=O)c21 ; CC(C)(C)c1ccc2c(=O)n(-c3ccc(Cl)c4c(NS(C)(=O)=O)nn(CC(F)(F)F)c34)c([C@H](Cc3cc(F)cc(F)c3)NC(=O)Cn3nc(C(F)F)c4c3C(F)(F)[C@@H]3C[C@H]43)nc2c1]

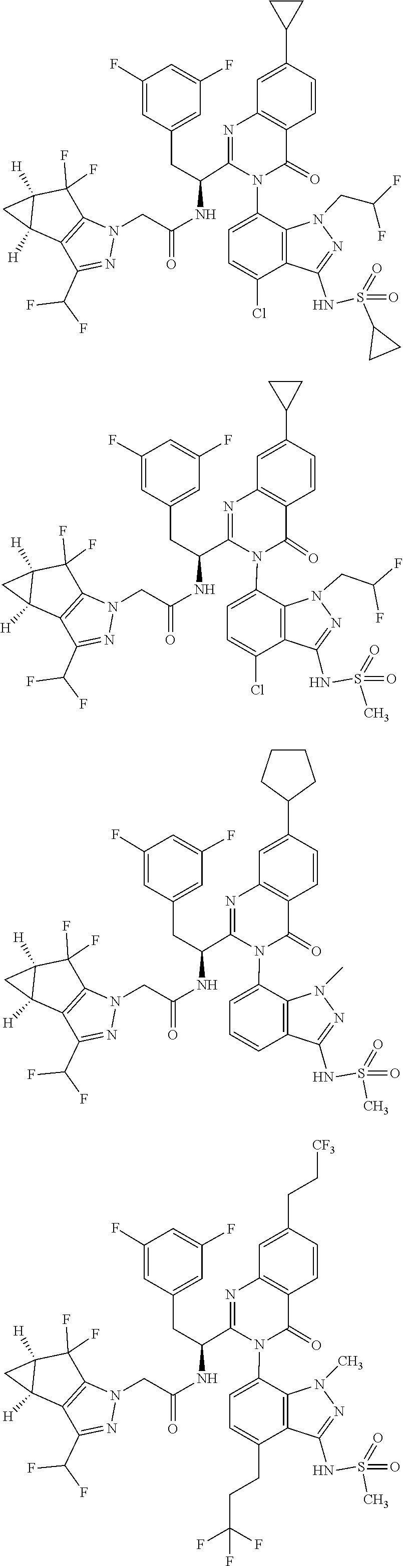

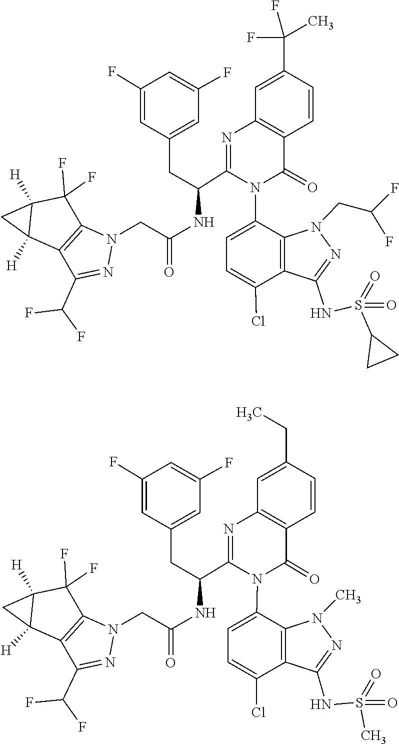 [SMILES: CC(F)(F)c1ccc2c(=O)n(-c3ccc(Cl)c4c(NS(=O)(=O)C5CC5)nn(CC(F)F)c34)c([C@H](Cc3cc(F)cc(F)c3)NC(=O)Cn3nc(C(F)F)c4c3C(F)(F)[C@@H]3C[C@H]43)nc2c1 ; CCc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

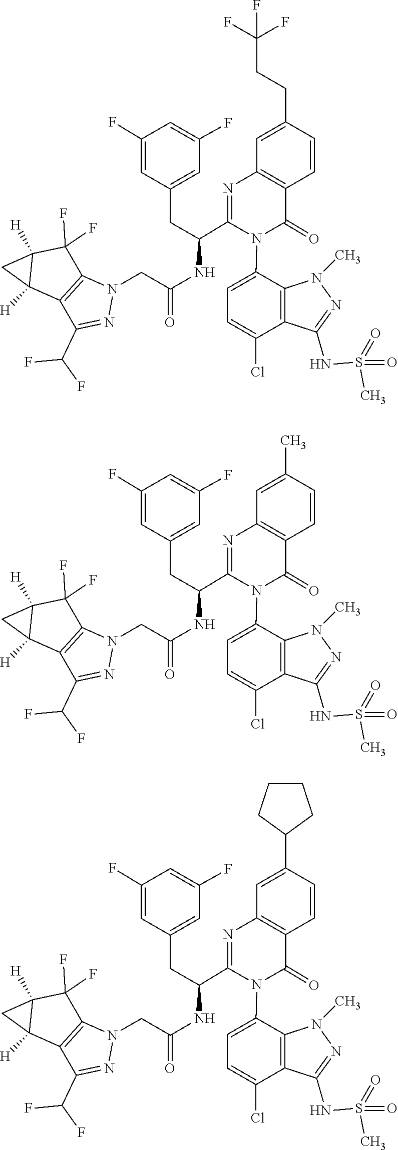 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(CCC(F)(F)F)ccc4c3=O)c21 ; Cc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1 ; Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(C5CCCC5)ccc4c3=O)c21]

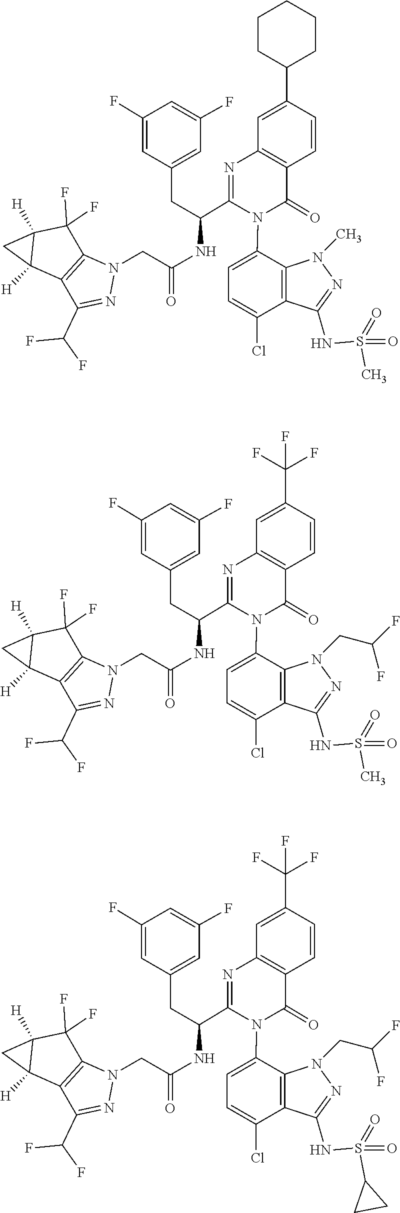 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(C5CCCCC5)ccc4c3=O)c21 ; CS(=O)(=O)Nc1nn(CC(F)F)c2c(-n3c([C@H](Cc4cc(F)cc(F)c4)NC(=O)Cn4nc(C(F)F)c5c4C(F)(F)[C@@H]4C[C@H]54)nc4cc(C(F)(F)F)ccc4c3=O)ccc(Cl)c12 ; O=C(Cn1nc(C(F)F)c2c1C(F)(F)[C@@H]1C[C@H]21)N[C@@H](Cc1cc(F)cc(F)c1)c1nc2cc(C(F)(F)F)ccc2c(=O)n1-c1ccc(Cl)c2c(NS(=O)(=O)C3CC3)nn(CC(F)F)c12]

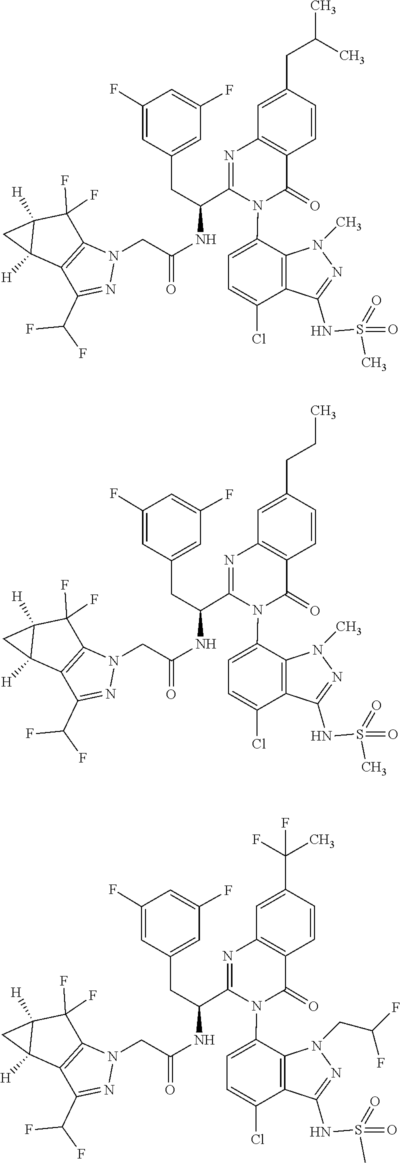 [SMILES: CC(C)Cc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1 ; CCCc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1 ; CC(F)(F)c1ccc2c(=O)n(-c3ccc(Cl)c4c(NS(C)(=O)=O)nn(CC(F)F)c34)c([C@H](Cc3cc(F)cc(F)c3)NC(=O)Cn3nc(C(F)F)c4c3C(F)(F)[C@@H]3C[C@H]43)nc2c1]

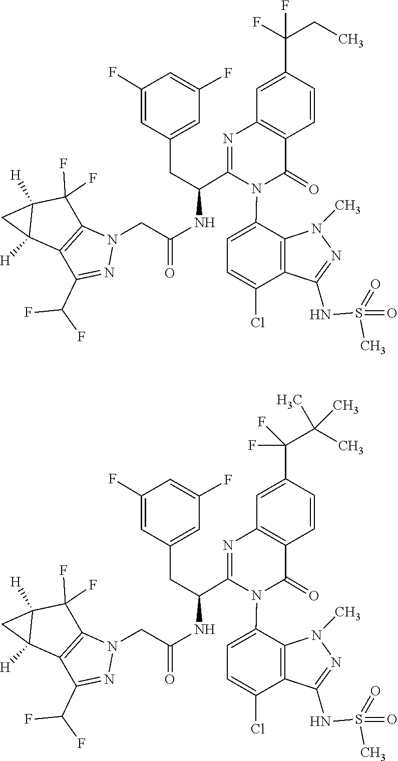 [SMILES: CCC(F)(F)c1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1 ; Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(C(F)(F)C(C)(C)C)ccc4c3=O)c21]

and pharmaceutically acceptable salts thereof.

In one embodiment, the present invention discloses compounds and salts selected from the group consisting of

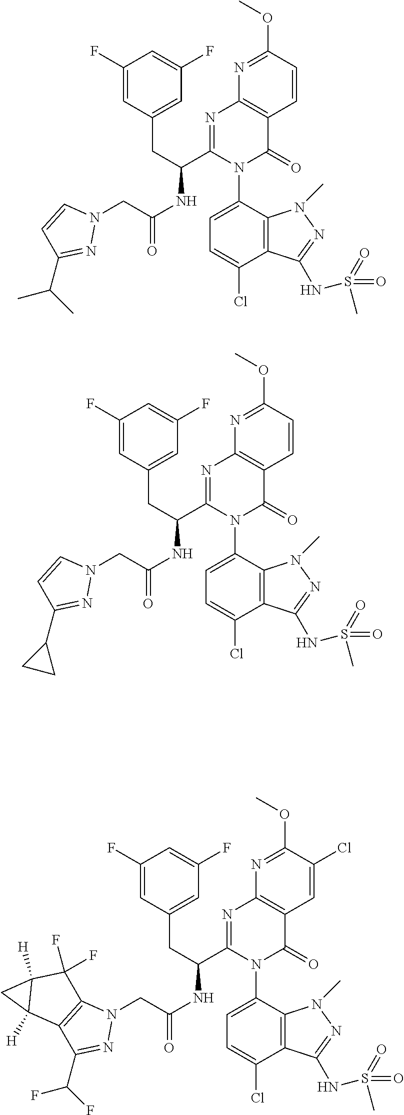 [SMILES: COc1ccc2c(=O)n(-c3ccc(Cl)c4c(NS(C)(=O)=O)nn(C)c34)c([C@H](Cc3cc(F)cc(F)c3)NC(=O)Cn3ccc(C(C)C)n3)nc2n1 ; COc1ccc2c(=O)n(-c3ccc(Cl)c4c(NS(C)(=O)=O)nn(C)c34)c([C@H](Cc3cc(F)cc(F)c3)NC(=O)Cn3ccc(C4CC4)n3)nc2n1 ; COc1nc2nc([C@H](Cc3cc(F)cc(F)c3)NC(=O)Cn3nc(C(F)F)c4c3C(F)(F)[C@@H]3C[C@H]43)n(-c3ccc(Cl)c4c(NS(C)(=O)=O)nn(C)c34)c(=O)c2cc1Cl]

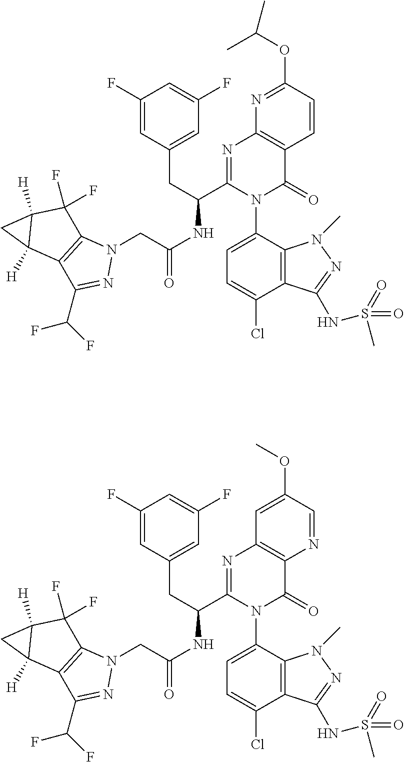 [SMILES: CC(C)Oc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2n1 ; COc1cnc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

and pharmaceutically acceptable salts thereof.

In one embodiment, the present invention discloses compounds and salts selected from the group consisting of

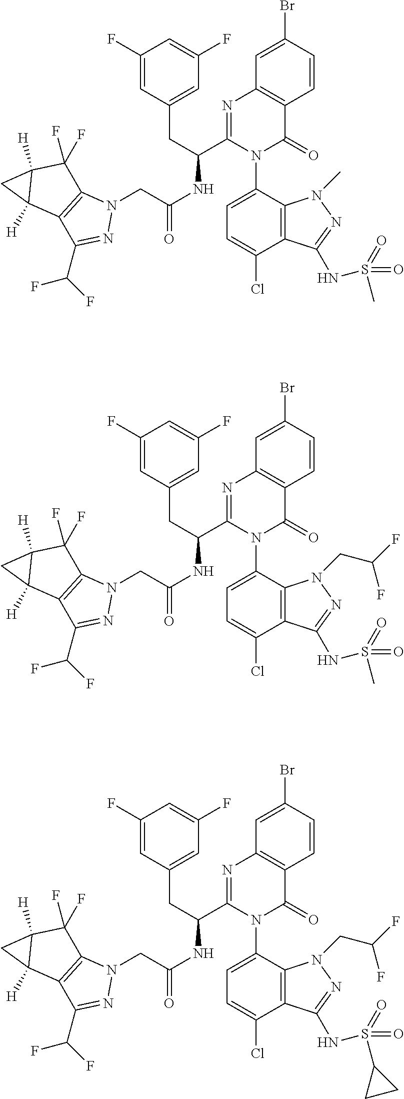 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(Br)ccc4c3=O)c21 ; CS(=O)(=O)Nc1nn(CC(F)F)c2c(-n3c([C@H](Cc4cc(F)cc(F)c4)NC(=O)Cn4nc(C(F)F)c5c4C(F)(F)[C@@H]4C[C@H]54)nc4cc(Br)ccc4c3=O)ccc(Cl)c12 ; O=C(Cn1nc(C(F)F)c2c1C(F)(F)[C@@H]1C[C@H]21)N[C@@H](Cc1cc(F)cc(F)c1)c1nc2cc(Br)ccc2c(=O)n1-c1ccc(Cl)c2c(NS(=O)(=O)C3CC3)nn(CC(F)F)c12]

and pharmaceutically acceptable salts thereof.

In one aspect, the present invention discloses compounds, and pharmaceutically acceptable salts thereof, wherein the stereochemistry of the indazole bond is as depicted below:

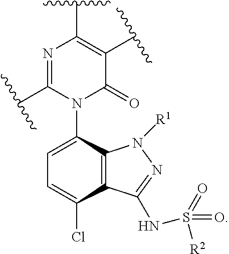

In one embodiment, the present invention discloses compounds and salts selected from the group consisting of

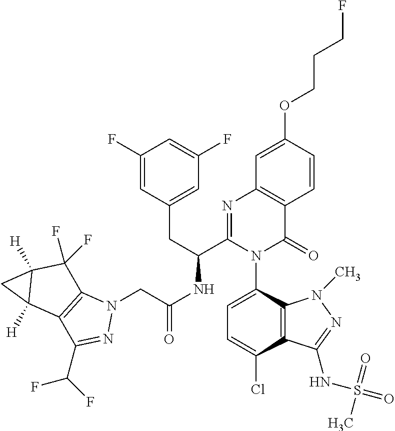

and pharmaceutically acceptable salts thereof.

In one embodiment, the present invention discloses compounds and salts selected from the group consisting of

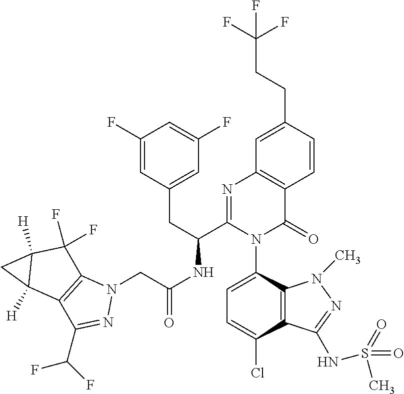

and pharmaceutically acceptable salts thereof.

In one embodiment, the present invention discloses compounds and salts selected from the group consisting of

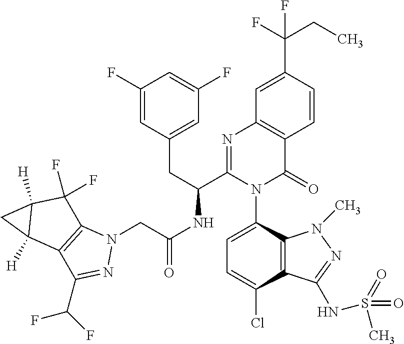

and pharmaceutically acceptable salts thereof.

In one embodiment, the present invention discloses compounds and salts selected from the group consisting of

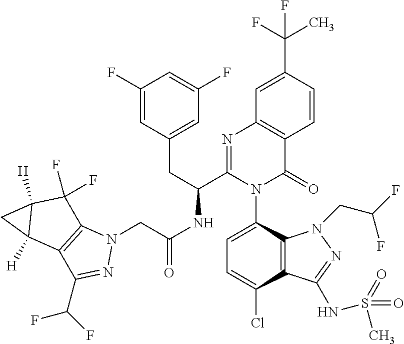

and pharmaceutically acceptable salts thereof.

In one embodiment, the present invention discloses compounds and salts selected from the group consisting of

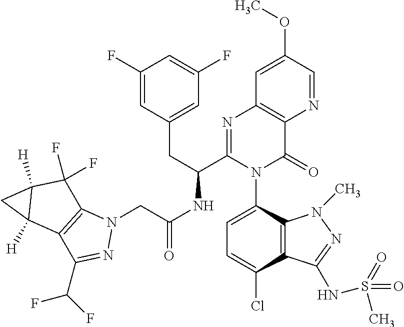

and pharmaceutically acceptable salts thereof.

In one embodiment, the present invention discloses compounds and salts selected from the group consisting of

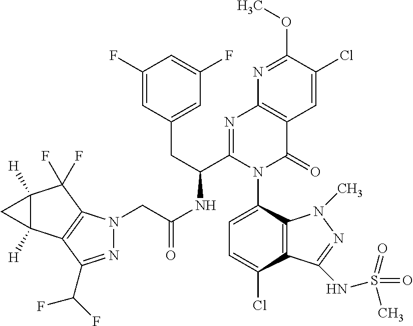

and pharmaceutically acceptable salts thereof.

In one embodiment, the present invention discloses compounds and salts selected from the group consisting of

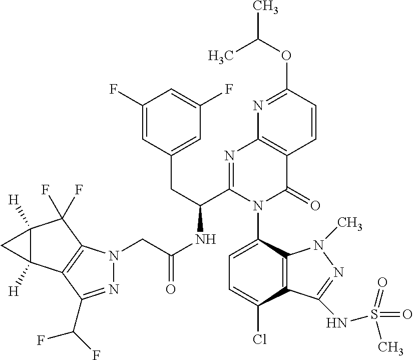

and pharmaceutically acceptable salts thereof.

The salts of compounds of this invention are pharmaceutically acceptable. Such salts may be acid addition salts or base addition salts. For a review of suitable pharmaceutically acceptable salts see Berge et al, J. Pharm, Sci., 66, 1-19, 1977. In an embodiment, acid addition salts are selected from the hydrochloride, hydrobromide, hydroiodide, sulphate, bisulfate, nitrate, phosphate, hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate. In an embodiment, base addition salts include metal salts (such as sodium, potassium, aluminium, calcium, magnesium and zinc) and ammonium salts (such as isopropylamine, diethylamine, diethanolamine salts). Other salts (such as trifluoroacetates and oxalates) may be used in the manufacture of compounds of this invention and their pharmaceutically acceptable salts and are included within the scope of the invention. All possible stoichiometric and non-stoichiometric forms of the salts of compounds of this invention are included within the scope of the invention. Acid and base addition salts may be prepared by the skilled chemist, by treating a compound of this invention with the appropriate acid or base in a suitable solvent, followed by crystallisation and filtration.

Some of the compounds of the invention exist in stereoisomeric forms. The invention includes all stereoisomeric forms of the compounds including enantiomers and diastereomers including atropisomers. The term homochiral is used as a descriptor, per accepted convention, to describe a structure which is a single stereoisomer. Absolute stereochemistry was not assigned in all cases. Thus the compound is drawn at the chiral center as unspecified but labelled as homochiral and in the procedures it is identified by its properties such as for example first eluting off a normal or chiral column per the conventions of chemists. It should be noted that the provided experimental procedures teach how to make the exact compound even if not drawn with absolute configuration. Methods of making and separating stereoisomers are known in the art. The invention includes all tautomeric forms of the compounds. The invention includes atropisomers and rotational isomers.

In the method of this invention, preferred routes of administration are oral and by injection to deliver subcutaneously. Therefore, preferred pharmaceutical compositions include composition suitable for oral administration (for example tablets) and formulations suitable for injection.

The compounds and salts of this invention are believed to act as Capsid Inhibitors.

The compounds and salts of the present invention may be employed alone or in combination with other therapeutic agents. The compounds and salts of the present invention and any other pharmaceutically active agent(s) may be administered together or separately and, when administered separately, administration may occur simultaneously or sequentially, in any order. The amounts of the compounds of the present invention and the other pharmaceutically active agent(s) and the relative timings of administration will be selected in order to achieve the desired combined therapeutic effect. The administration in combination of a compound and salts of the present invention may be in combination by administration concomitantly in: (1) a unitary pharmaceutical composition including multiple active agents; or (2) separate pharmaceutical compositions each including one of the active agents. Alternatively, the combination may be administered separately in a sequential manner wherein one treatment agent is administered first and the other second or vice versa, and the different agents could be administered on different schedules if appropriate. Such sequential administration may be close in time or remote in time. The amounts of the compound or salt of this invention and the other pharmaceutically active agent(s) and the relative timings of administration will be selected in order to achieve the desired combined therapeutic effect.

As such, the compounds and salts of the present invention may be used in combination with one or more agents useful in the prevention or treatment of HIV.

### EXAMPLES

As used below, “vac/fill×3” indicates that the reaction vessel is placed under high vacuum using a Schlenk line and then the vacuum source is exchanged for an argon source to fill the evacuated reaction vessel with argon to atmospheric pressure; the process is repeated three times. When solvent is present in the reaction vessel the vacuum is maintained only to the point that the solvent mildly boils for approximately 5-10 seconds, then the vacuum source is exchanged for the argon source.

### General Procedure A:

To an appropriately sized glass vial (typically a 5 mL microwave vial) equipped with a stir bar was added N—((S)-1-(7-bromo-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1 equiv, typically 50-100 mg), K₃PO₄ (3 equiv), and dichloro[9,9-dimethyl-4,5-bis(diphenylphosphino)xanthene]palladium(II) (0.05-0.1 equiv). To the vial was added the indicated boronic acid or pinacol boronate (3 equiv). The vial sealed with a septum cap and then placed under argon atmosphere (vac/fill×3). To the vial was added THF:water (4:1) in a volume necessary to achieve a concentration of 0.05M in bromide. The mixture was again placed under argon atmosphere (vac/fill×3). The reaction was stirred at either ambient temperature or 60° C. overnight (˜18 h). Upon cooling to ambient temperature, the reaction mixture was concentrated, and the residue was subjected to HPLC purification to afford the indicated product.

### General Procedure E:

To an appropriately sized glass vial (typically a 5 mL microwave vial) equipped with a stir bar was added N—((S)-1-(7-bromo-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1 equiv, typically 50-100 mg), the indicated trifluoroborate (2.5 equiv), cesium carbonate (3 equiv), and RuPhos Pd G3 (0.2 equiv). To the vial was added toluene:water (10:1) in a volume necessary to achieve a concentration of 0.05M in bromide. The vial sealed with a septum cap and then placed under argon atmosphere (vac/fill×3). The mixture was stirred at 100° C. for 2 h. Upon cooling to ambient temperature, the reaction mixture was concentrated, and the resulting residue was subjected to HPLC purification to afford the indicated product. The structure of “RuPhos Pd G3” (CAS: 1445085-77-7) is:

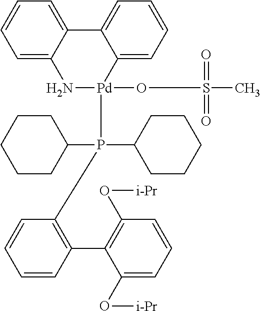

### General Procedure L:

The indicated alkene (1 equiv, typically 50 mg) was dissolved in methanol:acetic acid (1:1) to a concentration of 0.05M. The solution was degassed using argon. To the solution was added Pd—C (0.5 equiv) (10% Degussa). The reaction vessel was evacuated and then refilled with H₂ (g) introduced via a balloon. The reaction was stirred for 3-5 hr at room temperature under a balloon-pressure atmosphere of H₂ (g). The atmosphere was then replaced with Ar (g), then Celite was added to the reaction mixture and the slurry was filtered through a pad of Celite washing with DCM. The filtrate was concentrated, and the resulting residue was subjected to HPLC purification to afford the indicated product.

### General Procedure N:

General Procedure N follows the method of General Procedure E exactly except N—((S)-1-(7-bromo-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide is used as the bromide.

### General Procedure O:

General Procedure O follows the method of General Procedure E exactly except N—((S)-1-(7-bromo-3-(4-chloro-3-(cyclopropanesulfonamido)-1-(2,2-difluoroethyl)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide is used as the bromide.

### Mitsunobu General Procedure:

In the below procedure, N—((S)-1-(3-(4-chloro-3-(N-(4-methoxybenzyl)methylsulfonamido)-1-methyl-1H-indazol-7-yl)-7-hydroxy-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (“acetamide”) is the limiting reagent and all equivalents are in reference to this amount. To a stirred solution of N—((S)-1-(3-(4-chloro-3-(N-(4-methoxybenzyl)methylsulfonamido)-1-methyl-1H-indazol-7-yl)-7-hydroxy-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1 equiv, typically 25 mg), the indicated alcohol (3 equiv), and triphenylphosphine (3 equiv) in THF (volume necessary to achieve a 0.11M concentration in acetamide) was added a solution of diisopropyl (E)-diazene-1,2-dicarboxylate (“DIAD”, 3 equiv) in THF (16.1 mg DIAD/0.25 mL THF, 5.1 M). The total reaction volume was approximately 0.50 mL when conducted using 25 mg of acetamide. The solution was stirred at room temperature either overnight (˜18 h), 72 h (time not optimized), or until the reaction was deemed complete by LCMS. The volatiles were evaporated under a N₂ (g) stream and the resulting residue was then placed under high vacuum for 10 minutes. The crude residue was taken up in DCM:TFA (1:1) to achieve a concentration of 0.11M based on the acetamide input used above. To the solution was added triflic acid (3 equiv) and resulting solution was stirred at room temperature for 10 minutes. The reaction volatiles were evaporated under reduced pressure. The residue was taken up in EtOAc and washed with aq. K₃PO₄ (0.75 M). The organic layer was isolated and then concentrated under a stream of N₂ (g). The resulting residue was subjected to HPLC purification to afford the indicated product.

### LCMS Method A:

Wavelength1: 220 nm, Wavelength2: 254 nm, Injection Vol.: 5.00 μl, Stop Time: 4.00, Grad. Time: 3.0, Start % B: 0, End % B: 100, Total Flow: 0.80 ml/min, Solvent A: 95:5 Water:MeCN 0.1% TFA, Solvent B: 5:95 Water:MeCN 0.1% TFA, Column: Acquity UPLC BEH C18, 2.1×50 mm, 1.7 μm particles.

### LCMS Method C:

Column: Acquity UPLC BEH C18, 2.1×30 mm, 1.7 μm particles; Solvent A=0.1% Formic acid in 100% Water. Solvent B=0.1% Formic Acid in 100% Acetonitrile. Flow Rate=0.8 mL/min. Start % B=5. Final % B=95. Gradient Time=1.6 min, then a 0.25 min hold at 95% B. Wavelength=215 nm.

### LCMS Method F:

Column: Acquity BEH C18, 2.1×30 mm, 1.7 μm particles; Solvent A=0.1% Formic acid in 100% Water. Solvent B=0.1% Formic Acid in 100% Acetonitrile. Flow Rate=0.8 mL/min. Start % B=5. Final % B=95. Gradient Time=1.7 min, then a 0.2 min hold at 95% B. Wavelength=215 and 254 nm.

### HPLC Purification:

HPLC purification was performed using one of the conditions indicated below, optionally followed by a second HPLC purification using a different condition indicated below. Based on analytical HPLC data obtained on the crude reaction mixture, the purification condition was optimized for each target compound by modifying the initial Solvent A:Solvent B ratio, the gradient time, the final Solvent A:Solvent B ratio, and the hold time at the final Solvent A:Solvent B concentration.

HPLC Condition A: Column: Zorbax Eclipse Plus C18, 21.2×100 mm, 5 μm particles; Solvent A=0.1% Formic Acid in 100% Water. Solvent B=Acetonitrile. Flow Rate=40 mL/min. Wavelength=215 and 254 nm. ESI+Range: 150 to 1500 dalton.

HPLC Condition B: Column: Sunfire prep C18 OBD, 30×100 mm, 5 μm particles; Solvent A: water:MeCN 95:5 w/ 0.1% TFA, Solvent B: MeCN:water 95:5 w/ 0.1% TFA. Flow Rate=42 mL/min. Wavelength=220 and 254 nm.

HPLC Condition C: Column: Waters Xterra C18, 19×100 mm, 10 μm particles; Solvent A=0.1% NH4OH in 100% Water. Solvent B=Acetonitrile. Flow Rate=40 mL/min. Wavelength=215 and 254 nm. ESI+Range: 150 to 1500 dalton.

### 3-bromo-6-chloro-2-fluorobenzaldehyde

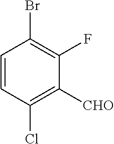 [SMILES: O=Cc1c(Cl)ccc(Br)c1F]

To a stirred solution of 1-bromo-4-chloro-2-fluorobenzene (200 g, 0.955 mol, 1.0 equiv.) in anhydrous THF (2.0 L) was added a solution of LDA in THF (2.0 M, 620 mL, 1.24 mol, 1.3 equiv.) at −50° C. The reaction mixture was allowed to warm to −20° C. and was stirred for 1 h. The mixture was cooled to −50° C. and slowly to the mixture was added DMF (184.8 mL, 2.48 mol, 2.6 equiv.) maintaining a temperature of −50° C. The mixture was allowed to warm to 0° C. and was stirred for 30-45 min at the same temperature (0° C.). The mixture was quenched via the slow addition of ice cold water (2.0 L). The reaction mixture was diluted with ethyl acetate (2.0 L) and stirred for 15 min at room temperature. The organic layer was separated and reserved; the aqueous layer was extracted with ethyl acetate (2×1.0 L). The combined organic layers were washed with water (2×1.0 L); 1.0 N HCl (1.0 L) and then 15% NaCl solution (2.0 L). The organic solution was dried over Na₂SO₄; filtered; and then concentrated in vacuo. The resultant crude solid was used directly in the next step without further purification. Yield for the crude product: 210.0 g (93%).

### 3-bromo-6-chloro-2-fluorobenzonitrile

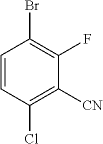 [SMILES: N#Cc1c(Cl)ccc(Br)c1F]

To a stirred solution of 3-bromo-6-chloro-2-fluorobenzaldehyde (210.0 g, 0.89 mol, 1.0 equiv.) in water (2.1 L) at room temperature was added hydroxylamine-O-sulfonic acid (175.15 g, 1.55 mol, 1.75 equiv.). The reaction mixture was heated to 50° C. and stirred for 18 h). The mixture was cooled to room temperature and stirred for 1-1.5 h. The solids were isolated via filtration and were then washed with water. The wet solid was dried under vacuum at 50° C. for 12-15 h to afford 3-bromo-6-chloro-2-fluorobenzaldehyde, 190.0 g (91%).

### 7-bromo-4-chloro-1H-indazol-3-amine

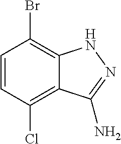 [SMILES: Nc1n[nH]c2c(Br)ccc(Cl)c12]

To a 3 L three neck round bottom flask fitted with a water-cooled condenser, a thermometer and a mechanical stirrer was added 3-bromo-6-chloro-2-fluorobenzonitrile (100 g, 427 mmol) and ethanol (500 mL). To the solution was added hydrazine hydrate (104 ml, 2133 mmol) at room temperature. The solution was heated to 80° C. and was maintained at that temperature for 1 h upon which the mixture became a homogeneous solution and LCMS analysis indicated the reaction was complete. The solution was allowed to cool to 45° C. and then water (1 L) was added slowly to produce a white ppt. as a thick slurry. Following the addition the mixture was stirred for 30 minutes. The solids were isolated via filtration. The solids were washed with water (1 L) and then dried under vacuum at 45° C. to afford 7-bromo-4-chloro-1H-indazol-3-amine as a pale orange solid, 103 g (98%). ¹H NMR (400 MHz, DMSO-d₆): δ 12.21 (bs, 1H), 7.41 (d, J=7.8 Hz, 1H), 6.84 (d, J=7.8 Hz, 1H), 5.34 (bs, 2H) ppm.

### 7-bromo-4-chloro-1-methyl-1H-indazol-3-amine

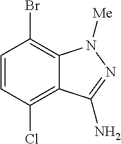 [SMILES: Cn1nc(N)c2c(Cl)ccc(Br)c21]

To a solution of 3-bromo-6-chloro-2-fluorobenzonitrile (360.0 g, 1.55 mol, 1.0 equiv.) in ethanol (1.08 L) was added methylhydrazine sulphate (1.11 kg, 7.73 mol, 5.0 equiv.) followed by the addition of triethylamine (1.3 L, 9.3 mol, 6.0 equiv.) at 25-35° C. The reaction mixture was heated to 110° C. and maintained at that temperature for 15 h. The mixture was cooled to room temperature and to the mixture was added water (3.0 L). The mixture was stirred at room temperature for 1 h. The solids were isolated via filtration and were washed with water. The wet solid was dried under vacuum at 50° C. for 12-15 hours. The material was subjected to silica gel column chromatography (hexanes:EtOAc 90:10→60:40) to afford 7-bromo-4-chloro-1-methyl-1H-indazol-3-amine as a pale yellow solid, 185.0 g (46%).

### 7-bromo-4-chloro-1-(2,2-difluoroethyl)-1H-indazol-3-amine

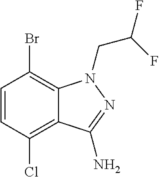 [SMILES: Nc1nn(CC(F)F)c2c(Br)ccc(Cl)c12]

To a stirred solution of 7-bromo-4-chloro-1H-indazol-3-amine (128.0 g, 0.52 mol, 1.0 equiv.) in dry THF (1.92 L) at 0° C. was added ^{t}BuOK (76 g, 0.67 mol, 1.3 equiv.) in portions. The reaction mixture was stirred for 10 min at 0° C.; then to the solution was slowly added 2,2-difluoroethyl trifluoro-methanesulfonate (122.5 g, 0.57 mol, 1.1 equiv.) at 0° C. The mixture was slowly warmed to room temperature and then was stirred for 2 h. The mixture was diluted with ice-cold water (3.0 L) and MTBE (2×1.5 L). The organic layer was separated, washed with water (2×1.2 L), dried over Na₂SO₄, filtered, and then concentrated in vacuo. The resulting crude material was subjected to silica gel chromatography (hexanes:EtOAc 95:5490:10). Product-containing fractions contaminated with the undesired regioisomer were concentrated and then triturated with DCM (5 mL/g) to afford the pure desired product which was then combined with fractions of the pure material. This process afforded 7-bromo-4-chloro-1-(2,2-difluoroethyl)-1H-indazol-3-amine as a light yellow solid, 110 g (68%). ¹H NMR (DMSO-d₆, 500 MHz) δ 7.55 (d, 1H, J=7.9 Hz), 6.96 (d, 1H, J=7.9 Hz), 6.1-6.5 (m, 1H), 5.62 (s, 2H), 4.94 (dt, 2H, J=3.8, 14.1 Hz).

### 7-bromo-4-chloro-1-(2,2,2-trifluoroethyl)-1H-indazol-3-amine

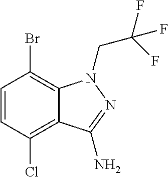 [SMILES: Nc1nn(CC(F)(F)F)c2c(Br)ccc(Cl)c12]

To a stirred solution of 7-bromo-4-chloro-1H-indazol-3-amine (70 g, 284 mmol, 1.0 equiv.) in dry DMF (700 mL) at room temperature was added in portions Cs₂CO₃ (184 g, 568 mmol, 2 equiv.). The reaction mixture was stirred for 10 min at room temperature. To the reaction mixture was added slowly at room temperature 2,2,2-trifluoroethyl trifluoromethanesulfonate (72.5 g, 312 mmol, 1.10 equiv.). After completion of the reaction (monitored by TLC), the mixture was diluted with ice cold water (700 mL) upon which a precipitate was formed. The mixture was allowed to warm to room temperature and then was stirred for 30 minutes at room temperature. The solids were isolated via filtration and then were washed with water (500 mL). The wet product was dissolved in DMF (350 mL) and then was diluted with water (350 mL) at room temperature. The mass was stirred for 30 min., then the solids were collected via filtration and were washed with water (200 mL) followed by hexanes (700 mL). The wet solids were dried under vacuum at 50-55° C. for 18-20 h to afford 7-bromo-4-chloro-1-(2,2,2-trifluoroethyl)-1H-indazol-3-amine (4) as a light yellow solid, 64.0 g (69%).

### N-(7-bromo-4-chloro-1-methyl-1H-indazol-3-yl)methanesulfonamide

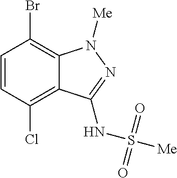 [SMILES: Cn1nc(NS(C)(=O)=O)c2c(Cl)ccc(Br)c21]

To a solution of 7-bromo-4-chloro-1-methyl-1H-indazol-3-amine (90 g, 0.34 mol, 1.0 equiv.) in CH₂Cl₂ (900 mL) was added diisopropylethylamine (“DIPEA”, 180.4 mL, 1.04 mol, 3.0 equiv.) and 4-dimethylaminopyridine (“DMAP”, 2.07 g, 0.017 mol, 0.05 equiv.). The mixture was stirred for 5 min, then was cooled to 0° C. and methanesulfonyl chloride (67.7 mL, 0.87 mol, 2.5 equiv.) was added resulting in a noted exotherm. The reaction mixture was warmed to room temperature and stirred at that temperature 3 h upon which a precipitate formed. The mixture was diluted with dichloromethane (1.0 L) and then was washed with water (2.0 L) followed by aq. HCl (1.0M, 1.0 L), and then brine (1.5 L). The organic solution was dried over Na₂SO₄; filtered, and then concentrated in vacuo. The crude residue was dissolved in EtOH (1.8 L). To the solution was added aq. NaOH (20%, 650 mL) at room temperature upon which a slight exotherm was noted. The resulting mixture was stirred for 2 h upon which the mixture became a homogeneous solution. The solution was diluted with water (2.0 L) and the pH was adjusted to pH 2-3 using aq. HCl (1.0M, app. 3.0 L). The precipitate that was formed was collected by filtration. The solids were washed with water and then dried in vacuo to afford N-(7-bromo-4-chloro-1-methyl-1H-indazol-3-yl)methanesulfonamide as an off-white solid, 96 g (82%). ¹H NMR (500 MHz, CDCl₃) δ 7.48 (d, J=7.9 Hz, 1H), 7.24 (br s, 1H), 6.95 (d, J=7.9 Hz, 1H), 4.38 (s, 3H), 3.42 (s, 3H). LC/MS (M+H)⁺=337.80.

### N-(7-bromo-4-chloro-1-(2,2-difluoroethyl)-1H-indazol-3-yl)methanesulfonamide

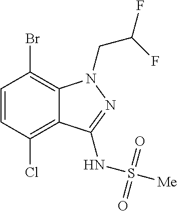 [SMILES: CS(=O)(=O)Nc1nn(CC(F)F)c2c(Br)ccc(Cl)c12]

To a stirred solution of 7-bromo-4-chloro-1-(2,2-difluoroethyl)-1H-indazol-3-amine (40.0 g, 0.12 mol, 1.0 equiv.) in dry DCM (400 mL) was added DIPEA (67 mL, 0.38 mol, 3.0 equiv.) and DMAP (0.78 g, 0.0064 mol, 0.05 equiv.). The solution was stirred for 5 min, then the reaction mixture was cooled to 0° C. and to the mixture was slowly added methanesulfonyl chloride (31.0 mL, 0.38 mol, 3.0 equiv.). The reaction mixture was allowed to warm to room temperature and was then stirred for 2 h. After completion of the reaction (monitored by TLC), the mixture was diluted with DCM (2×2.5 L) and water (2.0 L). The organic layer was separated and was washed with water (2×1.5 L); brine (1.5 L); dried over Na₂SO₄; filtered; and was concentrated in vacuo. The residue was dissolved in ethanol (320 mL) and to the solution was aq. NaOH (20% w/w, 320 mL). The reaction mixture was stirred at room temperature for 2 h. After completion of the reaction (monitored by TLC), the mixture was diluted with water (1.0 L) and acidified to pH 2-3 using aq. HCl (1.0 M). The resulting solids were collected via filtration. The solids were triturated with hexanes:EtOAc (95:5, 10 V) and again isolated via filtration. The wet solids were dried under vacuum at 50° C. to afford N-(7-bromo-4-chloro-1-(2,2-difluoroethyl)-1H-indazol-3-yl)methane sulfonamide (5) as a light yellow solid, 45.7 g (91%). ¹H NMR (400 MHz, CDCl₃): δ 7.52 (d, J=8.0 Hz, 1H), 7.41 (bs, 1H), 7.00 (d, J=8.0 Hz, 1H), 6.16 (tt, J₁=4.3 Hz, J₂=8.6 Hz, J₃=55.4 Hz, 1H), 5.15 (td, J₁=4.3 Hz, J₂=12.7 Hz, 2H), 3.41 (s, 3H).

### N-(7-bromo-4-chloro-1-(2,2-difluoroethyl)-1H-indazol-3-yl)cyclopropanesulfonamide

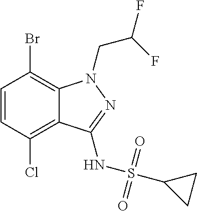 [SMILES: O=S(=O)(Nc1nn(CC(F)F)c2c(Br)ccc(Cl)c12)C1CC1]

To a stirred solution of 7-bromo-4-chloro-1-(2,2-difluoroethyl)-1H-indazol-3-amine (10 g, 0.032 mol, 1.0 equiv.) in dry pyridine (100 mL) was added cyclopropylsulfonyl chloride (18.1 g, 0.128 mol, 4.0 equiv.). The reaction mixture was stirred at room temperature for 48 h. The mixture was diluted with water (400 mL) and extracted with MTBE (2×100 mL). The combined organic layers were washed with water (3×300 mL), brine (300 mL), dried over Na₂SO₄, filtered and concentrated in vacuo. The resulting crude material was triturated with hexanes (15 V) to obtain N-(7-Bromo-4-chloro-1-(2,2-difluoroethyl)-1H-indazol-3-yl)cyclopropanesulfonamide as a light-red solid, 11.1 g (82%).

### N-(7-bromo-4-chloro-1-(2,2,2-trifluoroethyl)-1H-indazol-3-yl)methanesulfonamide

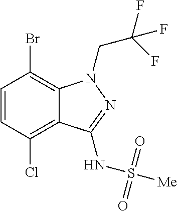 [SMILES: CS(=O)(=O)Nc1nn(CC(F)(F)F)c2c(Br)ccc(Cl)c12]

To a stirred solution of 7-bromo-4-chloro-1-(2,2,2-trifluoroethyl)-1H-indazol-3-amine (60 g, 182.64 mmol, 1.0 equiv.) in dry DCM (600 mL, 10 V) was added DIPEA (94.8 ml, 547.92 mmol, 3.0 equiv.) and DMAP (1.11 g, 9.13 mmol, 0.05 equiv.). After being stirring for 15 min the solution was cooled to 0° C. To the solution was slowly added methanesulfonyl chloride (52.3 g, 456.6 mmol, 3.0 equiv.). The reaction mixture was then allowed to warm to room temperature and was stirred at room temperature for 2 h. The progress of the reaction (bis-mesylation) was monitored by TLC. After the reaction was determined to be complete the mixture was diluted with DCM (200 mL) and water (200 mL). The organic layer was isolated and washed with water (500 mL), brine (300 mL), dried over Na₂SO₄, filtered and concentrated in vacuo. The resulting residue was dissolved in ethanol (600 mL) and to the solution was aq. NaOH (20% w/w, 600 mL). The reaction mixture was stirred for 2 h at room temperature. After completion of the reaction (mono demesylation, monitored by TLC) the solution was diluted with water (300 mL) and acidified to pH 2-3 using aq. HCl (1.0 M). The resulting solids were isolated via filtration and were then washed with water. The solids were dried under vacuum at 50-55° C. The solid material was further purified by trituration using hexanes:EtOAc (95:5, 15V) to afford N-(7-Bromo-4-chloro-1-(2,2,2-trifluoroethyl)-1H-indazol-3-yl)methanesulfonamide as a light yellow solid, 55.1 g (75%).

### N-(7-bromo-4-chloro-1-methyl-1H-indazol-3-yl)-N-(4-methoxybenzyl)methanesulfonamide

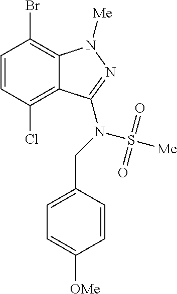 [SMILES: COc1ccc(CN(c2nn(C)c3c(Br)ccc(Cl)c23)S(C)(=O)=O)cc1]

To a mixture of N-(7-bromo-4-chloro-1-methyl-1H-indazol-3-yl)methanesulfonamide (49 g, 0.144 mol, 1.0 equiv.) in DMF (980 mL) was added 1-(chloromethyl)-4-methoxybenzene (23.54 mL, 0.17 mol, 1.2 equiv.). To the mixture was added cesium carbonate (61.3 g, 0.18 mol, 1.3 equiv.). The mixture was heated to 80° C. and maintained at that temperature for 2 h. After completion of the reaction (monitored by TLC) the mixture was poured into water (2.0 L). The mixture was extracted with EtOAc (2×1.5 L). The combined organic layers were washed with brine (1.0 L); dried over Na₂SO₄; filtered and then concentrated in vacuo. The residue was crystallised from hexanes:EtOAc (9:1, 120 mL) to afford the desired product N-(7-Bromo-4-chloro-1-methyl-1H-indazol-3-yl)-N-(4-methoxybenzyl) methane sulfonamide as a white solid. Yield: 62 g (94%). ¹H NMR (500 MHz, CDCl₃) δ 7.44 (d, J=7.9 Hz, 1H), 7.31 (d, J=8.5 Hz, 2H), 6.99 (d, J=7.9 Hz, 1H), 6.84 (d, J=8.5 Hz, 2H), 4.99 (br s, 1H), 4.76 (br s, 1H), 4.40 (s, 3H), 3.80 (s, 3H), 3.01 (s, 3H).

### N-(7-bromo-4-chloro-1-(2,2-difluoroethyl)-1H-indazol-3-yl)-N-(4-methoxybenzyl)methanesulfonamide

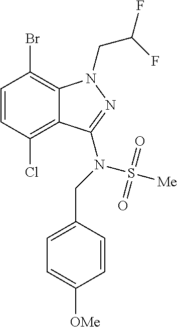 [SMILES: COc1ccc(CN(c2nn(CC(F)F)c3c(Br)ccc(Cl)c23)S(C)(=O)=O)cc1]

To a stirred solution of N-(7-bromo-4-chloro-1-(2,2-difluoroethyl)-1H-indazol-3-yl)methanesulfonamide (45.7 g, 0.117 mol, 1.0 equiv.) and 1-(chloromethyl)-4-methoxybenzene (22.1 g, 0.141 mol, 1.2 equiv.) in DMF (460 mL, 10 V) was added cesium carbonate (49.8 g, 0.152 mol, 1.3 equiv.). The reaction mixture was heated to 80° C. and stirred for 2 h at the same temperature. After completion of the reaction (monitored by TLC), the mixture was cooled to room temperature and then poured into water (2.0 L). The mixture was extracted with EtOAc (2×1.5 L). The combined organic layers were washed with brine (1.0 L), dried over Na₂SO₄, filtered and concentrated in vacuo. The resulting crude material was subjected to silica gel column purification (eluting with hexanes:EtOAc 85:15→75:25) to afford N-(7-bromo-4-chloro-1-(2,2-difluoroethyl)-1H-indazol-3-yl)-N-(4-methoxy benzyl)methanesulfonamide as a light yellow solid, 56 g (93%).

### N-(7-bromo-4-chloro-1-(2,2-difluoroethyl)-1H-indazol-3-yl)-N-(4-methoxybenzyl)cyclopropanesulfonamide

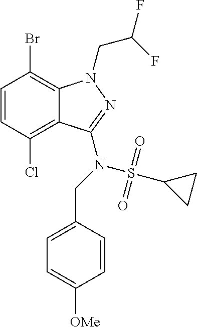 [SMILES: COc1ccc(CN(c2nn(CC(F)F)c3c(Br)ccc(Cl)c23)S(=O)(=O)C2CC2)cc1]

To a stirred mixture of N-(7-bromo-4-chloro-1-(2,2-difluoroethyl)-1H-indazol-3-yl)cyclo-propanesulfonamide (15 g, 0.036 mol, 1.0 equiv.) and 1-(chloromethyl)-4-methoxybenzene (6.79 g, 0.043 mol, 1.2 equiv.) in DMF (150 mL) was added cesium carbonate (15.32 g, 0.047 mol, 1.3 equiv.). The reaction mixture was heated to 80° C. and stirred at that temperature for 2 h. After completion of the reaction (monitored by TLC), the mixture was poured into water (300 mL) and the product was extracted with MTBE (2×200 mL). The combined organic layers were washed with brine (300 mL), dried over Na₂SO₄, filtered and concentrated in vacuo. The resulting crude material was subjected to silica gel column purification (hexanes:EtOAc 80:20→75:25) to afford N-(7-Bromo-4-chloro-1-(2,2-difluoroethyl)-1H-indazol-3-yl)-N-(4-methoxybenzyl)methanesulfonamide as a gummy liquid, 16.5 g (86%).

### N-(7-bromo-4-chloro-1-(2,2,2-trifluoroethyl)-1H-indazol-3-yl)-N-(4-methoxybenzyl)methanesulfonamide

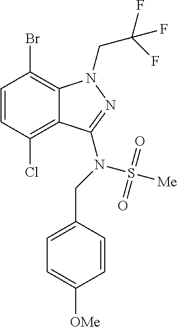 [SMILES: COc1ccc(CN(c2nn(CC(F)(F)F)c3c(Br)ccc(Cl)c23)S(C)(=O)=O)cc1]

To a stirred solution of N-(7-Bromo-4-chloro-1-(2,2,2-trifluoroethyl)-1H-indazol-3-yl)methanesulfonamide (6.0 g, 14.77 mmol, 1.0 equiv.) in dry DMF (60 mL, 10 V) at room temperature was added in portions Cs₂CO₃ (6.25 g, 19.20 mmol, 1.3 equiv.). The mixture was stirred for 10 min at room temperature, then to the mixture was slowly added 1-(chloromethyl)-4-methoxybenzene (2.77 g, 17.724 mmol, 1.2 equiv.). The reaction mixture was heated to 80° C. and maintained at that temperature for 2 h. After completion of the reaction (monitored by TLC), the mixture was cooled to room temperature and then was diluted with ice cold water (60 mL) and ethyl acetate (60 mL). The organic layer was isolated; washed with water (40 mL); dried over Na₂SO₄; filtered and concentrated in vacuo. The resulting crude material was triturated using hexanes:EtOAc (97:3, 15V) to afford N-(7-bromo-4-chloro-1-(2,2,2-trifluoroethyl)-1H-indazol-3-yl)-N-(4-methoxybenzyl)methanesulfonamide as a light yellow solid, 7.0 g (90%).

### N-(7-amino-4-chloro-1-methyl-1H-indazol-3-yl)-N-(4-methoxybenzyl)methanesulfonamide

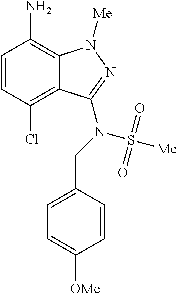 [SMILES: COc1ccc(CN(c2nn(C)c3c(N)ccc(Cl)c23)S(C)(=O)=O)cc1]

To a stirred solution of N-(7-Bromo-4-chloro-1-methyl-1H-indazol-3-yl)-N-(4-methoxybenzyl) methanesulfonamide (55 g, 0.12 mol, 1.0 equiv.) in NMP (900 mL) at room temperature was added copper (I) iodide (4.57 g, 0.024 mol, 0.2 equiv.), sodium ascorbate (47.4 g, 0.24 mol, 2 equiv.) and (1R, 2R)—N₁,N₂-dimethylcyclohexane-1,2-diamine (8.52 g, 0.06 mol, 0.5 equiv.) were added at room temperature. Then a solution of sodium azide (23.3 g, 0.36 mol, 3.0 equiv.) in water (182 mL). The mixture was heated to 100° C. and maintained at that temperature for 12 h. The reaction mixture was cooled to room temperature and diluted with ethyl acetate (1.5 L), then filtered through a pad of Celite. The filter pad was extracted with EtOAc (500 mL). The combined filtrate was diluted with water (2.0 L) and the organic layer was isolated and reserved. The aqueous phase was extracted with EtOAc (2×1.0 L). The combined organic layers were washed with water (1.0 L); brine (1.0 L); dried over Na₂SO₄; filtered; and concentrated in vacuo. The crude material was purified by silica column chromatography (hexanes:EtOAc 100:0→80:20) to afford the title compound, N-(7-Amino-4-chloro-1-methyl-1H-indazol-3-yl)-N-(4-methoxybenzyl) methanesulfonamide, as an off-white solid, 27.0 g (57%). ¹H NMR (400 MHz, CDCl₃) δ 7.33-7.29 (m, 2H), 6.89 (d, J=7.8 Hz, 1H), 6.85-6.79 (m, 2H), 6.48 (d, J=7.8 Hz, 1H), 5.11 (br.s, 1H), 4.81 (br.s, 1H), 4.30 (s, 3H), 3.80 (br s, 2H), 3.79 (s, 3H), 2.99 (s, 3H). LC/MS (M+H)⁺=395.00.

### N-(7-amino-4-chloro-1-(2,2-difluoroethyl)-1H-indazol-3-yl)-N-(4-methoxybenzyl)methanesulfonamide

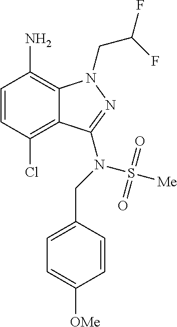 [SMILES: COc1ccc(CN(c2nn(CC(F)F)c3c(N)ccc(Cl)c23)S(C)(=O)=O)cc1]

To a stirred solution of N-(7-bromo-4-chloro-1-(2,2-difluoroethyl)-1H-indazol-3-yl)-N-(4-methoxybenzyl)methanesulfonamide (62 g, 0.12 mol, 1.0 equiv.) in NMP (745 mL) at room temperature was added copper (I) iodide (4.64 g, 0.024 mol, 0.2 equiv.), sodium ascorbate (48.3 g, 0.24 mol, 2 equiv.) and (1R, 2R)—N₁,N₂-dimethylcyclohexane-1,2-diamine (8.7 g, 0.06 mol, 0.5 equiv.). To the mixture was added a solution of sodium azide (23.8 g, 0.36 mol, 3.0 equiv.) in water (204 mL). The mixture was heated to 100° C. and maintained at that temperature for 15 h. The mixture was cooled to room temperature and was then diluted with ethyl acetate (1.5 L). The mixture was filtered through a pad of Celite and the filter pad was extracted with EtOAc (500 mL). The combined filtrate was diluted with water (2.0 L), organic layer was separated, and aqueous layer extracted with EtOAc (2×1.0 L). The combined organic layers were washed with water (1.2 L), brine (1.0 L), dried over Na₂SO₄, filtered and then concentrated in vacuo. The resulting residue was subjected to silica gel column chromatography (hexanes:EtOAc 100:0-475:25) to afford the title compound, N-(7-amino-4-chloro-1-(2,2-difluoroethyl)-1H-indazol-3-yl)-N-(4-methoxybenzyl)methanesulfonamide, as an off-white solid, 23.0 g, (44%).

### N-(7-amino-4-chloro-1-(2,2-difluoroethyl)-1H-indazol-3-yl)-N-(4-methoxybenzyl)cyclopropanesulfonamide

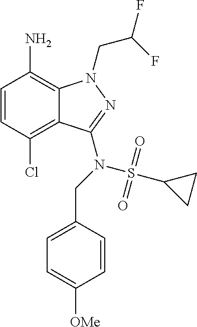 [SMILES: COc1ccc(CN(c2nn(CC(F)F)c3c(N)ccc(Cl)c23)S(=O)(=O)C2CC2)cc1]

To a stirred solution of N-(7-bromo-4-chloro-1-(2,2-difluoroethyl)-1H-indazol-3-yl)-N-(4-methoxybenzyl)cyclopropanesulfonamide (32 g, 0.059 mol, 1.0 equiv.) in NMP (512 mL) at room temperature was added copper (I) iodide (2.27 g, 0.012 mol, 0.2 equiv.), sodium ascorbate (23.7 g, 0.12 mol, 2 equiv.) and (1R, 2R)—N₁,N₂-dimethylcyclohexane-1,2-diamine (4.25 g, 0.03 mol, 0.5 equiv.). To the mixture was added a solution of sodium azide (11.6 g, 0.18 mol, 3.0 equiv.) in water (112 mL). The reaction was heated to 100° C. and stirred for 18 h the same temperature. The mixture was cooled to room temperature and diluted with ethyl acetate (1.2 L). The mixture was filtered through a pad of Celite, extracting with EtOAc (300 mL). The combined filtrate was poured into water (1.5 L) and the organic layer was isolated and reserved. The aqueous layer was extracted with EtOAc (2×0.8 L). The combined organic layers were washed with water (0.8 L), brine (0.8 L), dried over Na₂SO₄, filtered and then concentrated in vacuo. The crude residue was subjected to silica gel column chromatography (hexanes:EtOAc 100:0→80:20) to afford the title compound, N-(7-amino-4-chloro-1-(2,2-difluoroethyl)-1H-indazol-3-yl)-N-(4-methoxybenzyl)cyclopropanesulfonamide as an off-white solid, 14.2 g (50%).

### N-(7-amino-4-chloro-1-(2,2,2-trifluoroethyl)-1H-indazol-3-yl)-N-(4-methoxybenzyl)methanesulfonamide

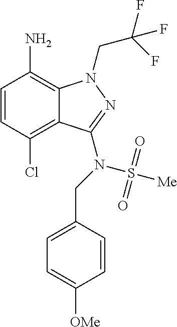 [SMILES: COc1ccc(CN(c2nn(CC(F)(F)F)c3c(N)ccc(Cl)c23)S(C)(=O)=O)cc1]

To a stirred solution of N-(7-bromo-4-chloro-1-(2,2,2-trifluoroethyl)-1H-indazol-3-yl)-N-(4-methoxybenzyl)methanesulfonamide (3 g, 5.69 mmol, 1.0 equiv.) in NMP (45 mL) was added at room temperature copper (I) iodide (0.22 g, 1.13 mmol, 0.2 equiv.), sodium ascorbate (2.25 g, 11.38 mmol, 2 equiv.) and (1R, 2R)—N₁,N₂-dimethylcyclohexane-1,2-diamine (0.4 g, 2.84 mmol, 0.5 equiv.). To the mixture was added a solution of sodium azide (1.1 g, 17.07 mmol) in water (15 mL). The mixture was heated to 100° C. and maintained at that temperature for 13 h. The reaction mixture was cooled to room temperature and was then diluted with ethyl acetate (50 mL). The mixture was filtered through a pad of Celite bed extracting with EtOAc (30 mL). The combined filtrate was poured into water (50 mL) and the organic layer was isolated and reserved. The aqueous phase was extracted with EtOAc (2×30 mL). The combined organics were washed with water (50 mL), brine (40 mL), dried over Na₂SO₄, filtered and concentrated in vacuo. The resulting residue was subjected to silica gel column chromatography (hexanes:EtOAc 100:0→75:25) to afford the title compound, N-(7-amino-4-chloro-1-(2,2,2-trifluoroethyl)-1H-indazol-3-yl)-N-(4-methoxybenzyl)methanesulfonamide as an off-white solid, 1.6 g (61%).

### bicyclo[3.1.0]hexan-3-ol

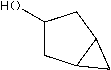

To a stirred solution of cyclopent-3-enol (130 g, 1545 mmol) in DCM (1200 mL) under N₂ atmosphere at 0-5° C. was added dropwise a solution of diethyl zinc in hexane (1.0 M, 3091 mL, 3091 mmol) over a period of 3 h. To the solution at 0° C. was added dropwise a solution of diiodomethane (249 mL, 3091 mmol) in DCM (300 mL) over a period of 1 h. The reaction mixture was allowed to warm to 27° C. upon which formation of a white precipitation was observed. The mixture stirred for 16 h. Progress of the reaction was monitored by TLC (SiO₂, 20% EtOAc/pet, Rf=0.3, UV-inactive, PMA-active). The reaction mixture was quenched via the careful addition of aq. saturated NH₄Cl solution (1.5 L). The mixture was filtered through pad of Celite. The aqueous layer was extracted with DCM (2×1 L). The combined organic layers were dried over anhydrous Na₂SO₄, filtered and then concentrated under reduced pressure to afford crude bicyclo[3.1.0]hexan-3-ol as red liquid, 180 g. ¹H NMR (400 MHz, CDCl₃) δ=4.41-4.35 (m, 1H), 2.18-2.05 (m, 2H), 1.73 (d, J=13.9 Hz, 2H), 1.35-1.25 (m, 2H), 1.21-1.14 (m, 1H), 0.57-0.43 (m, 2H). GCMS: m/z=98.1).

### bicyclo[3.1.0]hexan-3-one

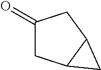

To a stirred solution of bicyclo[3.1.0]hexan-3-ol (210 g, 2054 mmol) in DCM (5000 mL) under N₂ atmosphere at 0° C. was added portion-wise Dess-Martin periodinane (954 g, 225 mmol). The mixture was allowed to warm to 27° C. and was then stirred for 16 h. Progress of the reaction was monitored by TLC (SiO₂, 20% Acetone/Hex, Rf=0.3, UV in-active, PMA-active). The reaction mixture was filtered through pad of Celite and the filtrate was washed with aq. NaOH (1N, 8×1 L). The combined aqueous phases were extracted with DCM (5×1 L). The combined organic layers were dried over anhydrous Na₂SO₄, filtered, and then concentrated under reduced pressure (bath temperature: 20° C.) to afford crude bicyclo[3.1.0]hexan-3-one as brown liquid. The liquid was further purified by downward distillation at 70° C. to afford bicyclo[3.1.0]hexan-3-one as a pale yellow viscous liquid, 125 g (62%). ¹H NMR (400 MHz, CDCl₃) δ=2.61-2.54 (m, 2H), 2.17-2.12 (m, 2H), 1.54-1.46 (m, 2H), 0.92-0.86 (m, 1H), −0.01-−0.08 (m, 1H); GCMS: M/Z=96.1.

### 2-(2,2-difluoroacetyl)bicyclo[3.1.0]hexan-3-one

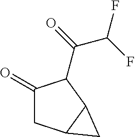 [SMILES: O=C1CC2CC2C1C(=O)C(F)F]

To a stirred solution of bicyclo[3.1.0]hexan-3-one (125 g, 1274 mmol) in THF (1500 mL) under N₂ atmosphere at −78° C. was added LDA (2.0 M in THF, 0.701 L, 1402 mmol). The solution was stirred for 1 h at −78° C. To the solution was added slowly over 30 minutes a solution of ethyldifluoroacetate (174 g, 1402 mmol) in THF (300 mL) maintaining a temperature of −78° C. The reaction mixture was allowed to warm to 27° C. and was then stirred for 1 h. Progress of the reaction was monitored by TLC (SiO₂, 20% Acetone/Hexane, Rf=0.3, UV-active). The reaction mixture was quenched via the addition of aq. HCl (1N, 2000 mL). The mixture was stirred for 30 min. and then was extracted with EtOAc (3×1000 mL). The combined organic layers were washed with brine (1000 mL), dried over anhydrous Na₂SO₄ and filtered. The filtrate was concentrated under reduced pressure to afford 2-(2,2-difluoroacetyl)bicyclo[3.1.0]hexan-3-one as a pale yellow viscous liquid, 180 g (71%). ¹H NMR (400 MHz, CDCl₃) δ=6.18 (t, J=54.8 Hz, 1H), 2.70-2.62 (m, 1H), 2.35 (d, J=19.4 Hz, 1H), 2.14 (br s, 1H), 1.26-1.21 (m, 1H), 1.04-1.03 (m, 1H), 0.22-0.21 (m, 1H), LCMS: M/Z=173.17).

### ethyl 2-(3-(difluoromethyl)-3b,4,4a,S-tetrahydro-1H-cyclopropa[3,4]cyclopenta[1,2-c]pyrazol-1-yl)acetate

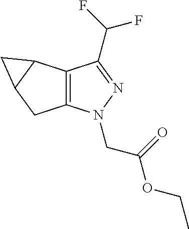 [SMILES: CCOC(=O)Cn1nc(C(F)F)c2c1CC1CC21]

To a stirred solution of 2-(2,2-difluoroacetyl)bicyclo[3.1.0]hexan-3-one (180 g, 910 mmol) in ethanol (2 L) under N₂ atmosphere at 27° C. was added ethyl 2-hydrazinylacetate hydrochloride (422 g, 2729 mmol) followed by sulfuric acid (20 mL, 375 mmol). The mixture was stirred for 30 min. and then was heated to 100° C. and stirred for 16 h. Progress of the reaction was monitored by TLC (SiO₂, 20% Acetone/Hexane, Rf=0.3, UV-active). The reaction mixture was concentrated under reduced pressure. The residue was dissolved in EtOAc (2000 mL) and was washed with water (2×1 L), brine (1.0 L), dried over anhydrous Na₂SO₄, filtered, and then was concentrated under reduced pressure. The resulting residue was subjected to silica gel column chromatography (pet.:acetone 100:0→98:2) to afford ethyl 2-(3-(difluoromethyl)-3b,4,4a,5-tetrahydro-1H-cyclopropa[3,4]cyclopenta[1,2-c]pyrazol-1-yl)acetate as an off-white solid, 110 g (46%). ¹H NMR (400 MHz, DMSO-d₆) δ=6.86 (t, J=54.8 Hz, 1H), 4.93 (s, 2H), 4.14 (q, J=7.2 Hz, 2H), 2.88-2.79 (m, 1H), 2.76-2.68 (m, 1H), 2.14-2.04 (m, 2H), 1.19 (t, J=7.2 Hz, 3H), 1.10-1.03 (m, 1H), 0.14 (q, J=4.3 Hz, 1H).

### ethyl 2-(3-(difluoromethyl)-S-oxo-3b,4,4a,5-tetrahydro-1H-cyclopropa[3,4]cyclopenta[1,2-c]pyrazol-1-yl)acetate

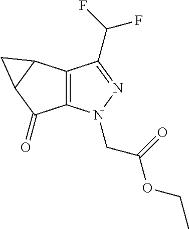 [SMILES: CCOC(=O)Cn1nc(C(F)F)c2c1C(=O)C1CC21]

To a stirred solution of ethyl 2-(3-(difluoromethyl)-3b,4,4a,5-tetrahydro-1H-cyclopropa[3,4]cyclopenta[1,2-c]pyrazol-1-yl)acetate (110 g, 422 mmol) and Celite (395 g) in cyclohexane (3.5 L) at 0° C. was added portionwise pyridinium dichromate (794 g, 2110 mmol). To the mixture under nitrogen atmosphere was added dropwise tert-butyl hydroperoxide (355 mL, 2130 mmol) over a period of 10 min. The reaction mixture was warmed to 27° C. and was then stirred at that temperature for 48 h. Progress of the reaction was monitored by TLC (SiO₂, 30% Acetone/pet, Rf=0.4, UV-active). The reaction mixture was filtered, and the filter cake was extracted with EtOAc (1000 mL). The filtrate was washed with saturated aq. Na₂S₂O₃ (2×500 mL); saturated aq. FeSO₄ (300 mL); and then brine (500 mL). The organic layer was dried over anhydrous Na₂SO₄; filtered and concentrated under reduced pressure to obtain the crude title compound (150 g).

### ethyl 2-(3-(difluoromethyl)-4,4a-dihydrospiro[cyclopropa[3,4]cyclopenta[1,2-c]pyrazole-5,2′-[1,3]dithiolane]-1(3bH)-yl)acetate

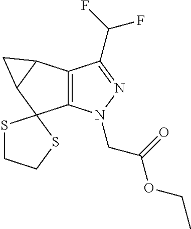 [SMILES: CCOC(=O)Cn1nc(C(F)F)c2c1C1(SCCS1)C1CC21]

To a stirred solution of ethyl 2-(3-(difluoromethyl)-5-oxo-3b,4,4a,5-tetrahydro-1H-cyclopropa[3,4]cyclopenta[1,2-c]pyrazol-1-yl)acetate (75 g, 269 mmol) in DCM (1500 mL) at 27° C. under nitrogen atmosphere was added ethane-1,2-dithiol (43.0 mL, 511 mmol) followed by the addition of boron trifluoride acetic acid (72.6 mL, 511 mmol). The solution was stirred for 16 h. Progress of the reaction was monitored by TLC (SiO₂, 20% Acetone/Pet, Rf=0.35, UV-Active). After completion, the reaction mixture was cooled to 0° C. and quenched via the addition of aq. saturated NaHCO₃(500 mL). The mixture was extracted with DCM (2×1000 mL). The combined organics were washed with brine (1000 mL), dried over anhydrous Na₂SO₄, filtered and concentrated under reduced pressure to obtain a brown liquid. This material was subjected to silica gel column chromatography (Pet.:EtOAc 95:5→90:10) to afford ethyl 2-(3-(difluoromethyl)-4,4a-dihydrospiro[cyclopropa[3,4]cyclopenta[1,2-c]pyrazole-5,2′-[1,3]dithiolane]-1(3bH)-yl)acetate as an off-white solid, 80 g (74%). ¹H-NMR (400 MHz, CDCl₃) δ=6.61 (t, J=55.2 Hz, 1H), 5.00-4.85 (m, 2H), 4.29-4.19 (m, 2H), 3.55-3.46 (m, 4H), 2.63-2.53 (m, 1H), 2.49-2.38 (m, 1H), 1.30-1.24 (m, 4H), 0.65-0.60 (m, 1H). LCMS M+H=346.9.

### ethyl 2-(3-(difluoromethyl)-5,5-difluoro-3b,4,4a,S-tetrahydro-1H-cyclopropa[3,4]cyclopenta[1,2-c]pyrazol-1-yl)acetate

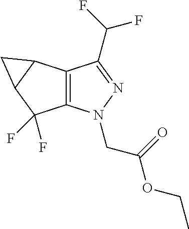 [SMILES: CCOC(=O)Cn1nc(C(F)F)c2c1C(F)(F)C1CC21]

To a stirred solution of 1,3-dibromo-5,5-dimethylimidazolidine-2,4-dione (26.3 g, 92 mmol) in DCM (20 mL) at −70° C. under N₂ atmosphere was added HF-pyridine (2.460 g, 24.83 mmol). The solution was for 30 min. To the solution was added a solution of ethyl 2-(3-(difluoromethyl)-4,4a-dihydrospiro[cyclopropa[3,4]cyclopenta[1,2-c]pyrazole-5,2′-1,3]dithiolane]-1(3bH)-yl)acetate (10 g, 25 mmol) in DCM (20 mL). The reaction mixture was allowed to warm to −40° C. and then was stirred at that temperature for 1 h. Progress of the reaction was monitored by TLC (SiO₂, 30% EtOAc/Pet, Rf=0.3, UV in-active). The reaction mixture was quenched via the addition of aq. sat. NaHCO₃(200 mL). The mixture was warmed to room temperature and was then extracted with EtOAc (2×100 mL). The combined organics were washed with brine (50 mL); dried over anhydrous Na₂SO₄; filtered; and were concentrated under reduced pressure to afford a brown solid. This material was subjected to silica gel column chromatography (Pet.:EtOAc 100:0→75-25) to afford ethyl 2-(3-(difluoromethyl)-5,5-difluoro-3b,4,4a,5-tetrahydro-1H-cyclopropa[3,4]cyclopenta[1,2-c]pyrazol-1-yl)acetate as a pale yellow solid, 8.5 g (91%). ¹H NMR (400 MHz, CDCl₃) δ=6.62 (t, J=55.2 Hz, 1H), 4.82 (s, 2H), 4.30-4.18 (m, 2H), 2.51-2.37 (m, 2H), 1.42-1.35 (m, 1H), 1.31-1.23 (m, 3H), 1.14-1.08 (m, 1H). LCMS M+H=293.07.

### 2-(3-(difluoromethyl)-5,5-difluoro-3b,4,4a,S-tetrahydro-1H-cyclopropa[3,4]cyclopenta[1,2-c]pyrazol-1-yl)acetic acid

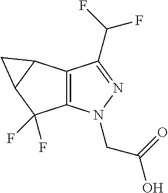 [SMILES: O=C(O)Cn1nc(C(F)F)c2c1C(F)(F)C1CC21]

To a stirred solution of ethyl 2-(3-(difluoromethyl)-5,5-difluoro-3b,4,4a,5-tetrahydro-1H-cyclopropa[3,4]cyclopenta[1,2-c]pyrazol-1-yl)acetate (15 g, 50 mmol) in THF (17 mL) and MeOH (66 mL) at 0° C. under N₂ atmosphere was added a solution of LiOH (1.788 g, 74.7 mmol) in water (66 mL). The reaction mixture was allowed to warm to 27° C. and was then stirred for 3 h at that temperature. Progress of the reaction was monitored by TLC (SiO₂, 5% MeOH/DCM, Rf=0.2, UV Active). After completion, the reaction mixture was concentrated under reduced pressure; diluted with water (50 mL); and washed with EtOAc (2×250 mL) to remove impurities. The aqueous layer was adjusted to pH 2-3 using aq. HCl (1M), then was extracted with EtOAc (3×1000 mL). The combined organics were dried over anhydrous Na₂SO₄; filtered; and concentrated under reduced pressure to afford 2-(3-(difluoromethyl)-5,5-difluoro-3b,4,4a,5-tetrahydro-1H-cyclopropa[3,4]cyclopenta[1,2-c]pyrazol-1-yl)acetic acid as an off white solid, 14 g (98%). LCMS M+H=265.15.

### 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid and 2-((3bR,4aS)-3-(difluoromethyl)-5,5-difluoro-3b,4,4a,5-tetrahydro-1H-cyclopropa[3,4]cyclopenta[1,2-c]pyrazol-1-yl)acetic acid

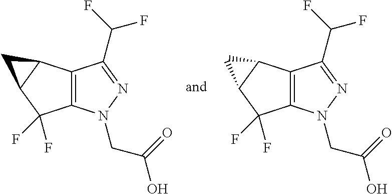 [SMILES: O=C(O)Cn1nc(C(F)F)c2c1C(F)(F)[C@H]1C[C@@H]21 ; O=C(O)Cn1nc(C(F)F)c2c1C(F)(F)[C@@H]1C[C@H]21]

2-(3-(difluoromethyl)-5,5-difluoro-3b,4,4a,5-tetrahydro-1H-cyclopropa[3,4]cyclopenta[1,2-c]pyrazol-1-yl)acetic acid (5.5 g) was dissolved in isopropanol (20 mL). The solution was subjected portion-wise to SFC chiral separation as follows: Instrument=Thar 80; column=Chiralpak IC 30×250 mm, 5 micron; solvent A=super critical CO₂; solvent B=isopropanol with 0.5% isopropylamine (v/v); eluent composition=70% A:30% B; flow-rate=65 g/min; back-pressure=100 bar; temperature=30° C.; injection volume=2.5 mL; detection=220 nm. 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid was collected as peak eluting from 7.5 min. to 14 min; 2-((3bR,4aS)-3-(difluoromethyl)-5,5-difluoro-3b,4,4a,5-tetrahydro-1H-cyclopropa[3,4]cyclopenta[1,2-c]pyrazol-1-yl)acetic acid was collected as a peak eluting from 2.7 min. to 5.8 min. For each enantiomer, the resulting solution was concentrated under reduced pressure and the resulting solids were dissolved in EtOAc, then twice washed with aq. citric acid (1M) followed by water followed by brine. The organic solution was dried over Na₂SO₄; filtered; then concentrated in vacuo to afforded the separated enantiomer in 80-90% recovery.

### 2-(2,2,2-trifluoroacetyl)bicyclo[3.1.0]hexan-3-one

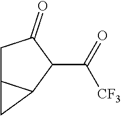 [SMILES: O=C1CC2CC2C1C(=O)C(F)(F)F]

To a solution of bicyclo[3.1.0]hexan-3-one (77.0 g, 800 mmol, 1 equiv) in THF (385 mL) at −78° C. was added LDA (842 mL of a 1 M solution in THF, 840 mmol, 1.05 equiv). After stirring 1 h, ethyl trifluoroacetate (136.5 g, 960 mmol, 1.2 equiv) was added dropwise. The reaction was stirred for 1 h at −78° C. and then warmed to ambient temperature. After stirring for 3 h, the temperature was cooled to 0° C. and quenched by addition of 6 N HCl. The reaction was extracted with MTBE. The MTBE extract was washed with 5% brine and the organics were carefully distilled to leave the crude product (218 g) which was carried on without further purification. 3-(trifluoromethyl)-3b,4,4a,5-tetrahydro-1H-cyclopropa[3,4]cyclopenta[1,2-c]pyrazole

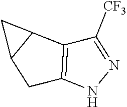 [SMILES: FC(F)(F)c1n[nH]c2c1C1CC1C2]

To a solution of crude 2-(2,2,2-trifluoroacetyl)bicyclo[3.1.0]hexan-3-one (218 g, ˜154 g active, −800 mmol, 1 equiv) from the previous step in EtOH (770 mL) was added slowly hydrazine monohydrate (48.1 g, 960 mmol, 1.2 equiv). The reaction was heated to 75° C. for 8 h. Upon cooling to ambient temperature, the reaction was concentrated in vacuo to provide the crude product which was purified by silica gel flash chromatography (20-25% EtOAc/hexane) to provide the product (102.5 g, 68% over 2 steps). ¹H NMR (400 MHz, CDCl₃): δ 13.18 (bs, 1H), 3.02-2.97 (m, 1H), 2.87-2.83 (m, 1H), 2.15-2.09 (m, 2H), 1.14-1.09 (m, 1H), 0.32-0.29 (m, 1H). MS (m/z): 189 [M+H]⁺. Purity by HPLC: 98.99%.

### ethyl 2-(3-(trifluoromethyl)-3b,4,4a,5-tetrahydro-1H-cyclopropa[3,4]cyclopenta[1,2-c]pyrazol-1-yl)acetate

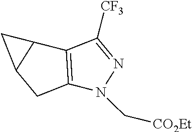 [SMILES: CCOC(=O)Cn1nc(C(F)(F)F)c2c1CC1CC21]

To a solution of 3-(trifluoromethyl)-3b,4,4a,5-tetrahydro-1H-cyclopropa[3,4]cyclopenta[1,2-c]pyrazole (205 g, 1090 mmol, 1 equiv) in acetonitrile (1640 mL) at 0° C. was added K₂CO₃ (180.5 g, 1300 mmol, 1.2 equiv) followed 5 min later by benzyl triethylammonium chloride (12.1 g, 54 mmol, 0.05 equiv). To this mixture was added dropwise ethyl bromoacetate (218 g, 1300 mmol, 1.2 equiv). After complete addition, the reaction was heated to 70° C. for 12 h. Upon cooling to ambient temperature, the reaction was filtered and the filtrate was concentrated in vacuo. The residue was partitioned between ethyl acetate and water. After separation, the ethyl acetate layer was washed with brine, dried (Na₂SO₄), and concentrated in vacuo. The crude product was triturated in hexane for 20 h and then cooled to 0° C. and filtered. The resultant solid was washed with hexane to provide the product (176 g, 59%) which was contaminated with 2% of the minor alkylation isomer. ¹H NMR (400 MHz, CDCl₃): δ 4.74 (ABq, J₁=17.50 Hz, J₂=11.36 Hz, 2H), 4.22 (q, J=7.0 Hz, 2H), 2.94-2.88 (m, 1H), 2.75-2.70 (m, 1H), 2.14-2.09 (m, 2H), 1.28 (t, J=7.0 Hz, 3H), 1.14-1.09 (m, 1H), 0.34-0.33 (m, 1H). MS (m/z): 275 [M+H]⁺. Purity by HPLC: 97.08%.

### ethyl 2-(5-oxo-3-(trifluoromethyl)-3b,4,4a,5-tetrahydro-1H-cyclopropa[3,4]cyclopenta[1,2-c]pyrazol-1-yl)acetate

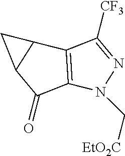 [SMILES: CCOC(=O)Cn1nc(C(F)(F)F)c2c1C(=O)C1CC21]

To a solution of ethyl 2-(3-(trifluoromethyl)-3b,4,4a,5-tetrahydro-1H-cyclopropa[3,4]cyclopenta[1,2-c]pyrazol-1-yl)acetate (80.0 g, 290 mmol, 1 equiv) in acetone (800 mL) at 0° C. was added N-hydroxyphthalimide (4.8 g, 29 mmol, 0.1 equiv) followed by Co(OAc)₂.4H₂O (7.3 g, 29 mmol, 0.1 equiv). 70% tert-butyl hydrogen peroxide (225 g, 1750 mmol, 6 equiv) was added slowly. After warming to ambient temperature, the reaction was stirred for 24 h. The reaction was then added to cold 10% Na₂S₂O₃ (800 mL) followed by EtOAc (800 mL). After 20 min, the layers were separated and the EtOAc layer was washed with water, 10% aqueous sodium bicarbonate, and 10% brine. The EtOAc layer was then dried (Na₂SO₄) and concentrated in vacuo. The crude product was purified by silica gel flash chromatography (10-15% EtOAc/hexane) to provide the product (54.5 g, 65%). ¹H NMR (400 MHz, CDCl₃): δ 4.97 (s, 2H), 4.25-4.21 (m, 2H), 2.79-2.76 (m, 1H), 2.62-2.58 (m, 1H), 1.73-1.67 (m, 2H), 1.30-1.26 (m, 3H). MS (m/z): 289 [M+H]⁺. Purity by HPLC: 93.97%.

### ethyl 2-(3-(trifluoromethyl)-4,4a-dihydrospiro[cyclopropa[3,4]cyclopenta[1,2-c]pyrazole-5,2′-[1,3]dithiolane]-1(3bH)-yl)acetate

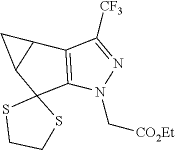 [SMILES: CCOC(=O)Cn1nc(C(F)(F)F)c2c1C1(SCCS1)C1CC21]

To a solution of ethyl 2-(5-oxo-3-(trifluoromethyl)-3b,4,4a,5-tetrahydro-1H-cyclopropa[3,4]cyclopenta[1,2-c]pyrazol-1-yl)acetate (55.0 g, 190 mmol, 1 equiv) in DCM (1320 mL) at 0° C. was added 1,2-ethanedithiol (32.1 mL, 380 mmol, 2 equiv). After 5 min, BF₃.2AcOH (52.8 mL, 380 mmol, 2 equiv) was added slowly. After warming to ambient temperature, the solution was stirred for 16 h. The reaction was cooled to 0° C. and a 10% aqueous sodium bicarbonate (880 mL) was added slowly. The biphasic mixture was stirred at ambient temperature for 30 min. The organic layer was separated and washed with 10% aqueous sodium bicarbonate, 10% aqueous Na₂S₂O₃, water, and 15% brine. The organic layer was concentrated in vacuo and the crude residue was purified by silica gel flash chromatography (10-15% EtOAc/hexane) to provide the product (57 g, 82%). ¹H NMR (400 MHz, CDCl₃): δ 4.99-4.86 (m, 2H), 4.25-4.18 (m, 2H), 3.51-3.44 (m, 4H), 2.63-2.59 (m, 1H), 2.43-2.39 (m, 1H), 1.33-1.23 (m, 4H), 0.66-0.62 (m, 1H). MS (m/z): 365 [M+H]⁺. Purity by HPLC: 92.59%.

### ethyl 2-(5,5-difluoro-3-(trifluoromethyl)-3b,4,4a,5-tetrahydro-1H-cyclopropa[3,4]cyclopenta[1,2-c]pyrazol-1-yl)acetate

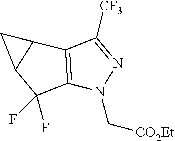 [SMILES: CCOC(=O)Cn1nc(C(F)(F)F)c2c1C(F)(F)C1CC21]

To a solution of 1,3-dibromo-5,5-dimethyl-hydantoin (85.05 g, 290 mmol, 3 equiv) in DCM (450 mL) at −78° C. was added 70% HF urea (108 mL). After 1 h, a solution of ethyl 2-(3-(trifluoromethyl)-4,4a-dihydrospiro[cyclopropa[3,4]cyclopenta[1,2-c]pyrazole-5,2′-[1,3]dithiolane]-1(3bH)-yl)acetate (36.0 g, 98 mmol, 1 equiv) in DCM (450 mL) was added slowly. The reaction was stirred at −78° C. for 1 h and then at −40° C. for 40 min. The reaction solution was then slowly added to 20% ammonia aqueous solution at −20° C. (235 mL). The reaction was stirred 20 min and then allowed to warm to ambient temperature. The organic layer was separated and washed with 10% aqueous Na₂S₂O₃ and 15% brine. The organic layer was concentrated in vacuo and the crude residue was purified by silica gel flash chromatography (10-15% EtOAc/hexane) to provide the product (24.3 g, 79%). ¹H NMR (400 MHz, CDCl₃): δ 4.86 (s, 2H), 4.22 (q, J=8.0 Hz, 2H), 2.53-2.45 (m, 2H), 1.45-1.38 (m, 1H), 1.28 (t, J=8.0 Hz, 3H), 1.18-1.12 (m, 1H). MS (m/z): 311 [M+H]⁺. Purity by HPLC: 87.75%

### 2-(5,5-difluoro-3-(trifluoromethyl)-3b,4,4a,5-tetrahydro-1H-cyclopropa[3,4]cyclopenta[1,2-c]pyrazol-1-yl)acetic acid

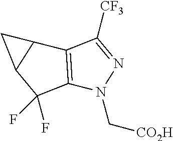 [SMILES: O=C(O)Cn1nc(C(F)(F)F)c2c1C(F)(F)C1CC21]

To a solution of ethyl 2-(5,5-difluoro-3-(trifluoromethyl)-3b,4,4a,5-tetrahydro-1H-cyclopropa[3,4]cyclopenta[1,2-c]pyrazol-1-yl)acetate (17.5 g, 56 mmol, 1 equiv) in THF (140 mL) at −5° C. was added a solution of LiOH monohydrate (2.37 g, 56 mmol, 1 equiv) in water (140 mL). After 30 min, water (35 mL) was added and the reaction was adjusted to pH 2-3 with 6 n HCl (35 mL). The resultant slurry was extracted with EtOAc (×2). The combined EtOAc layers were washed with 15% brine and concentrated in vacuo. The crude product was purified by silica gel flash chromatography (20-30% EtOAc/hexane) to provide the product (13.5 g, 84%). ¹H NMR (400 MHz, CDCl₃): δ 8.50 (bs, 1H), 4.93 (s, 2H), 2.53-2.46 (m, 2H), 1.43-1.38 (m, 1H), 1.20-1.15 (m, 1H). MS (m/z): 283 [M+H]⁺. Purity by HPLC: 98.01%.

### 2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid

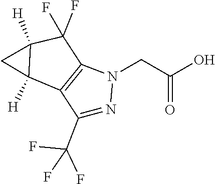 [SMILES: O=C(O)Cn1nc(C(F)(F)F)c2c1C(F)(F)[C@@H]1C[C@H]21]

2-(5,5-difluoro-3-(trifluoromethyl)-3b,4,4a,5-tetrahydro-1H-cyclopropa[3,4]cyclopenta[1,2-c]pyrazol-1-yl)acetic acid (5 g) was dissolved in isopropanol (20 mL). The solution was subjected portion-wise to SFC chiral separation as follows: Instrument=Thar 80; column=Chiralpak IC 30×250 mm, 5 micron; solvent A=super critical CO₂; solvent B=isopropanol with 0.5% isopropylamine (v/v); eluent composition=70% A:30% B; flow-rate=65 g/min; back-pressure=100 bar; temperature=30° C.; injection volume=2.5 mL; detection=220 nm. 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid was collected as peak eluting from 7.5 min. to 14 min; 2-((3bR,4aS)-3-(difluoromethyl)-5,5-difluoro-3b,4,4a,5-tetrahydro-1H-cyclopropa[3,4]cyclopenta[1,2-c]pyrazol-1-yl)acetic acid was collected as a peak eluting from 2.7 min. to 5.8 min. For each enantiomer, the resulting solution was concentrated under reduced pressure and the resulting solids were dissolved in EtOAc, then twice washed with aq. citric acid (1M) followed by water followed by brine. The organic solution was dried over Na₂SO₄; filtered; then concentrated in vacuo to afforded the separated enantiomer in 80-90% recovery.

### ethyl 2-(3-isopropyl-1H-pyrazol-1-yl)acetate

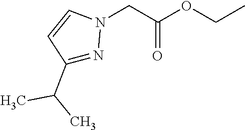

To a stirred solution of 3-isopropyl-1H-pyrazole (8.9 g, 81 mmol) in CH₃CN (90 mL) was added DIPEA (28.2 mL, 162 mmol) and ethyl bromoacetate (9.00 mL, 81 mmol) at 27° C. The reaction mixture was heated to 55° C. and stirred for 16 h. The progress of the reaction was monitored by TLC (SiO₂, 30% EtOAc/Pet., Rf=0.5, KMnO₄-active). Upon completion, the reaction mixture was concentrated under reduced pressure, diluted with water (100 mL) and extracted with EtOAc (2×200 mL). The combined organic layers were washed with brine (200 mL), dried over anhydrous Na₂SO₄, filtered and concentrated under vacuum to obtain the crude compound as brown oil. The crude residue was purified by silica gel chromatography (12 g silica gel column) eluting with 10-16% EtOAc/Pet. The fractions containing the product were collected and concentrated under reduced pressure to afford ethyl 2-(3-isopropyl-1H-pyrazol-1-yl)acetate (6.5 g, Yield=40%, pale yellow oil). ¹HNMR (400 MHz, DMSO-d₆) δ=7.57 (d, J=2.2 Hz, 1H), 6.09 (d, J=2.2 Hz, 1H), 4.94 (s, 2H), 4.13 (q, J=7.2 Hz, 2H), 2.95-2.78 (m, 1H), 1.31-1.10 (m, 9H). LCMS: RT=2.23 mins, (M+H)=197.23, Purity=98%.

### 2-(3-isopropyl-1H-pyrazol-1-yl)acetic acid

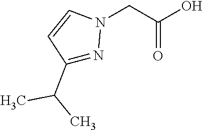

To a stirred solution of ethyl 2-(3-isopropyl-1H-pyrazol-1-yl)acetate (3.5 g, 17.83 mmol) in THF (35 mL) and water (3.5 mL) was added lithium hydroxide (1.497 g, 35.7 mmol) at 27° C. The reaction mixture was stirred at 27° C. for 4 h. The progress of the reaction was monitored by TLC (SiO₂, EtOAc, Rf=0.1, KMnO₄-active). Upon completion, the reaction mixture was concentrated under reduced pressure, dissolved in water (100 mL) and washed with EtOAc (100 mL). The aqueous layer was acidified with 2N HCl (until pH ˜2) at 0° C. and then was extracted with EtOAc (3×150 mL). The combined organic layers were washed brine (150 mL), dried over anhydrous Na₂SO₄, filtered and concentrated under reduced pressure to afford 2-(3-isopropyl-1H-pyrazol-1-yl)acetic acid (2.3 g, yield=76%, white solid). ¹HNMR (400 MHz, DMSO-d₆) δ=12.91 (brs, 1H), 7.55 (d, J=2.2 Hz, 1H), 6.07 (d, J=2.2 Hz, 1H), 4.84 (s, 2H), 2.90-2.80 (m, 1H), 1.17 (d, J=7.0 Hz, 6H). LCMS: RT=1.42 mins, (M+H)=169.24, Purity=99%.

### (E)-1-cyclopropyl-3-(dimethylamino)prop-2-en-1-one

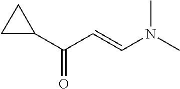

A stirred mixture of 1-cyclopropylethanone (20 g, 238 mmol) and DMF-DMA (31.8 ml, 238 mmol) was heated to 120° C. and stirred for 48 h. The progress of the reaction was monitored by TLC (SiO₂, 50% EtOAc/Pet., Rf=0.3, Ninhydrin-active). Upon completion, the reaction mixture was concentrated under reduced pressure to afford (E)-1-cyclopropyl-3-(dimethylamino) prop-2-en-1-one (11.5 g, yield=33%, yellow colour oil). ¹HNMR (400 MHz, CDCl₃) δ=7.56 (d, J=12.6 Hz, 1H), 5.20 (d, J=12.6 Hz, 1H), 3.08-2.76 (m, 6H), 1.85-1.75 (m, 1H), 1.06-0.96 (m, 2H), 0.78-0.70 (m, 2H). LCMS: RT=1.75 mins, (M+H)=140.20, Purity=95%.

### 3-cyclopropyl-1H-pyrazole

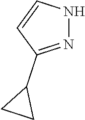

To a stirred solution of (E)-1-cyclopropyl-3-(dimethylamino)prop-2-en-1-one (10.5 g, 71.7 mmol) in EtOH (100 mL) was added hydrazine hydrate (6.77 mL, 107 mmol) and catalytic sulfuric acid (0.02 mL, 0.375 mmol) at 27° C. The reaction mixture was stirred at 85° C. for 2 h. The progress of the reaction was monitored by TLC (SiO₂, 50% EtOAc/Pet., Rf=0.3, KMnO₄-active). Upon completion, the reaction mixture was concentrated under reduced pressure and the residue was diluted with water (120 mL) and extracted with EtOAc (3×250 mL). The combined organic layers were washed with brine (200 mL), dried over anhydrous Na₂SO₄, filtered and concentrated under reduced pressure to afford 3-cyclopropyl-1H-pyrazole (7.25 g, yield=78%, yellow oil). ¹HNMR (400 MHz, CDCl₃) δ=7.46 (d, J=2.0 Hz, 1H), 5.96 (d, J=2.1 Hz, 1H), 1.98-1.92 (m, 1H), 0.99-0.91 (m, 2H), 0.77-0.70 (m, 2H). LCMS: RT=1.53 mins, (M+H)=109.13, Purity=82%.

### ethyl 2-(3-cyclopropyl-1H-pyrazol-1-yl)acetate

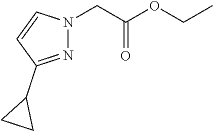

To a stirred solution of 3-cyclopropyl-1H-pyrazole (7.25 g, 67.0 mmol) in CH₃CN (75 mL) were added DIPEA (23.42 mL, 134 mmol) and ethyl bromoacetate (7.46 mL, 67.0 mmol) at 27° C. The reaction mixture was heated to 55° C. and stirred for 16 h. The progress of the reaction was monitored by TLC (SiO₂, 30% EtOAc/Pet., Rf=0.5, KMnO₄-active). On completion, the reaction mixture was concentrated and the residue was partitioned between water (100 mL) and EtOAc (300 mL). The organic layer was washed with brine (200 mL), dried over anhydrous Na₂SO₄, filtered and concentrated under reduced pressure to give the crude compound as brown oil which was then purified by silica gel column chromatography (12 g column) eluting with 10-16% EtOAc/Pet. The fractions containing product were collected and concentrated under reduced pressure to afford ethyl 2-(3-cyclopropyl-1H-pyrazol-1-yl)acetate (5.3 g, Yield: 38%, pale yellow oil). ¹HNMR (400 MHz, DMSO-d₆) δ=7.54 (d, J=2.2 Hz, 1H), 5.94 (d, J=2.2 Hz, 1H), 4.91 (s, 2H), 4.13 (q, J=7.2 Hz, 2H), 1.90-1.76 (m, 1H), 1.20 (t, J=7.1 Hz, 3H), 0.86-0.79 (m, 2H), 0.62-0.57 (m, 2H). LCMS: RT=2.00 mins, (M+H)=195.21, Purity=95%.

### 2-(3-cyclopropyl-1H-pyrazol-1-yl)acetic acid

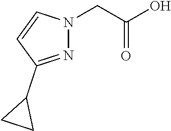 [SMILES: O=C(O)Cn1ccc(C2CC2)n1]

To a stirred solution of ethyl 2-(3-cyclopropyl-1H-pyrazol-1-yl)acetate (3 g, 15.45 mmol) in THF (30 mL) and water (3 mL) was added lithium hydroxide (1.296 g, 30.9 mmol) at 27° C. The reaction mixture was stirred for 4 h. The progress of the reaction was monitored by TLC (SiO₂, EtOAc, Rf=0.1, KMnO₄-active). Upon completion, the reaction mixture was concentrated under reduced pressure and the residue was dissolved in water (100 mL), and then washed with EtOAc (100 mL). The aqueous layer was cooled to 0° C., acidified with 2 N HCl (to pH ˜2) and extracted with EtOAc (3×150 mL). The combined organic layers were washed with brine (150 mL), dried over anhydrous Na₂SO₄, filtered and concentrated under reduced pressure to afford 2-(3-cyclopropyl-1H-pyrazol-1-yl)acetic acid (1.3 g, Yield: 50%, off-white solid). ¹HNMR (400 MHz, DMSO-d₆) δ=12.91 (brs, 1H), 7.52 (d, J=2.2 Hz, 1H), 5.92 (d, J=2.2 Hz, 1H), 4.80 (s, 2H), 1.87-1.80 (m, 1H), 0.90-0.75 (m, 2H), 0.65-0.53 (m, 2H). LCMS: RT=1.28 mins, (M+H)=167.13, Purity=99%.

### ethyl 2-(3-cyclobutyl-1H-pyrazol-1-yl)acetate

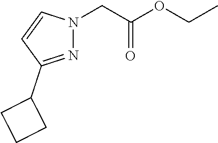

To a solution of 3-cyclobutyl-1H-pyrazole (0.5 g, 4.09 mmol, 1 equiv) and methyl 2-bromoacetate (0.426 mL, 4.50 mmol, 1.1 equiv) in DMF (10 mL) was added potassium carbonate (0.679 g, 4.91 mmol, 1.2 equiv). After stirring for 18 h, the reaction was diluted with water and extracted with EtOAc. The organic layer was washed with brine, dried (MgSO₄), concentrated in vacuo to afford the crude product. The crude product was purified via silica gel flash chromatography (0-100% EtOAc:Hex) to afford the product methyl 2-(3-cyclobutyl-1H-pyrazol-1-yl)acetate (477 mg, 60.0%) as a clear oil. The other regioisomer methyl 2-(5-cyclobutyl-1H-pyrazol-1-yl)acetate was also isolated but was impure. ¹H NMR (500 MHz, CHLOROFORM-d) δ ppm 7.39 (d, J=2.09 Hz, 1H), 6.23 (d, J=2.09 Hz, 1H), 4.88 (s, 2H), 3.78 (s, 3H), 3.58 (quin, J=8.72 Hz, 1H), 2.31-2.39 (m, 2H), 2.17-2.27 (m, 2H), 1.99-2.06 (m, 1H), 1.86-1.94 (m, 1H).

### 2-(3-cyclobutyl-1H-pyrazol-1-yl)acetic acid

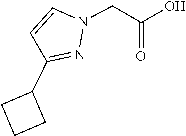 [SMILES: O=C(O)Cn1ccc(C2CCC2)n1]

To a solution of methyl 2-(3-cyclobutyl-1H-pyrazol-1-yl)acetate (477 mg, 2.456 mmol, 1 equiv) in methanol (10 mL) was added sodium hydroxide (1.473 mL, 7.37 mmol, 3 equiv). After 4 h, the LCMS indicated that starting material was consumed. The reaction was concentrated in vacuo. The crude residue was diluted with aq. HCl (7.61 mL, 7.61 mmol), water (25 mL), and EtOAc (50 mL). The organic layer was washed with brine, dried (MgSO₄), and concentrated in vacuo to afford the product 2-(3-cyclobutyl-1H-pyrazol-1-yl)acetic acid (364 mg, 2.020 mmol, 82%) as a white solid. ¹H NMR (500 MHz, CHLOROFORM-d) δ ppm 7.38 (d, J=2.38 Hz, 1H), 6.23 (d, J=2.38 Hz, 1H), 4.93 (s, 2H), 3.59 (quin, J=8.64 Hz, 1H), 2.30-2.41 (m, 2H), 2.20 (quind, J=9.20, 9.20, 9.20, 9.20, 2.53 Hz, 2H), 1.98-2.10 (m, 1H), 1.84-1.96 (m, 1H).

### tert-butyl (S)-(1-(7-bromo-3-(4-chloro-3-(N-(4-methoxybenzyl)methylsulfonamido)-1-methyl-1H-indazol-7-yl)-4-oxo-3,4-dihydroquinazolin-2-yl)-2-(3,5-difluorophenyl)ethyl)carbamate

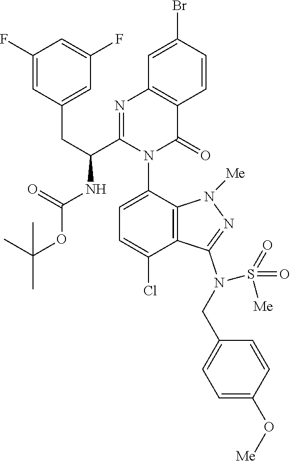 [SMILES: COc1ccc(CN(c2nn(C)c3c(-n4c([C@H](Cc5cc(F)cc(F)c5)NC(=O)OC(C)(C)C)nc5cc(Br)ccc5c4=O)ccc(Cl)c23)S(C)(=O)=O)cc1]

To a solution of (S)-2-((tert-butoxycarbonyl)amino)-3-(3,5-difluorophenyl)propanoic acid (3.82 g, 12.66 mmol), 2-amino-4-bromobenzoic acid (3.01 g, 13.93 mmol) and N-(7-amino-4-chloro-1-methyl-1H-indazol-3-yl)-N-(4-methoxybenzyl)methanesulfonamide (5 g, 12.66 mmol) in pyridine (50 mL) was added diphenyl phosphite (9.80 mL, 50.6 mmol). The resulting mixture was placed on a preheated oil bath (70° C.) and heated at 70° C. for 16 h. The mixture was cooled to room temperature and then concentrated under reduced pressure. The mixture was then diluted with EtOAc (approximately 500 mL) and washed with aqueous citric acid (0.5M, 2×50 mL), then aqueous NaOH (1M, 3×50 mL), dried over Na₂SO₄, filtered, and concentrated. The residue was then purified via silica gel chromatography (330 g silica gel column, gradient of hexanes:EtOAc 0:100→50:50) to afford tert-butyl (S)-(1-(7-bromo-3-(4-chloro-3-(N-(4-methoxybenzyl)methylsulfonamido)-1-methyl-1H-indazol-7-yl)-4-oxo-3,4-dihydroquinazolin-2-yl)-2-(3,5-difluorophenyl)ethyl)carbamate (6.2 g, 7.22 mmol, 57.1% yield) as pale yellow solid foam (inseparable mixture of atropisomers). LC/MS: m/z=801.10 [M−tBu].

### (S)—N-(7-(2-(1-amino-2-(3,5-difluorophenyl)ethyl)-7-bromo-4-oxoquinazolin-3(4H)-yl)-4-chloro-1-methyl-1H-indazol-3-yl)methanesulfonamide

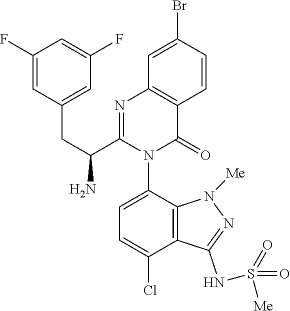 [SMILES: Cn1nc(NS(C)(=O)=O)c2c(Cl)ccc(-n3c([C@@H](N)Cc4cc(F)cc(F)c4)nc4cc(Br)ccc4c3=O)c21]

To a stirred solution of tert-butyl (S)-(1-(7-bromo-3-(4-chloro-3-(N-(4-methoxybenzyl)methylsulfonamido)-1-methyl-1H-indazol-7-yl)-4-oxo-3,4-dihydroquinazolin-2-yl)-2-(3,5-difluorophenyl)ethyl)carbamate (6.2 g, 7.22 mmol) in dichloromethane (DCM) (50 mL) was added trifluoroacetic acid (20 mL, 260 mmol) followed by trifluoromethanesulfonic acid (0.770 mL, 8.67 mmol). The resulting dark red solution was stirred at room temperature for 1 h. LCMS at this point indicates two peaks containing the desired product mass, consistent with the presence of two diastereomeric atropisomers (ratio of approximately 30:70). The mixture was concentrated in vacuo and the resulting residue was partitioned between EtOAc (300 mL) and aq. NaOH (1M, 30 mL). The aq. phase was tested and determined to be pH >=8.0. The organic phase was isolated and dried over Na₂SO₄, filtered, and then concentrated in vacuo. The residue was purified in three approximately equal portions via C18 chromatography (275 g RediSep Gold Column, Mobile Phase A: 5:95 acetonitrile:water with 0.1% TFA; Mobile Phase B: 95:5 acetonitrile:water with 0.1% TFA; gradient of 10-60% B over 30 min). Fractions containing the major atropisomer (second eluting) were combined, adjusted to pH 8 via addition of aq. 1M NaOH; extracted with ethyl acetate; washed with brine (sat. aq. NaCl); dried over Na₂SO₄; filtered; and then concentrated to afford the desired major atropisomer (S)—N-(7-(2-(1-amino-2-(3,5-difluorophenyl)ethyl)-7-bromo-4-oxoquinazolin-3(4H)-yl)-4-chloro-1-methyl-1H-indazol-3-yl)methanesulfonamide (2.4 g, 3.76 mmol, 52% yield). ¹H NMR (500 MHz, DMSO-d₆) δ ppm 8.11 (d, J=8.55 Hz, 1H), 8.06 (d, J=1.53 Hz, 1H), 7.81 (dd, J=8.55, 1.83 Hz, 1H), 7.33 (s, 2H), 6.96-7.05 (m, 1H), 6.75 (br d, J=7.02 Hz, 2H), 3.67 (s, 3H), 3.56 (dd, J=7.63, 5.19 Hz, 1H), 3.25-3.29 (m, 1H), 3.21 (s, 3H), 2.81 (dd, J=13.43, 8.24 Hz, 1H). LCMS: m/z=637.05 [M+H]⁺.

### N—((S)-1-(7-bromo-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 1)

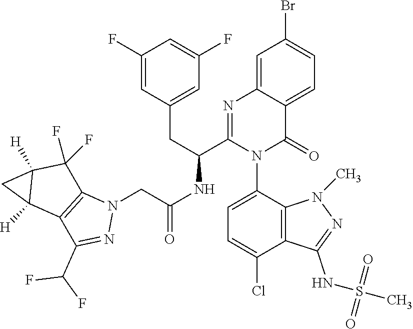 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(Br)ccc4c3=O)c21]

To a solution of (S)—N-(7-(2-(1-amino-2-(3,5-difluorophenyl)ethyl)-7-bromo-4-oxoquinazolin-3(4H)-yl)-4-chloro-1-methyl-1H-indazol-3-yl)methanesulfonamide (2.08 g, 3.26 mmol), 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.861 g, 3.26 mmol) and diisopropylethylamine (“DIPEA”) (1.709 mL, 9.78 mmol) in tetrahydrofuran (THF) (30 mL) was added HATU (1.364 g, 3.59 mmol). The resulting mixture was stirred at room temp for 3 h. To the mixture was added ammonia in methanol (2M, 3 mL). The mixture was stirred at room temp for 30 min. Water was then added and the mixture was extracted with ethyl acetate; washed with brine; dried over Na₂SO₄, filtered; and concentrated in vacuo. The resulting residue was subjected to silica gel chromatography (hexanes:EtOAc 100:0→30:70) to afford N—((S)-1-(7-bromo-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (2.5 g, 2.83 mmol, 87% yield). ¹H NMR (500 MHz, CDCl₃) δ ppm 8.18 (d, J=8.24 Hz, 1H), 7.88 (d, J=1.53 Hz, 1H), 7.72 (dd, J=8.55, 1.83 Hz, 1H), 7.33 (s, 1H), 7.16 (d, J=7.63 Hz, 1H), 6.57-6.83 (m, 4H), 6.38 (br d, J=5.80 Hz, 2H), 4.71-4.80 (m, 1H), 4.63 (d, J=6.71 Hz, 2H), 3.56 (s, 3H), 3.40 (s, 3H), 3.18 (dd, J=13.73, 6.10 Hz, 1H), 2.86 (dd, J=13.58, 7.48 Hz, 1H), 2.52-2.61 (m, 1H), 2.41-2.50 (m, 1H), 1.42-1.50 (m, 1H), 1.09-1.16 (m, 1H). LCMS: m/z=883.05 [M+H]⁺.

### tert-Butyl (S)-(1-(7-bromo-3-(4-chloro-1-(2,2-difluoroethyl)-3-(N-(4-methoxybenzyl)methylsulfonamido)-1H-indazol-7-yl)-4-oxo-3,4-dihydroquinazolin-2-yl)-2-(3,5-difluorophenyl)ethyl)carbamate

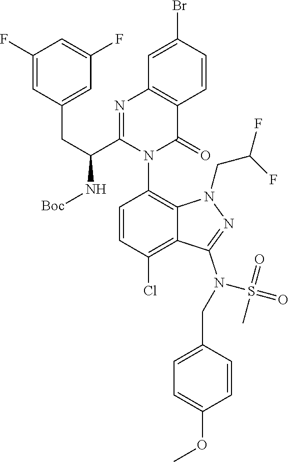 [SMILES: COc1ccc(CN(c2nn(CC(F)F)c3c(-n4c([C@H](Cc5cc(F)cc(F)c5)NC(=O)OC(C)(C)C)nc5cc(Br)ccc5c4=O)ccc(Cl)c23)S(C)(=O)=O)cc1]

To a stirred solution of (S)-2-((tert-butoxycarbonyl)amino)-3-(3,5-difluorophenyl)propanoic acid (15 g, 49.8 mmol) and 2-amino-4-bromobenzoic acid (10.76 g, 49.8 mmol) in pyridine (150 mL) was added diphenyl phosphite (9.64 mL, 49.8 mmol) at 27° C. The mixture was flushed with argon and the flask was then sealed. The reaction mixture was heated to 80° C. and stirred at that temperature for 2 hr. The reaction mixture was cooled to 27° C. and to the mixture was added N-(7-amino-4-chloro-1-(2,2-difluoroethyl)-1H-indazol-3-yl)-N-(4-methoxybenzyl)methanesulfonamide. The flask was sealed and the mixture was heated at 80° C. for 16 hr. The progress of the reaction was monitored by TLC (SiO₂, 30% EtOAc/Pet., Rf=0.4, UV-active). The reaction mixture was allowed to cool to 27° C. and then was concentrated under reduced pressure. The resulting residue was subjected to silica gel column chromatography (Pet.:EtOAc 80:20→70:30) to afford tert-butyl (S)-(1-(7-bromo-3-(4-chloro-1-(2,2-difluoroethyl)-3-(N-(4-methoxybenzyl)methylsulfonamido)-1H-indazol-7-yl)-4-oxo-3,4-dihydroquinazolin-2-yl)-2-(3,5-difluorophenyl)ethyl)carbamate as an off-white solid, 18 g (35%). The isolated material is a mixture of stereoisomers. LCMS: M+H=907.18 and 909.12; purity=89%.

### (S)—N-(7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)methanesulfonamide

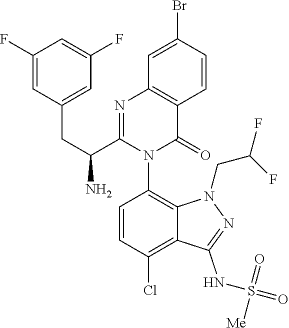 [SMILES: CS(=O)(=O)Nc1nn(CC(F)F)c2c(-n3c([C@@H](N)Cc4cc(F)cc(F)c4)nc4cc(Br)ccc4c3=O)ccc(Cl)c12]

To a stirred solution of tert-butyl (S)-(1-(7-bromo-3-(4-chloro-1-(2,2-difluoroethyl)-3-(N-(4-methoxybenzyl)methylsulfonamido)-1H-indazol-7-yl)-4-oxo-3,4-dihydroquinazolin-2-yl)-2-(3,5-difluorophenyl)ethyl)carbamate (N68085-33-A2, 15 g, 14.70 mmol) in DCM (150 mL) at 27° C. under N₂ atmosphere was added TFA (150 mL, 1947 mmol). The solution was stirred for 10 min. To the reaction mixture was added triflic acid (15 mL, 169 mmol). The solution was stirred for 1 h at 27° C. The progress of the reaction was monitored by TLC (SiO₂, 5% MeOH/DCM, Rf=0.4, UV-active). On completion, the solvent was removed under a gentle stream of nitrogen. The residue was dissolved in EtOAc (500 mL), washed with aq saturated NaHCO₃(2×250 mL), brine (150 mL), dried over Na₂SO₄ and filtered. The filtrate was concentrated under reduced pressure to afford an off-white solid. LCMS analysis of the solid found a 75.42%:21.47% ratio of diastereomers. The crude solid subjected to C18 reverse-phase column chromatography (Mobile Phase: A: 0.1% TFA in water and B: 0.1% TFA in MeCN). Pure fractions containing the major diastereomer (atropisomer) were combined concentrated under reduced pressure. The resulting aqueous solution was made basic via the addition of aq. sat. NaHCO₃; then was extracted with EtOAc (2×500 mL). The combined organic layers were washed with brine (200 mL), dried over Na₂SO₄, filtered and concentrated to afford (S)—N-(7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)methanesulfonamide as an off-white solid, 8.0 g (76%). LCMS: M+H=687.34, Purity=96%. This material was further purified to isolate the major enantiomer as follows: (S)—N-(7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)methanesulfonamide (4.5 g, 6.28 mmol) was dissolved in MeOH:MeCN (1:1, 170 mL). The solution was subjected portion-wise to SFC chiral separation as follows: column=(R, R) WHELK-01, 30×250 mm, 5 micron; solvent A=super critical CO₂; solvent B=methanol); eluent composition=50% A:50% B; flow-rate=100 g/min; back-pressure=90 bar; injection volume=1.1 mL; detection=214 nm; Stack time=6.8 min. For each isolated enantiomer, the resulting solution was concentrated under reduced pressure to afford an off-white solid. (S)—N-(7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)methanesulfonamide as was isolated as the peak eluting from 6 min to 8 min and afforded 2.1 g (48%). ¹H NMR (400 MHz, DMSO-d₆) δ=8.11-8.05 (m, 2H), 7.83-7.78 (m, 1H), 7.47-7.41 (m, 2H), 7.03-6.97 (m, 1H), 6.76-6.69 (m, 2H), 6.41-6.14 (m, 1H), 4.47-4.22 (m, 2H), 3.54-3.49 (m, 1H), 3.25-3.21 (m, 4H), 2.83-2.76 (m, 1H). LCMS: M+H=687.04, Purity=99%, Chiral HPLC Purity=96%.

### N—((S)-1-(7-Bromo-3-(4-chloro-1-(2,2-difluoroethyl)-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 2)

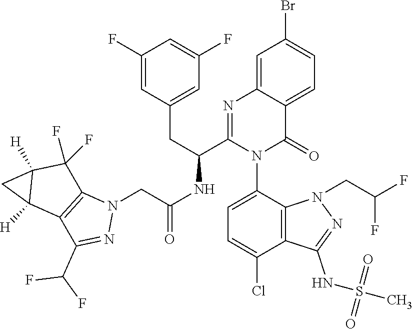 [SMILES: CS(=O)(=O)Nc1nn(CC(F)F)c2c(-n3c([C@H](Cc4cc(F)cc(F)c4)NC(=O)Cn4nc(C(F)F)c5c4C(F)(F)[C@@H]4C[C@H]54)nc4cc(Br)ccc4c3=O)ccc(Cl)c12]

To a solution of (S)—N-(7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)methanesulfonamide (1.75 g, 2.52 mmol), 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.739 g, 2.77 mmol), HOBt (0.424 g, 2.77 mmol) and EDC.HCl (0.579 g, 3.02 mmol) in DMF (15 mL) at 27° C. under nitrogen atmosphere was added N-methylmorpholine (2.215 mL, 20.15 mmol). The solution was stirred at 27° C. for 36 h. The progress of the reaction was monitored by TLC (SiO₂, 50% EtOAc/Pet. Rf=0.5, UV-active). The reaction mixture was diluted with ice cold water (50 mL), and stirred for 15 min. The precipitated solid was isolated via filtration, washed with water (50 mL), and dried under vacuum to obtain the crude product. This material was treated with EtOAc (20 mL), stirred for 15 min, and then the solids were isolated via filtration and dried under vacuum to afford N—((S)-1-(7-bromo-3-(4-chloro-1-(2,2-difluoroethyl)-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide as an off-white solid, 1.6 g (64%). ¹H NMR (400 MHz, DMSO-d₆) δ=10.00 (brs, 1H), 9.23 (d, J=8.1 Hz, 1H), 8.13 (d, J=8.6 Hz, 1H), 7.98 (d, J=2.0 Hz, 1H), 7.85 (dd, J=2.0, 2.1 Hz, 1H), 7.78 (d, J=7.9 Hz, 1H), 7.54 (d, J=7.9 Hz, 1H), 7.07-6.99 (m, 1H), 6.92 (t, J=51.7 Hz, 1H), 6.61 (d, J=6.3 Hz, 2H), 6.11 (t, J=54.6 Hz, 1H), 4.72-4.57 (m, 2H), 4.38 (tt, J=107, 2.9 Hz, 1H), 4.31-4.19 (m, 1H), 3.96-3.83 (m, 1H), 3.44-3.37 (m, 1H), 3.19 (s, 3H), 3.00-2.92 (m, 1H), 2.49-2.45 (m, 2H), 1.39-1.31 (m, 1H), 0.87-0.82 (m, 1H). LCMS: M+H=933.13, LCMS Purity=95%, HPLC Purity=96%, Chiral HPLC Purity=97%.

### tert-butyl(S)-(1-(7-bromo-3-(4-chloro-1-(2,2-difluoroethyl)-3-(N-(4-methoxybenzyl) cyclopropanesulfonamido)-1H-indazol-7-yl)-4-oxo-3,4-dihydroquinazolin-2-yl)-2-(3,5-difluorophenyl)ethyl)carbamate

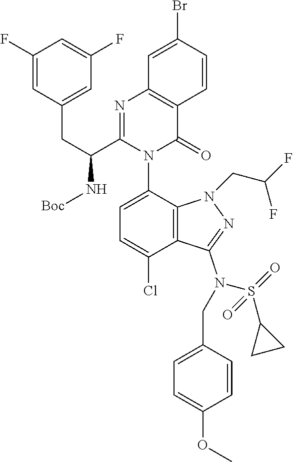 [SMILES: COc1ccc(CN(c2nn(CC(F)F)c3c(-n4c([C@H](Cc5cc(F)cc(F)c5)NC(=O)OC(C)(C)C)nc5cc(Br)ccc5c4=O)ccc(Cl)c23)S(=O)(=O)C2CC2)cc1]

To a stirred solution of (S)-2-((tert-butoxycarbonyl)amino)-3-(3,5-difluorophenyl)propanoic acid (15 g, 49.8 mmol) and 2-amino-4-bromobenzoic acid (12.91 g, 59.7 mmol) in pyridine (150 mL) in a sealed tube at 26° C. was added diphenyl phosphite (35.7 mL, 184 mmol). The reaction mixture was degassed with N₂ bubbling for each addition of reagents. The reaction mixture was heated to 80° C. and stirred for 2 hr. The reaction mixture was cooled to 26° C., then N-(7-amino-4-chloro-1-(2,2-difluoroethyl)-1H-indazol-3-yl)-N-(4-methoxybenzyl)cyclopropanesulfonamide (N66734-90-A2, 20.49 g, 34.9 mmol) was added. The mixture was heated at 80° C. for 16 h. The progress of the reaction was monitored by TLC (SiO₂, 30% EtOAc/Pet. Rf=0.3). The reaction mixture was cooled to 26° C. and then was concentrated under reduced pressure. The residue was diluted with water (150 mL) and extracted with ethyl acetate (2×500 mL). The combined organic layers were washed with aq. citric acid (5% w/v, 2×150 mL), then brine (250 mL); dried over anhydrous Na₂SO₄; filtered; and concentrated under reduced pressure to afford a brown gummy liquid (40 g). The above procedure was repeated, and the crude product of both iterations was combined. This material was then subjected to silica gel column chromatography (pet.:EtOAc, 60:40→55:45) to afforded tert-butyl (S)-(1-(7-bromo-3-(4-chloro-1-(2,2-difluoroethyl)-3-(N-(4-methoxybenzyl)cyclopropanesulfonamido)-1H-indazol-7-yl)-4-oxo-3,4-dihydroquinazolin-2-yl)-2-(3,5-difluorophenyl)ethyl)carbamate (mixture of diastereomers) as a yellow solid (42 g, 98%). LCMS: M+H=933.88 & 935.88; purity=76.91%.

### (S)—N-(7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)cyclopropanesulfonamide

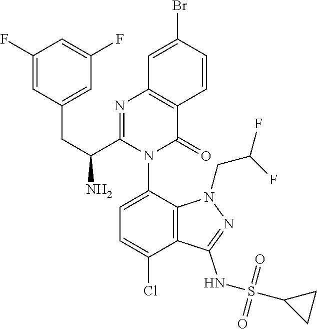 [SMILES: N[C@@H](Cc1cc(F)cc(F)c1)c1nc2cc(Br)ccc2c(=O)n1-c1ccc(Cl)c2c(NS(=O)(=O)C3CC3)nn(CC(F)F)c12]

To a stirred solution of tert-butyl (S)-(1-(7-bromo-3-(4-chloro-1-(2,2-difluoroethyl)-3-(N-(4-methoxybenzyl)cyclopropanesulfonamido)-1H-indazol-7-yl)-4-oxo-3,4-dihydroquinazolin-2-yl)-2-(3,5-difluorophenyl)ethyl)carbamate (14 g, 11.53 mmol) in DCM (140 mL) at 27° C. under N₂ atmosphere was added TFA (140 mL). The solution was stirred for 10 min. To the solution was added trifluoromethanesulfonic acid (7.16 mL, 81 mmol). The reaction mixture was stirred for 1 h at 27° C. The progress of the reaction was monitored by TLC (SiO₂, 50% EtOAc/pet, Rf=0.2). The solvent was removed under a gentle stream of nitrogen. The residue was dissolved in EtOAc (500 mL) and the organic layer was washed with aq. saturated NaHCO₃(2×150 mL), brine (50 mL), dried over Na₂SO₄, filtered and concentrated to dryness to the crude compound as an off white solid (12 g). The above procedure was repeated twice more and the additional crude solids (2×14 g) were combined with the above. The combined material was dissolved in dichloromethane (500 mL) and concentrated to afford a homogeneous crude solid. This material was washed with pet. ether:EtOAc (80:20) and then dried under vacuum to afford a brown solid (30 g). This material was then subjected to C18 reverse phase chromatography under the following conditions: Column=RediSep Gold HP C18 275 g; Mobile Phase A=Water:MeCN:TFA (950:50:1); Mobile Phase B=Water:MeCN:TFA (50:950:1); flow rate=80 mL/min; gradient profile (time/% B)=5/5, 5/10, 5/15, 10/20, 15/30, 20/40, 15/45, 10/50; temperature=ambient. Fractions of the major peak were pooled and concentrated under reduced pressure to remove the non-aqueous solvent. The resulting aq. solution was neutralized via the addition of sat. aq. NaHCO₃(1000 mL), then was extracted with EtOAc (4×500 mL). The combined organics were washed with brine (500 mL), dried over anhydrous Na₂SO₄, filtered and concentrated to afford (S)—N-(7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)cyclopropanesulfonamide (single diastereomer) as an off white solid. The material was then subjected to SFC purification under the following conditions: Column/dimensions=Chiralpak OX-H (30×250 mm), 5μ; Solvent A=liquid CO₂; Solvent B=Methanol with 0.5% diethyl amine; Eluent=A:B (70:30); Flow-rate=100.0 g/min; Back Pressure=100.0 bar; Detection=UV (214 nm); injection volume=1.3 mL (93 mg/injection); 160 injections. Two peaks were collected separately and the major peak was concentrated under reduced pressure to afford (S)—N-(7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)cyclopropanesulfonamide (single stereoisomer) as a pale yellow solid, 7.5 g (20%). ¹H NMR (400 MHz, DMSO-d₆) δ=8.11-8.04 (m, 2H), 7.82-7.78 (m, 1H), 7.47-7.39 (m, 2H), 7.02-6.95 (m, 1H), 6.76-6.69 (m, 2H), 6.38-6.19 (m, 1H), 4.48-4.37 (m, 1H), 4.32-4.24 (m, 1H), 3.54-3.48 (m, 1H), 3.3-3.20 (m, 1H), 2.97-2.90 (m, 1H), 2.83-2.76 (m, 1H), 1.05-0.99 (m, 4H). LCMS: M+H=712.94 and 714.94; purity=98.37%, chiral HPLC purity=96%.

### N—((S)-1-(7-bromo-3-(4-chloro-3-(cyclopropanesulfonamido)-1-(2,2-difluoroethyl)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 3)

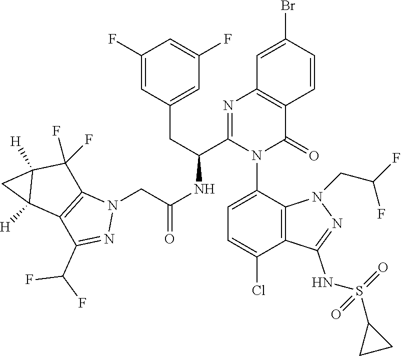 [SMILES: O=C(Cn1nc(C(F)F)c2c1C(F)(F)[C@@H]1C[C@H]21)N[C@@H](Cc1cc(F)cc(F)c1)c1nc2cc(Br)ccc2c(=O)n1-c1ccc(Cl)c2c(NS(=O)(=O)C3CC3)nn(CC(F)F)c12]

To a stirred solution of (S)—N-(7-(2-(1-amino-2-(3,5-difluorophenyl)ethyl)-7-bromo-4-oxoquinazolin-3(4H)-yl)-4-chloro-1-(2,2-difluoroethyl)-1H-indazol-3-yl)cyclopropanesulfonamide (500 mg, 0.700 mmol), 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (N68084-15-A1, 185 mg, 0.700 mmol), and HOBt (42.9 mg, 0.280 mmol) in DMF (5 mL) at 27° C. was added N-methylmorpholine (0.308 mL, 2.80 mmol) and N-(3-dimethylaminopropyl)-N′-ethylcarbodiimide hydrochloride (242 mg, 1.261 mmol). The reaction mixture was stirred at 27° C. for 16 h. The progress of the reaction was monitored by TLC (SiO₂, 50% EtOAc/Pet., Rf=0.3, UV-active). On completion, the reaction mixture was diluted with ice cold water (70 mL) and then stirred for 15 min at 27° C. The precipitated solids were collected by filtration and then dried under vacuum to obtain the crude compound as an off-white solid. The crude compound was subjected to silica gel chromatography (pet.:EtOAc (98:2→50:50) to afford N—((S)-1-(7-bromo-3-(4-chloro-3-(cyclopropanesulfonamido)-1-(2,2-difluoroethyl)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide as an off-white solid, 550 mg (80%). ¹H NMR (400 MHz, DMSO-d₆) δ=9.99 (s, 1H), 9.24 (d, J=8.1 Hz, 1H), 8.13 (d, J=8.8 Hz, 1H), 7.97 (d, J=1.8 Hz, 1H), 7.87-7.83 (m, 1H), 7.77 (d, J=7.9 Hz, 1H), 7.54 (d, J=7.9 Hz, 1H), 7.06-6.79 (m, 2H), 6.64-6.58 (m, 2H), 6.23-5.98 (m, 1H), 4.74-4.57 (m, 2H), 4.41-4.35 (m, 1H), 4.29-4.16 (m, 1H), 3.94-3.84 (m, 1H), 3.38-3.34 (m, 1H), 3.02-2.93 (m, 1H), 2.90-2.83 (m, 1H), 2.48-2.35 (m, 2H), 1.37-1.30 (m, 1H), 1.02-0.90 (m, 4H), 0.87-0.82 (m, 1H). LCMS analysis method F: RT=6.74 mins, (M+H)=959.0 and 961.0; LCMS Purity=98%; Chiral HPLC Purity=98%.

### N—((S)-1-(3-(4-chloro-1-methyl-3-(methylsulfonamido)-1H-indazol-7-yl)-4-oxo-7-(4,4,5,5-tetramethyl-1,3,2-dioxaborolan-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

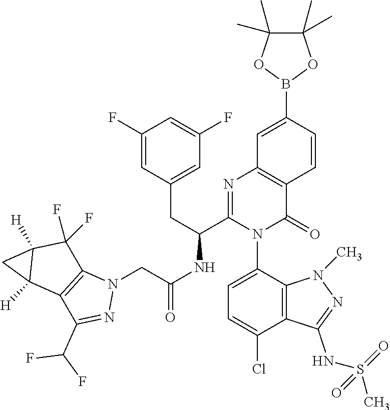 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(B5OC(C)(C)C(C)(C)O5)ccc4c3=O)c21]

To a round bottom flask equipped with a stir bar was added N—((S)-1-(7-bromo-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.00 g, 1.13 mmol), 4,4,4′,4′,5,5,5′,5′-octamethyl-2,2′-bi(1,3,2-dioxaborolane) (431 mg, 1.70 mmol), potassium acetate (333 mg, 3.39 mmol), and [1,1′-bis(diphenylphosphino)ferrocene]dichloropalladium(II) (“Pd(dppf)Cl₂”) (83 mg, 0.113 mmol). The flask was sealed with a rubber septum, and then was placed under an argon atmosphere. To the flask was added dioxane (23 mL). The reaction mixture was degassed with argon, then the reaction mixture was stirred at 60° C. for 16 h. The reaction mixture was concentrated in vacuo and adsorbed onto Celite. The resulting powder was subjected to silica gel chromatography (hexanes:EtOAc 100:0→0:100 over 10 column volumes) to afford N—((S)-1-(3-(4-chloro-1-methyl-3-(methylsulfonamido)-1H-indazol-7-yl)-4-oxo-7-(4,4,5,5-tetramethyl-1,3,2-dioxaborolan-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.2 g, quantitative yield). LCMS: During LCMS analysis both the boronic acid and boronate were observed. Conditions: Wavelength: 220 nm, Wavelength2: 254 nm, Injection Vol.: 5.00 μl, Stop Time: 4.00, Grad. Time: 3.0, Start % B: 0, End % B: 100, Total Flow: 0.80 ml/min, Solvent A: 95:5 Water:MeCN 0.1% TFA, Solvent B: 5:95 Water:MeCN 0.1% TFA, Column: Acquity UPLC BEH C18 1.7 um; Result: retention time (boronic acid): 2.112 min., mass found: 849.15 (M+H); retention time (boronic ester): 2.733 min., mass found: 931.25 (M+H). ¹H NMR (CDCl₃, 500 MHz) δ 8.26 (d, 1H, J=7.6 Hz), 8.11 (s, 1H), 7.95 (d, 1H, J=7.6 Hz), 7.3-7.3 (m, 1H), 7.14 (d, 1H, J=7.9 Hz), 6.7-6.7 (m, 3H), 6.35 (d, 2H, J=6.8 Hz), 4.7-4.8 (m, 1H), 4.1-4.2 (m, 1H), 3.70 (s, 1H), 3.47 (s, 3H), 3.37 (s, 3H), 3.1-3.2 (m, 1H), 2.8-2.9 (m, 1H), 2.6-2.7 (m, 1H), 2.3-2.5 (m, 1H), 1.8-1.9 (m, 2H), 1.24 (s, 12H), 1.1-1.2 (m, 1H)

### N—((S)-1-(3-(4-chloro-1-(2,2-difluoroethyl)-3-(methylsulfonamido)-1H-indazol-7-yl)-4-oxo-7-(4,4,5,5-tetramethyl-1,3,2-dioxaborolan-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

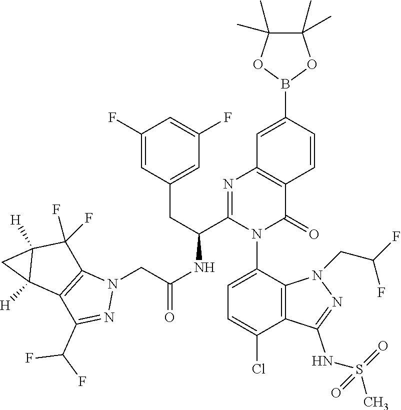 [SMILES: CC1(C)OB(c2ccc3c(=O)n(-c4ccc(Cl)c5c(NS(C)(=O)=O)nn(CC(F)F)c45)c([C@H](Cc4cc(F)cc(F)c4)NC(=O)Cn4nc(C(F)F)c5c4C(F)(F)[C@@H]4C[C@H]54)nc3c2)OC1(C)C]

To a dry round-bottom flask equipped with a stir bar was added N—((S)-1-(7-bromo-3-(4-chloro-1-(2,2-difluoroethyl)-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (500 mg, 0.535 mmol), 4,4,4′,4′,5,5,5′,5′-octamethyl-2,2′-bi(1,3,2-dioxaborolane) (204 mg, 0.803 mmol), potassium acetate (158 mg, 1.606 mmol), and PdCl₂(dppf) (39.2 mg, 0.054 mmol). The flask was sealed with a septum and then placed under argon atmosphere (vac/fill×3). To the flask was added 1,4-dioxane (14 mL). The mixture was degassed (vac/fill with argon×3). The mixture was then stirred at 60° C. for overnight (16 h). The reaction mixture was concentrated under reduced pressure. The resulting residue was adsorbed onto Celite. The resulting powder was subjected to silica gel column chromatography (40 g silica gel column, hexanes:EtOAc 100:0→50:50 over 10 column volumes). The fractions containing the product were collected and concentrated in vacuo to afford N—((S)-1-(3-(4-chloro-1-(2,2-difluoroethyl)-3-(methylsulfonamido)-1H-indazol-7-yl)-4-oxo-7-(4,4,5,5-tetramethyl-1,3,2-dioxaborolan-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide, 520 mg (99%). ¹H NMR (METHANOL-d₄, 500 MHz) δ 8.2-8.3 (m, 2H), 7.97 (d, 1H, J=7.7 Hz), 7.40 (d, 1H, J=8.0 Hz), 7.28 (d, 1H, J=8.0 Hz), 6.5-6.9 (m, 4H), 6.00 (tt, 1H, J=4.1, 55.2 Hz), 4.75 (dd, 1H, J=4.8, 9.2 Hz), 4.6-4.7 (m, 2H), 4.38 (dtd, 1H, J=4.2, 13.3, 15.2 Hz), 4.12 (q, 1H, J=7.2 Hz), 3.9-4.0 (m, 1H), 3.3-3.5 (m, 1H), 3.3-3.3 (m, 3H), 3.06 (dd, 1H, J=9.2, 14.0 Hz), 2.4-2.5 (m, 2H), 2.0-2.0 (m, 2H), 1.3-1.4 (m, 2H), 1.22 (s, 12H), 1.0-1.1 (m, 1H).

### N—((S)-1-(3-(4-chloro-3-(cyclopropanesulfonamido)-1-(2,2-difluoroethyl)-1H-indazol-7-yl)-4-oxo-7-(4,4,5,5-tetramethyl-1,3,2-dioxaborolan-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

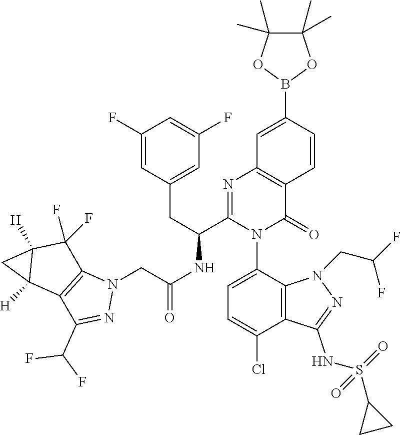 [SMILES: CC1(C)OB(c2ccc3c(=O)n(-c4ccc(Cl)c5c(NS(=O)(=O)C6CC6)nn(CC(F)F)c45)c([C@H](Cc4cc(F)cc(F)c4)NC(=O)Cn4nc(C(F)F)c5c4C(F)(F)[C@@H]4C[C@H]54)nc3c2)OC1(C)C]

To a dry r.b. flask equipped with a stir bar was added N—((S)-1-(7-bromo-3-(4-chloro-3-(cyclopropanesulfonamido)-1-(2,2-difluoroethyl)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (300 mg, 0.312 mmol), 4,4,4′,4′,5,5,5′,5′-octamethyl-2,2′-bi(1,3,2-dioxaborolane) (119 mg, 0.469 mmol), potassium acetate (92 mg, 0.937 mmol) and PdCl₂(dppf) (22.86 mg, 0.031 mmol). The flask was sealed with a septum and then placed under argon atmosphere (vac/fill×3). To the flask was added dioxane (6.3 mL). The flask was again placed under argon atmosphere (vac/fill×3). The resulting mixture was stirred at 60° C. for 16 h overnight. Upon cooling to ambient temperature the reaction was concentrated in vacuo and the resulting residue was adsorbed onto Celite. The resulting powder was subjected to silica gel column chromatography (hexanes:EtOAc 100:0→0:100 over 10 CV) to afford N—((S)-1-(3-(4-chloro-3-(cyclopropanesulfonamido)-1-(2,2-difluoroethyl)-1H-indazol-7-yl)-4-oxo-7-(4,4,5,5-tetramethyl-1,3,2-dioxaborolan-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide, 220 mg (70%). ¹H NMR (METHANOL-d₄, 500 MHz) δ 8.27 (d, 2H, J=6.2 Hz), 8.26 (s, 1H), 7.97 (dd, 1H, J=1.0, 7.9 Hz), 7.41 (d, 1H, J=7.7 Hz), 7.29 (d, 1H, J=7.7 Hz), 6.8-6.8 (m, 1H), 6.70 (br t, 1H, J=54.8 Hz), 6.55 (dd, 2H, J=2.1, 8.0 Hz), 6.01 (t, 1H, J=55.3 Hz), 4.74 (dd, 1H, J=4.8, 9.5 Hz), 4.68 (d, 1H, J=16.4 Hz), 4.59 (d, 1H, J=16.4 Hz), 4.38 (dd, 1H, J=4.2, 15.2 Hz), 4.12 (q, 1H, J=7.2 Hz), 3.91 (dd, 1H, J=3.9, 15.2 Hz), 3.68 (s, 1H), 3.06 (dd, 1H, J=9.4, 14.2 Hz), 2.9-2.9 (m, 1H), 2.4-2.5 (m, 2H), 2.03 (s, 2H), 1.45 (s, 12H), 1.1-1.1 (m, 2H), 1.0-1.0 (m, 3H).

### N—((S)-1-(3-(4-chloro-1-methyl-3-(methylsulfonamido)-1H-indazol-7-yl)-7-hydroxy-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 4)

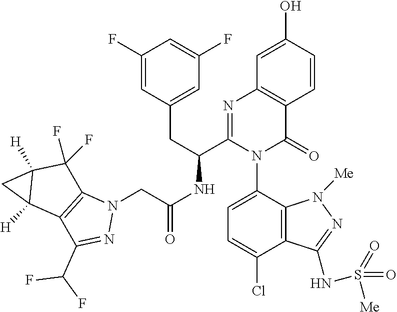 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(O)ccc4c3=O)c21]

To a stirred solution of (S)—N-(7-(2-(1-amino-2-(3,5-difluorophenyl)ethyl)-7-hydroxy-4-oxoquinazolin-3(4H)-yl)-4-chloro-1-methyl-1H-indazol-3-yl)methanesulfonamide (473 mg, 0.823 mmol), (S)—N-(7-(2-(1-amino-2-(3,5-difluorophenyl)ethyl)-7-hydroxy-4-oxoquinazolin-3(4H)-yl)-4-chloro-1-methyl-1H-indazol-3-yl)methanesulfonamide (473 mg, 0.823 mmol), and diisopropylethylamine (“DIEA”, 431 μl, 2.468 mmol) in THF (8.2 mL) at room temperature was added HATU (313 mg, 0.823 mmol). The resulting mixture was stirred at room temp for 1 h. To the mixture was added ammonia in methanol (2M, 3 mL) (to break down any acyl sulfonamide that may result from over-coupling). The mixture was stirred for 30 min and then was concentrated in vacuo. The resulting residue was combined with a similar crude material prepared by the same procedure but using 25 mg of (S)—N-(7-(2-(1-amino-2-(3,5-difluorophenyl)ethyl)-7-hydroxy-4-oxoquinazolin-3(4H)-yl)-4-chloro-1-methyl-1H-indazol-3-yl)methanesulfonamide where all other reagents and solvents were scaled accordingly. The combined crude material was subjected to silica gel column chromatography (hexanes:EtOAc 100:0→0:100 over 10 CV) to afford N—((S)-1-(3-(4-chloro-1-methyl-3-(methylsulfonamido)-1H-indazol-7-yl)-7-hydroxy-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide, 472 mg (70%). ¹H NMR (METHANOL-d₄, 500 MHz) δ 8.1-8.2 (m, 1H), 7.3-7.3 (m, 1H), 7.19 (d, 1H, J=1.5 Hz), 7.11 (br d, 2H, J=8.2 Hz), 6.7-6.8 (m, 2H), 6.5-6.6 (m, 2H), 3.61 (s, 3H), 2.4-2.5 (m, 3H), 1.3-1.4 (m, 2H), 1.2-1.3 (m, 6H), 1.1-1.2 (m, 1H), 1.0-1.0 (m, 1H).

### N—((S)-1-(7-bromo-3-(4-chloro-3-(N-(4-methoxybenzyl)methylsulfonamido)-1-methyl-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

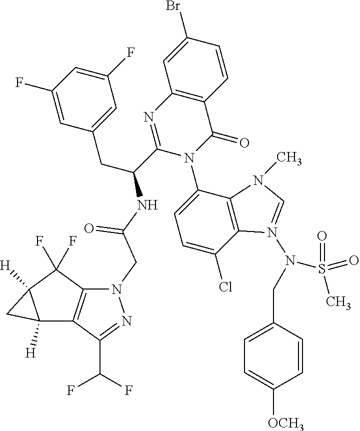

1-(chloromethyl)-4-methoxybenzene (0.276 mL, 2.036 mmol) was added to a stirred solution of N—((S)-1-(7-bromo-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.5 g, 1.697 mmol) and cesium carbonate (0.553 g, 1.697 mmol) in N,N-Dimethylformamide (DMF) (10 mL) and the resulting mixture was stirred at room temp for 16 h. Water was then added and the mixture was extracted with ethyl acetate, washed with brine, dried (Na₂SO₄), filtered and concentrated. The residue was then subjected to silica gel column chromatography (hexanes:EtOAc 95:5→70:30) to afford N—((S)-1-(7-bromo-3-(4-chloro-3-(N-(4-methoxybenzyl)methylsulfonamido)-1-methyl-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide, 1.4 g (82%). LCMS analysis conditions: Wavelength1: 220 nm; Wavelength2: 254 nm; Injection Vol.: 5.00 μl; Stop Time: 4.50 min; Grad. Time: 3.50 min; Start % B: 0; End % B: 100; Total Flow: 0.80 ml/min; Solvent A: 95:5 Water:MeCN with 0.1% TFA; Solvent B: 5:95 Water:MeCN with 0.1% TFA; Column=Acquity UPLC BEH C18, 2.1×100 mm, 1.7 μm. LCMS analysis result: retention time: 3.536 min, M+H: 1003.05.

### N—((S)-1-(3-(4-chloro-3-(N-(4-methoxybenzyl)methylsulfonamido)-1-methyl-1H-indazol-7-yl)-4-oxo-7-(4,4,5,5-tetramethyl-1,3,2-dioxaborolan-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

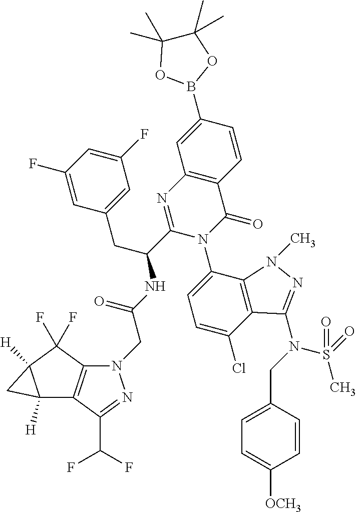 [SMILES: COc1ccc(CN(c2nn(C)c3c(-n4c([C@H](Cc5cc(F)cc(F)c5)NC(=O)Cn5nc(C(F)F)c6c5C(F)(F)[C@@H]5C[C@H]65)nc5cc(B6OC(C)(C)C(C)(C)O6)ccc5c4=O)ccc(Cl)c23)S(C)(=O)=O)cc1]

N—((S)-1-(7-bromo-3-(4-chloro-3-(N-(4-methoxybenzyl)methylsulfonamido)-1-methyl-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (954 mg, 0.950 mmol), 4,4,4′,4′,5,5,5′,5′-octamethyl-2,2′-bi(1,3,2-dioxaborolane) (362 mg, 1.425 mmol), potassium acetate (280 mg, 2.85 mmol) and PdCl₂(dppf) (69.5 mg, 0.095 mmol) were combined dry and degassed with Ar. Then they were taken up in dioxane (19 mL) and degassed again with argon and the resulting mixture was stirred at 60° C. overnight (16 h). The reaction mixture was concentrated, adsorbed onto Celite and, the resulting powder was subjected to silica gel column chromatography (hexanes:EtOAc 100:0→0:100 over 10 CVs) to afford N—((S)-1-(3-(4-chloro-3-(N-(4-methoxybenzyl)methylsulfonamido)-1-methyl-1H-indazol-7-yl)-4-oxo-7-(4,4,5,5-tetramethyl-1,3,2-dioxaborolan-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide, 709 mg (71%). Under LCMS analysis both the boronic ester and the boronic acid are observed. However, 1H-NMR indicates that the product is entirely the boronic ester. LCMS analysis conditions: Wavelength1: 220 nm; Wavelength2: 254 nm; Injection Vol.: 5.00 μl; Stop Time: 2.50 min; Grad. Time: 1.50 min; Start % B: 0; End % B: 100; Total Flow: 0.80 ml/min; Solvent A: 95:5 Water:MeCN with 0.1% TFA; Solvent B: 5:95 Water:MeCN with 0.1% TFA; Column=Acquity UPLC BEH C18, 2.1×50 mm, 1.7 um. LCMS analysis result: retention time: 1.495 min, M+H: 969.15; retention time: 1.760 min, M+H: 1051.25.

### N—((S)-1-(3-(4-chloro-3-(N-(4-methoxybenzyl)methylsulfonamido)-1-methyl-1H-indazol-7-yl)-7-hydroxy-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

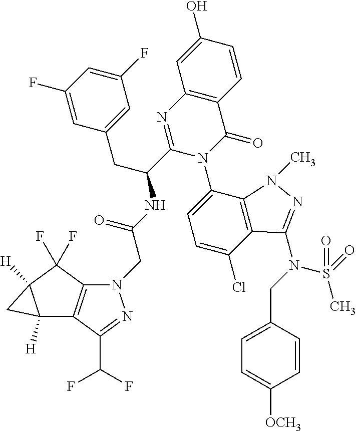 [SMILES: COc1ccc(CN(c2nn(C)c3c(-n4c([C@H](Cc5cc(F)cc(F)c5)NC(=O)Cn5nc(C(F)F)c6c5C(F)(F)[C@@H]5C[C@H]65)nc5cc(O)ccc5c4=O)ccc(Cl)c23)S(C)(=O)=O)cc1]

To a 20 mL scintillation vial charged with a solution of N—((S)-1-(3-(4-chloro-3-(N-(4-methoxybenzyl)methylsulfonamido)-1-methyl-1H-indazol-7-yl)-4-oxo-7-(4,4,5,5-tetramethyl-1,3,2-dioxaborolan-2-yl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (709 mg, 0.674 mmol) in THF (6.7 mL) was added aq. H₂O₂ (30%, 303 μL, 2.97 mmol). The mixture was stirred at room temperature for 1 h. The mixture was diluted with EtOAc and then washed with aq sat. sodium metabisulfite solution. The organic phase isolated, then concentrated in vacuo. The resulting residue was dissolved/suspended in DCM and then adsorbed onto Celite. The resulting powder was subjected to silica gel column chromatography (DCM:MeOH 100:0→90:10 over 10 CVs) to afford N—((S)-1-(3-(4-chloro-3-(N-(4-methoxybenzyl)methylsulfonamido)-1-methyl-1H-indazol-7-yl)-7-hydroxy-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide, 630 mg (99%). LCMS analysis conditions: Wavelength1: 220 nm; Wavelength2: 254 nm; Injection Vol.: 5.00 μl; Stop Time: 2.50 min; Grad. Time: 1.50 min; Start % B: 0; End % B: 100; Total Flow: 0.80 ml/min; Solvent A: 95:5 Water:MeCN with 0.1% TFA; Solvent B: 5:95 Water:MeCN with 0.1% TFA; Column=Acquity UPLC BEH, 2.1×50 mm, 1.7 μm. LCMS analysis result=retention time: 1.534 min, M+H: 941.10.

### (S)-tert-Butyl (1-(3-(4-chloro-3-(N-(4-methoxybenzyl)methylsulfonamido)-1-methyl-1H-indazol-7-yl)-7-methoxy-4-oxo-3,4-dihydroquinazolin-2-yl)-2-(3,5-difluorophenyl)ethyl)carbamate

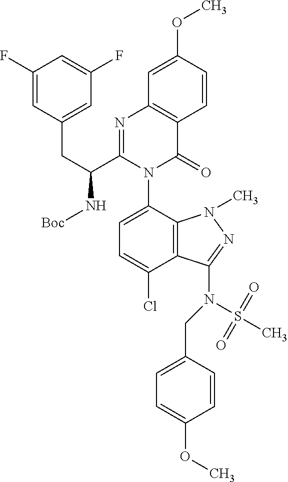 [SMILES: COc1ccc(CN(c2nn(C)c3c(-n4c([C@H](Cc5cc(F)cc(F)c5)NC(=O)OC(C)(C)C)nc5cc(OC)ccc5c4=O)ccc(Cl)c23)S(C)(=O)=O)cc1]

To a stirred solution of (S)-2-((tert-butoxycarbonyl)amino)-3-(3,5-difluorophenyl)propanoic acid (5 g, 16.60 mmol) and 2-amino-4-methoxybenzoic acid (2.95 g, 16.93 mmol) in pyridine (75 mL) was added diphenyl phosphite (11.24 mL, 58.1 mmol) at 27° C. The flask was flushed with argon, sealed, and then stirred at 70° C. for 2 hr. The reaction mixture was allowed to cool to 27° C., then to the mixture was added N-(7-amino-4-chloro-1-methyl-1H-indazol-3-yl)-N-(4-methoxybenzyl)methanesulfonamide (5.52 g, 13.28 mmol). The reaction vessel was resealed under argon and the mixture was stirred at 70° C. for 16 hr. The progress of the reaction was monitored by TLC (SiO₂, 30% EtOAc/Pet., Rf=0.4, UV-active). The reaction mixture was concentrated under reduced pressure to get crude compound, which was purified by column chromatography on silica gel (SiO₂, 100-200 mesh) with 20-30% EtOAc/Pet. The fractions containing product were collected and concentrated under reduced pressure to afford (S)-tert-butyl (1-(3-(4-chloro-3-(N-(4-methoxybenzyl)methylsulfonamido)-1-methyl-1H-indazol-7-yl)-7-methoxy-4-oxo-3,4-dihydroquinazolin-2-yl)-2-(3,5-difluorophenyl)ethyl)carbamate (5 g, Yield: 34%, off-white solid) as a mixture of stereoisomers. LCMS: RT=2.46 min, (M+H)=809.14, Purity=93%.

### (S)—N-(7-(2-(1-amino-2-(3,5-difluorophenyl)ethyl)-7-methoxy-4-oxoquinazolin-3(4H)-yl)-4-chloro-1-methyl-1H-indazol-3-yl)methanesulfonamide

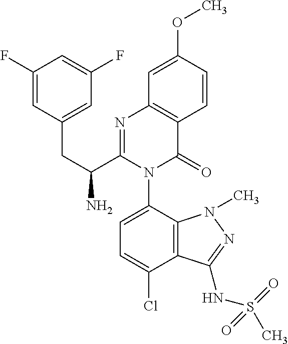 [SMILES: COc1ccc2c(=O)n(-c3ccc(Cl)c4c(NS(C)(=O)=O)nn(C)c34)c([C@@H](N)Cc3cc(F)cc(F)c3)nc2c1]

To a stirred solution of (S)-tert-butyl (1-(3-(4-chloro-3-(N-(4-methoxybenzyl)methylsulfonamido)-1-methyl-1H-indazol-7-yl)-7-methoxy-4-oxo-3,4-dihydroquinazolin-2-yl)-2-(3,5-difluorophenyl)ethyl)carbamate (2.3 g, 2.64 mmol) in DCM (60 mL) was added TFA (115 mL, 1493 mmol) at 27° C. The solution was stirred for 10 min. To the solution was added trifluoromethanesulfonic acid (1.725 mL, 19.43 mmol). The solution was stirred at 27° C. for 1 hr under nitrogen atmosphere. The progress of the reaction was monitored by TLC (SiO₂, 5% MeOH/DCM, Rf=0.4, UV-active). The solvent was removed under a gentle stream of nitrogen gas. The residue was dissolved in EtOAc (500 mL), washed with aq. saturated NaHCO₃(2×250 mL), brine (150 mL), dried over Na₂SO₄, filtered and concentered under reduced pressure to obtain the crude compound (1.5 g, off-white solid). The crude compound is a mixture of stereoisomers. LCMS: RT=4.557 mins, (M+H)=589.1, Purity=75% and RT=5.109 mins, (M+H)=589.1, Purity=23%. Multiple lots of (S)—N-(7-(2-(1-amino-2-(3,5-difluorophenyl)ethyl)-7-methoxy-4-oxoquinazolin-3(4H)-yl)-4-chloro-1-methyl-1H-indazol-3-yl)methanesulfonamide prepared as described above were combined prior to separation of the stereoisomers. The crude solids of each lot were dissolved in EtOAc. The solution was concentrated under reduced pressure to afford the crude compound (2 g, 3.23 mmol). The crude was purified on SFC by the following method: Column/dimensions=Chiralpak IC (30×250 mm), 5μ; CO₂ (%)=65.0%; Co-solvent (%)=35.0%; Co-Solvent=0.5% diethylamine in methanol; Total Flow=90.0 g/min; Back Pressure=100.0 bar; UV detection=214 nm; Stack time=6.7 min; Load/Inj=42.0 mg; Load solvent=Methanol+ACN+DCM+THF+DEA (60+60+10+5+3) mL; Number of injections=60. The major peak isolated from the SFC purification was collected and concentrated under reduced pressure to afford (S)—N-(7-(2-(1-amino-2-(3,5-difluorophenyl)ethyl)-7-methoxy-4-oxoquinazolin-3(4H)-yl)-4-chloro-1-methyl-1H-indazol-3-yl)methanesulfonamide (900 mg, Yield: 46%, off-white solid). ¹HNMR (400 MHz, CDCl₃) δ=8.19 (d, J=8.8 Hz, 1H), 7.21-7.18 (m, 1H), 7.14-7.04 (m, 2H), 6.68 (tt, J=8.9, 2.2 Hz, 1H), 6.56-6.37 (m, 3H), 4.00 (s, 3H), 3.80-3.66 (m, 3H), 3.65-3.59 (m, 1H), 3.42-3.35 (m, 3H), 3.30 (dd, J=13.0, 6.9 Hz, 1H), 2.88-2.76 (m, 1H). LCMS: RT=2.698 mins, (M+H)=589.1, LCMS Purity=98%, Chiral HPLC Purity=97%.

### 2-Chloro-6-methoxynicotinic acid

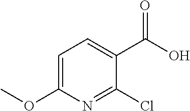

To a stirred solution of 2,6-dichloronicotinic acid (40 g, 208 mmol) in MeOH (800 mL) was added potassium tert-butoxide (117 g, 1042 mmol) portion wise at 27° C. The reaction mixture was slowly heated to 80° C. and stirred for 16 h. The progress of the reaction was monitored by TLC (SiO₂, 10% MeOH/DCM, Rf=0.4, UV-active). Upon completion, the reaction mixture was concentrated under reduced pressure and then diluted with water (100 mL) and acidified with 1N HCl (to pH ˜3, 1000 mL) until white precipitate was obtained. The precipitated solid was collected via filtration and then dried under vacuum to afford 2-chloro-6-methoxynicotinic acid (48 g, Yield: 97%, off-white solid). ¹HNMR (400 MHz, DMSO-d₆) δ=8.18 (d, J=8.2 Hz, 1H), 6.92 (d, J=8.3 Hz, 1H), 3.91 (s, 3H). LCMS: RT=1.45 mins, (M+H)=186.2, Purity=76%.

### methyl 2-chloro-6-methoxynicotinate

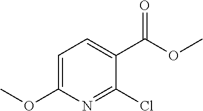

To a stirred solution of 2-chloro-6-methoxynicotinic acid (48 g, 202 mmol) in MeOH (240 mL) was added thionyl chloride (93.6 mL, 1282 mmol) dropwise at 0° C. The reaction mixture was heated to 80° C. and stirred for 16 h. The progress of the reaction was monitored by TLC (SiO₂, 10% MeOH/DCM, Rf=0.4, UV-active). Upon completion, the reaction mixture was allowed to cool to 27° C. and then was quenched with ice-cold water (500 mL) and extracted with Pet. ether (4×500 mL). The combined organic layers were washed with NaHCO₃(2×700 mL), dried over Na₂SO₄ and filtered. The filtrate was concentrated under reduced pressure to afford methyl 2-chloro-6-methoxynicotinate (30 g, Yield: 70%, off-white solid). ¹HNMR (400 MHz, DMSO-d₆) δ=8.20 (d, J=8.4 Hz, 1H), 6.95 (d, J=8.5 Hz, 1H), 3.93 (s, 3H), 3.84 (s, 3H). LCMS: RT=1.82 mins, (M+H)=202.15, Purity=95%.

### methyl 6-methoxy-2-((4-methoxybenzyl)amino)nicotinate

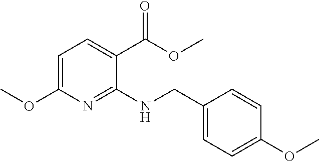

To a stirred solution of methyl 2-chloro-6-methoxynicotinate (30 g, 141 mmol) in DMF (150 mL) was added 4-methoxybenzylamine (22.16 mL, 170 mmol) and potassium carbonate (29.3 g, 212 mmol) at 0° C. The reaction mixture was heated to 100° C. and stirred for 10 h. The progress of the reaction was monitored by TLC (SiO₂, 10% EtOAc/Pet., Rf=0.5, UV-active). Upon completion, the reaction mixture was allowed to cool to 27° C., and then was quenched with ice-cold water (100 mL) and stirred 15 min. The precipitated solid was collected via filtration and then dried under vacuum to obtain the crude compound. This material was purified by column chromatography on silica gel (SiO₂, 100-200 mesh) eluting with 0-5% EtOAc/Pet. The fractions containing product were collected and concentrated under reduced pressure to afford methyl 6-methoxy-2-((4-methoxybenzyl)amino)nicotinate (32 g, Yield: 72%, off-white solid). ¹HNMR (400 MHz, DMSO-d₆) δ=8.52-8.46 (m, 1H), 7.96 (d, J=8.6 Hz, 1H), 7.31-7.27 (m, 2H), 6.90-6.87 (m, 2H), 6.00 (d, J=8.5 Hz, 1H), 4.60 (d, J=5.8 Hz, 2H), 3.83 (s, 3H), 3.76 (s, 3H), 3.72 (s, 3H). LCMS: RT=2.81 mins, (M+H)=303.10, Purity=96%.

### Preparation of methyl 2-amino-6-methoxynicotinate

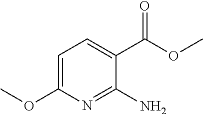

To a stirred solution of methyl 6-methoxy-2-((4-methoxybenzyl) amino)nicotinate (16 g, 51.3 mmol) in EtOH (150 mL) at 27° C. under nitrogen atmosphere was added Pd—C (16.39 g, 15.40 mmol) portion-wise. The reaction mixture was purged with hydrogen gas and then stirred under balloon-pressure hydrogen atmosphere for 16 h. The progress of the reaction was monitored by TLC (SiO₂, 30% EtOAc/Pet., Rf=0.3, UV-active). Upon completion, the reaction mixture was filtered through a small pad of Celite and the filter cake was extracted with DCM (200 mL). The filtrate was concentrated under reduced pressure to obtain methyl 2-amino-6-methoxynicotinate (15 g, off-white solid). This procedure was repeated to afford a second batch of crude product. Both batches were dissolved in EtOAc and blended. The solution was concentrated under reduced pressure to afford the combined crude compound (32 g, 79 mmol). This material was purified by column chromatography on silica gel (SiO₂, 100-200 mesh) eluting with 0-30% EtOAc/Pet. The fractions containing product were collected and concentrated under reduced procedure to afford methyl 2-amino-6-methoxynicotinate (14 g, Yield: 97%, off-white solid). ¹HNMR (400 MHz, DMSO-d₆) δ=7.93 (d, J=8.6 Hz, 1H), 7.28 (br s, 2H), 6.01 (d, J=8.6 Hz, 1H), 3.82 (s, 3H), 3.76 (s, 3H). LCMS: RT=1.89 mins, (M+H)=183.21, Purity=99%.

### 2-Amino-6-methoxynicotinic acid

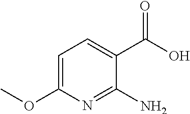

To a stirred solution of methyl 2-amino-6-methoxynicotinate (14 g, 76 mmol) in MeOH (60 mL) and THF (60 mL) at 27° C. was added a solution of LiOH (7.29 g, 304 mmol) in water (60 mL). The reaction mixture was stirred for 16 h. The progress of reaction was monitored by TLC (SiO₂, 50% EtOAc/Pet., Rf=0.1, UV-active). Upon completion, the reaction mixture was concentrated under reduced pressure to obtain the crude residue. The crude residue was diluted with water (20 mL) and neutralised with 1N HCl (100 mL). The mixture was stirred for 10 min. The precipitated solid were collected via filtration and dried under vacuum to afford of 2-amino-6-methoxynicotinic acid (10 g, Yield: 77%, off white solid). ¹HNMR (400 MHz, DMSO-d₆) δ=12.42 (br s, 1H), 7.91 (d, J=8.5 Hz, 1H), 7.31 (br s, 2H), 5.99 (d, J=8.6 Hz, 1H), 3.82 (s, 3H). LCMS: RT=1.30 mins, (M+H)=169.15, Purity=98%.

### 2-amino-5-chloro-6-methoxynicotinic acid

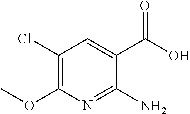

To a slurry of 2-amino-6-methoxynicotinic acid (0.61 g, 3.63 mmol, 1 equiv) in AcOH (12 mL) was added NCS (0.51 g, 3.81 mmol, 1.05 equiv). After 18 h, more NCS (0.30 g, 2.25 mmol, 0.6 equiv) was added. After stirring 1 d, the mixture was added slowly to water (400 mL). The tan precipitate was filtered to provide the product (0.48 g, 65%). 1H NMR (500 MHz, DMSO-d₆) δ 12.74 (br s, 1H), 7.93 (s, 1H), 7.40 (bs, 2H), 3.91 (s, 3H). LCMS (M+1): 203.10.

### 2-amino-6-isopropoxynicotinic acid

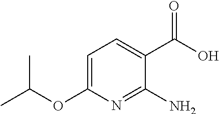

A solution of 2-amino-6-chloronicotinic acid (4 g, 23.2 mmol, 1 equiv) and KOtBu (7.8 g, 69.5 mmol, 3 equiv) in isopropanol (77 mL) was heated in a sealed tube at 120° C. for 22 h. Upon cooling to ambient temperature, the slurry was added to water and washed with ether (×2). The aqueous layer was then neutralized with AcOH (8 mL). The resultant precipitate was filtered to provide the product (4.4 g, 97%) as a tan solid. ¹H NMR (500 MHz, DMSO-d₆) δ ppm 12.12-12.59 (m, 1H), 7.75-8.04 (m, 1H), 6.79-7.50 (m, 2H), 5.77-6.07 (m, 1H), 5.07-5.38 (m, 1H), 1.07-1.43 (m, 6H). LCMS (M+MeCN): 238.15.

### (S)—N-(1-(3-(4-chloro-1-methyl-3-(methylsulfonamido)-1H-indazol-7-yl)-7-methoxy-4-oxo-3,4-dihydroquinazolin-2-yl)-2-(3,5-difluorophenyl)ethyl)-2-(3-isopropyl-1H-pyrazol-1-yl)acetamide (Example 5)

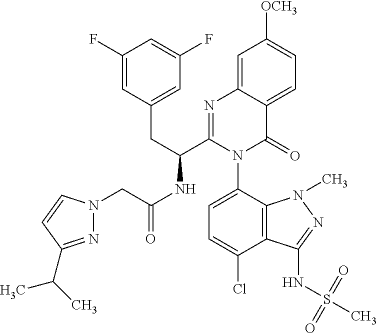 [SMILES: COc1ccc2c(=O)n(-c3ccc(Cl)c4c(NS(C)(=O)=O)nn(C)c34)c([C@H](Cc3cc(F)cc(F)c3)NC(=O)Cn3ccc(C(C)C)n3)nc2c1]

To a stirred solution of (S)—N-(7-(2-(1-amino-2-(3,5-difluorophenyl)ethyl)-7-methoxy-4-oxoquinazolin-3(4H)-yl)-4-chloro-1-methyl-1H-indazol-3-yl)methanesulfonamide (50 mg, 0.083 mmol), 2-(3-isopropyl-1H-pyrazol-1-yl)acetic acid (15.55 mg, 0.092 mmol), HOBt (14.01 mg, 0.092 mmol) and EDC.HCl (19.14 mg, 0.100 mmol) in DMF (2 mL) was added N-methylmorpholine (0.073 mL, 0.666 mmol) at 25° C. The reaction mixture was stirred at 27° C. for 16 hr under nitrogen atmosphere. The progress of the reaction was monitored by TLC (SiO₂, 50% EtOAc/Pet., Rf=0.6, UV-active). On completion, the reaction mixture was diluted with ice cold water (10 mL) and then stirred for 15 min. The precipitated solid was collection by filtration and dried under vacuum to get crude compound. The crude compound was purified by column chromatography on silica gel (SiO₂, 100-200 mesh) with 35-40% EtOAc/Pet. The fractions containing pure product were collected and concentrated under reduced pressure to afford (S)—N-(1-(3-(4-chloro-1-methyl-3-(methylsulfonamido)-1H-indazol-7-yl)-7-methoxy-4-oxo-3,4-dihydroquinazolin-2-yl)-2-(3,5-difluorophenyl)ethyl)-2-(3-isopropyl-1H-pyrazol-1-yl)acetamide (31 mg, Yield: 49%, off-white solid). ¹H NMR (400 MHz, DMSO-d₆) δ=9.87 (br s, 1H), 8.79 (d, J=8.3 Hz, 1H), 8.11 (d, J=8.8 Hz, 1H), 7.62 (d, J=7.9 Hz, 1H), 7.38 (d, J=7.7 Hz, 1H), 7.28-7.20 (m, 3H), 6.99 (t, J=9.5 Hz, 1H), 6.68 (d, J=6.6 Hz, 2H), 5.96 (d, J=1.9 Hz, 1H), 4.64-4.56 (m, 1H), 4.41 (d, J=15.8 Hz, 1H), 4.29 (d, J=15.8 Hz, 1H), 3.97 (s, 3H), 3.51 (s, 3H), 3.44-3.35 (m, 1H), 3.18 (s, 3H), 3.05-2.96 (m, 1H), 2.79-2.73 (m, 1H), 1.08 (d, J=7.0 Hz, 6H). LCMS: RT=2.60 mins, (M+H)=739.12, Purity: 97%, HPLC Purity=97%, Chiral HPLC Purity=98%.

### (S)—N-(1-(3-(4-chloro-1-methyl-3-(methylsulfonamido)-1H-indazol-7-yl)-7-methoxy-4-oxo-3,4-dihydroquinazolin-2-yl)-2-(3,5-difluorophenyl)ethyl)-2-(3-cyclopropyl-1H-pyrazol-1-yl)acetamide (Example 6)

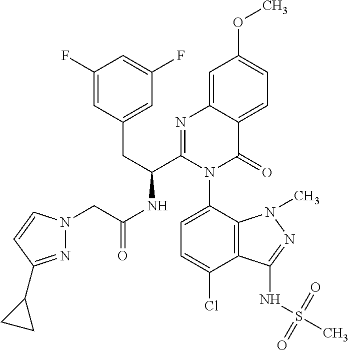 [SMILES: COc1ccc2c(=O)n(-c3ccc(Cl)c4c(NS(C)(=O)=O)nn(C)c34)c([C@H](Cc3cc(F)cc(F)c3)NC(=O)Cn3ccc(C4CC4)n3)nc2c1]

To a stirred solution of (S)—N-(7-(2-(1-amino-2-(3,5-difluorophenyl)ethyl)-7-methoxy-4-oxoquinazolin-3(4H)-yl)-4-chloro-1-methyl-1H-indazol-3-yl)methanesulfonamide (50 mg, 0.083 mmol), 2-(3-cyclopropyl-1H-pyrazol-1-yl)acetic acid (15.36 mg, 0.092 mmol), HOBt (13.38 mg, 0.087 mmol) and EDC.HCl (19.14 mg, 0.100 mmol) in DMF (2 mL) was added N-methylmorpholine (0.073 mL, 0.666 mmol) at 27° C. The reaction mixture was stirred for 16 hr under nitrogen atmosphere. The progress of the reaction was monitored by TLC (SiO₂, 50% EtOAc/Pet., Rf=0.5, UV-active). On completion, the reaction mixture was diluted with ice cold water (10 mL) and then stirred for 15 min. The precipitated solid was isolated by filtration, washed with water (10 mL), and dried under vacuum to get crude compound. The crude compound was purified by column chromatography on silica gel (SiO₂, 100-200 mesh) with 35-40% EtOAc/Pet. The fractions containing product were collected and concentrated under reduced pressure to afford (S)—N-(1-(3-(4-chloro-1-methyl-3-(methylsulfonamido)-1H-indazol-7-yl)-7-methoxy-4-oxo-3,4-dihydroquinazolin-2-yl)-2-(3,5-difluorophenyl)ethyl)-2-(3-cyclopropyl-1H-pyrazol-1-yl)acetamide (40 mg, Yield: 65%, off-white solid). ¹H NMR (400 MHz, DMSO-d₆) δ=9.87 (s, 1H), 8.79 (d, J=8.1 Hz, 1H), 8.12 (d, J=8.7 Hz, 1H), 7.64 (d, J=7.9 Hz, 1H), 7.38 (d, J=7.9 Hz, 1H), 7.26-7.21 (m, 3H), 7.05-6.96 (m, 1H), 6.68 (d, J=7.9 Hz, 2H), 5.84 (d, J=2.2 Hz, 1H), 4.62-4.54 (m, 1H), 4.37 (d, J=5.8 Hz, 1H), 4.26 (d, J=5.8 Hz, 1H), 3.98 (s, 3H), 3.53 (s, 3H), 3.42-3.39 (m, 1H), 3.20 (s, 3H), 3.05-2.97 (m, 1H), 1.79-1.71 (m, 1H), 0.80-0.72 (m, 2H), 0.56-0.48 (m, 2H). LCMS: RT=2.51 mins, (M+H)=737.05, Purity=99%, HPLC Purity=98%, Chiral HPLC Purity=99%.

### (S)—N-(1-(3-(4-chloro-1-methyl-3-(methylsulfonamido)-1H-indazol-7-yl)-7-methoxy-4-oxo-3,4-dihydroquinazolin-2-yl)-2-(3,5-difluorophenyl)ethyl)-2-(3-cyclobutyl-1H-pyrazol-1-yl)acetamide (Example 7)

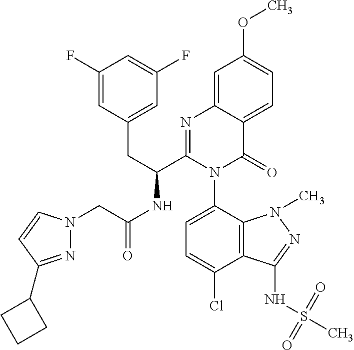 [SMILES: COc1ccc2c(=O)n(-c3ccc(Cl)c4c(NS(C)(=O)=O)nn(C)c34)c([C@H](Cc3cc(F)cc(F)c3)NC(=O)Cn3ccc(C4CCC4)n3)nc2c1]

To a solution of 2-(3-cyclobutyl-1H-pyrazol-1-yl)acetic acid (6.73 mg, 0.037 mmol), (S)—N-(7-(2-(1-amino-2-(3,5-difluorophenyl)ethyl)-7-methoxy-4-oxoquinazolin-3(4H)-yl)-4-chloro-1-methyl-1H-indazol-3-yl)methanesulfonamide (20 mg, 0.034 mmol), and HATU (15.49 mg, 0.041 mmol) in THF (0.5 mL) was added DIPEA (0.018 mL, 0.102 mmol). The resulting mixture was stirred for 18 h. The reaction was then concentrated and the residue was treated with 2 M ammonia in MeOH (1 mL) and stirred for 10 min (to cleave off any over coupled acid to the sulfonamide). The reaction concentrated under a stream of nitrogen. The crude product was purified by preparatory HPLC using the following conditions: Column: Zorbax Eclipse Plus C18, 21.2×100 mm, 5 m particles; Solvent A=0.1% Formic Acid in 100% Water. Solvent B=Acetonitrile. Flow Rate=40 mL/min. Start % B=53.3 Final % B=73.3. Gradient Time=7 min, then a 2 min hold at 98% B. Wavelength=215 and 254 nm. ESI+Range: 150 to 1500 dalton. Sample was loaded at 25% B. The product was isolated (16 mg, 64%). ¹H NMR (500 MHz, METHANOL-d₄) δ ppm 8.17 (d, J=8.94 Hz, 1H), 7.36 (d, J=2.09 Hz, 1H), 7.31 (d, J=2.38 Hz, 1H), 7.27 (d, J=7.75 Hz, 1H), 7.23 (dd, J=8.94, 2.38 Hz, 1H), 7.13 (d, J=7.75 Hz, 1 H), 6.71-6.77 (m, 1H), 6.64 (dd, J=8.35, 2.09 Hz, 2H), 6.13 (d, J=2.09 Hz, 1H), 4.91 (dd, J=8.49, 5.81 Hz, 1H), 4.38-4.43 (m, 1H), 4.28-4.33 (m, 1H), 4.01 (s, 3H), 3.55 (s, 3H), 3.44-3.51 (m, 2H), 3.25 (s, 3H), 3.04 (dd, J=13.86, 8.49 Hz, 1H), 2.21-2.29 (m, 2H), 2.08-2.18 (m, 2H), 1.94-2.03 (m, 1H), 1.80-1.88 (m, 1H). LCMS: Column: Acquity UPLC BEH C18, 2.1×30 mm, 1.7 m particles; Solvent A=0.1% Formic acid in 100% Water. Solvent B=0.10% Formic Acid in 100% Acetonitrile. Flow Rate=0.8 mL/min. Start % B=5. Final % B=95. Gradient Time=1.7 min, then a 0.2 min hold at 95% B. Wavelength=215 nm and 254 nm. Mass Range: 150 to 1500 Dalton. Retention Time: 1.37 min., 751.5 (M+H)⁺.

### (S)-tert-butyl (1-(3-(4-chloro-1-(2,2-difluoroethyl)-3-(N-(4-methoxybenzyl)cyclopropanesulfonamido)-1H-indazol-7-yl)-5-fluoro-7-methoxy-4-oxo-3,4-dihydroquinazolin-2-yl)-2-(3,5-difluorophenyl)ethyl)carbamate

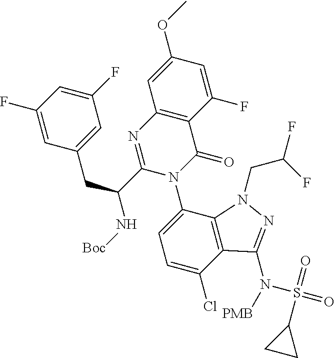

A mixture of(S)-2-((tert-butoxycarbonyl)amino)-3-(3,5-difluorophenyl)propanoic acid (66 mg, 0.219 mmol), 2-amino-6-fluoro-4-methoxybenzoic acid (65.5 mg, 0.219 mmol) and diphenyl phosphite (0.283 mL, 1.314 mmol) in Pyridine (2 mL) was sealed and heated in an oil bath at 70° C. for 2 h. LC/MS suggested the anhydride intermediate was formed. Opened the vessel, added N-(7-amino-4-chloro-1-(2,2-difluoroethyl)-1H-indazol-3-yl)-N-(4-methoxybenzyl)cyclopropanesulfonamide (103 mg, 0.219 mmol), sealed the vessel, heated again in the oil bath at 85° C. for another 2 h. At the same time LC/MS suggested the desired product was formed. Removed the solvent by a steady stream of N₂. The residue was partitioned between water (20 mL), extracted with EtOAc (10 mL, ×2). The separated organic layer was washed with 5% citric acid, 1.5 M K3PO4 and brine, dried it over MgSO₄, filtered, concentrated in vacuo. The residue was purified by silica gel flash chromatography eluting with gradient 0˜50% EtOAc-hexanes to afford the desired product tert-butyl (S)-(1-(3-(4-chloro-1-(2,2-difluoroethyl)-3-(N-(4-methoxybenzyl)cyclopropanesulfonamido)-1H-indazol-7-yl)-5-fluoro-7-methoxy-4-oxo-3,4-dihydroquinazolin-2-yl)-2-(3,5-difluorophenyl)ethyl)carbamate (88 mg, 0.097 mmol, 44.5% yield) as a white foam. LCMS (M+1): 903.2.

### (S)—N-(7-(2-(1-amino-2-(3,5-difluorophenyl)ethyl)-5-fluoro-7-methoxy-4-oxoquinazolin-3(4H)-yl)-4-chloro-1-(2,2-difluoroethyl)-1H-indazol-3-yl)cyclopropanesulfonamide

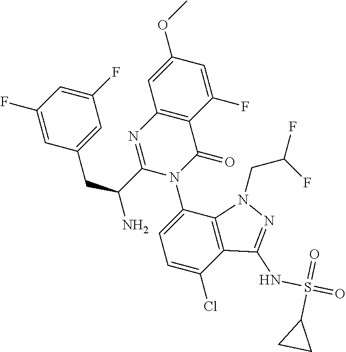 [SMILES: COc1cc(F)c2c(=O)n(-c3ccc(Cl)c4c(NS(=O)(=O)C5CC5)nn(CC(F)F)c34)c([C@@H](N)Cc3cc(F)cc(F)c3)nc2c1]

To an ice bath cooled solution of tert-butyl (S)-(1-(3-(4-chloro-1-(2,2-difluoroethyl)-3-(N-(4-methoxybenzyl)cyclopropanesulfonamido)-1H-indazol-7-yl)-5-fluoro-7-methoxy-4-oxo-3,4-dihydroquinazolin-2-yl)-2-(3,5-difluorophenyl)ethyl)carbamate (88 mg, 0.097 mmol) in DCM (0.5 mL) was added TFA (0.500 mL) and triflic acid (0.043 mL, 0.487 mmol). The resulted orange solution was stirred for 1 h. The reaction was concentrated with a steady stream of N₂, and the residue was partitioned between EtOAc (5 mL) and sat. NaHCO₃(10 mL). The separated organic layer was washed with brine, dried (MgSO₄), and concentrated in vacuo to afford the desired product (S)—N-(7-(2-(1-amino-2-(3,5-difluorophenyl)ethyl)-5-fluoro-7-methoxy-4-oxoquinazolin-3(4H)-yl)-4-chloro-1-(2,2-difluoroethyl)-1Hindazol-3-yl)cyclopropanesulfonamide (63 mg, 0.092 mmol, 95% yield) as an off-white foam. This material was used for the next coupling without further purification. LCMS (M+1): 684.0.

### N—((S)-1-(3-(4-chloro-3-(cyclopropanesulfonamido)-1-(2,2-difluoroethyl)-1H-indazol-7-yl)-5-fluoro-7-methoxy-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 8)

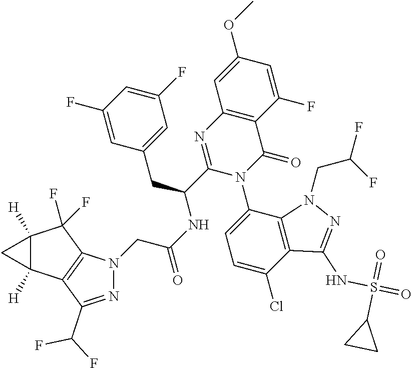 [SMILES: COc1cc(F)c2c(=O)n(-c3ccc(Cl)c4c(NS(=O)(=O)C5CC5)nn(CC(F)F)c34)c([C@H](Cc3cc(F)cc(F)c3)NC(=O)Cn3nc(C(F)F)c4c3C(F)(F)[C@@H]3C[C@H]43)nc2c1]

To a solution of (S)—N-(7-(2-(1-amino-2-(3,5-difluorophenyl)ethyl)-5-fluoro-7-methoxy-4-oxoquinazolin-3(4H)-yl)-4-chloro-1-(2,2-difluoroethyl)-1H-indazol-3-yl)cyclopropanesulfonamide (63 mg, 0.088 mmol), 2-((3bS,4aR)-3-(difluoromethyl)-5,5-difluoro-3b,4,4a,5-tetrahydro-1Hcyclopropa[3,4]cyclopenta[1,2-c]pyrazol-1-yl)acetic acid (23.15 mg, 0.088 mmol), and 1 M HOAt in DMA (0.044 mL, 0.044 mmol) in DMF (1.5 mL) was added EDC (18.48 mg, 0.096 mmol) and N-methylmorpholine (0.040 mL, 0.36 mmol). After stirring 18 h, reaction mixture was purified directly by preparative LC/MS with the following conditions: Column: XBridge C18, 200 mm×19 mm, 5-m particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 42% B, 42-82% B over 30 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 3.4 mg, and its purity was 100%. Material was further purified by preparatory SFC using the following conditions: Instrument: Waters 100 Prep SFC, Column: Chiral AD, 30×250 mm. 5 micron, Mobile Phase: 75% CO₂/25% IPA w/0.1% DEA, Flow Conditions: 100 mL/min, Detector Wavelength: 220 nm, Injection Details: 1000 μL 34.7 mg dissolved in 3 mL MeOH/CAN. The product was isolated (10.6 mg, 13%). Analytical LC/MS was used to determine the final purity. Injection 1 conditions: Column: Waters XBridge C18, 2.1 mm×50 mm, 1.7 m particles; Mobile Phase A: 5:95 acetonitrile:water with 10 mM ammonium acetate; Mobile Phase B: 95:5 acetonitrile:water with 10 mM ammonium acetate; temperature: 50° C.; Gradient: 0% B to 100% B over 3 min, then a 0.75 min hold at 100% B; Flow: 1 mL/min; Detection: MS and UV (220 nm). Injection 1 results: Purity: 100.0%; Observed Mass: 928.9; Retention Time: 2.23 min. Injection 2 conditions: Column: Waters XBridge C18, 2.1 mm×50 mm, 1.7 m particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.75 min hold at 100% B; Flow: 1 mL/min; Detection: MS and UV (220 nm). Injection 2 results: Purity: 100.0%; Observed Mass: 929.15; Retention Time: 2.18 min.

### N—((S)-1-(3-(4-chloro-1-methyl-3-(methylsulfonamido)-1H-indazol-7-yl)-7-(3-fluoropropoxy)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 9)

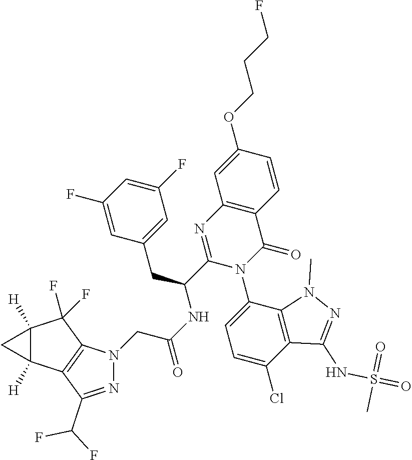 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(OCCCF)ccc4c3=O)c21]

The title compound was prepared according to Mitsunobu General Procedure using 3-fluoropropan-1-ol as the partner. The experiment afforded the title compound, N—((S)-1-(3-(4-chloro-1-methyl-3-(methylsulfonamido)-1H-indazol-7-yl)-7-(3-fluoropropoxy)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.39 min.; observed ion=881.4 (M+H). 1H NMR (500 MHz, METHANOL-d4) δ ppm 8.20 (d, J=8.94 Hz, 1H), 7.35 (d, J=2.68 Hz, 1H), 7.23-7.31 (m, 2H), 7.14 (d, J=7.75 Hz, 1H), 6.58-6.83 (m, 4H), 4.83 (dd, J=9.09, 5.22 Hz, 1H), 4.75 (t, J=5.81 Hz, 1H), 4.66 (t, J=5.66 Hz, 1H), 4.53 (d, J=2.09 Hz, 2H), 4.36 (t, J=6.11 Hz, 2H), 3.60 (s, 3H), 3.44-3.50 (m, 1H), 3.24 (s, 3H), 3.09 (dd, J=14.01, 9.24 Hz, 1H), 2.44 (ddd, J=11.18, 7.60, 4.17 Hz, 2H), 2.32 (quin, J=6.04 Hz, 1H), 2.27 (quin, J=5.96 Hz, 1H), 1.34-1.43 (m, 1H), 0.98-1.04 (m, 1H).

### N—((S)-1-(3-(4-chloro-1-methyl-3-(methylsulfonamido)-1H-indazol-7-yl)-7-isopropoxy-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 10)

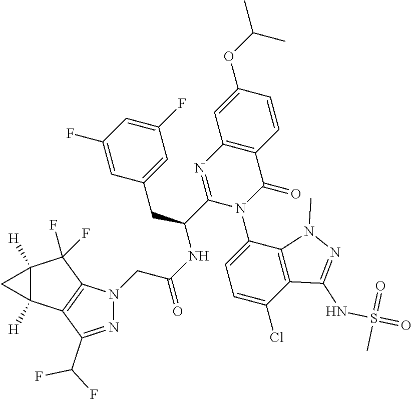 [SMILES: CC(C)Oc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

The title compound was prepared according to Mitsunobu General Procedure using propan-2-ol as the partner. The experiment afforded the title compound, N—((S)-1-(3-(4-chloro-1-methyl-3-(methylsulfonamido)-1H-indazol-7-yl)-7-isopropoxy-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

The sample was analyzed using LCMS Method F: retention time=1.45 min.; observed ion=863.4 (M+H). 1H NMR (500 MHz, METHANOL-d4) δ ppm 8.18 (d, J=8.94 Hz, 1H), 7.26-7.31 (m, 2H), 7.21 (dd, J=8.94, 2.68 Hz, 1H), 7.14 (d, J=7.75 Hz, 1H), 6.57-6.83 (m, 4H), 4.83 (dd, J=8.94, 5.07 Hz, 1H), 4.53 (d, J=2.09 Hz, 2H), 3.61 (s, 3H), 3.43-3.49 (m, 1H), 3.24 (s, 3H), 3.08 (dd, J=13.86, 9.09 Hz, 1H), 2.44 (ddd, J=11.25, 7.67, 4.02 Hz, 2H), 1.47 (d, J=5.96 Hz, 6H), 1.33-1.41 (m, 1H), 0.99-1.05 (m, 1H). The isopropyl CH proton was not observed due to solvent overlap.

### N—((S)-1-(7-butoxy-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 11)

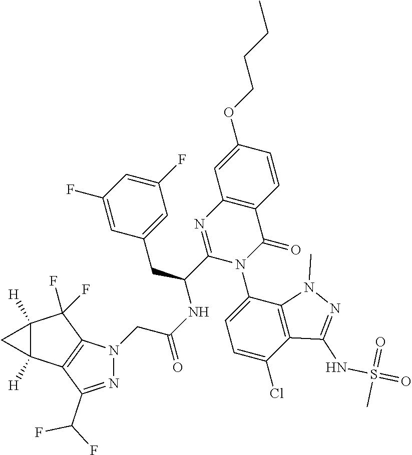 [SMILES: CCCCOc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

The title compound was prepared according to Mitsunobu General Procedure using butan-1-ol as the partner. The experiment afforded the title compound, N—((S)-1-(7-butoxy-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide.

The sample was analyzed using LCMS Method F: retention time=1.53 min.; observed ion=877.4 (M+H). 1H NMR (500 MHz, METHANOL-d4) δ ppm 8.19 (d, J=8.94 Hz, 1H), 7.31 (d, J=2.38 Hz, 1H), 7.28 (d, J=7.75 Hz, 1H), 7.24 (dd, J=8.94, 2.68 Hz, 1H), 7.13 (d, J=7.75 Hz, 1H), 6.59-6.82 (m, 4H), 4.83 (dd, J=9.09, 5.22 Hz, 1H), 4.53 (d, J=2.09 Hz, 2H), 4.23 (t, J=6.56 Hz, 2H), 3.60 (s, 3H), 3.44-3.48 (m, 1H), 3.24 (s, 3H), 3.08 (dd, J=13.86, 9.09 Hz, 1H), 2.44 (ddd, J=11.25, 7.53, 3.87 Hz, 2H), 1.86-1.93 (m, 2H), 1.56-1.65 (m, 2H), 1.35-1.40 (m, 1H), 1.07 (t, J=7.30 Hz, 3H), 0.99-1.04 (m, 1H).

### N—((S)-1-(3-(4-chloro-1-methyl-3-(methylsulfonamido)-1H-indazol-7-yl)-7-isobutoxy-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 12)

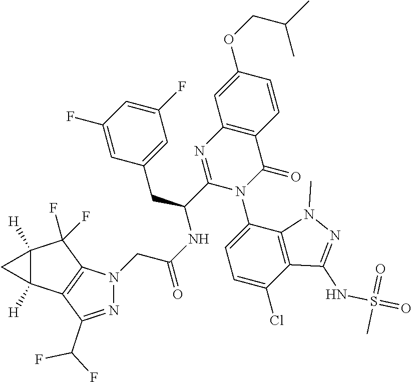 [SMILES: CC(C)COc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

The title compound was prepared according to the Mitsunobu General Procedure using 2-methylpropan-1-ol as the partner. The experiment afforded the title compound, N—((S)-1-(3-(4-chloro-1-methyl-3-(methylsulfonamido)-1H-indazol-7-yl)-7-isobutoxy-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.53 min.; observed ion=877.4 (M+H). 1H NMR (500 MHz, METHANOL-d4) δ ppm 8.19 (d, J=8.94 Hz, 1H), 7.31 (d, J=2.38 Hz, 1H), 7.29 (d, J=8.05 Hz, 1H), 7.25 (dd, J=8.79, 2.53 Hz, 1H), 7.14 (d, J=8.05 Hz, 1H), 6.56-6.83 (m, 4H), 4.83 (dd, J=9.09, 5.22 Hz, 1H), 4.53 (d, J=2.09 Hz, 2H), 4.00 (d, J=6.56 Hz, 2H), 3.60 (s, 3H), 3.43-3.49 (m, 1H), 3.24 (s, 3H), 3.08 (dd, J=14.01, 9.24 Hz, 1H), 2.40-2.48 (m, 2H), 2.21 (dquin, J=13.32, 6.65, 6.65, 6.65, 6.65 Hz, 1H), 1.34-1.41 (m, 1H), 1.13 (d, J=6.56 Hz, 6H), 1.02 (dtd, J=7.64, 4.00, 4.00, 2.24 Hz, 1H).

### N—((S)-1-(3-(4-chloro-1-methyl-3-(methylsulfonamido)-1H-indazol-7-yl)-7-(((S)-3-methylbutan-2-yl)oxy)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 13)

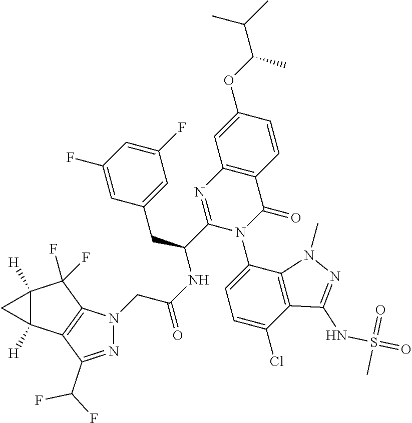 [SMILES: CC(C)[C@H](C)Oc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

The title compound was prepared according to Mitsunobu General Procedure using (2R)-3-methylbutan-2-ol as the partner. The experiment afforded the title compound, N—((S)-1-(3-(4-chloro-1-methyl-3-(methylsulfonamido)-1H-indazol-7-yl)-7-(((S)-3-methylbutan-2-yl)oxy)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.56 min.; observed ion=891.4 (M+H). 1H NMR (500 MHz, METHANOL-d4) δ ppm 8.18 (d, J=8.64 Hz, 1H), 7.26-7.32 (m, 2H), 7.22 (dd, J=8.94, 2.38 Hz, 1H), 7.13 (d, J=8.05 Hz, 1H), 6.58-6.86 (m, 4H), 4.82 (dd, J=8.94, 5.36 Hz, 1H), 4.50-4.61 (m, 3H), 3.61 (s, 3H), 3.42-3.48 (m, 1H), 3.24 (s, 3H), 3.08 (dd, J=14.01, 8.94 Hz, 1H), 2.44 (ddd, J=11.33, 7.60, 4.02 Hz, 2H), 2.01-2.09 (m, 1H), 1.39 (d, J=6.26 Hz, 4H), 1.05-1.12 (m, 6H), 0.98-1.04 (m, 1H).

### N—((S)-1-(3-(4-chloro-1-methyl-3-(methylsulfonamido)-1H-indazol-7-yl)-7-(((R)-3-methylbutan-2-yl)oxy)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 14)

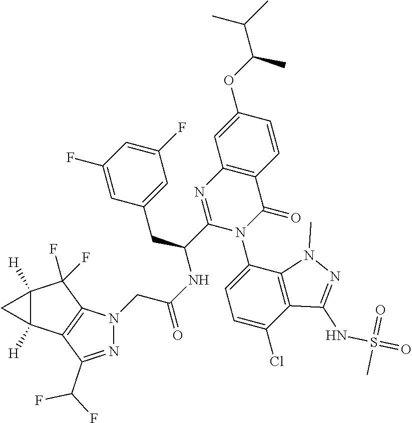 [SMILES: CC(C)[C@@H](C)Oc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

The title compound was prepared according to Mitsunobu General Procedure using (2S)-3-methylbutan-2-ol as the partner. The experiment afforded the title compound, N—((S)-1-(3-(4-chloro-1-methyl-3-(methylsulfonamido)-1H-indazol-7-yl)-7-(((R)-3-methylbutan-2-yl)oxy)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.56 min.; observed ion=891.4 (M+H). 1H NMR (500 MHz, METHANOL-d4) δ ppm 8.18 (d, J=8.94 Hz, 1H), 7.25-7.31 (m, 2H), 7.22 (dd, J=8.94, 2.38 Hz, 1H), 7.15 (d, J=7.75 Hz, 1H), 6.55-6.83 (m, 5H), 4.82 (dd, J=9.09, 5.22 Hz, 1H), 4.50-4.56 (m, 3H), 3.59 (s, 3H), 3.43-3.48 (m, 1H), 3.23 (s, 3H), 3.08 (dd, J=14.01, 9.24 Hz, 1H), 2.40-2.48 (m, 2H), 2.02-2.10 (m, 1H), 1.39 (d, J=6.26 Hz, 4H), 1.08 (dd, J=15.65, 6.71 Hz, 5H), 0.98-1.04 (m, 1H).

### N—((S)-1-(3-(4-chloro-1-methyl-3-(methylsulfonamido)-1H-indazol-7-yl)-7-methyl-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 15)

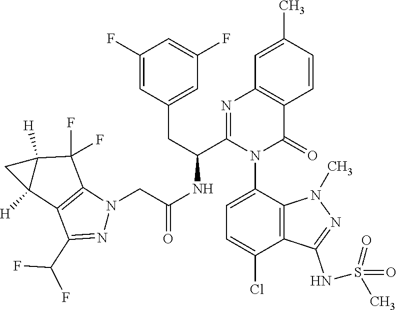 [SMILES: Cc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

The title compound was prepared according to General Procedure A using 2,4,6-trimethyl-1,3,5,2,4,6-trioxatriborinane as the coupling partner. The experiment afforded the title compound, N—((S)-1-(3-(4-chloro-1-methyl-3-(methylsulfonamido)-1H-indazol-7-yl)-7-methyl-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method C: retention time=1.37 min.; observed ion=819.2 (M+H). 1H NMR (METHANOL-d4, 500 MHz) δ 8.18 (d, 1H, J=8.0 Hz), 7.72 (s, 1H), 7.52 (dd, 1H, J=1.5, 8.3 Hz), 7.29 (d, 1H, J=8.0 Hz), 7.14 (d, 1H, J=7.7 Hz), 6.8-6.8 (m, 1H), 6.62 (dd, 2H, J=2.2, 8.2 Hz), 6.69 (t, 1H, J=54.8 Hz), 4.8-4.9 (m, 1H), 4.52 (s, 2H), 3.59 (s, 3H), 3.4-3.5 (m, 1H), 3.2-3.3 (m, 3H), 3.08 (dd, 1H, J=8.9, 14.0 Hz), 2.61 (s, 3H), 2.4-2.5 (m, 2H), 1.3-1.4 (m, 1H), 1.01 (dt, 1H, J=1.9, 3.7 Hz).

### N—((S)-1-(3-(4-chloro-1-methyl-3-(methylsulfonamido)-H-indazol-7-yl)-7-ethyl-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 16)

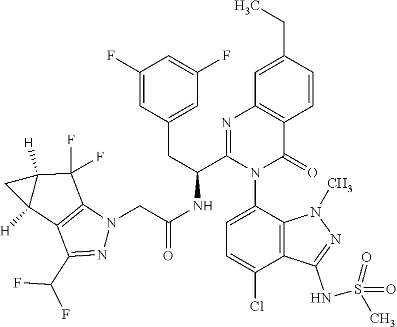 [SMILES: CCc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

The title compound was prepared according to General Procedure E using ethyltrifluoroborate as the coupling partner. The experiment afforded the title compound, N—((S)-1-(3-(4-chloro-1-methyl-3-(methylsulfonamido)-1H-indazol-7-yl)-7-ethyl-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method C: retention time=1.42 min.; observed ion=833.4 (M+H). 1H NMR (METHANOL-d4, 500 MHz) δ 8.21 (d, 1H, J=8.2 Hz), 7.74 (d, 1H, J=0.9 Hz), 7.55 (dd, 1H, J=1.6, 8.2 Hz), 7.29 (d, 1H, J=7.9 Hz), 7.15 (d, 1H, J=7.9 Hz), 6.8-6.8 (m, 1H), 6.62 (dd, 2H, J=2.2, 8.2 Hz), 6.69 (t, 1H, J=54.7 Hz), 4.8-4.8 (m, 1H), 4.53 (s, 2H), 3.59 (s, 3H), 3.4-3.5 (m, 1H), 3.25 (s, 3H), 3.09 (dd, 1H, J=9.1, 13.9 Hz), 2.92 (q, 2H, J=7.6 Hz), 2.4-2.5 (m, 2H), 1.4-1.4 (m, 4H), 1.01 (dt, 1H, J=1.9, 3.6 Hz).

### tert-butyl (S)-(1-(7-acetyl-3-(4-chloro-1-(2,2-difluoroethyl)-3-(N-(4-methoxybenzyl)methylsulfonamido)-1H-indazol-7-yl)-4-oxo-3,4-dihydro quinazolin-2-yl)-2-(3,5-difluorophenyl)ethyl)carbamate

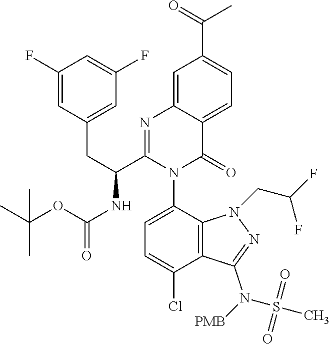

A solution of tert-butyl (S)-(1-(7-bromo-3-(4-chloro-1-(2,2-difluoroethyl)-3-(N-(4-methoxybenzyl)methylsulfonamido)-1H-indazol-7-yl)-4-oxo-3,4-dihydroquinazolin-2-yl)-2-(3,5-difluorophenyl)ethyl)carbamate (220 mg, 0.242 mmol), 1-ethoxyvinyltri-n-butyltin (0.099 mL, 0.291 mmol), and bis(triphenylphosphine)nickel(II) chloride (15.85 mg, 0.024 mmol) in toluene (5 mL) was heated at 100° C. for 5 h. The mixture was then cooled and filtered through a pad of Celite. The filtrate was concentrated and the crude material was redissolved in THF (10 mL) and 1 N HCl (0.25 mL) was added to the mixture. The resulting mixture was then stirred for 1 h. Water was then added and the mixture was extracted with ethyl acetate, dried (Na₂SO₄), filtered, and concentrated in vacuo. The residue was then purified by silica gel flash chromatography (5-50% EtOAc/hexane) to afford tert-butyl (S)-(1-(7-acetyl-3-(4-chloro-1-(2,2-difluoroethyl)-3-(N-(4-methoxybenzyl)methylsulfonamido)-1H-indazol-7-yl)-4-oxo-3,4-dihydroquinazolin-2-yl)-2-(3,5-difluorophenyl)ethyl)carbamate (140 mg, 0.161 mmol, 66.3% yield) which was a mixture of stereoisomers. ¹H NMR (500 MHz, CDCl₃) δ ppm 8.42 (d, J=8.34 Hz, 1H), 8.38 (d, J=1.19 Hz, 1H), 8.14 (dd, J=8.34, 1.49 Hz, 1H), 7.27 (s, 1H), 7.22 (br d, J=8.34 Hz, 1H), 6.81 (br d, J=8.05 Hz, 2H), 6.72 (tt, J=8.90, 2.27 Hz, 1H), 6.34-6.58 (m, 3H), 5.73-6.08 (m, 1H), 4.67-5.48 (m, 3H), 4.28-4.54 (m, 2H), 3.98-4.11 (m, 1H), 3.77 (br s, 3H), 3.14-3.32 (m, 1H), 2.95-2.98 (m, 3H), 2.90-2.95 (m, 1H), 2.80 (s, 3H), 1.41 (br s, 9H). LC/MS: m/z=893.20 [M+Na].

### tert-butyl (S)-(1-(3-(4-chloro-1-(2,2-difluoroethyl)-3-(N-(4-methoxybenzyl)methylsulfonamido)-1H-indazol-7-yl)-7-(1,1-difluoroethyl)-4-oxo-3,4-dihydroquinazolin-2-yl)-2-(3,5-difluorophenyl)ethyl)carbamate

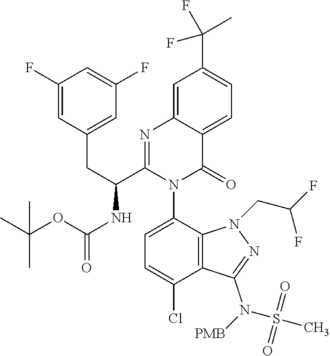

To a solution of tert-butyl (S)-(1-(7-acetyl-3-(4-chloro-1-(2,2-difluoroethyl)-3-(N-(4-methoxybenzyl)methylsulfonamido)-1H-indazol-7-yl)-4-oxo-3,4-dihydroquinazolin-2-yl)-2-(3,5-difluorophenyl)ethyl)carbamate (140 mg, 0.161 mmol) in DCM (3 mL) in a sealed tube was added DAST (0.637 mL, 4.82 mmol). The tube was then sealed and the resulting mixture was heated at 60° C. for 16 h. After cooling, the reaction mixture was poured into a sat. NaHCO₃ solution and extracted with DCM, dried (Na₂SO₄), filtered, and concentrated in vacuo. The residue was then purified by silica gel flash chromatography (5-40% EtOAc/hexane) to afford the title compound tert-butyl (S)-(1-(3-(4-chloro-1-(2,2-difluoroethyl)-3-(N-(4-methoxybenzyl)methylsulfonamido)-1H-indazol-7-yl)-7-(1,1-difluoroethyl)-4-oxo-3,4-dihydroquinazolin-2-yl)-2-(3,5-difluorophenyl)ethyl)carbamate (77 mg, 0.086 mmol, 53.6% yield). LC/MS: m/z=893.2 [M+H]⁺.

### (S)—N-(7-(2-(1-amino-2-(3,5-difluorophenyl)ethyl)-7-(1,1-difluoroethyl)-4-oxoquinazolin-3(4H)-yl)-4-chloro-1-(2,2-difluoroethyl)-1H-indazol-3-yl)methanesulfonamide

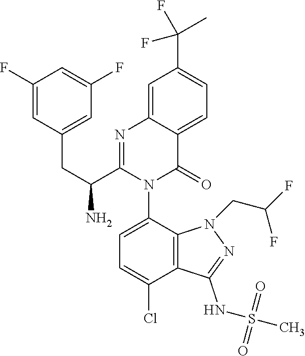 [SMILES: CC(F)(F)c1ccc2c(=O)n(-c3ccc(Cl)c4c(NS(C)(=O)=O)nn(CC(F)F)c34)c([C@@H](N)Cc3cc(F)cc(F)c3)nc2c1]

To a stirring solution of tert-butyl (S)-(1-(3-(4-chloro-1-(2,2-difluoroethyl)-3-(N-(4-methoxybenzyl)methylsulfonamido)-1H-indazol-7-yl)-7-(1,1-difluoroethyl)-4-oxo-3,4-dihydroquinazolin-2-yl)-2-(3,5-difluorophenyl)ethyl)carbamate (75 mg, 0.084 mmol) in DCM (2 mL) was added TFA (1 mL, 12.98 mmol) followed by triflic acid (0.011 mL, 0.126 mmol). The resulting dark red solution was stirred for 2 h and then concentrated to a minimum under vacuum. The residue was partitioned between EtOAc (50 mL) and aq. NaOH (1 M, 5 mL). The organic phase was isolated and dried (Na₂SO₄), filtered, and then concentrated in vacuo. The residue was then purified by silica gel flash chromatography (5-100% EtOAC/hexane) to afford (S)—N-(7-(2-(1-amino-2-(3,5-difluorophenyl)ethyl)-7-(1,1-difluoroethyl)-4-oxoquinazolin-3(4H)-yl)-4-chloro-1-(2,2-difluoroethyl)-1H-indazol-3-yl)methanesulfonamide (52 mg, 0.077 mmol, 92% yield). ¹H NMR (500 MHz, DMSO-d₆) δ ppm 8.29 (d, J=8.05 Hz, 1H), 7.98 (s, 1H), 7.80 (br d, J=7.75 Hz, 1H), 7.41-7.51 (m, 2H), 7.00 (br t, J=9.39 Hz, 1H), 6.73 (br d, J=6.85 Hz, 2H), 6.13-6.43 (m, 1H), 4.38-4.52 (m, 1H), 4.20-4.36 (m, 1H), 3.53 (br dd, J=7.90, 4.32 Hz, 1H), 3.27 (br d, J=5.07 Hz, 1H), 3.24 (s, 3H), 2.83 (br dd, J=13.41, 8.64 Hz, 1H), 2.11 (t, J=19.07 Hz, 3H). LCMS (M+H)+=673.10.

### N—((S)-1-(3-(4-chloro-1-(2,2-difluoroethyl)-3-(methylsulfonamido)-1H-indazol-7-yl)-7-(1,1-difluoroethyl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 17)

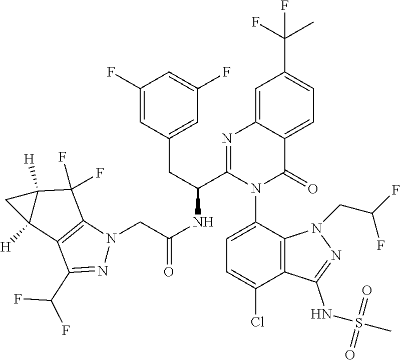 [SMILES: CC(F)(F)c1ccc2c(=O)n(-c3ccc(Cl)c4c(NS(C)(=O)=O)nn(CC(F)F)c34)c([C@H](Cc3cc(F)cc(F)c3)NC(=O)Cn3nc(C(F)F)c4c3C(F)(F)[C@@H]3C[C@H]43)nc2c1]

To a solution of 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (20.41 mg, 0.077 mmol) and (S)—N-(7-(2-(1-amino-2-(3,5-difluorophenyl)ethyl)-7-(1,1-difluoroethyl)-4-oxoquinazolin-3(4H)-yl)-4-chloro-1-(2,2-difluoroethyl)-1H-indazol-3-yl)methanesulfonamide (52 mg, 0.077 mmol) in THF (2 mL) was added DIPEA (0.040 mL, 0.232 mmol) followed by HATU (32.3 mg, 0.085 mmol). The resulting mixture was stirred for 3 h. 0.5 mL ammonia in methanol was then added and the mixture was stirred for 30 min. Water was then added and the mixture was extracted with ethyl acetate, washed with brine, dried (Na₂SO₄), filtered, and concentrated in vacuo. The residue was then purified by silica gel flash chromatography (5-50% EtOAc/hexane) and the material was further purified by preparatory HPLC using the following conditions: Column Chromega CC4 30×250 mm 5 micron (A=80% HEPTANE w/ 0/1% FORMIC ACID, B=20% ETHANOL w/ 0/1% FORMIC ACID Flow 45 ml/min). The product was isolated (21 mg, 0.022 mmol, 40%). ¹H NMR (500 MHz, METHANOL-d₄) δ ppm 8.26 (d, J=8.05 Hz, 1H), 7.92 (d, J=0.89 Hz, 1H), 7.70 (dd, J=8.34, 1.79 Hz, 1H), 7.28 (d, J=8.05 Hz, 1H), 7.19 (d, J=7.75 Hz, 1H), 6.40-6.72 (m, 4H), 5.71-6.04 (m, 1H), 4.62 (dd, J=9.54, 4.77 Hz, 1H), 4.43-4.57 (m, 2H), 4.19-4.33 (m, 1H), 3.75-3.88 (m, 1H), 3.24-3.33 (m, 1H), 3.15 (s, 3H), 2.95 (dd, J=14.31, 9.54 Hz, 1H), 2.27-2.37 (m, 2H), 1.96 (t, J=18.48 Hz, 3H), 1.16-1.28 (m, 2H), 0.85-0.93 (m, 1H). LCMS (M+H)+=919.05.

### tert-butyl (S)-(1-(7-acetyl-3-(4-chloro-1-(2,2-difluoroethyl)-3-(N-(4-methoxybenzyl)cyclopropanesulfonamido)-1H-indazol-7-yl)-4-oxo-3,4-dihydroquinazolin-2-yl)-2-(3,5-difluorophenyl)ethyl)carbamate

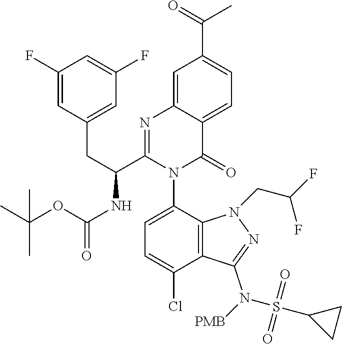

A solution of tert-butyl (S)-(1-(7-bromo-3-(4-chloro-1-(2,2-difluoroethyl)-3-(N-(4-methoxybenzyl)cyclopropanesulfonamido)-1H-indazol-7-yl)-4-oxo-3,4-dihydroquinazolin-2-yl)-2-(3,5-difluorophenyl)ethyl)carbamate (680 mg, 0.728 mmol), 1-ethoxyvinyltri-n-butyltin (0.298 mL, 0.873 mmol) and bis(triphenylphosphine)palladium(II) chloride (51.1 mg, 0.073 mmol) in Toluene (10 mL) was heated at 100° C. for 5 h. Mixture was then cooled and filtered through a pad of Celite. The filtrate was then concentrated and the crude material was redissolved in THF (10 mL) and 1N HCl (1 mL) was added to the mixture. The resulting mixture was then stirred at room temp for 1 h. Water was then added and the mixture was extracted with ethyl acetate, dried (Na₂SO₄), filtered and concentrated. The residue was then purified by Biotage (5-50% EtOAc/hexane) to afford tert-butyl (S)-(1-(7-acetyl-3-(4-chloro-1-(2,2-difluoroethyl)-3-(N-(4-methoxybenzyl)cyclopropanesulfonamido)-1H-indazol-7-yl)-4-oxo-3,4-dihydroquinazolin-2-yl)-2-(3,5-difluorophenyl)ethyl)carbamate (510 mg, 0.568 mmol, 78% yield). LCMS (M+H)+=919.25.

### tert-butyl (S)-(1-(3-(4-chloro-1-(2,2-difluoroethyl)-3-(N-(4-methoxybenzyl)cyclopropanesulfonamido)-1H-indazol-7-yl)-7-(1,1-difluoroethyl)-4-oxo-3,4-dihydroquinazolin-2-yl)-2-(3,5-difluorophenyl)ethyl)carbamate

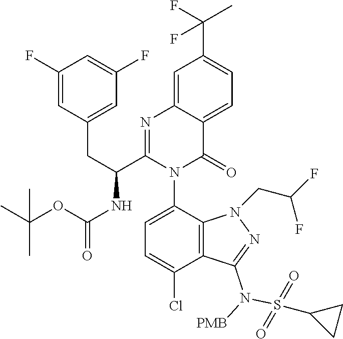

To a solution of tert-butyl (S)-(1-(7-acetyl-3-(4-chloro-1-(2,2-difluoroethyl)-3-(N-(4-methoxybenzyl)cyclopropanesulfonamido)-1H-indazol-7-yl)-4-oxo-3,4-dihydroquinazolin-2-yl)-2-(3,5-difluorophenyl)ethyl)carbamate (500 mg, 0.557 mmol) in Dichloromethane (DCM) (5 mL) in a sealed tube was added DAST (2.209 mL, 16.72 mmol). The tube was then sealed and the resulting mixture was heated at 60° C. for 16 h. After cooling to room temp, the reaction mixture was poured into a sat. NaHCO₃ solution and extracted with DCM, dried (Na₂SO₄), filtered and concentrated. The residue was then purified by Biotage (5-40% EtOAc/hexane) to afford the title compound tert-butyl (S)-(1-(3-(4-chloro-1-(2,2-difluoroethyl)-3-(N-(4-methoxybenzyl)cyclopropanesulfonamido)-1H-indazol-7-yl)-7-(1,1-difluoroethyl)-4-oxo-3,4-dihydroquinazolin-2-yl)-2-(3,5-difluorophenyl)ethyl)carbamate (260 mg, 0.283 mmol, 50.8% yield). LC/MS: m/z=941.15 [M+Na].

### (S)—N-(7-(2-(1-amino-2-(3,5-difluorophenyl)ethyl)-7-(1,1-difluoroethyl)-4-oxoquinazolin-3(4H)-yl)-4-chloro-1-(2,2-difluoroethyl)-1H-indazol-3-yl)cyclopropanesulfonamide

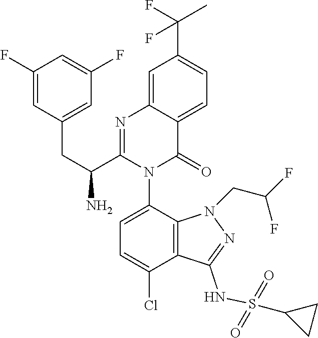 [SMILES: CC(F)(F)c1ccc2c(=O)n(-c3ccc(Cl)c4c(NS(=O)(=O)C5CC5)nn(CC(F)F)c34)c([C@@H](N)Cc3cc(F)cc(F)c3)nc2c1]

To a stirring solution of tert-butyl (S)-(1-(3-(4-chloro-1-(2,2-difluoroethyl)-3-(N-(4-methoxybenzyl)cyclopropanesulfonamido)-1H-indazol-7-yl)-7-(1,1-difluoroethyl)-4-oxo-3,4-dihydroquinazolin-2-yl)-2-(3,5-difluorophenyl)ethyl)carbamate (250 mg, 0.272 mmol) in Dichloromethane (DCM) (2 mL) was added TFA (1 mL, 12.98 mmol) followed by triflic acid (0.048 mL, 0.544 mmol) and the resulting dark red solution was stirred at room temp for 2 h and then concentrated to a minimum on the rotovap. The residue was partitioned between EtOAc (50 mL) and aq. NaOH (1M, 5 mL). The aq. phase was tested and determined to be pH >=8.0. The organic phase was isolated and dried over Na₂SO₄, filtered, and then concentrated. The residue was then purified by Biotage (5-100% EtOAC/hexane) to afford (S)—N-(7-(2-(1-amino-2-(3,5-difluorophenyl)ethyl)-7-(1,1-difluoroethyl)-4-oxoquinazolin-3(4H)-yl)-4-chloro-1-(2,2-difluoroethyl)-1H-indazol-3-yl)cyclopropanesulfonamide (140 mg, 0.200 mmol, 73.6% yield). ¹H NMR (500 MHz, DMSO-d₆) δ ppm 9.15-9.19 (m, 1H), 8.29 (d, J=8.34 Hz, 1H), 7.98 (d, J=0.89 Hz, 1H), 7.80 (dd, J=8.20, 1.64 Hz, 1H), 7.40-7.54 (m, 2H), 7.01 (tt, J=9.43, 2.35 Hz, 1H), 6.73 (br d, J=6.85 Hz, 2H), 6.11-6.43 (m, 1H), 4.37-4.52 (m, 1H), 4.21-4.35 (m, 1H), 3.47-3.58 (m, 1H), 3.23-3.31 (m, 1H), 2.90-3.00 (m, 1H), 2.83 (dd, J=13.56, 8.49 Hz, 1H), 2.11 (t, J=19.22 Hz, 3H), 0.99-1.06 (m, 4H). LCMS (M+H)+=699.10

### N—((S)-1-(3-(4-chloro-3-(cyclopropanesulfonamido)-1-(2,2-difluoroethyl)-1H-indazol-7-yl)-7-(1,1-difluoroethyl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 18)

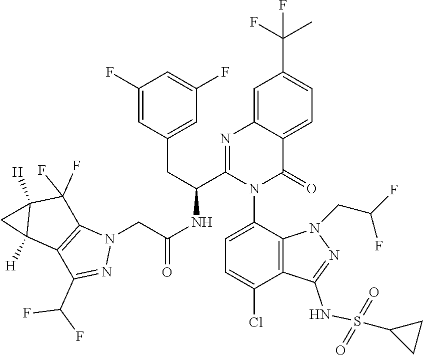 [SMILES: CC(F)(F)c1ccc2c(=O)n(-c3ccc(Cl)c4c(NS(=O)(=O)C5CC5)nn(CC(F)F)c34)c([C@H](Cc3cc(F)cc(F)c3)NC(=O)Cn3nc(C(F)F)c4c3C(F)(F)[C@@H]3C[C@H]43)nc2c1]

To a solution of 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (37.8 mg, 0.143 mmol) and (S)—N-(7-(2-(1-amino-2-(3,5-difluorophenyl)ethyl)-7-(1,1-difluoroethyl)-4-oxoquinazolin-3(4H)-yl)-4-chloro-1-(2,2-difluoroethyl)-1H-indazol-3-yl)cyclopropanesulfonamide (100 mg, 0.143 mmol) in Tetrahydrofuran (THF) (3 mL) was added DIEA (0.075 mL, 0.429 mmol) followed by HATU (59.8 mg, 0.157 mmol) and the resulting mixture was stirred at room temp for 3 h. 0.5 mL ammonia in methanol was then added and the mixture was stirred for 30 min. Water was then added and the mixture was extracted with ethyl acetate, washed with brine, dried (Na₂SO₄), filtered and concentrated. The residue was then purified by Biotage (5-50% EtOAc/hexane) and material was further purified by Jasco (Chiralpak IA preparative column, 10×250 mm, 5 m; Mobile Phase: 20% MeOH in CO₂, 150 bar, Temp: 40° C., Flow rate: 3.5 mL/min. in 10 min.) to afford desired isomer N—((S)-1-(3-(4-chloro-3-(cyclopropanesulfonamido)-1-(2,2-difluoroethyl)-1H-indazol-7-yl)-7-(1,1-difluoroethyl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (63 mg, 0.063 mmol, 44.3% yield). ¹H NMR (500 MHz, METHANOL-d₄) δ ppm 8.26 (d, J=8.34 Hz, 1H), 7.92 (d, J=1.79 Hz, 1H), 7.70 (dd, J=8.34, 1.49 Hz, 1H), 7.29 (d, J=8.05 Hz, 1H), 7.20 (d, J=7.75 Hz, 1H), 6.37-6.73 (m, 4H), 5.73-6.05 (m, 1H), 4.61 (dd, J=9.54, 4.47 Hz, 1H), 4.45-4.59 (m, 2H), 4.26 (dtd, J=15.16, 13.58, 13.58, 4.17 Hz, 1H), 3.75-3.86 (m, 1H), 3.28 (dd, J=14.31, 4.47 Hz, 1H), 2.95 (dd, J=14.01, 9.54 Hz, 1H), 2.79 (tt, J=7.94, 4.88 Hz, 1 H), 2.26-2.37 (m, 1H), 1.89-2.01 (m, 3H), 1.24 (td, J=7.60, 5.66 Hz, 1H), 0.93-1.02 (m, 2H), 0.83-0.91 (m, 3H). LC/MS: m/z=945.10 [M+H]⁺.

### N—((S)-1-(3-(4-chloro-3-(cyclopropanesulfonamido)-1-(2,2-difluoroethyl)-1H—N—((S)-1-(3-(4-chloro-1-methyl-3-(methylsulfonamido)-1H-indazol-7-yl)-4-oxo-7-propyl-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 19)

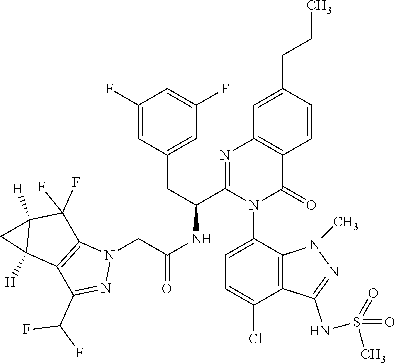 [SMILES: CCCc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

The title compound was prepared according to General Procedure E using trifluoro(propyl)borate as the coupling partner. The experiment afforded the title compound, N—((S)-1-(3-(4-chloro-1-methyl-3-(methylsulfonamido)-1H-indazol-7-yl)-4-oxo-7-propyl-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.49 min.; observed ion=847.4 (M+H). 1H NMR (METHANOL-d4, 500 MHz) δ 8.21 (d, 1H, J=8.0 Hz), 7.72 (d, 1H, J=1.2 Hz), 7.53 (dd, 1H, J=1.6, 8.2 Hz), 7.29 (d, 1H, J=7.7 Hz), 7.16 (d, 1H, J=7.7 Hz), 6.8-6.8 (m, 1H), 6.62 (dd, 2H, J=2.1, 8.0 Hz), 6.69 (br t, 1H, J=54.7 Hz), 4.84 (d, 1H, J=5.1 Hz), 4.53 (d, 2H, J=1.5 Hz), 3.60 (s, 3H), 3.4-3.5 (m, 1H), 3.2-3.3 (m, 3H), 3.09 (dd, 1H, J=8.9, 14.0 Hz), 2.8-2.9 (m, 2H), 2.4-2.5 (m, 2H), 1.8-1.9 (m, 2H), 1.37 (br d, 1H, J=7.5 Hz), 1.0-1.1 (m, 4H).

### N—((S)-1-(3-(4-chloro-3-(N-(4-methoxybenzyl)methylsulfonamido)-1-methyl-1H-indazol-7-yl)-4-oxo-7-(tributylstannyl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

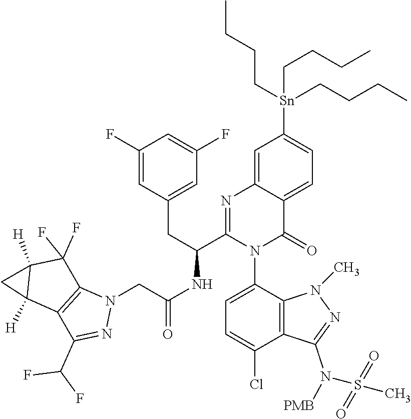

A solution of N—((S)-1-(7-bromo-3-(4-chloro-3-(N-(4-methoxybenzyl)methylsulfonamido)-1-methyl-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (1.1 g, 1.095 mmol), 1,1,1,2,2,2-hexabutyldistannane (0.763 g, 1.314 mmol) and bis(triphenylphosphine)palladium(II) chloride (0.077 g, 0.110 mmol) in Toluene (12 mL) was heated at 100° C. for 16 h. Mixture was then cooled and filtered through a pad of Celite. Water was then added and the mixture was extracted with ethyl acetate, dried (Na₂SO₄), filtered and concentrated. The residue was then purified by Biotage (5-50% EtOAc/hexane) to afford N—((S)-1-(3-(4-chloro-3-(N-(4-methoxybenzyl)methylsulfonamido)-1-methyl-1H-indazol-7-yl)-4-oxo-7-(tributylstannyl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (750 mg, 0.618 mmol, 56.4% yield). LCMS (M+H)+=1215.25

### N—((S)-1-(3-(4-chloro-3-(N-(4-methoxybenzyl)methylsulfonamido)-1-methyl-1H-indazol-7-yl)-4-oxo-7-propionyl-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

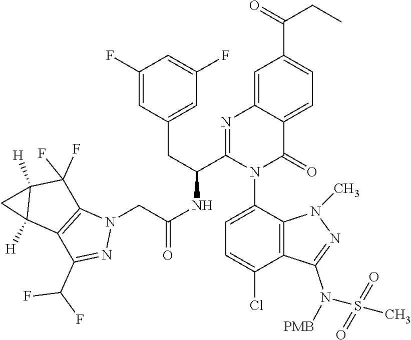

Bis(triphenylphosphine)palladium(II) chloride (5.78 mg, 8.23 μmol) was added to a solution of propionyl chloride (7.62 mg, 0.082 mmol) in Toluene (1 mL) and the mixture was stirred at room temp for 10 min. A solution of N—((S)-1-(3-(4-chloro-3-(N-(4-methoxybenzyl)methylsulfonamido)-1-methyl-1H-indazol-7-yl)-4-oxo-7-(tributylstannyl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (100 mg, 0.082 mmol) in Toluene (1 mL) was then added and the mixture was heated at 100° C. for 5 h. Mixture was then cooled, concentrated and purified by Biotage (5-40% EtOAc/hexane) to afford N—((S)-1-(3-(4-chloro-3-(N-(4-methoxybenzyl)methylsulfonamido)-1-methyl-1H-indazol-7-yl)-4-oxo-7-propionyl-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (20 mg, 0.020 mmol, 24.75% yield). LCMS (M+H)+=981.15

### N—((S)-1-(3-(4-chloro-1-methyl-3-(methylsulfonamido)-1H-indazol-7-yl)-7-(1,1-difluoropropyl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 20)

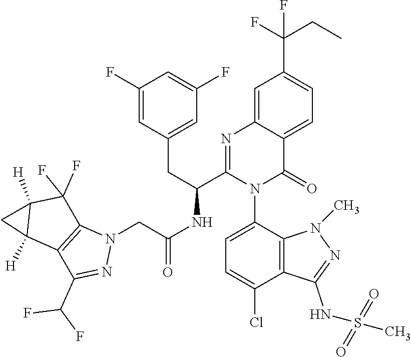 [SMILES: CCC(F)(F)c1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

To a solution of N—((S)-1-(3-(4-chloro-3-(N-(4-methoxybenzyl)methylsulfonamido)-1-methyl-1H-indazol-7-yl)-4-oxo-7-propionyl-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (20 mg, 0.020 mmol) in Dichloromethane (DCM) (0.5 mL) in a sealed tube was added DAST (0.081 mL, 0.611 mmol). The tube was then sealed and the resulting mixture was heated at 60° C. for 16 h. After cooling to room temp, the reaction mixture was poured into a sat. NaHCO₃ solution and extracted with DCM, dried (Na₂SO₄), filtered and concentrated. The residue was taken up in DCM (0.5 mL) and triflic acid (0.05 mL) and TFA (1 mL) were added. The mixture was stirred at rt for 1 h and then concentrated. The residue was then taken up in 2 mL of DMF and purified by prep HPLC (Column: Zorbax Eclipse Plus C18, 21.2×100 mm, 5 m particles; Solvent A=0.1% Formic Acid in 100% Water. Solvent B=Acetonitrile. Flow Rate=40 mL min. Start % B=59.1 Final % B=79.1. Gradient Time 7 min, then a 2 min hold at 98% B. Wavelength=215 and 254 nm. ESI+Range: 150 to 1500 dalton. Sample was loaded at 35% B.) to afford N—((S)-1-(3-(4-chloro-1-methyl-3-(methylsulfonamido)-1H-indazol-7-yl)-7-(1,1-difluoropropyl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (5.5 mg, 5.92 μmol, 29.0% yield). ¹H NMR (500 MHz, METHANOL-d₄) δ ppm 8.39 (d, J=8.05 Hz, 1H), 8.00 (d, J=1.19 Hz, 1H), 7.76 (dd, J=8.20, 1.64 Hz, 1H), 7.29-7.34 (m, 1H), 7.21-7.26 (m, 1H), 6.53-6.82 (m, 4H), 4.83-4.85 (m, 1H), 4.49-4.59 (m, 2H), 3.61 (s, 3H), 3.45-3.50 (m, 1H), 3.24 (s, 3H), 3.11 (dd, J=14.16, 9.39 Hz, 1H), 2.39-2.49 (m, 2H), 2.26-2.37 (m, 2H), 1.34-1.40 (m, 1H), 1.07 (t, J=7.45 Hz, 3H), 0.00-1.01 (m, 1H). LC/MS retention time=1.47 min; m z=883.1 [M+H]⁺. (Column: Acquity BEH C18, 2.1×30 mm, 1.7 m particles; Solvent A=0.10% Formic acid in 100% Water. Solvent B=0.10% Formic Acid in 100% Acetonitrile. Flow Rate=0.8 mL min. Start % B=5. Final % B=95. Gradient Time 1.7 min, then a 0.2 min hold at 95% B. Wavelength=215 and 254 nm. ESI+Range: 150 to 1500 Dalton. System: Agilent 1290 Infinity II)

### N—((S)-1-(3-(4-chloro-1-methyl-3-(methylsulfonamido)-1H-indazol-7-yl)-4-oxo-7-(3,3,3-trifluoropropyl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide and 2-((3bS,4aR)-3-(difluoromethyl)-5,5-difluoro-3b,4,4a,5-tetrahydro-1H-cyclopropa[3,4]cyclopenta[1,2-c]pyrazol-1-yl)-N—((S)-2-(3,5-difluorophenyl)-1-(3-(1-methyl-3-(methylsulfonamido)-4-(3,3,3-trifluoropropyl)-1H-indazol-7-yl)-4-oxo-7-(3,3,3-trifluoropropyl)-3,4-dihydroquinazolin-2-yl)ethyl)acetamide (Examples 21 and 22)

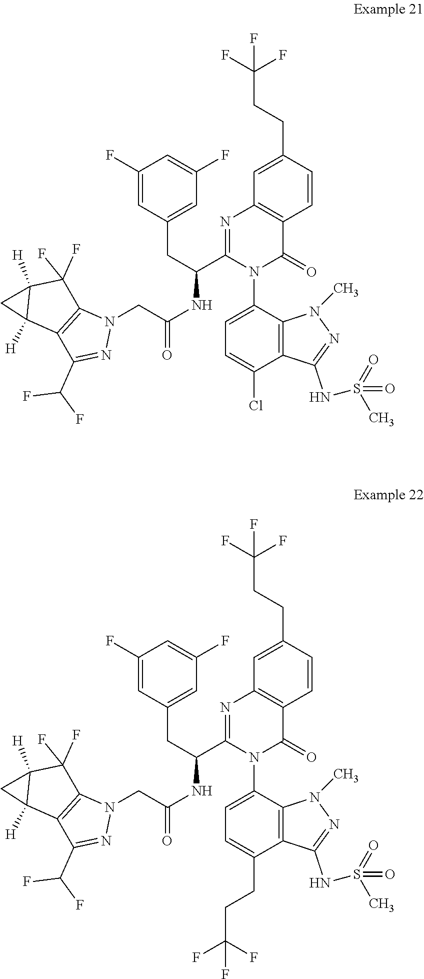 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(CCC(F)(F)F)ccc4c3=O)c21 ; Cn1nc(NS(C)(=O)=O)c2c(CCC(F)(F)F)ccc(-n3c([C@H](Cc4cc(F)cc(F)c4)NC(=O)Cn4nc(C(F)F)c5c4C(F)(F)[C@@H]4C[C@H]54)nc4cc(CCC(F)(F)F)ccc4c3=O)c21]

The title compound was prepared according to General Procedure E using potassium trifluoro(3,3,3-trifluoropropyl)borate as the coupling partner. The experiment afforded the title compound, N—((S)-1-(3-(4-chloro-1-methyl-3-(methylsulfonamido)-1H-indazol-7-yl)-4-oxo-7-(3,3,3-trifluoropropyl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method A: retention time=2.64 min.; observed ion=901.2 (M+H). 1H NMR (METHANOL-d4, 500 MHz) δ 8.76 (br d, 1H, J=8.5 Hz), 8.25 (d, 1H, J=8.2 Hz), 7.80 (s, 1H), 7.60 (br d, 1H, J=8.2 Hz), 7.30 (d, 1H, J=7.6 Hz), 7.16 (d, 1H, J=7.5 Hz), 6.7-6.8 (m, 1H), 6.69 (s, 1H), 6.6-6.6 (m, 2H), 4.8-4.9 (m, 1H), 4.53 (s, 2H), 3.60 (s, 3H), 3.4-3.5 (m, 1H), 3.2-3.3 (m, 3H), 3.1-3.2 (m, 3H), 2.6-2.7 (m, 2H), 2.4-2.5 (m, 2H), 1.3-1.4 (m, 1H), 1.0-1.1 (m, 1H). Example 22 was also isolated as a byproduct from the above reaction. The sample was analyzed using LCMS Method A: retention time=2.27 min.; observed ion=963.20 (M+H). ¹H NMR (METHANOL-d₄, 500 MHz) δ 8.13 (d, 1H, J=8.2 Hz), 7.67 (s, 1H), 7.48 (dd, 1H, J=1.6, 8.2 Hz), 7.1-7.1 (m, 2H), 6.6-6.7 (m, 1H), 6.56 (s, 1H), 6.55 (t, 1H, J=54.7 Hz), 6.4-6.5 (m, 1H), 4.6-4.7 (m, 1H), 4.4-4.5 (m, 2H), 3.5-3.5 (m, 4H), 3.3-3.4 (m, 2H), 3.0-3.1 (m, 5H), 2.9-3.0 (m, 1H), 2.5-2.6 (m, 4H), 2.3-2.3 (m, 2H), 1.2-1.3 (m, 1H), 0.8-0.9 (m, 1H).

### N—((S)-1-(3-(4-chloro-1-(2,2-difluoroethyl)-3-(methylsulfonamido)-1H-indazol-7-yl)-4-oxo-7-(3,3,3-trifluoropropyl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 23)

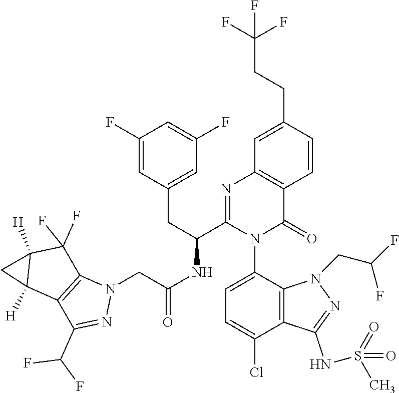 [SMILES: CS(=O)(=O)Nc1nn(CC(F)F)c2c(-n3c([C@H](Cc4cc(F)cc(F)c4)NC(=O)Cn4nc(C(F)F)c5c4C(F)(F)[C@@H]4C[C@H]54)nc4cc(CCC(F)(F)F)ccc4c3=O)ccc(Cl)c12]

The title compound was prepared according to General Procedure N using potassium trifluoro(3,3,3-trifluoropropyl)borate as the coupling partner. The experiment afforded the title compound, N—((S)-1-(3-(4-chloro-1-(2,2-difluoroethyl)-3-(methylsulfonamido)-1H-indazol-7-yl)-4-oxo-7-(3,3,3-trifluoropropyl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.47 min.; observed ion=917.4 (M+H). 1H NMR (METHANOL-d4, 500 MHz) δ 8.24 (d, 1H, J=8.3 Hz), 8.09 (d, 1H, J=2.3 Hz), 7.80 (s, 1H), 7.61 (dd, 1H, J=1.8, 8.0 Hz), 7.36 (d, 1H, J=7.7 Hz), 7.22 (d, 1H, J=8.0 Hz), 6.8-6.8 (m, 1H), 6.68 (s, 1H), 6.57 (s, 1H), 6.55 (br d, 1H, J=2.4 Hz), 6.02 (t, 1H, J=4.0 Hz), 4.9-4.9 (m, 1H), 4.74 (dd, 1H, J=4.9, 9.1 Hz), 4.6-4.7 (m, 2H), 3.40 (br d, 1H, J=4.8 Hz), 3.2-3.3 (m, 3H), 3.1-3.2 (m, 2H), 3.06 (dd, 1H, J=9.2, 14.0 Hz), 2.6-2.7 (m, 2H), 2.44 (td, 2H, J=3.8, 7.7 Hz), 1.3-1.4 (m, 1H), 1.01 (br dd, 1H, J=1.8, 3.6 Hz).

### N—((S)-1-(3-(4-chloro-3-(cyclopropanesulfonamido)-1-(2,2-difluoroethyl)-1H-indazol-7-yl)-4-oxo-7-(3,3,3-trifluoropropyl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 24)

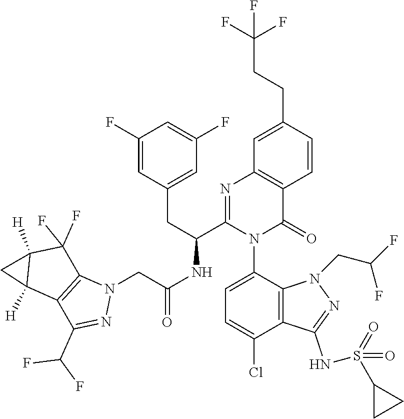 [SMILES: O=C(Cn1nc(C(F)F)c2c1C(F)(F)[C@@H]1C[C@H]21)N[C@@H](Cc1cc(F)cc(F)c1)c1nc2cc(CCC(F)(F)F)ccc2c(=O)n1-c1ccc(Cl)c2c(NS(=O)(=O)C3CC3)nn(CC(F)F)c12]

The title compound was prepared according to General Procedure O using potassium trifluoro(3,3,3-trifluoropropyl)borate as the coupling partner. The experiment afforded the title compound, N—((S)-1-(3-(4-chloro-3-(cyclopropanesulfonamido)-1-(2,2-difluoroethyl)-1H-indazol-7-yl)-4-oxo-7-(3,3,3-trifluoropropyl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.504 min.; observed ion=977.4 (M+H). 1H NMR (METHANOL-d4, 500 MHz) δ 8.24 (d, 1H, J=8.0 Hz), 7.80 (d, 1H, J=1.2 Hz), 7.61 (dd, 1H, J=1.6, 8.2 Hz), 7.37 (br d, 1H, J=7.7 Hz), 7.22 (d, 1H, J=7.7 Hz), 6.8-6.8 (m, 1H), 6.69 (t, 1H, J=54.8 Hz), 6.55 (dd, 2H, J=2.2, 8.2 Hz), 6.02 (t, 1H, J=4.0 Hz), 4.73 (dd, 1H, J=4.8, 9.2 Hz), 4.6-4.7 (m, 2H), 4.3-4.4 (m, 1H), 3.8-4.0 (m, 1H), 3.40 (dd, 1H, J=4.8, 14.0 Hz), 3.1-3.2 (m, 2H), 3.05 (dd, 1H, J=9.2, 14.0 Hz), 2.9-2.9 (m, 1H), 2.6-2.7 (m, 2H), 2.4-2.5 (m, 2H), 1.3-1.4 (m, 1H), 1.10 (dd, 2H, J=2.5, 4.9 Hz), 0.9-1.1 (m, 3H).

### N—((S)-1-(3-(4-chloro-1-methyl-3-(methylsulfonamido)-1H-indazol-7-yl)-7-isobutyl-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 25)

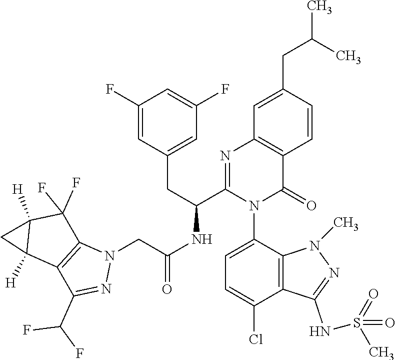 [SMILES: CC(C)Cc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1]

The title compound was prepared according to General Procedure E using trifluoro(isobutyl)borate as the coupling partner. The experiment afforded the title compound, N—((S)-1-(3-(4-chloro-1-methyl-3-(methylsulfonamido)-1H-indazol-7-yl)-7-isobutyl-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide. The sample was analyzed using LCMS Method F: retention time=1.54 min.; observed ion=861.4 (M+H). 1H NMR (METHANOL-d4, 500 MHz) δ 8.21 (d, 1H, J=8.0 Hz), 7.69 (d, 1H, J=1.2 Hz), 7.51 (dd, 1H, J=1.6, 8.2 Hz), 7.30 (d, 1H, J=7.7 Hz), 7.17 (d, 1H, J=7.7 Hz), 6.8-6.8 (m, 1H), 6.69 (br t, 1H, J=54.8 Hz), 6.62 (br dd, 2H, J=2.2, 8.2 Hz), 4.8-4.9 (m, 1H), 4.54 (d, 2H, J=2.4 Hz), 3.61 (s, 3H), 3.4-3.5 (m, 1H), 3.2-3.3 (m, 3H), 3.09 (dd, 1H, J=9.2, 14.0 Hz), 2.76 (d, 2H, J=7.5 Hz), 2.43 (dt, 2H, J=1.8, 4.8 Hz), 2.0-2.1 (m, 1H), 1.37 (br d, 1H, J=7.5 Hz), 1.0-1.0 (m, 7H).

### N—((S)-1-(7-(tert-butyl)-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide (Example 26)

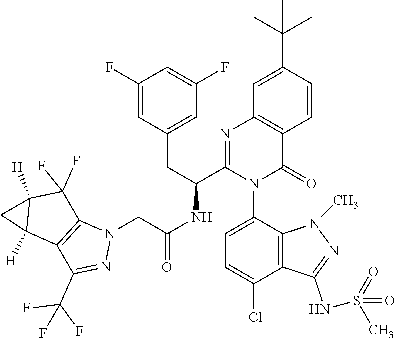 [SMILES: Cn1nc(NS(C)(=O)=O)c2c(Cl)ccc(-n3c([C@H](Cc4cc(F)cc(F)c4)NC(=O)Cn4nc(C(F)(F)F)c5c4C(F)(F)[C@@H]4C[C@H]54)nc4cc(C(C)(C)C)ccc4c3=O)c21]

To a solution of (S)—N-(7-(2-(1-amino-2-(3,5-difluorophenyl)ethyl)-7-(tert-butyl)-4-oxoquinazolin-3(4H)-yl)-4-chloro-1-methyl-1H-indazol-3-yl)methanesulfonamide (50 mg, 0.081 mmol),2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetic acid (22.94 mg, 0.081 mmol) in Tetrahydrofuran (THF) (1 mL) was added DIEA (0.043 mL, 0.244 mmol) followed by HATU (34.0 mg, 0.089 mmol) and the resulting mixture was stirred at room temp for 3 h. 2M ammonia in methanol (0.5 mL) was then added and the mixture was stirred at room temp for 30 min. Water was then added and the mixture was extracted with ethyl acetate, washed with brine, dried (Na₂SO₄), filtered and concentrated. The residue was then purified on C18 by Preparative HPLC using a gradient of 0.10% TFA buffered solvents: Water: Acetontirile. The fractions containing the expected product were concentrated by lyophilization to afford N—((S)-1-(7-(tert-butyl)-3-(4-chloro-1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide (33 mg, 0.036 mmol, 43.9% yield). ¹H NMR (500 MHz, METHANOL-d₄) δ ppm 8.23 (d, J=8.34 Hz, 1H), 7.91 (d, J=1.49 Hz, 1H), 7.79 (dd, J=8.49, 1.94 Hz, 1H), 7.31 (d, J=7.75 Hz, 1H), 7.17 (d, J=7.75 Hz, 1H), 6.79 (tt, J=9.20, 2.27 Hz, 1H), 6.61 (dd, J=8.20, 2.24 Hz, 2H), 4.80-4.84 (m, 1H), 4.52-4.68 (m, 2H), 3.59 (s, 3H), 3.44-3.51 (m, 1H), 3.26 (s, 3H), 3.25-3.28 (m, 1H), 3.10 (dd, J=14.01, 9.24 Hz, 1H), 2.43-2.53 (m, 2H), 1.49 (s, 9H), 1.37-1.43 (m, 1H), 1.04-1.10 (m, 1H). LC/MS retention time=3.48 min; m z=879.1 [M+H]⁺. (Column: Acquity UPLC BEH C18, 1.7 μm particles; Solvent A=95:5 Water:MeCN 0.1% TFA. Solvent B=5:95 Water:MeCN 0.1% TFA. Flow Rate=0.8 mL min. Start %=B=0. Final % B=100. Gradient Time 3 min, then a 1 min hold at 100% B. Wavelength=220 nm and 254 nm). UPLC: Gradient: Standard, Start % B: 0, Final % B: 100, Gradient Time: 15 min, Stop Time: 20 min, Flow Rate: 0.500 ml/min, Wavelength1: 220 Wavelength2: 254, Solvent A: water:MeCN 95:5 w/ 0.05% TFA, Solvent B: MeCN:water 95:5 w/ 0.05% TFA, Column: Acquity BEH C18 1.7 um, Oven Temperature: 40. rt: 13.13 min. purity=98%.

### tert-butyl (S)-(1-(7-(tert-butyl)-3-(4-chloro-1-(2,2-difluoroethyl)-3-(N-(4-methoxybenzyl)cyclopropanesulfonamido)-1H-indazol-7-yl)-4-oxo-3,4-dihydroquinazolin-2-yl)-2-(3,5-difluorophenyl)ethyl)carbamate

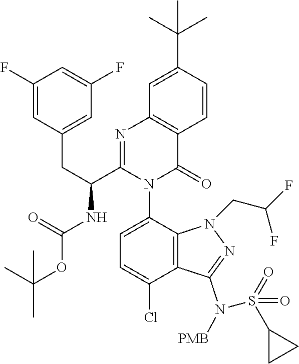

To a solution of (S)-2-((tert-butoxycarbonyl)amino)-3-(3,5-difluorophenyl)propanoic acid (0.640 g, 2.124 mmol), 2-amino-4-(tert-butyl)benzoic acid (0.451 g, 2.336 mmol) and N-(7-amino-4-chloro-1-(2,2-difluoroethyl)-1H-indazol-3-yl)-N-(4-methoxybenzyl)cyclopropanesulfonamide (1 g, 2.124 mmol) in Pyridine (8.49 ml) was added diphenyl phosphite (1.644 ml, 8.49 mmol) and the resulting mixture was heated at 70° C. for 16 h and then cooled and concentrated under reduced pressure. The mixture was then diluted with EtOAc (200 mL) and washed with 0.5 M citric acid (2×50 mL), 1N NaOH (2×50 mL), dried over Na₂SO₄ and concentrated. The residue was then purified on silica (80 g isco column) using 0-50% ethyl acetate in hexanes. Two atrope isomers were separable at this point. The desired fractions were concentrated to give First eluting major atropisomer: tert-butyl (S)-(1-(7-(tert-butyl)-3-(4-chloro-1-(2,2-difluoroethyl)-3-(N-(4-methoxybenzyl)cyclopropanesulfonamido)-1H-indazol-7-yl)-4-oxo-3,4-dihydroquinazolin-2-yl)-2-(3,5-difluorophenyl)ethyl)carbamate (692 mg, 0.759 mmol, 35.8% yield). 1H NMR (500 MHz, CDCl₃) δ ppm 8.24 (d, J=8.34 Hz, 1H), 7.80 (d, J=1.79 Hz, 1H), 7.67 (dd, J=8.49, 1.94 Hz, 1H), 7.30-7.33 (m, 2H), 7.15 (br d, J=7.45 Hz, 1H), 6.78 (br d, J=7.45 Hz, 2H), 6.70 (tt, J=8.87, 2.31 Hz, 1H), 6.33-6.49 (m, 3H), 5.66-6.14 (m, 1H), 5.21-5.43 (m, 1H), 4.74-5.13 (m, 2H), 4.28-4.48 (m, 2H), 3.97-4.12 (m, 1H), 3.75 (br s, 3H), 3.20 (br dd, J=13.56, 7.00 Hz, 1H), 2.90 (dd, J=13.41, 7.15 Hz, 1H), 2.44-2.65 (m, 1H), 1.49 (s, 9H), 1.34 (br s, 9H), 1.14 (br d, J=2.38 Hz, 2H), 0.89-0.97 (m, 2H). LCMS (M+Na)=933.25, LCMS RT=3.38 and second eluting minor atropisomer tert-butyl (S)-(1-(7-(tert-butyl)-3-(4-chloro-1-(2,2-difluoroethyl)-3-(N-(4-methoxybenzyl)cyclopropanesulfonamido)-1H-indazol-7-yl)-4-oxo-3,4-dihydroquinazolin-2-yl)-2-(3,5-difluorophenyl)ethyl)carbamate was not characterize.

### (S)—N-(7-(2-(1-amino-2-(3,5-difluorophenyl)ethyl)-7-(tert-butyl)-4-oxoquinazolin-3(4H)-yl)-4-chloro-1-(2,2-difluoroethyl)-1H-indazol-3-yl)cyclopropanesulfonamide

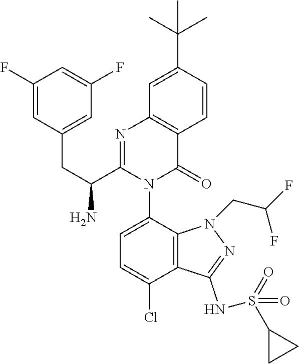 [SMILES: CC(C)(C)c1ccc2c(=O)n(-c3ccc(Cl)c4c(NS(=O)(=O)C5CC5)nn(CC(F)F)c34)c([C@@H](N)Cc3cc(F)cc(F)c3)nc2c1]

To a stirring solution of tert-butyl (S)-(1-(7-(tert-butyl)-3-(4-chloro-1-(2,2-difluoroethyl)-3-(N-(4-methoxybenzyl)cyclopropanesulfonamido)-1H-indazol-7-yl)-4-oxo-3,4-dihydroquinazolin-2-yl)-2-(3,5-difluorophenyl)ethyl)carbamate (690 mg, 0.757 mmol) in Dichloromethane (DCM) (8 mL) was added TFA (4 mL, 51.9 mmol) followed by triflic acid (0.101 mL, 1.136 mmol) at rt. and the resulting dark red solution was stirred at room temp for 2 h and then concentrated to a minimum on the rotovap. The residue was partitioned between EtOAc (200 mL) and aq. NaOH (1M, 20 mL). The aq. phase was tested and determined to be pH >=8.0. The organic phase was isolated and dried over Na2SO4, filtered, and then concentrated. The residue was then purified by Biotage (5-100% EtOAC/hexane) to afford (S)—N-(7-(2-(1-amino-2-(3,5-difluorophenyl)ethyl)-7-(tert-butyl)-4-oxoquinazolin-3(4H)-yl)-4-chloro-1-(2,2-difluoroethyl)-1H-indazol-3-yl)cyclopropanesulfonamide (440 mg, 0.637 mmol, 84% yield). 1H NMR (500 MHz, DMSO-d₆) δ ppm 8.12 (d, J=8.64 Hz, 1H), 7.77 (d, J=1.49 Hz, 1H), 7.73 (dd, J=8.49, 1.94 Hz, 1H), 7.35-7.49 (m, 2H), 7.00 (tt, J=9.54, 2.38 Hz, 1H), 6.71 (dd, J=8.64, 2.09 Hz, 2H), 6.14-6.44 (m, 1H), 4.35-4.47 (m, 1H), 4.21-4.35 (m, 1H), 3.49 (br dd, J=8.34, 4.47 Hz, 1H), 3.27 (dd, J=13.56, 4.32 Hz, 1H), 2.90-3.01 (m, 1H), 2.83 (dd, J=13.41, 8.64 Hz, 1H), 1.42 (s, 9H), 0.99-1.07 (m, 4H). LCMS (M+H)+=691.15

### N—((S)-1-(7-(tert-butyl)-3-(4-chloro-3-(cyclopropanesulfonamido)-1-(2,2-difluoroethyl)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 27)

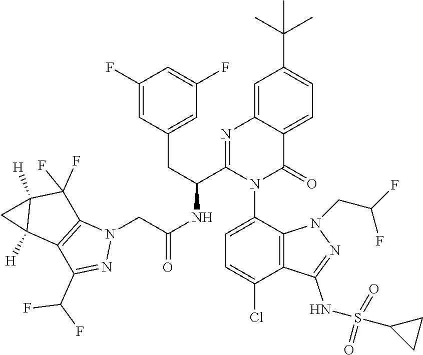 [SMILES: CC(C)(C)c1ccc2c(=O)n(-c3ccc(Cl)c4c(NS(=O)(=O)C5CC5)nn(CC(F)F)c34)c([C@H](Cc3cc(F)cc(F)c3)NC(=O)Cn3nc(C(F)F)c4c3C(F)(F)[C@@H]3C[C@H]43)nc2c1]

To a solution of (S)—N-(7-(2-(1-amino-2-(3,5-difluorophenyl)ethyl)-7-(tert-butyl)-4-oxoquinazolin-3(4H)-yl)-4-chloro-1-(2,2-difluoroethyl)-1H-indazol-3-yl)cyclopropanesulfonamide (100 mg, 0.145 mmol), 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (38.2 mg, 0.145 mmol) and DIEA (0.076 mL, 0.434 mmol) in Tetrahydrofuran (THF) (3 mL) was added HATU (60.5 mg, 0.159 mmol) and the resulting mixture was stirred at room temp for 3 h. 2M ammonia in methanol (0.5 mL) was then added and the mixture was stirred at room temp for 30 min. Water was then added and the mixture was extracted with ethyl acetate, washed with brine, dried (Na2SO4), filtered and concentrated. The residue was then purified via C18 prep HPLC (Column: Sunfire prep C18 OBD, 30×100 mm, 5 m particles; solvent A: water:MeCN 95:5 w/ 0.1% TFA, Solvent B: MeCN:water 95:5 w/ 0.1% TFA. Flow Rate=42 mL/min. Start % B=30 Final % B=100. Gradient Time=15 min, then a 5 min hold at 100% B. Wavelength=220 and 254 nm.) and product containing fractions were lyophilized to afford N—((S)-1-(7-(tert-butyl)-3-(4-chloro-3-(cyclopropanesulfonamido)-1-(2,2-difluoroethyl)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (70 mg, 0.071 mmol, 49.0% yield). ¹H NMR (500 MHz, METHANOL-d₄) b ppm 8.10 (d, J=8.64 Hz, 1H), 7.79 (d, J=1.79 Hz, 1H), 7.67 (dd, J=8.49, 1.94 Hz, 1H), 7.26 (d, J=7.75 Hz, 1H), 7.11 (d, J=7.75 Hz, 1H), 6.36-6.71 (m, 4H), 5.74-6.03 (m, 1H), 4.59-4.64 (m, 1H), 4.46-4.58 (m, 2H), 4.24 (dtd, J=15.20, 13.34, 13.34, 4.32 Hz, 1H), 3.80 (dtd, J=15.20, 13.56, 13.56, 3.87 Hz, 1H), 3.28 (dd, J=14.01, 4.77 Hz, 1H), 2.94 (dd, J=14.01, 9.24 Hz, 1H), 2.79 (tt, J=8.05, 4.77 Hz, 1H), 2.25-2.40 (m, 2H), 1.37 (s, 9H), 1.19-1.29 (m, 1H), 0.99 (dd, J=4.77, 2.38 Hz, 2H), 0.83-0.94 (m, 3H). LCMS (M+H)+=937.25

### (S)-tert-butyl (1-(7-(tert-butyl)-3-(4-chloro-3-(N-(4-methoxybenzyl)methylsulfonamido)-1-(2,2,2-trifluoroethyl)-1H-indazol-7-yl)-4-oxo-3,4-dihydroquinazolin-2-yl)-2-(3,5-difluorophenyl)ethyl)carbamate

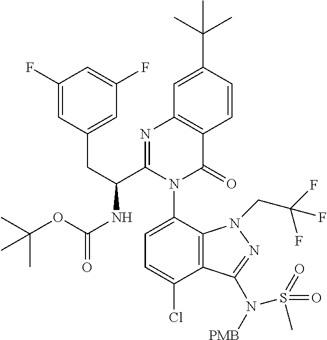

A mixture of 2-amino-4-(tert-butyl)benzoic acid (0.334 g, 1.728 mmol), (S)-2-((tert-butoxycarbonyl)amino)-3-(3,5-difluorophenyl)propanoic acid (0.521 g, 1.728 mmol), N-(7-amino-4-chloro-1-(2,2,2-trifluoroethyl)-1H-indazol-3-yl)-N-(4-methoxybenzyl)methanesulfonamide (0.8 g, 1.728 mmol) and diphenyl phosphonate (1.324 mL, 6.91 mmol) in pyridine (4.94 mL) was heated for 18 h at 75° C. Upon cooling to ambient temperature, the reaction was diluted with EtOAc (˜250 mL) and washed with 0.5 M citric acid. The organic layer was dried (Na₂SO₄) and concentrated in vacuo. The residue was purified by silica gel flash chromatography using 0-45% ethyl acetate in hexanes to give the product (0.63 g, 41%) as a pale yellow solid. LCMS (M+1): 903.20.

### (S)—N-(7-(2-(1-amino-2-(3,5-difluorophenyl)ethyl)-7-(tert-butyl)-4-oxoquinazolin-3(4H)-yl)-4-chloro-1-(2,2,2-trifluoroethyl)-H-indazol-3-yl)methanesulfonamide

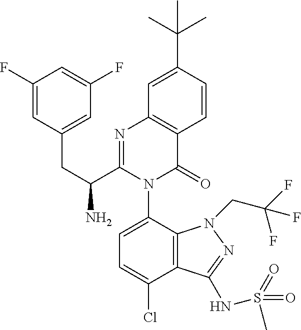 [SMILES: CC(C)(C)c1ccc2c(=O)n(-c3ccc(Cl)c4c(NS(C)(=O)=O)nn(CC(F)(F)F)c34)c([C@@H](N)Cc3cc(F)cc(F)c3)nc2c1]

Trifluoromethanesulfonic acid (0.235 ml, 2.65 mmol) was added to a solution of tert-butyl (S)-(1-(7-(tert-butyl)-3-(4-chloro-3-(N-(4-methoxybenzyl)methylsulfonamido)-1-(2,2,2-trifluoroethyl)-1H-indazol-7-yl)-4-oxo-3,4-dihydroquinazolin-2-yl)-2-(3,5-difluorophenyl)ethyl)carbamate (0.799 g, 0.884 mmol) in DCM (4.91 mL) and TFA (2.457 mL). The mixture was stirred for 1 h and concentrated in vacuo. The residue was taken up in ethyl acetate and 1 M NaOH was added until basic. The organic layer was separated, dried (Na₂SO₄), and concentrated in vacuo. The residue was purified by silica gel flash chromatography using 20-100% ethyl acetate in hexanes to give an off-white solid. The solid was then purified by C18 reverse phase flash chromatography using 10-60% 95:5 CH3CN:Water with 0.1% TFA in 95:5 Water:Acetonitrile with 0.1% TFA to separate the atropisomers. The second (major) eluting peak was concentrated and further purified by SFC chromatography using the following conditions: column: IG 5 micron, 21×250 mm; flow rate: 20 mL/min; solvents: 80:20 Heptane:EtOH; modifier: none; wavelength: 235 collect, 214 monitor; RT: 11.5 & 18.2 min; Length of run: 20 min (allowed the 2nd peak to not be collected, and was washed off the column during the end of the run, and the injection sequence. The product was isolated (0.388 g, 64%) as a white solid. ¹H NMR (500 MHz, METHANOL-d₄) δ 8.18 (d, J=8.64 Hz, 1H), 7.90 (d, J=1.49 Hz, 1H), 7.76 (dd, J=1.79, 10.43 Hz, 1H), 7.37 (d, J=7.75 Hz, 1H), 7.00 (d, J=8.05 Hz, 1H), 6.80 (tt, J=2.38, 9.24 Hz, 1H), 6.54 (dd, J=2.24, 8.20 Hz, 2H), 4.80-4.87 (m, 1H), 4.59-4.70 (m, 1H), 3.60 (dd, J=6.11, 7.30 Hz, 1H), 3.37 (s, 1H), 3.28-3.32 (m, 1H), 2.88 (dd, J=7.15, 13.41 Hz, 1H), 1.49 (s, 9H). LCMS (M+1): 683.10.

### N—((S)-1-(7-(tert-butyl)-3-(4-chloro-3-(methylsulfonamido)-1-(2,2,2-trifluoroethyl)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 28)

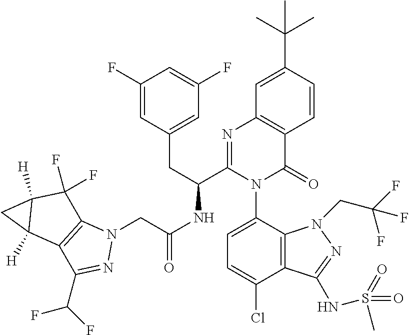 [SMILES: CC(C)(C)c1ccc2c(=O)n(-c3ccc(Cl)c4c(NS(C)(=O)=O)nn(CC(F)(F)F)c34)c([C@H](Cc3cc(F)cc(F)c3)NC(=O)Cn3nc(C(F)F)c4c3C(F)(F)[C@@H]3C[C@H]43)nc2c1]

To a stirred solution of (S)—N-(7-(2-(1-amino-2-(3,5-difluorophenyl)ethyl)-7-(tert-butyl)-4-oxoquinazolin-3(4H)-yl)-4-chloro-1-(2,2,2-trifluoroethyl)-1H-indazol-3-yl)methanesulfonamide (0.05 g, 0.073 mmol) in THF (1.464 mL) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.020 g, 0.077 mmol), 2-(3H-[1,2,3]triazolo[4,5-b]pyridin-3-yl)-1,1,3,3-tetramethylisouronium hexafluorophosphate(V) (0.033 g, 0.088 mmol), and DIPEA (0.032 ml, 0.183 mmol). The reaction mixture was stirred for 18 h and purified directly by silica gel flash chromatography using 0-100% ethyl acetate in hexanes to give N—((S)-1-(7-(tert-butyl)-3-(4-chloro-3-(methylsulfonamido)-1-(2,2,2-trifluoroethyl)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide as a white solid (51 mg) with some impurities. The solid was further purified by preparatory HPLC to give a white solid (0.034 g, 48%). ¹H NMR (500 MHz, METHANOL-d₄) δ ppm 8.75-8.93 (m, 1H) 8.15-8.25 (m, 1H) 7.86-7.92 (m, 1H) 7.75-7.81 (m, 1H) 7.41-7.47 (m, 1H) 7.27-7.33 (m, 1H) 6.46-6.84 (m, 4H) 4.65-4.79 (m, 4H) 4.14-4.23 (m, 1H) 3.24-3.29 (m, 3H) 2.97-3.06 (m, 1H) 2.41-2.49 (m, 2H) 1.46-1.53 (m, 9H) 1.35-1.43 (m, 1H) 0.97-1.04 (m, 1H). LC/MS retention time=3.44 min; m/z=929.2 [M+H]+Wavelength1: 220 nm, Wavelength2: 254 nm, Injection Vol.: 5.00 μl, Stop Time: 4.00, Grad. Time: 3.0, Start % B: 0, End % B: 100, Total Flow: 0.80 ml/min, Solvent A: 95:5 Water:MeCN 0.1% TFA, Solvent B: 5:95 Water:MeCN 0.1% TFA, Column: Acquity UPLC BEH C18, 2.1×50 mm, 1.7 m particles.

### N—((S)-1-(3-(4-chloro-3-(N-(4-methoxybenzyl)methylsulfonamido)-1-methyl-1H-indazol-7-yl)-4-oxo-7-pivaloyl-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

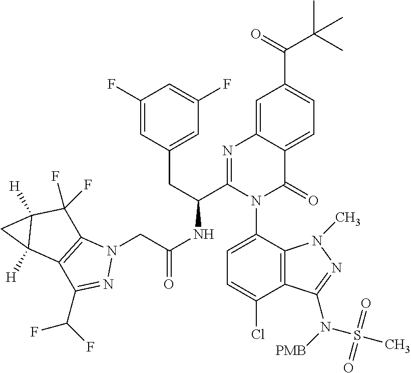

Bis(triphenylphosphine)palladium(II) chloride (5.78 mg, 8.23 μmol) was added to a solution of pivaloyl chloride (9.93 mg, 0.082 mmol) in Toluene (1 mL) and the mixture was stirred at room temp for 10 min. A solution of N—((S)-1-(3-(4-chloro-3-(N-(4-methoxybenzyl)methylsulfonamido)-1-methyl-1H-indazol-7-yl)-4-oxo-7-(tributylstannyl)-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (100 mg, 0.082 mmol) in Toluene (1 mL) was then added and the mixture was heated at 100° C. for 2 h. Mixture was then concentrated and the residue was purified by Biotage (5-40% EtOAc/hexane) to afford N—((S)-1-(3-(4-chloro-3-(N-(4-methoxybenzyl)methylsulfonamido)-1-methyl-1H-indazol-7-yl)-4-oxo-7-pivaloyl-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (29 mg, 0.029 mmol, 34.9% yield). LCMS (M+H)+=1009.20

### N—((S)-1-(3-(4-chloro-1-methyl-3-(methylsulfonamido)-1H-indazol-7-yl)-7-(1,1-difluoro-2,2-dimethylpropyl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 29)

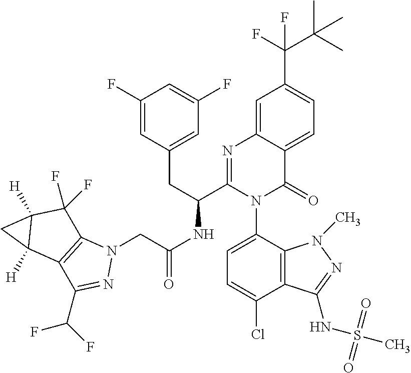 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(C(F)(F)C(C)(C)C)ccc4c3=O)c21]

To a solution of N—((S)-1-(3-(4-chloro-3-(N-(4-methoxybenzyl)methylsulfonamido)-1-methyl-1H-indazol-7-yl)-4-oxo-7-pivaloyl-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (27 mg, 0.027 mmol) in 1,2-Dichloroethane (DCE) (0.75 mL) in a sealed tube was added DAST (0.106 mL, 0.802 mmol). The tube was then sealed and the resulting mixture was heated at 80° C. for 16 h. After cooling to room temp, the reaction mixture was poured into a sat. NaHCO₃ solution and extracted with DCM, dried (Na₂SO₄), filtered and concentrated. The residue was taken up in DCM (0.5 mL) and triflic acid (0.05 mL) and TFA (1 mL) were added. The mixture was stirred at rt for 1 h and then concentrated. The residue was then taken up in 2 mL of DMF and purified by prep HPLC (Column: Zorbax Eclipse Plus C18, 21.2×100 mm, 5 μm particles; Solvent A=0.1% Formic Acid in 100% Water. Solvent B=Acetonitrile. Flow Rate=40 mL min. Start % B=62.9 Final % B=82.9. Gradient Time 7 min, then a 2 min hold at 98% B. Wavelength=215 and 254 nm. ESI+Range: 150 to 1500 dalton. Sample was loaded at 62.9% B.) afforded N—((S)-1-(3-(4-chloro-1-methyl-3-(methylsulfonamido)-1H-indazol-7-yl)-7-(1,1-difluoro-2,2-dimethylpropyl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (8.6 mg, 8.97 μmol, 33.5% yield). ¹H NMR (500 MHz, METHANOL-d₄) δ ppm 8.36 (d, J=8.34 Hz, 1H), 7.94 (d, J=1.49 Hz, 1H), 7.73 (dd, J=8.20, 1.64 Hz, 1H), 7.22-7.35 (m, 2H), 6.54-6.84 (m, 4H), 4.83-4.85 (m, 1H), 4.45-4.64 (m, 2H), 3.62 (s, 3H), 3.43-3.51 (m, 1H), 3.24 (s, 3H), 3.10 (dd, J=14.16, 9.39 Hz, 1H), 2.33-2.49 (m, 2H), 1.33-1.40 (m, 1H), 1.16 (s, 9H), 0.95-1.01 (m, 1H). LC/MS retention time=1.55 min; m z=911.4 [M+H]⁺. (Column: Acquity BEH C18, 2.1×30 mm, 1.7 m particles; Solvent A=0.10% Formic acid in 100% Water. Solvent B=0.10% Formic Acid in 100% Acetonitrile. Flow Rate=0.8 mL min. Start % B=5. Final % B=95. Gradient Time 1.7 min, then a 0.2 min hold at 95% B. Wavelength=215 and 254 nm. ESI+Range: 150 to 1500 Dalton. System: Agilent 1290 Infinity II)

### N—((S)-1-(3-(4-chloro-1-(2,2-difluoroethyl)-3-(methylsulfonamido)-1H-indazol-7-yl)-7-cyclopropyl-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 30)

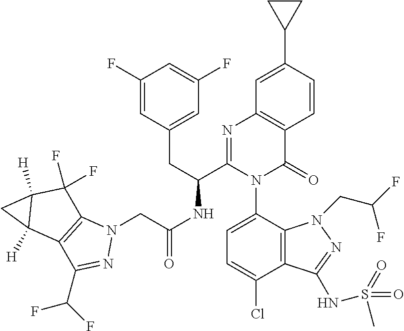 [SMILES: CS(=O)(=O)Nc1nn(CC(F)F)c2c(-n3c([C@H](Cc4cc(F)cc(F)c4)NC(=O)Cn4nc(C(F)F)c5c4C(F)(F)[C@@H]4C[C@H]54)nc4cc(C5CC5)ccc4c3=O)ccc(Cl)c12]

To a solution of N—((S)-1-(7-bromo-3-(4-chloro-1-(2,2-difluoroethyl)-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (50 mg, 0.054 mmol), cyclopropylboronic acid (18.39 mg, 0.214 mmol) and K₃PO₄ (45.5 mg, 0.214 mmol) in Tetrahydrofuran (THF) (2 mL)/Water (0.5 mL) was added Dichloro[9,9-dimethyl-4,5-bis(diphenylphosphino)xanthene]palladium(II) (4.05 mg, 5.35 μmol) and the resulting mixture was placed on a pre-heated heating block (100° C.) and stirred for 2 h. Mixture was then cooled concentrated and purified via C18 prep HPLC (Column: Sunfire prep C18 OBD, 30×100 mm, 5 m particles; solvent A: water:MeCN 95:5 w/ 0.1% TFA, Solvent B: MeCN:water 95:5 w/ 0.1% TFA. Flow Rate=42 mL/min. Start % B=30 Final % B=100. Gradient Time=15 min, then a 5 min hold at 100% B. Wavelength=220 and 254 nm.) and product containing fractions were lyophilized to afford N—((S)-1-(3-(4-chloro-1-(2,2-difluoroethyl)-3-(methylsulfonamido)-1H-indazol-7-yl)-7-cyclopropyl-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (23 mg, 0.024 mmol, 45.6% yield). ¹H NMR (500 MHz, METHANOL-d₄) δ ppm 8.03 (d, J=8.34 Hz, 1H), 7.44 (d, J=1.49 Hz, 1H), 7.23-7.29 (m, 2H), 7.09 (d, J=8.05 Hz, 1H), 6.41-6.72 (m, 4H), 5.75-6.03 (m, 1H), 4.58-4.65 (m, 1H), 4.42-4.54 (m, 2H), 4.18-4.30 (m, 1H), 3.73-3.87 (m, 1H), 3.29 (dd, J=14.16, 5.22 Hz, 1H), 3.15 (s, 3H), 2.93 (dd, J=14.01, 9.24 Hz, 1H), 2.33 (ddd, J=11.18, 7.45, 4.02 Hz, 2H), 2.03-2.13 (m, 1H), 1.22-1.30 (m, 1H), 1.09-1.16 (m, 2H), 0.87-0.92 (m, 1H), 0.83-0.87 (m, 2H). LCMS (M+H)+=895.10

### N—((S)-1-(3-(4-chloro-3-(cyclopropanesulfonamido)-1-(2,2-difluoroethyl)-1H-indazol-7-yl)-7-cyclopropyl-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 31)

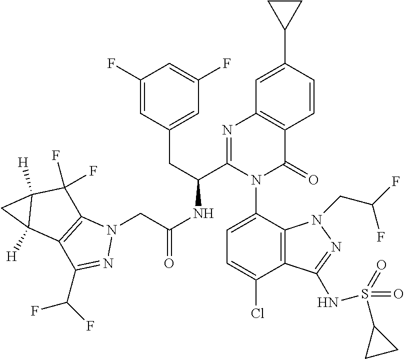 [SMILES: O=C(Cn1nc(C(F)F)c2c1C(F)(F)[C@@H]1C[C@H]21)N[C@@H](Cc1cc(F)cc(F)c1)c1nc2cc(C3CC3)ccc2c(=O)n1-c1ccc(Cl)c2c(NS(=O)(=O)C3CC3)nn(CC(F)F)c12]

To a solution of N—((S)-1-(7-bromo-3-(4-chloro-3-(cyclopropanesulfonamido)-1-(2,2-difluoroethyl)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (50 mg, 0.052 mmol), cyclopropylboronic acid (17.89 mg, 0.208 mmol) and K3PO4 (44.2 mg, 0.208 mmol) in Tetrahydrofuran (THF) (2 mL)/Water (0.5 mL) was added Dichloro[9,9-dimethyl-4,5-bis(diphenylphosphino)xanthene]palladium(II) (3.94 mg, 5.21 μmol) and the resulting mixture was placed on a pre-heated heating block (100° C.) and stirred for 2 h. Mixture was then cooled concentrated and purified via C18 prep HPLC (Column: Sunfire prep C18 OBD, 30×100 mm, 5 m particles; solvent A: water:MeCN 95:5 w/ 0.1% TFA, Solvent B: MeCN:water 95:5 w/ 0.1% TFA. Flow Rate=42 mL/min. Start % B=30 Final % B=100. Gradient Time=15 min, then a 5 min hold at 100% B. Wavelength=220 and 254 nm.) and product containing fractions were lyophilized to afford N—((S)-1-(3-(4-chloro-3-(cyclopropanesulfonamido)-1-(2,2-difluoroethyl)-1H-indazol-7-yl)-7-cyclopropyl-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (27 mg, 0.028 mmol, 53.5% yield

¹H NMR (500 MHz, METHANOL-d₄) δ ppm 8.03 (d, J=8.05 Hz, 1H), 7.44 (d, J=1.79 Hz, 1H), 7.23-7.30 (m, 2H), 7.10 (d, J=7.75 Hz, 1H), 6.39-6.72 (m, 4H), 5.75-6.03 (m, 1H), 4.61 (td, J=8.87, 4.92 Hz, 1H), 4.44-4.57 (m, 2H), 4.19-4.31 (m, 1H), 3.73-3.86 (m, 1H), 3.25-3.31 (m, 1H), 2.93 (dd, J=13.86, 9.09 Hz, 1H), 2.73-2.83 (m, 1H), 2.28-2.39 (m, 2H), 2.04-2.13 (m, 1H), 1.25 (td, J=7.60, 5.66 Hz, 1H), 1.09-1.15 (m, 2H), 0.95-1.03 (m, 2H), 0.82-0.91 (m, 5H). LCMS (M+H)+=921.10

### N—((S)-1-(3-(4-chloro-1-methyl-3-(methylsulfonamido)-1H-indazol-7-yl)-7-(cyclopent-1-en-1-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

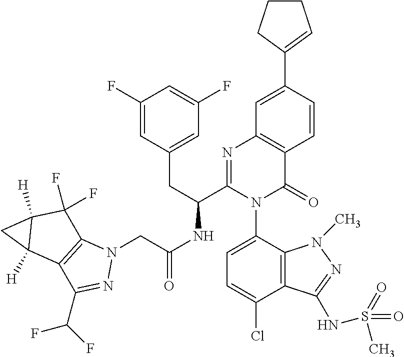 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(C5=CCCC5)ccc4c3=O)c21]

The title compound was prepared according to General Procedure L using N—((S)-1-(3-(4-chloro-1-methyl-3-(methylsulfonamido)-1H-indazol-7-yl)-7-(cyclopent-1-en-1-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide as the coupling partner. 1H NMR (METHANOL-d4, 500 MHz) δ 8.21 (d, 1H, J=8.3 Hz), 7.84 (s, 1H), 7.85 (d, 2H, J=9.2 Hz), 7.30 (d, 1H, J=7.7 Hz), 7.17 (d, 1H, J=7.7 Hz), 6.8-6.8 (m, 1H), 6.6-6.7 (m, 3H), 6.69 (t, 1H, J=54.8 Hz), 4.53 (d, 2H, J=2.4 Hz), 3.61 (s, 3H), 3.4-3.5 (m, 1H), 3.2-3.3 (m, 3H), 3.10 (dd, 1H, J=8.9, 14.0 Hz), 2.8-2.9 (m, 2H), 2.6-2.7 (m, 2H), 2.44 (ddd, 2H, J=4.0, 7.7, 11.3 Hz), 2.1-2.2 (m, 2H), 1.3-1.4 (m, 1H), 1.0-1.0 (m, 1H). LCMS (M+1): 871.4.

### N—((S)-1-(3-(4-chloro-1-methyl-3-(methylsulfonamido)-1H-indazol-7-yl)-7-cyclopentyl-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide and N—((S)-1-(7-cyclopentyl-3-(1-methyl-3-(methylsulfonamido)-1H-indazol-7-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Examples 32 and 33)

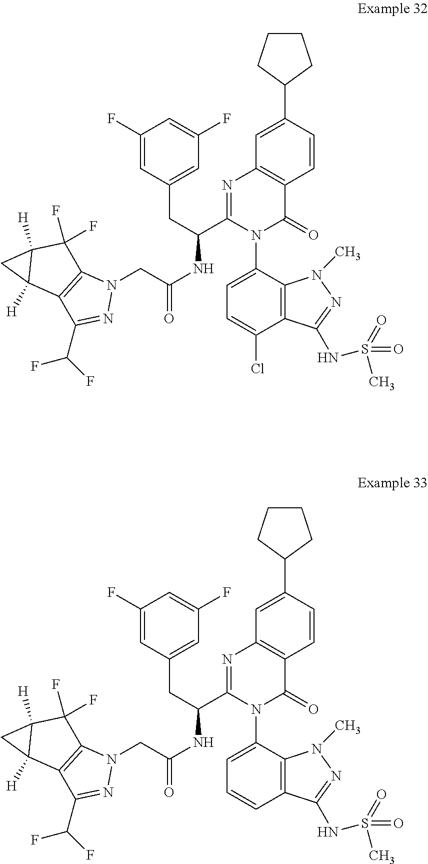 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(C5CCCC5)ccc4c3=O)c21 ; Cn1nc(NS(C)(=O)=O)c2cccc(-n3c([C@H](Cc4cc(F)cc(F)c4)NC(=O)Cn4nc(C(F)F)c5c4C(F)(F)[C@@H]4C[C@H]54)nc4cc(C5CCCC5)ccc4c3=O)c21]

10% Pd/C (30.5 mg, 0.029 mmol) was added to a degassed solution of N—((S)-1-(3-(4-chloro-1-methyl-3-(methylsulfonamido)-1H-indazol-7-yl)-7-(cyclopent-1-en-1-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (50 mg, 0.057 mmol) in methanol (0.57 mL) and acetic acid (0.57 mL). The reaction was evacuated and charged with H₂ via balloon. The reaction was stirred for 5 h. The reaction was flushed with Ar and celite was added to the reaction mixture. The slurry was filtered through a pad of celite washing with DCM. The filtrate was concentrated in vacuo. The crude product was purified by preparatory HPLC using the following conditions: Column: Zorbax Eclipse Plus C18, 21.2×100 mm, 5 m particles; Solvent A=0.10% Formic Acid in 100% Water. Solvent B=Acetonitrile. Flow Rate=40 mL/min. Start % B=63.2 Final % B=83.2. Gradient Time=7 min, then a 2 min hold at 98% B. Wavelength=215 and 254 nm. ESI+Range: 150 to 1500 dalton. Sample was loaded at 63.2% B. The major product (Example 32) was isolated (16 mg, 28%). The sample was analyzed using LCMS Method F: retention time=1.56 min.; observed ion=873.3 (M+H). 1H NMR (METHANOL-d4, 500 MHz) δ 8.21 (d, 1H, J=8.0 Hz), 8.03 (dd, 1H, J=0.9, 8.3 Hz), 7.77 (d, 1H, J=1.8 Hz), 7.60 (dd, 1H, J=1.8, 8.6 Hz), 7.29 (t, 1H, J=7.6 Hz), 7.18 (dd, 1H, J=0.9, 7.5 Hz), 6.7-6.8 (m, 1H), 6.68 (br t, 1H, J=54.7 Hz), 6.55 (dd, 2H, J=2.2, 8.2 Hz), 4.9-5.0 (m, 1H), 4.8-4.9 (m, 1H), 4.58 (d, 2H, J=1.8 Hz), 3.5-3.6 (m, 3H), 3.4-3.5 (m, 1H), 3.3-3.3 (m, 1H), 3.13 (s, 3H), 3.03 (dd, 1H, J=9.1, 13.9 Hz), 2.43 (brt, 2H, J=3.9 Hz), 2.24 (br d, 2H, J=1.5 Hz), 1.95 (brt, 2H, J=2.7 Hz), 1.7-1.9 (m, 4H), 1.01 (br s, 1H). The deschloro analog (Example 33) was also isolated from the same experiment. The sample was analyzed using LCMS Method F: retention time=1.54 min.; observed ion=839.4 (M+H). 1H NMR (METHANOL-d4, 500 MHz) δ 8.21 (d, 1H, J=8.0 Hz), 7.76 (d, 1H, J=1.8 Hz), 7.60 (dd, 1H, J=1.8, 8.3 Hz), 7.29 (d, 1H, J=7.7 Hz), 7.1-7.2 (m, 1H), 6.5-6.8 (m, 4H), 4.8-4.9 (m, 2H), 4.53 (d, 2H, J=2.1 Hz), 3.6-3.6 (m, 3H), 3.4-3.5 (m, 1H), 3.2-3.3 (m, 3H), 3.0-3.1 (m, 1H), 2.4-2.5 (m, 2H), 2.2-2.3 (m, 2H), 1.9-2.0 (m, 2H), 1.7-1.9 (m, 4H), 1.3-1.4 (m, 1H), 1.0-1.0 (m, 1H).

### N—((S)-1-(3-(4-chloro-1-methyl-3-(methylsulfonamido)-1H-indazol-7-yl)-7-(cyclohex-1-en-1-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide

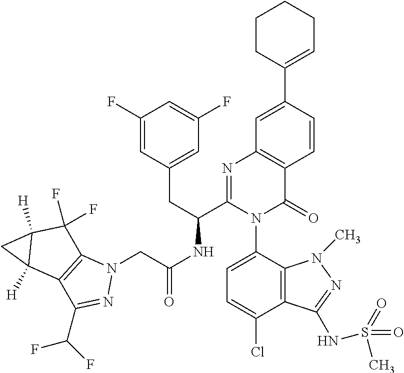 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(C5=CCCCC5)ccc4c3=O)c21]

The title compound was prepared according to General Procedure L using N—((S)-1-(3-(4-chloro-1-methyl-3-(methylsulfonamido)-1H-indazol-7-yl)-7-(cyclohex-1-en-1-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide as the coupling partner. 1H NMR (METHANOL-d4, 500 MHz) δ 8.20 (d, 1H, J=8.3 Hz), 7.85 (d, 1H, J=1.8 Hz), 7.76 (dd, 1H, J=1.8, 8.3 Hz), 7.29 (d, 1H, J=7.7 Hz), 7.16 (d, 1H, J=7.7 Hz), 6.8-6.8 (m, 1H), 6.6-6.6 (m, 2H), 6.69 (br t, 1H, J=54.7 Hz), 6.5-6.5 (m, 1H), 4.54 (d, 2H, J=3.0 Hz), 3.60 (s, 3H), 3.4-3.5 (m, 1H), 3.2-3.3 (m, 3H), 3.09 (dd, 1H, J=9.2, 14.0 Hz), 2.5-2.6 (m, 2H), 2.3-2.5 (m, 4H), 1.9-1.9 (m, 2H), 1.7-1.8 (m, 2H), 1.3-1.4 (m, 1H), 1.26 (s, 1H), 1.01 (td, 1H, J=2.1, 3.6 Hz). LCMS (M+1): 885.2.

### N—((S)-1-(3-(4-chloro-1-methyl-3-(methylsulfonamido)-1H-indazol-7-yl)-7-cyclohexyl-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 34)

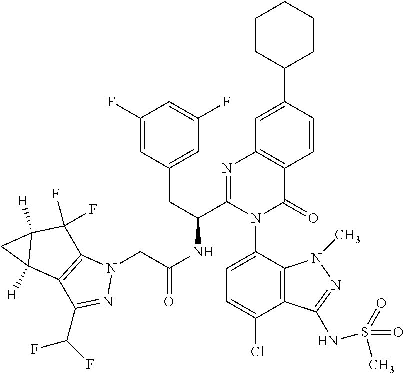 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(C5CCCCC5)ccc4c3=O)c21]

10% Pd/C (30.1 mg, 0.028 mmol) was added to a solution of N—((S)-1-(3-(4-chloro-1-methyl-3-(methylsulfonamido)-1H-indazol-7-yl)-7-(cyclohex-1-en-1-yl)-4-oxo-3,4-dihydroquinazolin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (50 mg, 0.056 mmol) in methanol (0.56 mL) and AcOH (0.56 mL). The reaction was evacuated and charged with H₂ via a balloon. The reaction was stirred for 5 h. The reaction was then filtered through a pad of Celite eluting with DCM. The filtrate was concentrated in vacuo. The crude product was purified by preparatory HPLC using the following conditions: Column: Zorbax Eclipse Plus C18, 21.2×100 mm, 5 m particles; Solvent A=0.1% Formic Acid in 100% Water. Solvent B=Acetonitrile. Flow Rate=40 mL/min. Start % B=66.1 Final % B=86.1. Gradient Time=7 min, then a 2 min hold at 98% B. Wavelength=215 and 254 nm. ESI+Range: 150 to 1500 dalton. Sample was loaded at 66.1% B. The product was isolated (19 mg, 37%). The product was analyzed using LCMS Method F: retention time=1.61 min.; observed ion=887.4 (M+H). 1H NMR (METHANOL-d4, 500 MHz) δ 8.21 (d, 1H, J=8.3 Hz), 7.73 (d, 1H, J=1.8 Hz), 7.57 (dd, 1H, J=1.5, 8.3 Hz), 7.29 (d, 1H, J=8.0 Hz), 7.15 (d, 1H, J=8.0 Hz), 6.8-6.8 (m, 1H), 6.6-6.6 (m, 2H), 6.69 (t, 1H, J=54.8 Hz), 4.8-4.9 (m, 1H), 4.54 (d, 2H, J=2.4 Hz), 3.58 (s, 3H), 3.4-3.5 (m, 1H), 3.24 (s, 3H), 3.09 (dd, 1H, J=8.9, 14.0 Hz), 2.8-2.8 (m, 1H), 2.44 (ddd, 2H, J=4.2, 7.5, 11.3 Hz), 1.9-2.0 (m, 4H), 1.85 (br d, 1H, J=12.8 Hz), 1.5-1.7 (m, 4H), 1.3-1.4 (m, 2H), 1.0-1.0 (m, 1H).

### (S)—N-(1-(3-(4-chloro-1-methyl-3-(methylsulfonamido)-1H-indazol-7-yl)-7-methoxy-4-oxo-3,4-dihydropyrido[2,3-d]pyrimidin-2-yl)-2-(3,5-difluorophenyl)ethyl)-2-(3-isopropyl-1H-pyrazol-1-yl)acetamide (Example 35)

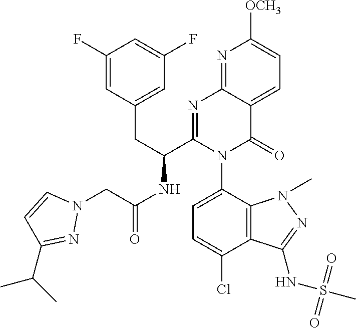 [SMILES: COc1ccc2c(=O)n(-c3ccc(Cl)c4c(NS(C)(=O)=O)nn(C)c34)c([C@H](Cc3cc(F)cc(F)c3)NC(=O)Cn3ccc(C(C)C)n3)nc2n1]

To a stirred solution of (S)—N-(7-(2-(1-amino-2-(3,5-difluorophenyl)ethyl)-7-methoxy-4-oxopyrido[2,3-d]pyrimidin-3(4H)-yl)-4-chloro-1-methyl-1H-indazol-3-yl)methanesulfonamide (90 mg, 0.153 mmol) and 2-(3-isopropyl-1H-pyrazol-1-yl)acetic acid (25.7 mg, 0.153 mmol) in DMF (2 mL) were added 1-hydroxybenzotriazole (6.18 mg, 0.046 mmol) and N-methylmorpholine (0.029 mL, 0.259 mmol) followed by N-(3-Dimethylaminopropyl)-N′-ethylcarbodiimide hydrochloride (49.7 mg, 0.259 mmol) at 27° C. The reaction mixture was stirred at 27° C. for 16 h. The progress of the reaction was monitored by TLC (SiO₂, 50% EtOAc/Pet., Rf=0.3, UV-active). Upon completion, the reaction mixture was diluted with EtOAc (50 mL) and washed with ice cold water (4×40 mL). The organic layer was dried over anhydrous Na₂SO₄, filtered and concentrated under reduced pressure to afford the crude compound which was purified by column chromatography on silica gel (SiO₂, 100-200 mesh) eluting with 40-55% EtOAc/Pet. The fractions containing product were collected and concentrated under reduced pressure to obtain an off-white solid (82 mg, LCMS purity=95%). The obtained off-white solid was further purified by Prepative TLC with 30-35% EtOAc/Pet. The fraction containing pure compound was collected, dissolved in 10% MeOH/DCM (10 mL) and filtered. The filtrate was concentrated under reduced pressure to obtain a colourless sticky solid which was then triturated with n-pentane (3×10 mL) to give an off-white solid. The obtained solid was dissolved in CH₃CN and water (1:1, 20 mL), freezed and lyophilized to afforded (S)—N-(1-(3-(4-chloro-1-methyl-3-(methylsulfonamido)-1H-indazol-7-yl)-7-methoxy-4-oxo-3,4-dihydropyrido[2,3-d]pyrimidin-2-yl)-2-(3,5-difluorophenyl)ethyl)-2-(3-isopropyl-1H-pyrazol-1-yl)acetamide (56 mg, Yield: 48%, off-white solid). ¹HNMR (400 MHz, DMSO-d₆) δ=9.85 (brs, 1H), 9.21 (d, J=8.3 Hz, 1H), 8.42 (d, J=8.6 Hz, 1H), 7.74 (d, J=7.9 Hz, 1H), 7.45 (d, J=7.9 Hz, 1H), 7.29 (d, J=2.2 Hz, 1H), 7.08 (d, J=8.5 Hz, 1H), 7.04-6.99 (m, 1H), 6.68 (d, J=6.2 Hz, 2H), 5.94 (d, J=2.2 Hz, 1H), 4.54-4.49 (m, 1H), 4.46 (s, 2H), 4.05 (s, 3H), 3.50 (s, 3H), 3.42-3.34 (m, 1H), 3.19 (s, 3H), 3.10-3.04 (m, 1H), 2.76-2.66 (m, 1H), 1.06 (dd, J=7.0, 1.1 Hz, 6H). LCMS: RT=2.45 mins, (M+H)=740.15, Purity=98%, HPLC Purity=98%, Chiral HPLC Purity=97%.

### (S)—N-(1-(3-(4-chloro-1-methyl-3-(methylsulfonamido)-1H-indazol-7-yl)-7-methoxy-4-oxo-3,4-dihydropyrido[2,3-d]pyrimidin-2-yl)-2-(3,5-difluorophenyl)ethyl)-2-(3-cyclopropyl-1H-pyrazol-1-yl)acetamide (Example 36)

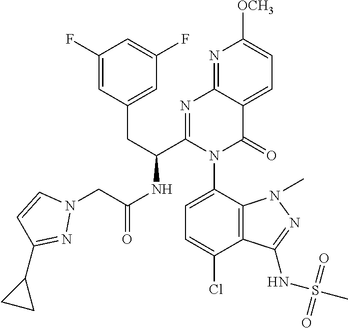 [SMILES: COc1ccc2c(=O)n(-c3ccc(Cl)c4c(NS(C)(=O)=O)nn(C)c34)c([C@H](Cc3cc(F)cc(F)c3)NC(=O)Cn3ccc(C4CC4)n3)nc2n1]

To a solution of (S)—N-(7-(2-(1-amino-2-(3,5-difluorophenyl)ethyl)-7-methoxy-4-oxopyrido[2,3-d]pyrimidin-3(4H)-yl)-4-chloro-1-methyl-1H-indazol-3-yl)methanesulfonamide (90 mg, 0.153 mmol) and 2-(3-cyclopropyl-1H-pyrazol-1-yl)acetic acid (25.3 mg, 0.153 mmol) in DMF (2 mL) were added 1-hydroxybenzotriazole (6.18 mg, 0.046 mmol), N-methylmorpholine (0.029 mL, 0.259 mmol) and EDC (49.7 mg, 0.259 mmol) at 27° C. The reaction mixture was stirred at 27° C. for 16 h. The progress of the reaction was monitored by TLC (SiO₂, 50% EtOAc/Pet., Rf=0.3, UV-active). Upon completion, the reaction mixture was diluted with EtOAc (50 mL) and washed with ice cold water (4×40 mL). The organic layer was dried over anhydrous Na₂SO₄, filtered and concentrated under reduced pressure to give the crude compound which was purified by column chromatography on silica gel (SiO₂, 100-200 mesh) eluting with 40-55% EtOAc/Pet. The fractions containing product were collected and concentrated under reduced pressure to afford an off-white solid (88 mg). The obtained solid was purified by preparative TLC with 30-35% EtOAc/Pet. After 3 times elution, the fraction containing pure compound was collected, dissolved in 10% MeOH/DCM (10 mL) and filtered. The filtrate was concentrated under reduced pressure to obtain a colourless sticky solid which was then triturated with n-pentane (3×10 mL) to give an off-white solid. The obtained solid was dissolved in CH₃CN and water (1:1, 20 mL), freezed and lyophilized to afford (S)—N-(1-(3-(4-chloro-1-methyl-3-(methylsulfonamido)-1H-indazol-7-yl)-7-methoxy-4-oxo-3,4-dihydropyrido[2,3-d]pyrimidin-2-yl)-2-(3,5-difluorophenyl)ethyl)-2-(3-cyclopropyl-1H-pyrazol-1-yl)acetamide (52 mg, Yield: 45%, off-white solid). 1HNMR (400 MHz, DMSO-d₆) δ=9.85 (br s, 1H), 9.19 (d, J=8.3 Hz, 1H), 8.42 (d, J=8.6 Hz, 1H), 7.75 (d, J=7.9 Hz, 1H), 7.45 (d, J=8.1 Hz, 1H), 7.26 (d, J=2.2 Hz, 1H), 7.08 (d, J=8.6 Hz, 1H), 7.05-7.00 (m, 1H), 6.67 (d, J=6.4 Hz, 2H), 5.81 (d, J=2.2 Hz, 1H), 4.52-4.46 (m, 1H), 4.43 (s, 2H), 4.06 (s, 3H), 3.52 (s, 3H), 3.42-3.38 (m, 1H), 3.19 (s, 3H), 3.09-3.02 (m, 1H), 1.73-1.69 (m, 1H), 0.72-0.70 (m, 2H), 0.50-0.49 (m, 2H). LCMS: RT=2.36 mins, (M+H)=738.05, Purity=97%, HPLC Purity=97%, Chiral HPLC Purity=97%.

### (S)-tert-butyl (1-(6-chloro-3-(4-chloro-3-(N-(4-methoxybenzyl)methylsulfonamido)-1-methyl-1H-indazol-7-yl)-7-methoxy-4-oxo-3,4-dihydropyrido[2,3-d]pyrimidin-2-yl)-2-(3,5-difluorophenyl)ethyl)carbamate

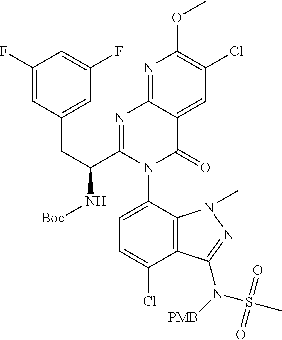

A solution of 2-amino-5-chloro-6-methoxynicotinic acid (0.49 g, 2.39 mmol, 1 equiv), (S)-2-((tert-butoxycarbonyl)amino)-3-(3,5-difluorophenyl)propanoic acid (0.79 g, 2.63 mmol, 1.1 equiv), N-(7-amino-4-chloro-1-methyl-1H-indazol-3-yl)-N-(4-methoxybenzyl)methanesulfonamide (1.04 g, 2.63 mmol, 1.1 equiv), and diphenyl phosphite (1.86 mL, 9.58 mmol, 4 equiv) in pyridine (10 mL) was heated at 70° C. for 4 h. Upon cooling to ambient temperature, the mixture was added to 0.5 M aqueous citric acid and extracted with EtOAc. The EtOAc layer was dried (Na₂SO₄) and concentrated in vacuo. The crude product was purified by silica gel flash chromatography (10-100% EtOAc/hexane) to provide the product (0.53 g, 26%). LCMS (M+1): 844.20.

### (S)—N-(7-(2-(1-amino-2-(3,5-difluorophenyl)ethyl)-6-chloro-7-methoxy-4-oxopyrido[2,3-d]pyrimidin-3(4H)-yl)-4-chloro-1-methyl-1H-indazol-3-yl)methanesulfonamide

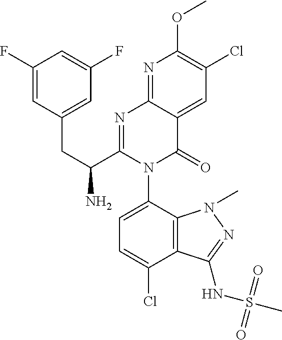 [SMILES: COc1nc2nc([C@@H](N)Cc3cc(F)cc(F)c3)n(-c3ccc(Cl)c4c(NS(C)(=O)=O)nn(C)c34)c(=O)c2cc1Cl]

Triflic acid (0.056 mL, 0.627 mmol) was added to a solution tert-butyl (S)-(1-(6-chloro-3-(4-chloro-3-(N-(4-methoxybenzyl)methylsulfonamido)-1-methyl-1H-indazol-7-yl)-7-methoxy-4-oxo-3,4-dihydropyrido[2,3-d]pyrimidin-2-yl)-2-(3,5-difluorophenyl)ethyl)carbamate (0.53 g, 0.627 mmol) in DCM (3.14 mL) and TFA (1.962 mL). The mixture was stirred for 1 h and concentrated in vacuo. The residue was taken up in ethyl acetate and 1 M NaOH was added until basic. The organic layer was dried (Na₂SO₄) and concentrated in vacuo. The residue was purified on by silica gel flash chromatography using 20-80% ethyl acetate in hexanes to give an off-white solid (0.257 g) which was purified by SFC chromatography using the following conditions: column: IA, 20×250 mm, 5u; Co-solvent: EtOH; Isocratic: 30% EtOH; Flow rate: 55 g/min; Back pressure: 120 Bar; UV wavelength: 220 nm; Temperature: 35° C.; Injection vol: 1 mL. The product was isolated (63 mg, 16%). LCMS (M+1): 624.15.

### N—((S)-1-(6-chloro-3-(4-chloro-1-methyl-3-(methylsulfonamido)-1H-indazol-7-yl)-7-methoxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 37)

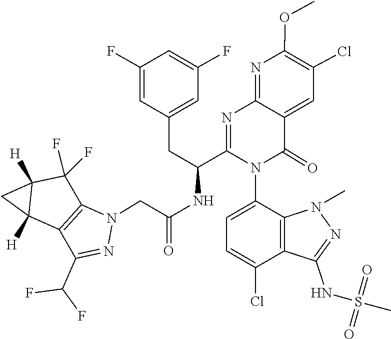 [SMILES: COc1nc2nc([C@H](Cc3cc(F)cc(F)c3)NC(=O)Cn3nc(C(F)F)c4c3C(F)(F)[C@H]3C[C@@H]43)n(-c3ccc(Cl)c4c(NS(C)(=O)=O)nn(C)c34)c(=O)c2cc1Cl]

To a stirred solution of (S)—N-(7-(2-(1-amino-2-(3,5-difluorophenyl)ethyl)-6-chloro-7-methoxy-4-oxopyrido[2,3-d]pyrimidin-3(4H)-yl)-4-chloro-1-methyl-1H-indazol-3-yl)methanesulfonamide 2,2,2-trifluoroacetate (0.06 g, 0.081 mmol) in DMF (1.625 mL) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.023 g, 0.085 mmol), 2-(3H-[1,2,3]triazolo[4,5-b]pyridin-3-yl)-1,1,3,3-tetramethylisouronium hexafluorophosphate(V) (0.037 g, 0.097 mmol), and DIPEA (0.050 ml, 0.284 mmol). The reaction mixture was stirred for 1 h, filtered, and purified by preparatory HPLC using the following conditions: Column: Zorbax Eclipse Plus C18, 21.2×100 mm, 5 m particles; Solvent A=0.1% Formic Acid in 100% Water. Solvent B=Acetonitrile. Flow Rate=40 mL/min. Start % B=52.2 Final % B=72.2. Gradient Time=7 min, then a 2 min hold at 98% B. Wavelength=215 and 254 nm. ESI+Range: 150 to 1500 dalton. Sample was loaded at 35% B and afforded N—((S)-1-(6-chloro-3-(4-chloro-1-methyl-3-(methylsulfonamido)-1H-indazol-7-yl)-7-methoxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.032 g, 43%). ¹H NMR (500 MHz, METHANOL-d₄) δ ppm 8.50-8.57 (m, 1H), 7.29-7.34 (m, 1H), 7.21-7.26 (m, 1H), 6.54-6.84 (m, 4H), 4.51-4.63 (m, 3H), 4.22-4.28 (m, 3H), 3.60-3.65 (m, 3H), 3.43-3.50 (m, 1H), 3.23-3.27 (m, 3H), 3.08-3.16 (m, 1H), 2.39-2.48 (m, 2H), 1.34-1.41 (m, 1H), 0.98-1.05 (m, 1H). LC/MS retention time=1.39 min; m/z=870.7 [M+H]⁺. Column: Acquity UPLC BEH C18, 2.1×30 mm, 1.7 m particles; Solvent A=0.10% Formic acid in 100% Water. Solvent B=0.10% Formic Acid in 100% Acetonitrile. Flow Rate=0.8 mL/min. Start % B=5. Final % B=95. Gradient Time=1.6 min, then a 0.25 min hold at 95% B. Wavelength=215 nm.

### (S)-tert-butyl (1-(3-(4-chloro-3-(N-(4-methoxybenzyl)methylsulfonamido)-1-methyl-1H-indazol-7-yl)-7-isopropoxy-4-oxo-3,4-dihydropyrido[2,3-d]pyrimidin-2-yl)-2-(3,5-difluorophenyl)ethyl)carbamate

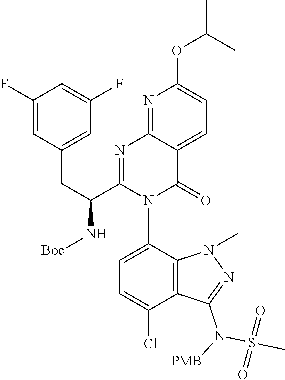

A mixture of(S)-2-((tert-butoxycarbonyl)amino)-3-(3,5-difluorophenyl)propanoic acid (0.038 g, 0.127 mmol), 2-amino-6-isopropoxynicotinic acid (0.025 g, 0.127 mmol), N-(7-amino-4-chloro-1-methyl-1H-indazol-3-yl)-N-(4-methoxybenzyl)methanesulfonamide (0.050 g, 0.127 mmol) and diphenyl phosphite (0.099 ml, 0.510 mmol) in pyridine (0.510 mL) was stirred 18 h. The reaction mixture was loaded directly onto a silica gel column and purified by flash chromatography using 0-65% ethyl acetate in hexanes to give the product (0.036 g, 34%) as a pale yellow solid. LCMS (M+1): 838.25.

### (S)—N-(7-(2-(1-amino-2-(3,5-difluorophenyl)ethyl)-7-isopropoxy-4-oxopyrido[2,3-d]pyrimidin-3(4H)-yl)-4-chloro-1-methyl-1H-indazol-3-yl)methanesulfonamide

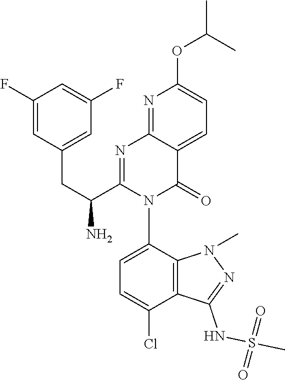 [SMILES: CC(C)Oc1ccc2c(=O)n(-c3ccc(Cl)c4c(NS(C)(=O)=O)nn(C)c34)c([C@@H](N)Cc3cc(F)cc(F)c3)nc2n1]

Triflic acid (0.121 mL, 1.360 mmol) was added to a solution of tert-butyl (S)-(1-(3-(4-chloro-3-(N-(4-methoxybenzyl)methylsulfonamido)-1-methyl-1H-indazol-7-yl)-7-isopropoxy-4-oxo-3,4-dihydropyrido[2,3-d]pyrimidin-2-yl)-2-(3,5-difluorophenyl)ethyl)carbamate (1.14 g, 1.360 mmol) in DCM (6.80 mL) and TFA (4.25 mL). The mixture was stirred 1 h and concentrated in vacuo. The residue was taken in ethyl acetate and 1 M NaOH was added until basic. The organic layer was separated, dried (Na₂SO₄), and concentrated in vacuo. The crude residue was purified by silica gel flash chromatography using 20-80% ethyl acetate in hexanes to give the product (0.41 g) as an off-white solid as a single atropisomer. This material was chirally purified by SFC chromatography using the following conditions: Instrument: Agilent Semi-prep 1100 series Column Chiralpak IG, (5 microns—30 mm×250 mm), 40:60 Ethanol:Heptane (isocratic), 220 nm UV, Temperature: Ambient, Flow 45 ml/min, Manual injection and collection. The column was already preconditioned with base from a previous purification so no pretreatment was required. LCMS (M+1): 618.05.

### N—((S)-1-(3-(4-chloro-1-methyl-3-(methylsulfonamido)-1H-indazol-7-yl)-7-isopropoxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 38)

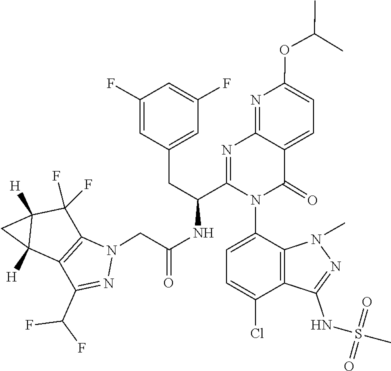 [SMILES: CC(C)Oc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@H]3C[C@@H]43)nc2n1]

To a stirred solution of (S)—N-(7-(2-(1-amino-2-(3,5-difluorophenyl)ethyl)-7-isopropoxy-4-oxopyrido[2,3-d]pyrimidin-3(4H)-yl)-4-chloro-1-methyl-1H-indazol-3-yl)methanesulfonamide 2,2,2-trifluoroacetate (0.06 g, 0.082 mmol) in DMF (1.639 mL) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.023 g, 0.086 mmol), 2-(3H-[1,2,3]triazolo[4,5-b]pyridin-3-yl)-1,1,3,3-tetramethylisouronium hexafluorophosphate(V) (0.037 g, 0.098 mmol), and DIPEA (0.050 ml, 0.287 mmol). After 1 h, the mixture was purified directly by preparatory HPLC using the following conditions: Column: Zorbax Eclipse Plus C18, 21.2×100 mm, 5 m particles; Solvent A=0.1% Formic Acid in 100% Water. Solvent B=Acetonitrile. Flow Rate=40 mL/min. Start % B=53.90 Final % B=73.9. Gradient Time=7 min, then a 2 min hold at 98% B. Wavelength=215 and 254 nm. ESI+Range: 150 to 1500 dalton. Sample was loaded at 35% B and afforded N—((S)-1-(3-(4-chloro-1-methyl-3-(methylsulfonamido)-1H-indazol-7-yl)-7-isopropoxy-4-oxo-3,4-dihydropyrido[2,3-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.034 g, 46% yield). ¹H NMR (500 MHz, METHANOL-d₄) δ ppm 8.42-8.50 (m, 1H), 7.29-7.34 (m, 1H), 7.20-7.25 (m, 1H), 6.96-7.04 (m, 1H), 6.55-6.83 (m, 4H), 5.58-5.66 (m, 1H), 4.52-4.63 (m, 3H), 3.60-3.64 (m, 3H), 3.43-3.50 (m, 1H), 3.23-3.27 (m, 3H), 3.08-3.16 (m, 1H), 2.39-2.47 (m, 2H), 1.45-1.51 (m, 6H), 1.34-1.40 (m, 1H), 0.97-1.04 (m, 1H). LC/MS retention time=1.39 min; m/z=770.2 [M+H]⁺. Column: Acquity UPLC BEH C18, 2.1×30 mm, 1.7 m particles; Solvent A=0.1% Formic acid in 100% Water. Solvent B=0.1% Formic Acid in 100% Acetonitrile. Flow Rate=0.8 mL/min. Start % B=5. Final % B=95. Gradient Time=1.6 min, then a 0.25 min hold at 95% B. Wavelength=215 nm.

### (S)-tert-butyl (1-(3-(4-chloro-3-(N-(4-methoxybenzyl)methylsulfonamido)-1-methyl-1H-indazol-7-yl)-7-methoxy-4-oxo-3,4-dihydropyrido[3,2-d]pyrimidin-2-yl)-2-(3,5-difluorophenyl)ethyl)carbamate

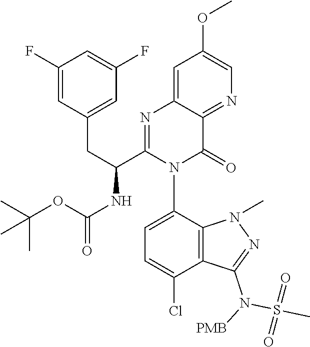

A mixture of(S)-2-((tert-butoxycarbonyl)amino)-3-(3,5-difluorophenyl)propanoic acid (0.896 g, 2.97 mmol), N-(7-amino-4-chloro-1-methyl-1H-indazol-3-yl)-N-(4-methoxybenzyl)methanesulfonamide (1.174 g, 2.97 mmol), and diphenyl phosphite (0.576 ml, 2.97 mmol) in pyridine (5.95 mL) was stirred at for 1 h. N-(7-amino-4-chloro-1-methyl-1H-indazol-3-yl)-N-(4-methoxybenzyl)methanesulfonamide (1.174 g, 2.97 mmol) was added and the reaction was then heated at 75° C. for 2.5 h. Upon cooling to ambient temperature, the reaction mixture was purified on directly by silica gel flash chromatography using 0-60% ethyl acetate in hexanes to give the product (0.52 g, 22%) as a pale yellow solid. LCMS (M+1): 810.

### (S)—N-(7-(2-(1-amino-2-(3,5-difluorophenyl)ethyl)-7-methoxy-4-oxopyrido[3,2-d]pyrimidin-3(4H)-yl)-4-chloro-1-methyl-1H-indazol-3-yl)methanesulfonamide

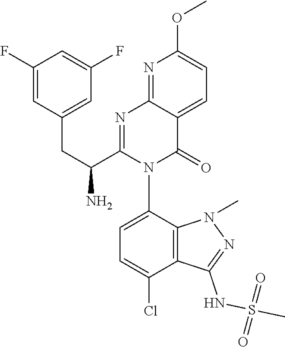 [SMILES: COc1ccc2c(=O)n(-c3ccc(Cl)c4c(NS(C)(=O)=O)nn(C)c34)c([C@@H](N)Cc3cc(F)cc(F)c3)nc2n1]

Triflic acid (0.171 ml, 1.925 mmol) was added to a solution tert-butyl (S)-(1-(3-(4-chloro-3-(N-(4-methoxybenzyl)methylsulfonamido)-1-methyl-1H-indazol-7-yl)-7-methoxy-4-oxo-3,4-dihydropyrido[3,2-d]pyrimidin-2-yl)-2-(3,5-difluorophenyl)ethyl)carbamate (0.52 g, 0.642 mmol) in DCM (3.21 mL) and TFA (2.007 mL. The mixture was stirred for 1 h and concentrated in vacuo. The residue was taken up in ethyl acetate and 1 M NaOH was added until basic. The organic layer was separated, dried (Na₂SO₄), and concentrated in vacuo. The off-white solid (0.338 g) was further purified by SFC chiral separation using the following conditions: Instrument: Agilent Semi-prep 1100 series; Column: Chiralpak IG, (5 microns—30 mm×250 mm); 75:25 Heptane:Ethanol (isocratic); 220 nm UV; Temperature: Ambient; Flow 45 ml/min; Manual injection and collection; Prior to sample dissolution the column was flushed extensively with Isopropanol w/ 0.1% IPAm to season the column with a base modifier. This was done to eliminate the formation of a salt but still afford enhanced resolution. After flushing for 30 minutes at 15 ml/min the neutral prep solvents were used at 75:25 Heptane:Ethanol. The product was isolated (97 mg, 26%). 1H NMR (500 MHz, DMSO-d6) δ ppm 8.14-8.30 (m, 1H), 7.47-7.55 (m, 1H), 6.99-7.23 (m, 2H), 6.85-6.95 (m, 1H), 6.55-6.67 (m, 2H), 3.97-4.09 (m, 3H), 3.35-3.51 (m, 4H), 3.07-3.18 (m, 1H), 2.84-2.99 (m, 3H), 2.60-2.76 (m, 1H). LCMS (M+1): 590.00.

### N—((S)-1-(3-(4-chloro-1-methyl-3-(methylsulfonamido)-1H-indazol-7-yl)-7-methoxy-4-oxo-3,4-dihydropyrido[3,2-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (Example 39)

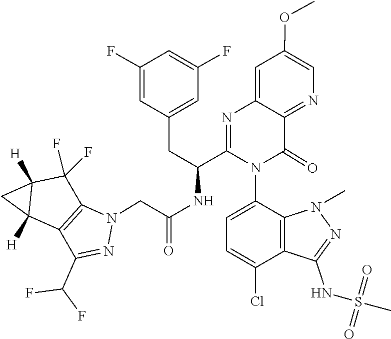 [SMILES: COc1cnc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@H]3C[C@@H]43)nc2c1]

To a stirred solution of (S)—N-(7-(2-(1-amino-2-(3,5-difluorophenyl)ethyl)-7-methoxy-4-oxopyrido[3,2-d]pyrimidin-3(4H)-yl)-4-chloro-1-methyl-1H-indazol-3-yl)methanesulfonamide (0.05 g, 0.085 mmol) in DMF (0.847 mL) was added 2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetic acid (0.022 g, 0.085 mmol), 2-(3H-[1,2,3]triazolo[4,5-b]pyridin-3-yl)-1,1,3,3-tetramethylisouronium hexafluorophosphate(V) (0.032 g, 0.085 mmol), and DIPEA (0.015 mL, 0.085 mmol). The reaction mixture was stirred for 2 h, diluted with DMF, and purified by preparatory HPLC using the following conditions: Column: Waters Xterra C18, 19×100 mm, 10 m particles; Solvent A=0.1% NH40H in 100% Water. Solvent B=Acetonitrile. Flow Rate=40 mL/min. Start % B=49. Final % B=69. Gradient Time=6 min, then a 2 min hold at 98% B. Wavelength=215 and 254 nm. ESI+Range: 150 to 1500 dalton. Sample was loaded at 35% B and afforded N—((S)-1-(3-(4-chloro-1-methyl-3-(methylsulfonamido)-1H-indazol-7-yl)-7-methoxy-4-oxo-3,4-dihydropyrido[3,2-d]pyrimidin-2-yl)-2-(3,5-difluorophenyl)ethyl)-2-((3bS,4aR)-3-(difluoromethyl)-5,5-difluoro-3b,4,4a,5-tetrahydro-1H-cyclopropa[3,4]cyclopenta[1,2-c]pyrazol-1-yl)acetamide (0.0369 g, 0.044 mmol, 52.1% yield). ¹H NMR (500 MHz, METHANOL-d₄) δ ppm 8.28-8.34 (m, 1H), 7.64-7.67 (m, 1H), 7.31-7.35 (m, 1H), 7.24-7.28 (m, 1H), 6.55-6.82 (m, 4H), 4.83-4.86 (m, 2H), 4.54-4.56 (m, 2H), 4.22-4.25 (m, 3H), 3.58-3.62 (m, 3H), 3.41-3.47 (m, 1H), 3.24-3.27 (m, 3H), 3.06-3.13 (m, 1H), 2.40-2.48 (m, 2H), 1.35-1.41 (m, 1H), 0.98-1.03 (m, 1H). LC/MS retention time=1.32 min; m/z=836.6 [M+H]+(Column: Acquity UPLC BEH C18, 2.1×30 mm, 1.7 m particles; Solvent A=0.1% Formic acid in 100% Water. Solvent B=0.1% Formic Acid in 100% Acetonitrile. Flow Rate=0.8 mL/min. Start % B=5. Final % B=95. Gradient Time=1.6 min, then a 0.25 min hold at 95% B. Wavelength=215 nm.

### Alternate preparation of N-(7-amino-4-chloro-1-methyl-1H-indazol-3-yl)-N-(4-methoxybenzyl)methanesulfonamide

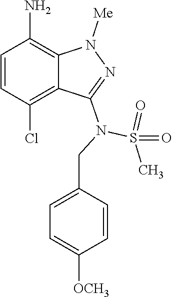 [SMILES: COc1ccc(CN(c2nn(C)c3c(N)ccc(Cl)c23)S(C)(=O)=O)cc1]

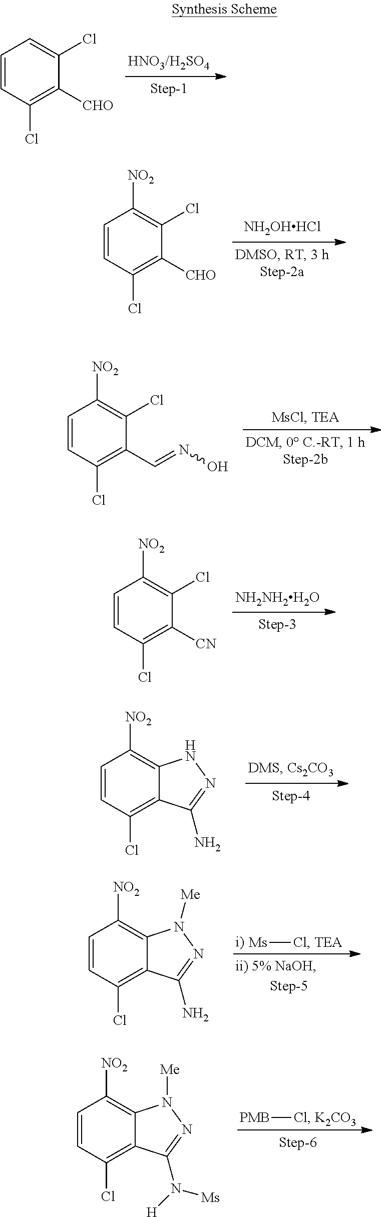

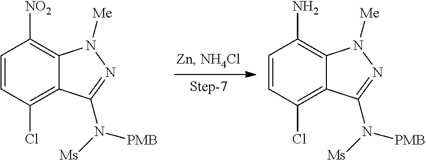

### Step 1: Preparation of 2,6-dichloro-3-nitrobenzaldehyde

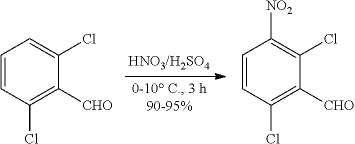 [SMILES: O=Cc1c(Cl)cccc1Cl ; O=Cc1c(Cl)ccc([N+](=O)[O-])c1Cl]

To a solution of sulfuric acid (H₂SO₄) (5.63 L, 4.5 V) in a round-bottom flask at 0-5° C. was added 2,6-dichlorobenzaldehyde (1.25 kg, 7.10 mol, 1.0 equiv.) in portions at below 15° C. The reaction mass was stirred at 0-5° C. for 30 min. A solution of freshly prepared nitration mixture [Prepared from Conc. H₂SO₄ (0.425 L, 0.34 V) and 70% HNO₃ (0.85 kg, 13.49 mol, 1.30 equiv.) at 0° C.] was added to the above reaction mixture at below 10° C. [Note: Reaction is slightly exothermic (3-6° C.); so that addition is preferred at lower temperature]. The reaction mixture was stirred at 5-10° C. for 2-3 h. After completion of the reaction (monitored by TLC), it was quenched with ice cold water (18.75 L, 15 V) at below 25° C. Then the reaction mass was allowed warm to room temperature and stirred for 2 h. The solids were isolated by filtration and then were washed with water (2.5 L, 2.0 V). Bulk residual water was removed from the solids by maintaining vacuum filtration for 60-90 min. The crude wet solid was initially dried under air atmosphere; then in a hot air oven at 50-55° C. for 10-12 h (until moisture content is not more than 5.0%) to get the dried title product, 2,6-dichloro-3-nitrobenzaldehyde (1.44 kg, 92% yield) as a yellow solid. ¹H NMR (400 MHz, CDCl₃): δ 10.44 (s, 1H), 7.88 (d, J=8.4 Hz, 1H), 7.56 (d, J=8.8 Hz, 1H).

### Step 2: Preparation of 2,6-dichloro-3-nitrobenzonitrile

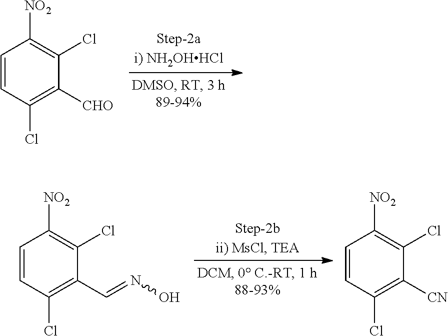

(Step-2a) To a solution of DMSO (5.9 L, 5.0 V)) in a round-bottom flask was added 2,6-dichloro-3-nitrobenzaldehyde (1.17 kg, 5.31 mol, 1.0 equiv.) at room temperature. After being stirred for 30 min at room temperature, hydroxylamine hydrochloride (0.63 kg, 9.04 mol, 1.70 equiv.) was added and the reaction mass was stirred at room temperature for 3 h. After completion of the reaction (monitored by TLC), the reaction mass was quenched by the addition of ice-cold water (18.0 L, 15.0 V) added at a rate sufficient to maintain the temperature below 30° C. (Observation: Solids formed upon water addition). The reaction mass was stirred at room temperature for 60-90 min. The solids were isolated by filtration; washed with water (2.5 L, 2.0 V); followed by washing with a mixture of acetone and hexanes (6.0 L, 1:1 ratio). Bulk residual water was removed from the solids by maintaining vacuum filtration for 60-90 min. The wet solid was initially air dried and then finally dried in a hot air oven at 50-55° C. for 10-12 h (until moisture content was not more than 1.0%) to get the dried target product, 2,6-dichloro-3-nitrobenzaldehyde oxime (1.22 kg, 92% yield) as an off-white solid. The crude product (which contains 10-20% of 2,6-dichloro-3-nitrobenzonitrile) was used directly in the next step without further purification.
(Step-2b) To a stirred solution of the crude oxime (preparation described above, 1.13 kg, 4.80 mol, 1.0 equiv.) in DCM (9.04 L, 8.0 V) at 0-5° C. was added triethylamine (“TEA”, 1.02 kg, 10.09 mol, 2.1 equiv.). After being stirred for 5 min, methanesulfonyl chloride (0.60 kg, 5.29 mol, 1.1 equiv.) was added (Observation: An exotherm is noted during the addition) slowly at 15° C. Then the reaction mass was stirred at room temperature for 30-45 min. After completion of the reaction (progress of reaction was monitored by TLC; mobile phase: 20% ethyl acetate in hexanes), the reaction mass was diluted with water (6.78 L, 6.0 V); the organic layer was separated; and the aqueous layer was extracted with DCM (3.4 L, 3.0 V). The combined organic layers were washed with brine (5.65 L, 5.0 V); dried over Na₂SO₄; and concentrated under vacuum. The resulting crude solids were triturated with hexanes (4.50 L, 4.0 V) at room temperature. The wet material was dried in a hot air oven at 50-55° C. for 5-6 h to get the dried product, 2,6-dichloro-3-nitrobenzonitrile (0.95 kg, 91% yield) as a yellow solid. ¹H NMR (400 MHz, CDCl₃): δ 8.07 (d, J=8.8 Hz, 1H), 7.63 (d, J=8.8 Hz, 1H).

### Step 3: Preparation of 4-chloro-7-nitro-1H-indazol-3-amine

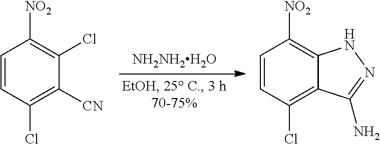

To a stirred solution of 2,6-dichloro-3-nitrobenzonitrile (750.0 g, 3.45 mol, 1.0 equiv.) in ethanol (7.5 L, 10.0 V) at 15-20° C. was slowly added hydrazine hydrate (519.0 g, 10.36 mol, 3.0 equiv.) while maintaining the reaction mass below 25° C. (Observation: Addition is slightly exothermic and solid formation will begin upon addition). The reaction mixture temperature was slowly raised to room temperature and then the mixture was stirred for 3 h (Observation: the quantity of solids will increase during this time). After completion of the reaction (monitored by TLC), the mixture was diluted with water (7.5 L, 10.0 V) and further stirred for 1 h at room temperature. The solids were isolated via filtration and then were washed with water (2.25 L, 3.0 V). The wet solid was washed with a 1:1 ratio mixture of acetone (1.875 L, 2.5 V) and hexanes (1.875 L, 2.5 V). Bulk residual water was removed from the solids by maintaining vacuum filtration for 60-90 min. The wet solid was finally dried in a hot air oven for 7-8 h at 50° C. (until moisture content reaches below 1.5%) to get the dried product, 4-chloro-7-nitro-1H-indazol-3-amine (549.0 g, 75% yield) as a brick red-colored solid. ¹H NMR (400 MHz, CDCl₃): δ 10.36 (bs, 1H), 8.20 (d, J=8.4 Hz, 1H), 7.07 (d, J=8.40 Hz, 1H), 4.73 (bs, 2H).

### Step 4: Preparation of 4-chloro-1-methyl-7-nitro-1H-indazol-3-amine

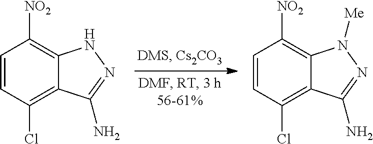 [SMILES: Nc1n[nH]c2c([N+](=O)[O-])ccc(Cl)c12 ; Cn1nc(N)c2c(Cl)ccc([N+](=O)[O-])c21]

To a stirred solution of 4-chloro-7-nitro-1H-indazol-3-amine (500 g, 0.42 mol, 1.0 equiv.) in DMF (5.0 L, 10.0 V) at 5-10° C. was slowly added cesium carbonate (Cs₂CO₃) (1.91 kg, 5.88 mol, 2.5 equiv.) while maintaining the reaction mass below 10° C. After being stirred for 5-10 min, dimethyl sulphate (326.3 g, 2.59 mol, 1.1 equiv.) was added while maintaining the reaction mass below 10° C. (Note: Slow addition is preferred for obtaining more favorable regio-selectivity). Then, the reaction temperature was slowly raised to room temperature and stirring was continued an additional 2 h at the same temperature. After completion of the reaction (monitored by TLC), the reaction mass was quenched by the addition of ice-cold water (15.0 L, 30.0 V) and the resulting mixture was then stirred for 6-8 h at room temperature. The solids were isolated via filtration and were then washed with water (1.5 L, 3.0 V). The wet solid was washed with IPA (1.5 L, 3.0 V) followed by hexanes (1.0 L, 2.0 V). Bulk residual water was removed from the solids by maintaining vacuum filtration for 60-90 min. The wet solid was dried in a hot air oven for 7-8 h at 50° C. (until moisture content is below 1.0%). The isolated material, 4-chloro-1-methyl-7-nitro-1H-indazol-3-amine (319.0 g, 60% yield), was used in the next step without further purification. ¹H NMR (400 MHz, CDCl₃): δ 7.97 (d, J=8.32 Hz, 1H), 6.97 (d, J=8.24 Hz, 1H), 4.63 (bs, 2H), 3.96 (s, 3H).

### Step 5: Preparation of N-(4-chloro-1-methyl-7-nitro-1H-indazol-3-yl)methanesulfonamide

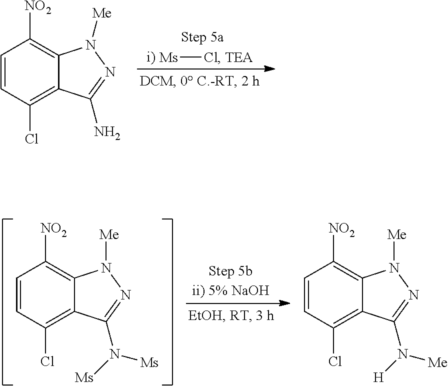

(Step 5a) To a solution of 4-chloro-1-methyl-7-nitro-1H-indazol-3-amine (625.0 g, 2.76 mol, 1.0 equiv.) in DCM (6.25 L, 10.0 V) at 0-5° C. was added triethylamine (TEA) (837.0 g, 8.27 mol, 3.0 equiv.); followed by the addition of 4-dimethylaminopyridine (DMAP) (20.60 g, 0.165 mol, 0.06 equiv.). The reaction mass was stirred for 5-10 min., then methanesulfonyl chloride (MsCl) (790.0 g, 6.89 mol, 2.5 equiv.) added slowly while maintaining the reaction mass below 10° C. The reaction mixture was allowed to warm to room temperature and was then stirred for 1.5-2.0 h. After completion of the reaction (monitored by TLC), the mixture was diluted with water (6.25 L, 10.0 V) and then stirred at room temperature for 15 min. The organic layer was separated, and the aqueous layer was extracted with DCM (6.25 L, 10.0 V). The combined organic layers were washed with brine (1.25 L, 2.0 V), dried over Na₂SO₄ and concentrated to get the crude solids. The solids were triturated with hexanes (1.25 L, 2.0 V) at room temperature to obtain the intermediate, N-(4-chloro-1-methyl-7-nitro-1H-indazol-3-yl)-N-(methylsulfonyl)methanesulfonamide, which was used directly in the next step.

(ii) To a stirred solution of N-(4-chloro-1-methyl-7-nitro-1H-indazol-3-yl)-N-(methylsulfonyl)methanesulfonamide (prepared above) in ethanol (10.5 L, 20.0 V) at room temperature was added slowly an aq. 5% NaOH solution (4.38 L, 7.0 V) [Note: Slow addition is preferred via dropping funnel]. The reaction mass was stirred at the same temperature for 3 h. After completion of the reaction (monitored by TLC) [Sample preparation for TLC analysis: ˜1.0 ml of sample acidified with aq. 2.0 N HCl to reach the pH: 2-3, extract it with ethyl acetate and analyze the organic layer by TLC], the reaction mass was cooled to 0-5° C. and the pH was adjusted to 2-3 by the addition of aq. 2.0 N HCl (3.13 L, 5.0 V) while maintain the reaction temperature below 10° C. [Note: Precipitation occurred upon addition of HCl and increased with stirring]. The reaction mixture was warmed to room temperature and then stirred for 1.5-2.0 h. Solids obtained were isolated via filtration and were then washed with water (1.25 L, 2.0 V); followed by washing with hexanes (1.25 L, 2.0 V). Bulk residual water was removed from the solids by maintaining vacuum filtration for 60-90 min. The wet material was dried in a hot air oven at 50° C. for 6-7 h (Until the moisture content is below 1.0%) to get the dried product, N-(4-chloro-1-methyl-7-nitro-1H-indazol-3-yl)methanesulfonamide (640.0 g, 76%) as a yellow solid. 1H NMR (400 MHz, CDCl₃): δ 8.05 (d, J=8.32 Hz, 1H), 7.32 (bs, 1H), 7.17 (d, J=8.28 Hz, 1H), 4.15 (s, 3H), 3.45 (s, 3H).

### Step 6: Preparation of N-(4-chloro-1-methyl-7-nitro-1H-indazol-3-yl)-N-(4-methoxybenzyl)methanesulfonamide

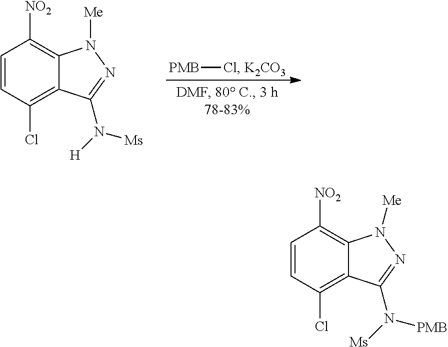

To a mixture of N-(4-chloro-1-methyl-7-nitro-1H-indazol-3-yl)methanesulfonamide (635.0 g, 2.08 mol, 1.0 equiv.) and 1-(chloromethyl)-4-methoxybenzene (359.0 g, 2.30 mol, 1.1 equiv.) in DMF (6.35 L, 10.0 V) at room temperature was added potassium carbonate (374.7 g, 2.70 mol, 1.3 equiv.). The reaction mixture was heated to 80-90° C. and maintained at that temperature for 3 h. After completion of the reaction (monitored by TLC), the mixture was poured into ice cold water (19.05 L, 30.0 V) [Note: Slow quenching with vigorous stirring is preferred to avoid clumping as the product precipitates]. The resulting solids were isolated via filtration and washed with water (1.90 L, 3.0 V); then the solids were washed with hexanes (1.27 L, 2.0 V). Bulk residual water was removed from the solids by maintaining vacuum filtration for 60-90 min. The isolated solid was dissolved in Ethyl acetate (12.7 L, 20.0 V) and charcoal was added (63.5 g). The mixture was heated to 60-70° C. and then stirred for 30-45 min. at that temperature. The mixture was filtered while still hot (40-50° C.) through a pad of Celite and the Celite pad was then extracted with ethyl acetate (3.17 L, 5.0 V). The combined filtrates were concentrated to dryness under reduced pressure at below 50° C. Ethyl acetate (0.635 L, 1.0 V) was added to the solids at room temperature. The resultant solid suspension was stirred for 30 min. The solids were isolated via filtration and then were washed with hexanes (1.27 L, 2.0 V). Residual water was removed from the solids by maintaining vacuum filtration for 45-60 min. to afford the product N-(4-chloro-1-methyl-7-nitro-1H-indazol-3-yl)-N-(4-methoxybenzyl) methane sulfonamide (705.0 g, 80% yield) as a yellow solid. ¹H NMR (400 MHz, CDCl₃): δ 7.99 (d, J=8.24 Hz, 1H), 7.27 (d, J=8.68 Hz, 2H), 7.19 (d, J=8.24 Hz, 1H), 6.80 (d, J=8.44 Hz, 2H), 4.95-4.76 (m, 2H), 4.17 (s, 3H), 3.76 (s, 3H), 3.01 (s, 3H).

### Step 7: Preparation of N-(7-Amino-4-chloro-1-methyl-1H-indazol-3-yl)-N-(4-methoxybenzyl)methanesulfonamide

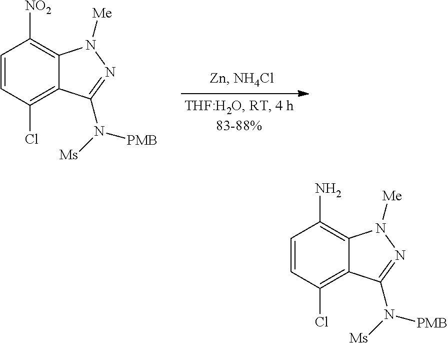

To a stirred suspension of zinc powder (540.0 g, 8.23 mol, 10.0 equiv.) in a mixture of THF (3.50 L, 10.0 V) and water (7.0 L, 20.0 V) at room temperature was added ammonium chloride (NH₄Cl) (449.0 g, 8.23 mol, 10.0 equiv.). To the mixture was added N-(4-chloro-1-methyl-7-nitro-1H-indazol-3-yl)-N-(4-methoxybenzyl)methanesulfonamide (350 g, 0.823 mol, 1.0 equiv.) in THF (7.0 L, 20.0 V). The reaction mixture was stirred at room temperature for 3-4 h. After completion of the reaction (monitored by in-process TLC/HPLC), the mixture was diluted with ethyl acetate (3.5 L, 10.0 V) and water (1.12 L, 2.5 V). The mixture was stirred for 15 min. The reaction mass was filtered through a pad of Celite bed washing with ethyl acetate (1.75 L, 5.0 V). The bi-phasic filtrate was collected, and the phases were separated. The aqueous layer was extracted with ethyl acetate (3.50 L, 10.0 V). The combined organic layers were washed with brine (3.50 L, 10 V), dried over Na₂SO₄, and then concentrated in vacuo to afford a crude solid. To the crude product was added MTBE (3.25 L, 10 V) and the suspension was stirred for 30 min at room temperature. The solids were isolated by filtration. Bulk residual water was removed from the solids by maintaining vacuum filtration for 30-45 min. The wet product was dried in a hot air oven (50° C.) for 2 h to afford the title product, N-(7-amino-4-chloro-1-methyl-1H-indazol-3-yl)-N-(4-methoxybenzyl)methanesulfonamide (276.0 g, 85% yield) as off-white solid. ¹H NMR (400 MHz, CDCl₃): δ 7.29-7.26 (m, 2H), 6.86-6.79 (m, 2H), 6.42 (d, J=7.80 Hz, 1H), 4.99-4.70 (m, 2H), 4.25 (s, 3H), 3.77 (s, 5H), 2.98 (s, 3H).

### Alternate preparation of N-(7-amino-4-chloro-1-(2,2-difluoroethyl)-1H-indazol-3-yl)-N-(4-methoxybenzyl)methanesulfonamide

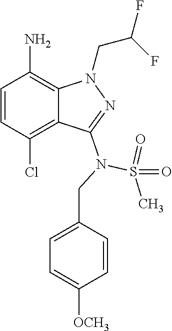 [SMILES: COc1ccc(CN(c2nn(CC(F)F)c3c(N)ccc(Cl)c23)S(C)(=O)=O)cc1]

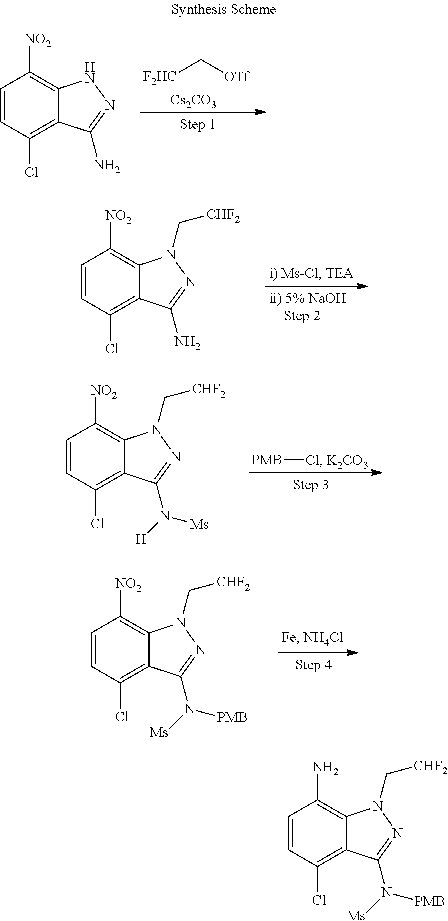

### Step 1: Preparation of 4-chloro-1-(2,2-difluoroethyl)-7-nitro-1H-indazol-3-amine

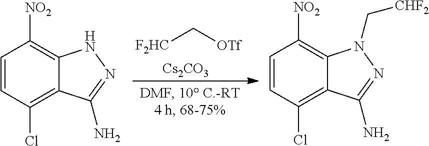

To a stirred solution of 4-chloro-7-nitro-1H-indazol-3-amine (180 g, 0.85 mol, 1.0 equiv.) in DMF (1.8 L, 10.0 V) at 10-15° C. was added cesium carbonate (Cs₂CO₃) (551 g, 1.70 mol, 2.0 equiv.) at a rate necessary to maintaining the reaction mass below 20° C. The mixture was stirred for 5-10 min, then to the stirred mixture at 10-15° C. was added 2,2-difluoroethyl trifluoromethanesulfonate (133 mL, 0.93 mol, 1.1 equiv.) at a rate necessary to maintain the reaction mass below 20° C. (Note: Slow addition is preferred to obtain more favorable regio-selectivity). The reaction mass was allowed to slowly warm to room temperature and was then stirred at the same temperature for 3 h. After completion of the reaction (monitored by TLC), the reaction mass was quenched by the addition of ice-cold water (5.4 L, 30.0 V) and the resulting mixture was allowed to warm to room temperature with stirring for 6-8 h. The solids were isolated via filtration and were then washed with water (540 mL, 3.0 V). The wet solid was washed with hexanes (0.9 L, 5.0 V). Bulk residual water was removed from the solids by maintaining vacuum filtration for 60-90 min. The wet solid was dried in a hot air oven for 7-8 h at 50° C. (until the moisture content was below 1.0%). The isolated material, 4-chloro-1-(2,2-difluoroethyl)-7-nitro-1H-indazol-3-amine (160 g, 71% yield), was used in the next step without further purification. 1H NMR (400 MHz, CDCl₃): δ 8.05 (d, J=8.4 Hz, 1H), 7.07 (d, J=8.4 Hz, 1H), 6.00 (tt, J₁=3.9 Hz, J₂=7.7 Hz, 1H), 4.76-4.84 (m, 4H).

### Step 2: Preparation of N-(4-chloro-1-(2,2-difluoroethyl)-7-nitro-1H-indazol-3-yl)methane sulfonamide

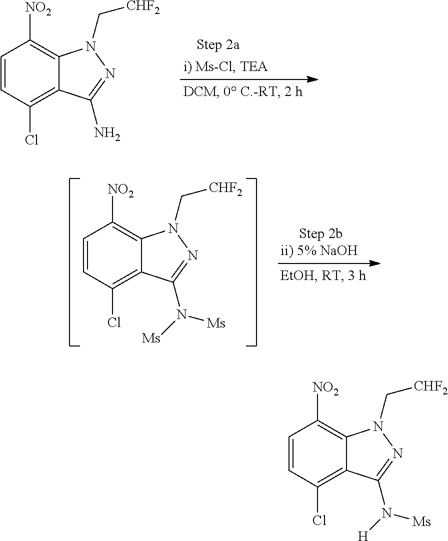

Step 2a: To a solution of 4-chloro-1-(2,2-difluoroethyl)-7-nitro-1H-indazol-3-amine (170.0 g, 0.96 mol, 1.0 equiv.) in DCM (1.7 L, 10.0 V) at 0-5° C. was added triethyl amine (264 mL, 2.88 mol, 3.0 equiv.), followed by 4-dimethylaminopyridine (3.4 g, 0.048 mol, 0.05 equiv.). The reaction mass was stirred for 5-10 min., then methanesulfonyl chloride (120 mL, 2.4 mol, 2.5 equiv.) was added slowly while maintaining the reaction mass below 10° C. The reaction mixture was allowed to warm to room temperature and then was stirred for 1.5-2.0 h. After completion of the reaction (monitored by TLC), the mixture was diluted with water (1.7 L, 10.0 V) and then stirred at room temperature for 15 min. The organic layer was separated, and the aqueous layer was extracted with DCM (1.7 L, 10.0 V). The combined organic layers were washed with 10% brine solution (340 mL, 2.0 V), dried over Na₂SO₄ and concentrated to afford the product as a crude solid. The solids were triturated with hexanes (340 mL, 2.0 V) at room temperature to obtain N-(4-chloro-1-(2,2-difluoroethyl)-7-nitro-1H-indazol-3-yl)-N-(methylsulfonyl) methanesulfonamide which was used directly in the next step.
Step 2b: To a stirred solution of N-(4-chloro-1-(2,2-difluoroethyl)-7-nitro-1H-indazol-3-yl)-N-(methylsulfonyl) methanesulfonamide (entirety of material prepared above) in ethanol (1.7 L, 10.0 V) at room temperature was added slowly aq. 5% NaOH solution (1.19 L, 7.0 V) [Note: Slow addition is preferred via dropping funnel]. The reaction mass was stirred at the same temperature for 3 h. After completion of the reaction [Sample preparation for TLC analysis: an aliquot of reaction solution (˜1 mL) was acidified with aq. 2.0 N HCl to reach the pH 2-3; then the mixture was extracted with ethyl acetate and organic layer was analyzed by TLC], the reaction mass was cooled to 0-5° C. and the pH was adjusted to 2-3 by the addition of aq. 2.0 N HCl (˜850 mL, 5.0 V) at below 10° C. [Note: Precipitation occurred upon addition of HCl and the solids increased gradually with stirring]. The reaction mixture was warmed to room temperature and then stirred for 1.5-2.0 h. Solids obtained were isolated via filtration and were then washed with water (340 mL, 2.0 V); followed by washing with hexanes (340 mL, 2.0 V). Bulk residual water was removed from the solids by maintaining vacuum filtration for 60-90 min. The wet material was dried in a hot air oven at 50° C. for 6-7 h (until the moisture content was below 1.0%) to afford the dried product, N-(4-chloro-1-(2,2-difluoroethyl)-7-nitro-1H-indazol-3-yl)methanesulfonamide (170.0 g, 75%) as a yellow solid. ¹H NMR (400 MHz, CDCl₃): δ 8.15 (d, J=8.3 Hz, 1H), 7.52 (bs, 1H), 7.24 (d, J=8.3 Hz, 1H), 6.04 (tt, J₁=3.7 Hz, J₂=7.9 Hz, 1H), 5.02 (td, J₁=3.9 Hz, J₂=14.3 Hz, 2H), 3.42 (s, 4H).

### Step 3: Preparation of N-(4-chloro-1-(2,2-difluoroethyl)-7-nitro-1H-indazol-3-yl)-N-(4-methoxy benzyl)methanesulfonamide

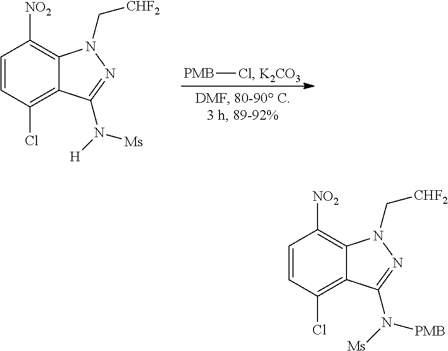

To a mixture of N-(4-chloro-1-(2,2-difluoroethyl)-7-nitro-1H-indazol-3-yl)methane sulfonamide (160.0 g, 0.45 mol, 1.0 equiv.) and 1-(chloromethyl)-4-methoxybenzene (67.6 mL, 0.5 mol, 1.1 equiv.) in DMF (1.6 L, 10.0 V) at room temperature was added potassium carbonate (93.8 g, 0.59 mol, 1.3 equiv.). The reaction mixture was heated to 80-90° C. and maintained at the same temperature for 3 h. After completion of the reaction (monitored by TLC), the mixture was poured into ice cold water (4.8 L, 60.0 V) [Note: Slow quenching with vigorous stirring is preferred to avoid clumping as the product precipitates]. The resulting solids were isolated via filtration and washed with water (480 mL, 3.0 V); then the solids were washed with hexanes (320 mL, 2.0 V). Bulk residual water was removed from the solids by maintaining vacuum filtration for 1-2 h. The isolated solid was dissolved in ethyl acetate (1.6 L, 10.0 V) and charcoal was added (16.0 g). The mixture was heated to 60-70° C. and then stirred for 30-45 min. at that temperature. The mixture was filtered while hot (40-50° C.) through a pad of Celite and the Celite pad was then extracted with ethyl acetate (800 mL, 5.0 V). The combined filtrates were concentrated to dryness under reduced pressure at below 50° C. To the resulting solids at room temperature was added ethyl acetate (160 mL, 1.0 V). The suspension was stirred for 30 min. The solids were isolated via filtration and then were washed with hexanes (320 mL, 2.0 V). Residual water was removed from the solids by maintaining vacuum filtration for 45-60 min. to afford the product N-(4-chloro-1-(2,2-difluoroethyl)-7-nitro-1H-indazol-3-yl)-N-(4-methoxybenzyl)methanesulfonamide (180.0 g, 92% yield) as a yellow solid. ¹H NMR (400 MHz, CDCl₃): δ 8.06 (d, J=8.4 Hz, 1H), 7.52 (bs, 1H), 7.27-7.21 (m, 4H), 6.77 (d, J=8.3 Hz, 2H), 6.01 (tt, J₁=3.8 Hz, J₂=7.9 Hz, 1H), 5.12-4.78 (m, 4H), 3.74 (s, 3H), 3.02 (s, 3H).

### Step 4: Preparation of N-(7-amino-4-chloro-1-(2,2-difluoroethyl)-1H-indazol-3-yl)-N-(4-methoxybenzyl)methanesulfonamide

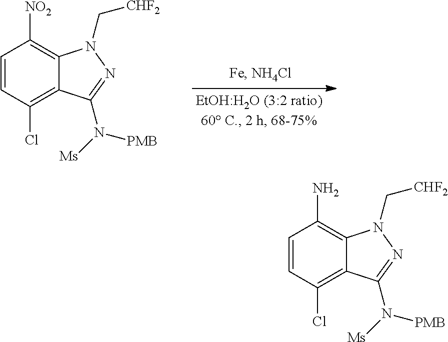

To a stirred suspension of iron powder (76.5 g, 1.37 mol, 5.0 equiv.) in a mixture of EtOH (650 mL, 5.0 V) and water (780 mL, 6.0 V) at room temperature was added ammonium chloride (118.0 g, 2.18 mol, 8.0 equiv.). To the mixture was added N-(4-chloro-1-(2,2-difluoroethyl)-7-nitro-1H-indazol-3-yl)-N-(4-methoxybenzyl)methanesulfonamide (130 g, 0.27 mol, 1.0 equiv.) in EtOH (520 mL, 4.0 V). The reaction mixture was heated to 60° C. and then stirred for 2 h. After completion of the reaction (monitored by in-process TLC/HPLC), the mixture was cooled to room temperature and diluted with ethyl acetate (1.3 L, 10.0 V) and water (390 mL, 3.0 V). The mixture was stirred for 15 min. The mixture was filtered through a pad of Celite and the Celite pad was then extracted with ethyl acetate (650 mL, 5.0 V). The bi-phasic filtrate was partitioned, and the organic phase was reserved while the aqueous layer was extracted with ethyl acetate (650 mL, 5.0 V). The combined organic layers were washed with brine (1.3 L, 10 V), dried over Na₂SO₄, and then concentrated in vacuo to afford a crude solid. To the crude product was added MTBE (650 mL, 5.0 V) and the suspension was stirred for 30 min. at room temperature. The solids were isolated via filtration. Bulk residual water was removed from the solids by maintaining vacuum filtration for 30-45 min. The wet product was dried in a hot air oven (50° C.) for 2 h to afford the title compound N-(7-amino-4-chloro-1-(2,2-difluoroethyl)-1H-indazol-3-yl)-N-(4-methoxy benzyl)methanesulfonamide (100.0 g, 70% yield) as off-white solid. ¹H NMR (400 MHz, CDCl₃): δ 7.21 (d, J=8.5 Hz, 2H), 6.87 (d, J=8.4 Hz, 1H), 6.78 (d, J=8.5 Hz, 2H), 6.52 (d, J=8.3 Hz, 1H), 6.01 (tt, J₁=3.8 Hz, J₂=7.7 Hz, 1H), 4.98-4.69 (m, 4H), 3.75 (s, 3H), 2.98 (s, 3H).

### Alternate preparation of N-(7-amino-4-chloro-1-(2,2-difluoroethyl)-1H-indazol-3-yl)-N-(4-methoxybenzyl)cyclopropanesulfonamide

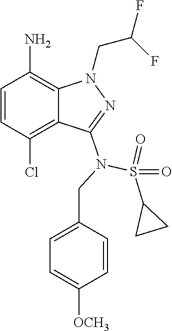 [SMILES: COc1ccc(CN(c2nn(CC(F)F)c3c(N)ccc(Cl)c23)S(=O)(=O)C2CC2)cc1]

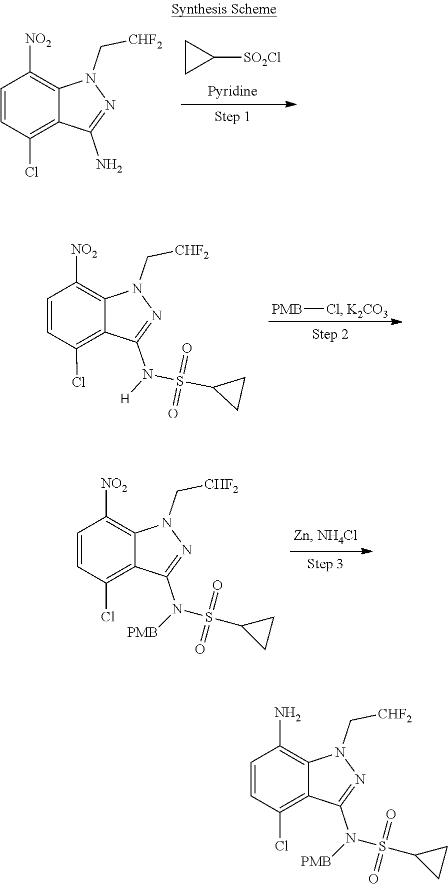

### Step 1: Preparation of N-(4-chloro-1-(2,2-difluoroethyl)-7-nitro-1H-indazol-3-yl)cyclopropanesulfonamide

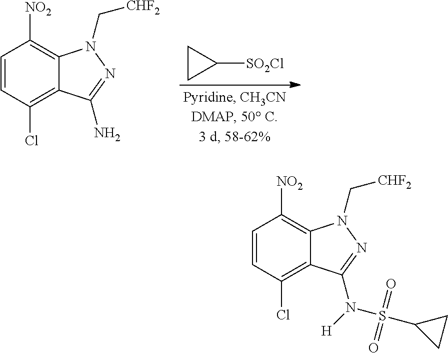 [SMILES: Nc1nn(CC(F)F)c2c([N+](=O)[O-])ccc(Cl)c12 ; O=S(=O)(Cl)C1CC1 ; O=[N+]([O-])c1ccc(Cl)c2c(NS(=O)(=O)C3CC3)nn(CC(F)F)c12]

To a stirred solution of 4-chloro-1-(2,2-difluoroethyl)-7-nitro-1H-indazol-3-amine (150.0 g, 0.54 mol, 1.0 equiv.) in acetonitrile (600 mL, 4.0 V) at room temperature was added pyridine (600 mL, 4.0 V), followed by the addition of 4-dimethylaminopyridine (30.0 g, 0.27 mol, 0.5 equiv.). The reaction mass was stirred for 5-10 min., then cyclopropylsulfonyl chloride (114 mL, 1.08 mol, 2.0 equiv.) was added at room temperature. The reaction mixture was heated to 50° C. and then stirred at that temperature for 3 days. After completion of the reaction (monitored by TLC), the mixture was cooled to room temperature and diluted with water (1.5 L, 10.0 V) and ethyl acetate (1.5 L, 10.0 V), then stirred at room temperature for 15 min. The organic layer was separated, and the aqueous layer was extracted with EtOAc (300 mL, 2.0 V). The combined organic layers were washed with aq. 1.0 N HCl (600 mL, 4.0 V), followed by 10% brine solution (1.5 L, 10.0 V). The organic layer was dried over Na₂SO₄, filtered, and then concentrated under reduced pressure to afford N-(4-chloro-1-(2,2-difluoroethyl)-7-nitro-1H-indazol-3-yl)cyclopropanesulfonamide (124.0 g, 61%) as a viscous liquid. ¹H NMR (400 MHz, CDCl₃): δ 8.11 (d, J=8.5 Hz, 1H), 7.25 (d, J=8.5 Hz, 1H), 6.04 (tt, J₁=3.8 Hz, J₂=7.7 Hz, 1H), 5.05 (td, J₁=3.8 Hz, J₂=14.4 Hz, 2H), 3.06-3.00 (m, 1H), 1.65-1.42 (m, 2H), 1.19-1.13 (m, 2H).

### Step 2: Preparation of N-(4-chloro-1-(2,2-difluoroethyl)-7-nitro-1H-indazol-3-yl)-N-(4-methoxybenzyl)cyclopropanesulfonamide

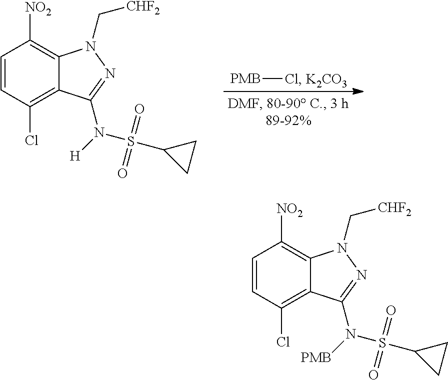

To a mixture of N-(4-chloro-1-(2,2-difluoroethyl)-7-nitro-1H-indazol-3-yl)cyclopropanesulfonamide (100.0 g, 0.20 mol, 1.0 equiv.) and 1-(chloromethyl)-4-methoxybenzene (39.2 mL, 0.22 mol, 1.1 equiv.) in DMF (1.0 L, 10.0 V) at room temperature was added potassium carbonate (128 g, 0.33 mol, 1.3 equiv.). The reaction mixture was heated to 80-90° C. and maintained at that temperature for 3 h. After completion of the reaction (monitored by TLC), the mixture was poured into ice cold water (3.0 L, 30.0 V) [Note: Slow quenching with vigorous stirring is preferred to avoid clumping as the product precipitates]. The resulting solids were isolated via filtration and washed with water (300 mL, 3.0 V); then the solids were washed with hexanes (300 mL, 3.0 V). Bulk residual water was removed from the solids by maintaining vacuum filtration for 1-2 h. The wet solid was dissolved in ethyl acetate (500 mL, 5.0 V) and charcoal was added (10.0 g). The mixture was heated to 60-70° C. and then stirred for 30-45 minutes at that temperature. The mixture was filtered while hot (40-50° C.) through a pad of Celite and the Celite pad was extracted with ethyl acetate (500 mL, 5.0 V). The combined filtrates were concentrated to dryness under reduced pressure at below 50° C. to afford N-(4-chloro-1-(2,2-difluoroethyl)-7-nitro-1H-indazol-3-yl)-N-(4-methoxy-benzyl)cyclopropanesulfonamide (122.0 g, 92% yield) as a yellow solid. ¹H NMR (400 MHz, CDCl₃): δ 8.05 (d, J=8.6 Hz, 1H), 7.26-7.22 (m, 3H), 6.73 (d, J=8.5 Hz, 2H), 5.98 (tt, J₁=3.7 Hz, J₂=7.8 Hz, 1H), 5.09-4.88 (m, 4H), 3.72 (s, 3H), 2.65-2.60 (m, 1H), 1.15-1.06 (m, 2H), 0.89-0.86 (m, 2H).

### Step 3: Preparation of N-(7-amino-4-chloro-1-(2,2-difluoroethyl)-1H-indazol-3-yl)-N-(4-methoxybenzyl)cyclopropanesulfonamide

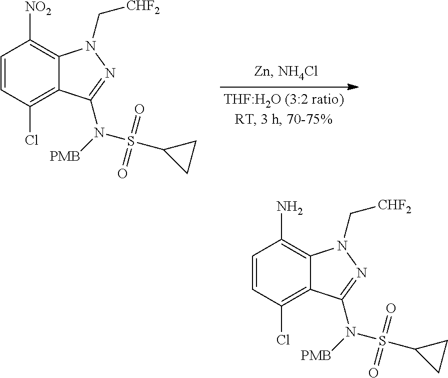

To a stirred suspension of zinc powder (156.0 g, 2.4 mol, 10.0 equiv.) in a mixture of THF (1.2 L, 10.0 V) and water (2.4 L, 20.0 V) at room temperature was added ammonium chloride (129.0 g, 2.40 mol, 10.0 equiv.). To the mixture was added N-(4-chloro-1-(2,2-difluoroethyl)-7-nitro-1H-indazol-3-yl)-N-(4-methoxybenzyl)cyclopropanesulfonamide (120 g, 0.2 mol, 1.0 equiv.) in THF (2.4 L, 20.0 V). The reaction mixture was stirred at room temperature for 2 h. After completion of the reaction (monitored by in-process TLC/HPLC), the mixture was diluted with ethyl acetate (1.2 L, 10.0 V) and water (360 mL, 3.0 V). The mixture was stirred for 15 min. The mixture was filtered through Celite and the Celite pad was extracted with ethyl acetate (600 mL, 5.0 V). The bi-phasic filtrate was partitioned, and the organic phase was reserved while the aqueous layer was extracted with ethyl acetate (600 mL, 5.0 V). The combined organic layers were washed with 10% brine solution (1.2 L, 10 V), dried over Na₂SO₄, filtered, and then concentrated in vacuo to afford a crude solid. To the crude product was added MTBE (600 mL, 5.0 V) and the suspension was stirred for 30-45 min. at room temperature. The solids were isolated by filtration and then bulk residual water was removed from the solids by maintaining vacuum filtration for 30-45 min. The wet product was dried in a hot air oven (50° C.) for 2 h to afford the product, N-(7-amino-4-chloro-1-(2,2-difluoroethyl)-1H-indazol-3-yl)-N-(4-methoxybenzyl)cyclopropanesulfonamide (81.0 g, 73% yield) as off-white solid. ¹H NMR (400 MHz, CDCl₃): δ 7.25 (d, J=8.5 Hz, 2H), 6.93 (d, J=8.4 Hz, 1H), 6.75 (d, J=8.3 Hz, 2H), 6.57 (d, J=8.4 Hz, 1H), 6.03 (tt, J₁=3.7 Hz, J₂=7.9 Hz, 1H), 4.80-4.95 (m, 4H), 3.74 (s, 3H), 2.67-2.61 (m, 1H), 1.14 (d, J=2.4 Hz, 2H), 0.96 (d, J=2.3 Hz, 2H).

### Alternate preparation of N-(7-amino-4-chloro-1-(2,2,2-trifluoroethyl)-1H-indazol-3-yl)-N-(4-methoxybenzyl)methanesulfonamide

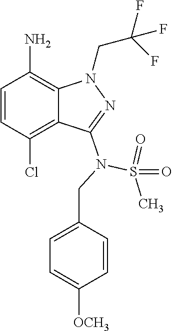 [SMILES: COc1ccc(CN(c2nn(CC(F)(F)F)c3c(N)ccc(Cl)c23)S(C)(=O)=O)cc1]

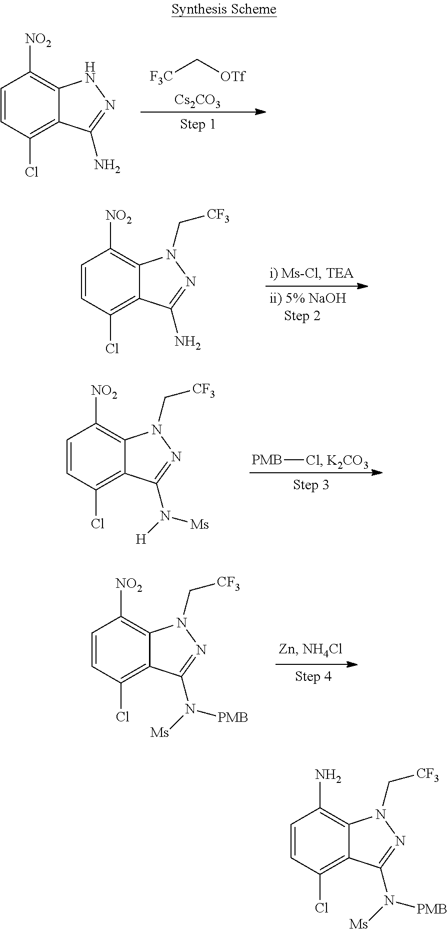

### Step 1: Preparation of 4-chloro-7-nitro-1-(2,2,2-trifluoroethyl)-1H-indazol-3-amine

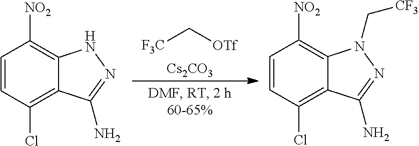

To a stirred solution of 4-chloro-7-nitro-1H-indazol-3-amine (50 g, 0.23 mol, 1.0 equiv.) in DMF (500 mL, 10.0 V) at 10-15° C. was added cesium carbonate (Cs₂CO₃) (153.3 g, 0.47 mol, 2.0 equiv.) at a rate sufficient to maintain the reaction mass below 20° C. The mixture was stirred for 5-10 min, then to the stirred mixture at 10-15° C. was added 2,2,2-trifluoroethyl trifluoromethanesulfonate (60.18 g, 0.26 mol, 1.1 equiv.) at a rate sufficient to maintain the reaction mass below 20° C. (Note: slow addition is preferred for obtaining more favorable regio-selectivity). The reaction mass was allowed to slowly warm to room temperature and was then stirred at the same temperature for 2 h. After completion of the reaction (monitored by TLC), the reaction mass was quenched via the addition of ice-cold water (1.5 L, 30.0 V) and the resulting mixture was allowed to warm to room temperature with stirring for 6-8 h. The solids were isolated via filtration and were then washed with water (150 mL, 3.0 V). The wet solid was washed with hexanes (250 mL, 5.0 V) and then bulk residual water was removed from the solids by maintaining vacuum filtration for 60-90 min. The wet solid was dried in a hot air oven for 7-8 h at 50° C. (until the moisture content was below 1.0%). The isolated material, 4-chloro-7-nitro-1-(2,2,2-trifluoroethyl)-1H-indazol-3-amine (45.0 g, 60% yield), was used directly in the next step without further purification. ¹H-NMR (400 MHz, CDCl₃): δ 8.09 (d, J=8.40 Hz, 1H), 7.12 (d, J=8.40 Hz, 1H), 5.14 (q, J=8.52 Hz, 2H), 4.77 (bs, H).

### Step 2: Preparation of N-(4-chloro-7-nitro-1-(2,2,2-trifluoroethyl)-1H-indazol-3-yl)methanesulfonamide

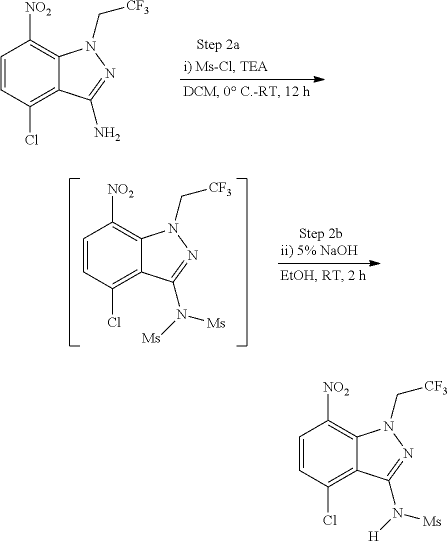

(Step 2a): To a solution of 4-chloro-7-nitro-1-(2,2,2-trifluoroethyl)-1H-indazol-3-amine (20.0 g, 0.068 mol, 1.0 equiv.) in DCM (200 mL, 10.0 V) at 0-5° C. was added triethylamine (29.0 mL, 0.204 mol, 3.0 equiv.), followed by the addition of 4-dimethylaminopyridine (415 mg, 0.03 mol, 0.05 equiv.). The reaction mass was stirred for 5-10 min., then to the mixture was added methanesulfonyl chloride (13.25 mL, 0.17 mol, 2.5 equiv) at a rate sufficient to maintain the reaction mass below 10° C. The reaction mixture was allowed to warm to room temperature with stirring for 12 h. After completion of the reaction (monitored by TLC), the mixture was diluted with water (200 mL, 10.0 V) and then stirred at room temperature for 15 min. The organic layer was separated, and the aqueous layer was extracted with DCM (200 mL, 10.0 V). The combined organic layers were washed with 10% brine solution (60 mL, 3.0 V), dried over Na₂SO₄, filtered, and concentrated to afford the crude solids. The solids were triturated with hexanes (60 mL, 3.0 V) at room temperature to obtain the intermediate, N-(4-chloro-7-nitro-1-(2,2,2-trifluoroethyl)-1H-indazol-3-yl)-N-(methylsulfonyl)methanesulfonamide, which was used directly in the next step.
(Step 2b): To a stirred solution of N-(4-chloro-7-nitro-1-(2,2,2-trifluoroethyl)-1H-indazol-3-yl)-N-(methylsulfonyl)methanesulfonamide (entirety of the material prepared above) in ethanol (200 mL, 10.0 V) at room temperature was added slowly aq. 5% NaOH solution (140 mL, 7.0 V) [Note: Slow addition is preferred via dropping funnel]. The reaction mass was stirred at the same temperature for 2 h. After completion of the reaction [Sample preparation for TLC analysis: An aliquot of the reaction solution (˜1.0 ml) was acidified by the addition of aq. 2.0 N HCl to reach pH 2-3; then the mixture was extracted with ethyl acetate and the organic phase was analyzed by TLC], the reaction mass was cooled to 0-5° C. and the pH was adjusted to 2-3 by the addition of aq. 2.0 N HCl (100 mL, 5.0 V) while maintain the temperature below 10° C. [Note: Precipitation occurred upon addition of HCl and increased with stirring]. The reaction mixture was warmed to room temperature and then stirred for 1.5-2.0 h. The solids were isolated via filtration and were then washed with water (60 mL, 3.0 V), followed by washing with hexanes (60 mL, 3.0 V). Bulk residual water was removed from the solids by maintaining vacuum filtration for 60-90 min. The wet material was dried in a hot air oven at 50° C. for 6-7 h (until the moisture content was below 1.0%) to afford N-(4-chloro-7-nitro-1-(2,2,2-trifluoroethyl)-1H-indazol-3-yl)methanesulfonamide (22.1 g, 87%) as a yellow solid. ¹H NMR (400 MHz, CDCl₃): δ 8.19 (d, J=8.40 Hz, 1H), 7.56 (bs, 1H), 7.30 (d, J=8.40 Hz, 1H), 5.34 (q, J=8.30 Hz, 2H), 3.46 (s, 3H).

### Step 3: Preparation of N-(4-chloro-7-nitro-1-(2,2,2-trifluoroethyl)-1H-indazol-3-yl)-N-(4-methoxybenzyl)methanesulfonamide

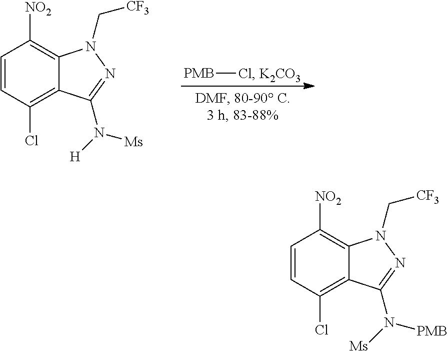

To a mixture of N-(4-chloro-7-nitro-1-(2,2,2-trifluoroethyl)-1H-indazol-3-yl)methanesulfonamide (50.0 g, 0.134 mol, 1.0 equiv.) and 1-(chloromethyl)-4-methoxybenzene (23.0 g, 0.147 mol, 1.1 equiv.) in DMF (500 mL, 10.0 V) at room temperature was added potassium carbonate (27.8 g, 0.201 mol, 1.5 equiv.). The reaction mixture was heated to 80-90° C. and maintained at that temperature for 3 h. After completion of the reaction (monitored by TLC), the mixture was poured into ice cold water (2.0 L, 40.0 V) [Note: Slow quenching with vigorous stirring is preferred to avoid clumping as the product precipitates]. The resulting solids were isolated via filtration and washed with water (150 mL, 3.0 V); then the solids were washed with hexanes (150 mL, 3.0 V). Bulk residual water was removed from the solids by maintaining vacuum filtration for 1-2 h. The solids were dissolved in ethyl acetate (500 mL, 10.0 V) and to the solution was added charcoal (5.0 g). The mixture was heated to 60-70° C. and then stirred at that temperature for 30-45 min. The mixture was filtered while hot (40-50° C.) through a pad of Celite and the Celite pad was extracted with ethyl acetate (250 mL, 5.0 V). The combined filtrate was concentrated to dryness under reduced pressure at below 50° C. The solids were combined with ethyl acetate (50 mL, 1.0 V) at room temperature. The resulting suspension was stirred for 30 min. The solids were isolated via filtration and then were washed with hexanes (100 mL, 2.0 V). Residual water was removed from the solids by maintaining vacuum filtration for 45-60 min. to afford N-(4-chloro-7-nitro-1-(2,2,2-trifluoroethyl)-1H-indazol-3-yl)-N-(4-methoxybenzyl)methanesulfonamide (56.0 g, 85% yield) as a yellow solid. ¹H NMR (400 MHz, CDCl₃): δ 8.12 (d, J=8.36 Hz, 1H), 7.31 (d, J=8.36 Hz, 1H), 7.22 (d, J=8.44 Hz, 2H), 6.77 (d, J=8.44 Hz, 2H), 5.50-5.25 (m, 2H), 4.94-4.79 (m, 2H), 3.75 (s, 3H), 3.02 (s, 3H).

### Step 4: Preparation of N-(7-amino-4-chloro-1-(2,2,2-trifluoroethyl)-1H-indazol-3-yl)-N-(4-methoxybenzyl)methanesulfonamide

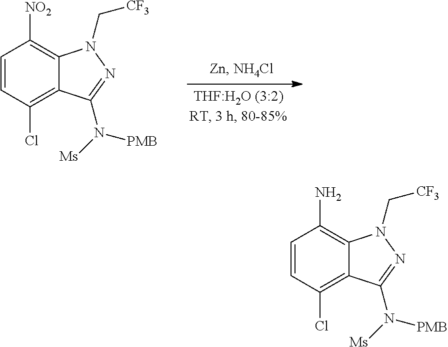

To a stirred suspension of zinc powder (66.31 g, 1.01 mol, 10.0 equiv.) in THF (500 mL, 10.0 V) and water (1.0 L, 20.0 V) at room temperature was added ammonium chloride (54.78 g, 1.01 mol, 10.0 equiv.). To the mixture was added a solution of N-(4-chloro-7-nitro-1-(2,2,2-trifluoroethyl)-1H-indazol-3-yl)-N-(4-methoxybenzyl)methanesulfonamide (50.0 g, 0.101 mol, 1.0 equiv.) in THF (1.0 L, 20.0 V). The reaction mixture was stirred at room temperature for 3 h. After completion of the reaction (monitored by in-process TLC/HPLC), the mixture was diluted with ethyl acetate (1.0 L, 20.0 V) and water (250 mL, 5.0 V). The mixture was stirred for 15 min. The mixture was filtered through a pad of Celite and the Celite pad was extracted with ethyl acetate (250 mL, 5.0 V). The bi-phasic filtrate was partition and the organic layer was reserved while the aqueous layer was extracted with ethyl acetate (500 mL, 10.0 V). The combined organic layers were washed with 10% brine solution (500 mL, 10.0 V), dried over Na₂SO₄, filtered, and then concentrated in vacuo to afford a crude solid. To the crude product was added MTBE (250 mL, 5.0 V) and the resulting suspension was stirred for 30 min. at room temperature. The solids were isolated by filtration and then bulk residual water was removed from the solids by maintaining vacuum filtration for 30-45 min. The wet product was dried in a hot air oven (50° C.) for 2 h to afford the title product N-(7-amino-4-chloro-1-(2,2,2-trifluoroethyl)-1H-indazol-3-yl)-N-(4-methoxybenzyl)methanesulfonamide (39.0 g, 83% yield) as off-white solid. ¹H NMR (400 MHz, CDCl₃): δ 7.25 (d, J=8.48 Hz, 2H), 6.98 (d, J=7.80 Hz, 1H), 6.79 (d, J=8.48 Hz, 2H), 6.66 (d, J=7.84 Hz, 1H), 5.35-4.75 (m, 4H), 3.77 (s, 3H), 3.56 (bs, 2H), 2.98 (s, 3H).

### Biological Methods:

HIV cell culture assay—MT-2 cells, 293T cells and the proviral DNA clone of NL₄₋₃ virus were obtained from the NIH AIDS Research and Reference Reagent Program. MT-2 cells were propagated in RPMI 1640 media supplemented with 10% heat inactivated fetal bovine serum (FBS), 100 mg/ml penicillin G and up to 100 units/mL streptomycin. The 293T cells were propagated in DMEM media supplemented with 10% heat inactivated FBS, 100 mg/mL penicillin G and 100 mg/mL streptomycin. A recombinant NL₄₋₃ proviral clone, in which a section of the nef gene was replaced with the Renilla luciferase gene, was used to make the reference virus used in these studies. The recombinant virus was prepared through transfection of the recombinant NL₄₋₃ proviral clone into 293T cells using Transit-293 Transfection Reagent from Mirus Bio LLC (Madison, Wis.). Supernatent was harvested after 2-3 days and the amount of virus present was titered in MT-2 cells using luciferase enzyme activity as a marker by measuring luciferase enzyme activity. Luciferase was quantitated using the EnduRen Live Cell Substrate from Promega (Madison, Wis.). Antiviral activities of compounds toward the recombinant virus were quantified by measuring luciferase activity in MT-2 cells infected for 4-5 days with the recombinant virus in the presence of serial dilutions of the compound.

The 50% effective concentration (EC₅₀) was calculated by using the exponential form of the median effect equation where (Fa)=1/[1+(ED₅₀/drug conc.)m] (Johnson V A, Byington RT. Infectivity Assay. In Techniques in HIV Research. ed. Aldovini A, Walker B D. 71-76. New York: Stockton Press. 1990). The 50% inhibitory concentration (EC₅₀) was calculated by using the exponential form of the median effect equation where percent inhibition=1/[1+(EC₅₀/drug concentration)m], where m is a parameter that reflects the slope of the concentration-response curve.

Compound cytotoxicity and the corresponding CC50 values were determined using the same protocol as described in the antiviral assay except that uninfected cells were used. Cytotoxicity was assessed on day 4 in uninfected MT2 cells by using a XTT (2,3-bis[2-Methoxy-4-nitro-5-sulfophenyl]-2H-tetrazolium-5-carboxyanilide inner salt)-based colorimetric assay (Sigma-Aldrich, St Louis, Mo.).

| Example | EC₅₀ nM | CC₅₀ μM |
|---|---|---|
| Example 1 | 0.046 | >1 |
| Example 2 | | |
| Example 3 | | |
| Example 4 | 0.273 | >1 |
| Example 5 | 0.758 | >1 |
| Example 6 | 0.347 | >1 |
| Example 7 | 0.899 | >1 |
| Example 8 | 0.150 | >1 |
| Example 9 | 0.049 | >1 |
| Example 10 | 0.107 | >1 |
| Example 11 | 0.208 | >1 |
| Example 12 | 0.109 | >1 |
| Example 13 | 0.239 | >1 |
| Example 14 | | |
| Example 15 | 0.071 | >1 |
| Example 16 | 0.084 | >1 |
| Example 17 | 0.083 | >1 |
| Example 18 | 0.069 | >1 |
| Example 19 | 0.063 | >1 |
| Example 20 | 0.122 | >1 |
| Example 21 | 0.122 | >1 |
| Example 22 | 0.573 | >1 |
| Example 23 | 0.094 | >1 |
| Example 24 | 0.155 | >1 |
| Example 25 | 0.167 | >1 |
| Example 26 | 0.443 | >1 |
| Example 27 | 0.610 | >1 |
| Example 28 | 0.242 | >1 |
| Example 28 | 0.646 | >1 |
| Example 29 | 0.115 | >1 |
| Example 30 | 0.097 | >1 |
| Example 31 | 0.046 | >1 |
| Example 32 | 0.285 | >1 |
| Example 33 | 1.262 | >1 |
| Example 34 | 0.597 | >1 |
| Example 35 | 0.235 | >1 |
| Example 36 | 0.131 | >1 |
| Example 37 | 0.044 | >1 |
| Example 38 | 0.045 | >1 |
| Example 39 | 0.016 | >1 |

The disclosure is not limited to the foregoing illustrative examples and the examples should be considered in all respects as illustrative and not restrictive, reference being made to the appended claims, rather than to the foregoing examples, and all changes which come within the meaning and range of equivalency of the claims are therefore intended to be embraced.

1. A compound or salt selected from the group consisting of: 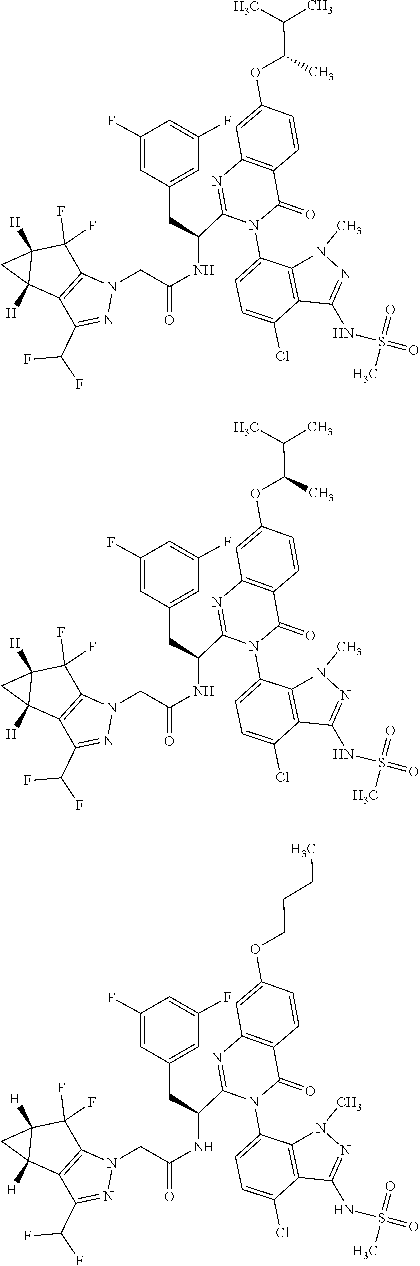 [SMILES: CC(C)[C@H](C)Oc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@H]3C[C@@H]43)nc2c1 ; CC(C)[C@@H](C)Oc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@H]3C[C@@H]43)nc2c1 ; CCCCOc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@H]3C[C@@H]43)nc2c1] 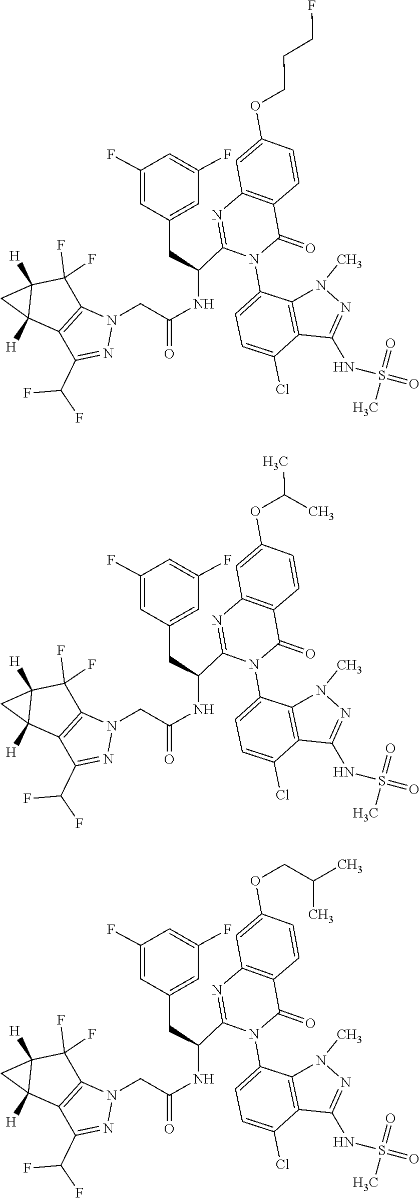 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@H]4C[C@@H]54)nc4cc(OCCCF)ccc4c3=O)c21 ; CC(C)Oc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@H]3C[C@@H]43)nc2c1 ; CC(C)COc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@H]3C[C@@H]43)nc2c1] 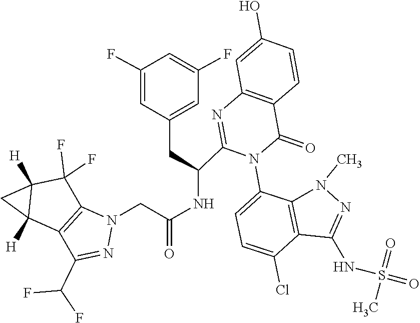 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@H]4C[C@@H]54)nc4cc(O)ccc4c3=O)c21]
and pharmaceutically acceptable salts thereof.

2. A compound or salt selected from the group consisting of: 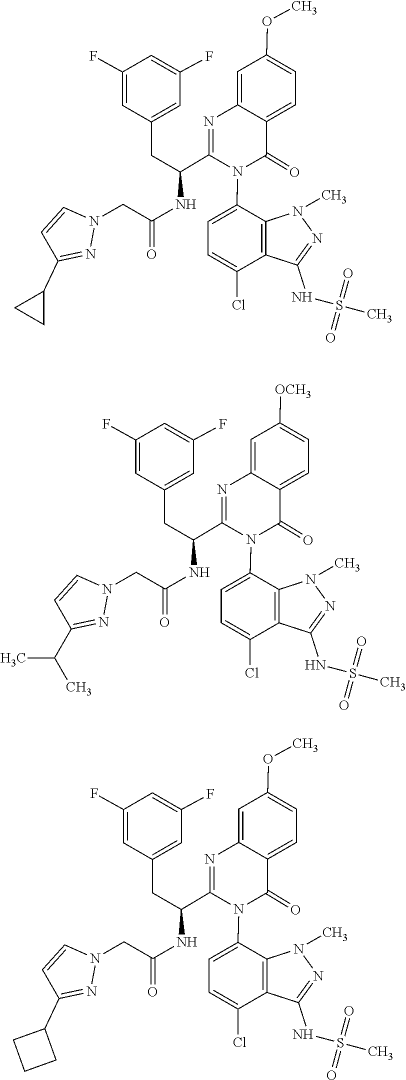
and pharmaceutically acceptable salts thereof.

3. A compound or salt selected from the group consisting of: 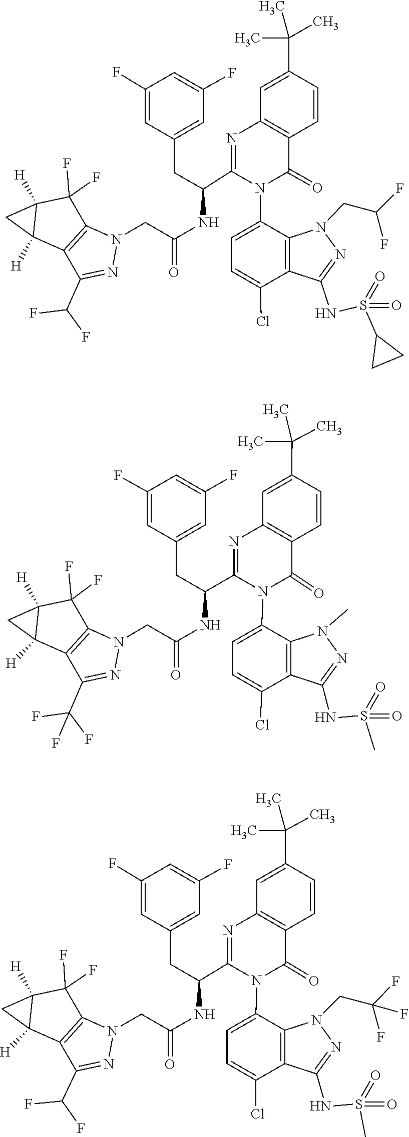 [SMILES: CC(C)(C)c1ccc2c(=O)n(-c3ccc(Cl)c4c(NS(=O)(=O)C5CC5)nn(CC(F)F)c34)c([C@H](Cc3cc(F)cc(F)c3)NC(=O)Cn3nc(C(F)F)c4c3C(F)(F)[C@@H]3C[C@H]43)nc2c1 ; Cn1nc(NS(C)(=O)=O)c2c(Cl)ccc(-n3c([C@H](Cc4cc(F)cc(F)c4)NC(=O)Cn4nc(C(F)(F)F)c5c4C(F)(F)[C@@H]4C[C@H]54)nc4cc(C(C)(C)C)ccc4c3=O)c21 ; CC(C)(C)c1ccc2c(=O)n(-c3ccc(Cl)c4c(NS(C)(=O)=O)nn(CC(F)(F)F)c34)c([C@H](Cc3cc(F)cc(F)c3)NC(=O)Cn3nc(C(F)F)c4c3C(F)(F)[C@@H]3C[C@H]43)nc2c1] 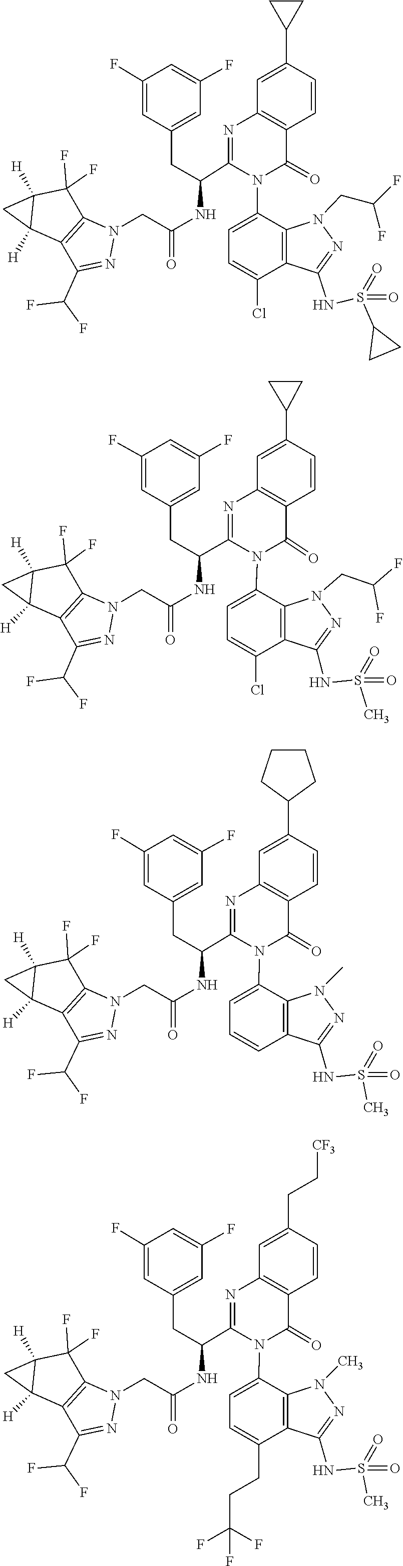 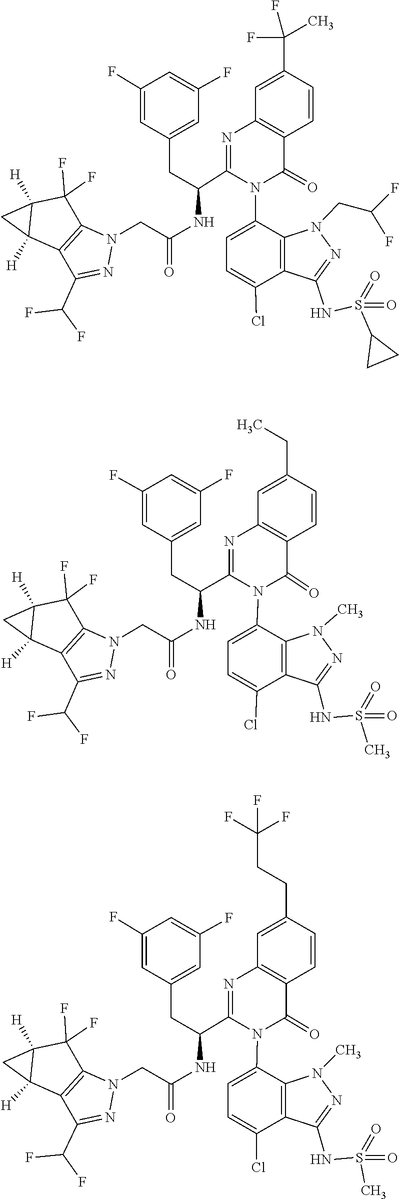 [SMILES: CC(F)(F)c1ccc2c(=O)n(-c3ccc(Cl)c4c(NS(=O)(=O)C5CC5)nn(CC(F)F)c34)c([C@H](Cc3cc(F)cc(F)c3)NC(=O)Cn3nc(C(F)F)c4c3C(F)(F)[C@@H]3C[C@H]43)nc2c1 ; CCc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1 ; Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(CCC(F)(F)F)ccc4c3=O)c21] 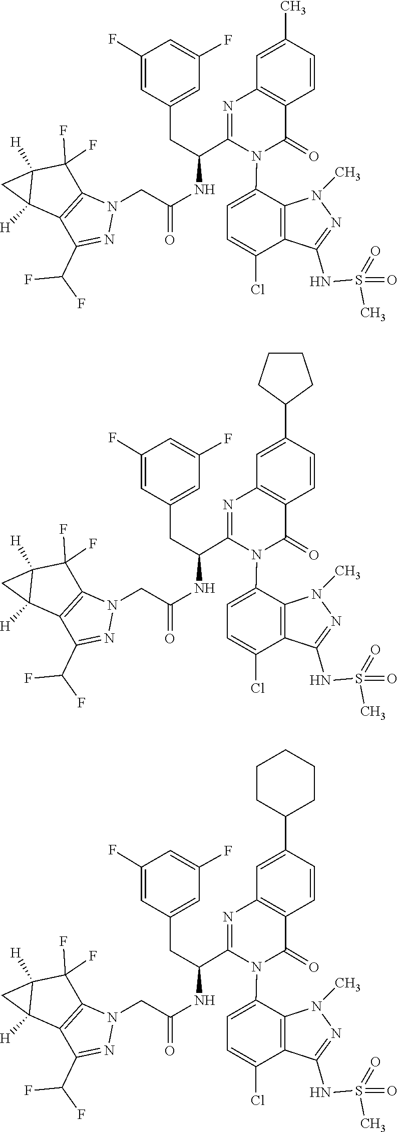 [SMILES: Cc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1 ; Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(C5CCCC5)ccc4c3=O)c21 ; Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@H]4C[C@H]54)nc4cc(C5CCCCC5)ccc4c3=O)c21] 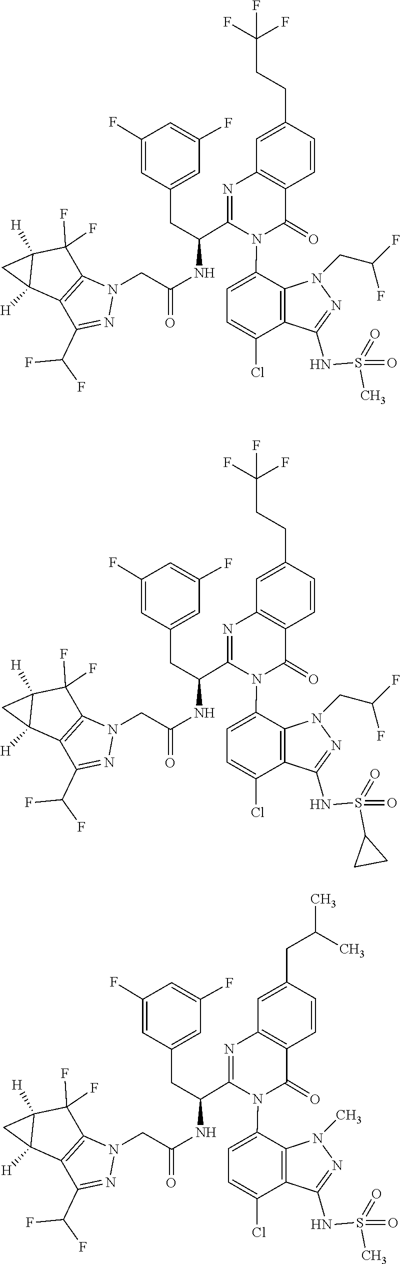 [SMILES: CS(=O)(=O)Nc1nn(CC(F)F)c2c(-n3c([C@H](Cc4cc(F)cc(F)c4)NC(=O)Cn4nc(C(F)F)c5c4C(F)(F)[C@@H]4C[C@H]54)nc4cc(CCC(F)(F)F)ccc4c3=O)ccc(Cl)c12 ; O=C(Cn1nc(C(F)F)c2c1C(F)(F)[C@@H]1C[C@H]21)N[C@@H](Cc1cc(F)cc(F)c1)c1nc2cc(CCC(F)(F)F)ccc2c(=O)n1-c1ccc(Cl)c2c(NS(=O)(=O)C3CC3)nn(CC(F)F)c12 ; CC(C)Cc1ccc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@H]3C[C@H]43)nc2c1] 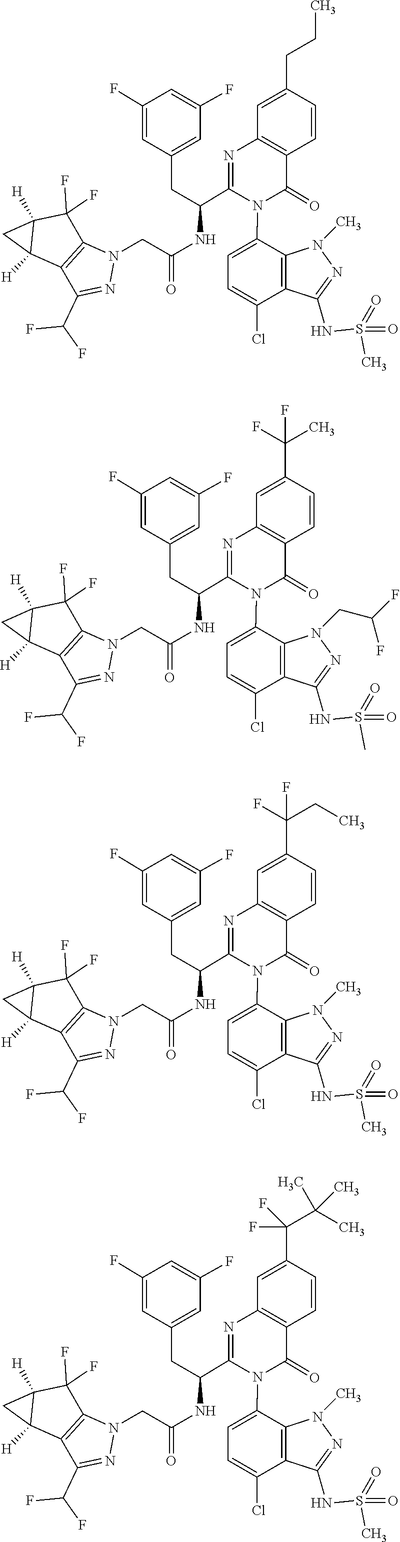
and pharmaceutically acceptable salts thereof.

4. A compound or salt selected from the group consisting of: 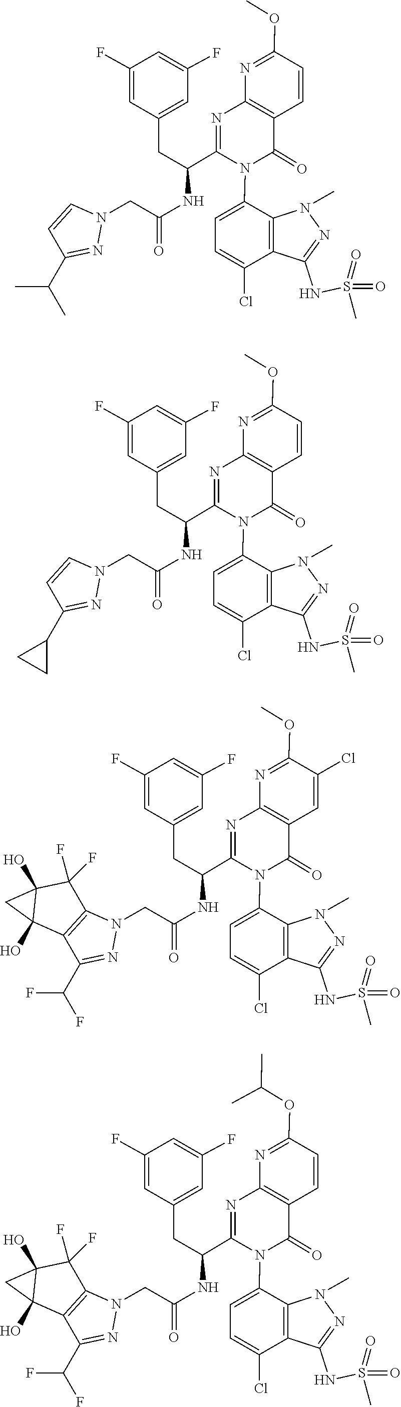 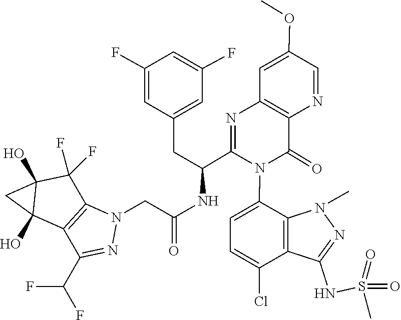 [SMILES: COc1cnc2c(=O)n(-c3ccc(Cl)c4c(NS(C)(=O)=O)nn(C)c34)c([C@H](Cc3cc(F)cc(F)c3)NC(=O)Cn3nc(C(F)F)c4c3C(F)(F)[C@@]3(O)C[C@@]43O)nc2c1]
and pharmaceutically acceptable salts thereof.

5. A compound or salt selected from the group consisting of: 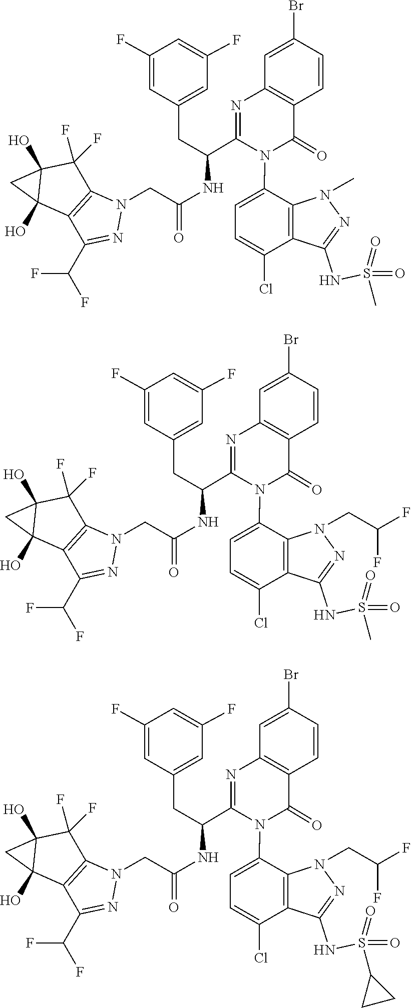 [SMILES: Cn1nc(NS(C)(=O)=O)c2c(Cl)ccc(-n3c([C@H](Cc4cc(F)cc(F)c4)NC(=O)Cn4nc(C(F)F)c5c4C(F)(F)[C@@]4(O)C[C@@]54O)nc4cc(Br)ccc4c3=O)c21 ; CS(=O)(=O)Nc1nn(CC(F)F)c2c(-n3c([C@H](Cc4cc(F)cc(F)c4)NC(=O)Cn4nc(C(F)F)c5c4C(F)(F)[C@@]4(O)C[C@@]54O)nc4cc(Br)ccc4c3=O)ccc(Cl)c12 ; O=C(Cn1nc(C(F)F)c2c1C(F)(F)[C@@]1(O)C[C@@]21O)N[C@@H](Cc1cc(F)cc(F)c1)c1nc2cc(Br)ccc2c(=O)n1-c1ccc(Cl)c2c(NS(=O)(=O)C3CC3)nn(CC(F)F)c12]
and pharmaceutically acceptable salts thereof.

6. A pharmaceutical composition comprising a compound or salt according to claim 1.

7. A composition according to claim 6 further comprising a pharmaceutically acceptable carrier, excipient, and/or diluent.

8. A method of treating HIV infection in a human comprising administering a composition according to claim 6 to a patient in need thereof.

9. The method of claim 8 wherein said administration is oral.

10. The method of claim 8 wherein said administration comprises administering by injection or subcutaneously.

11. The method of claim 8 wherein said method further comprises administration of at least one other agent used for treatment of AIDS or HIV infection selected from the group consisting of nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

12-14. (canceled)
